(19) United States
(12) Patent Application Publication
(10) Pub. No.: US 2025/0092403 A1
(43) Pub. Date: Mar. 20, 2025

(54) COMPOSITIONS AND METHODS FOR INHIBITING KETOHEXOKINASE (KHK)

(71) Applicant: Dicerna Pharmaceuticals, Inc., Lexington, MA (US)

(72) Inventors: Bob Dale BROWN, Littleton, MA (US); Henryk T. DUDEK, Belmont, MA (US); Utsav SAXENA, Watertown, MA (US); Jihye PARK, Cambridge, MA (US); Marc ABRAMS, Natick, MA (US); Martin Lee KOSER, Westford, MA (US)

(21) Appl. No.: 18/641,868

(22) Filed: Apr. 22, 2024

Related U.S. Application Data
(63) Continuation of application No. 17/717,174, filed on Apr. 11, 2022, now abandoned.
(60) Provisional application No. 63/182,277, filed on Apr. 30, 2021.
(60) Provisional application No. 63/173,775, filed on Apr. 12, 2021.

(30) Foreign Application Priority Data
Sep. 15, 2021 (EP) 21196784.9

(51) Int. Cl. C12N 15/113; A61P 1/16

(52) U.S. Cl. CPC C12N 15/1137; A61P 1/16; C12N 2310/14; C12N 2310/315; C12N 2310/321; C12N 2310/322; C12N 2310/351

(57) ABSTRACT

Oligonucleotides are provided herein that inhibit KHK expression. Also provided are compositions including the same and uses thereof, particularly uses relating to treating diseases, disorders and/or conditions associated with KHK expression.

### CROSS REFERENCE TO RELATED APPLICATIONS

This Application is a Continuation of application Ser. No. 17/717,174 filed on Apr. 11, 2022. Application Ser. No. 17/717,174 claims the benefit of U.S. Provisional Application 63/182,277 filed on Apr. 30, 2021 and also claims the benefit of U.S. Provisional Application 63/173,775 filed on Apr. 12, 2021. The entire contents of these applications are incorporated herein by reference in their entirety.

### REFERENCE TO ELECTRONIC SEQUENCE LISTING

This application contains a Sequence Listing which has been submitted electronically in.XML format and is hereby incorporated by reference in its entirety. Said.XML copy, created on Dec. 2, 2024, is named “DCY_07902.xml” and is 2,997,164 bytes in size. The sequence listing contained in this.xml file is part of the specification and is hereby incorporated by reference herein in its entirety.

### FIELD OF THE INVENTION

The invention relates to oligonucleotides that inhibit KHK expression, compositions including the same and uses thereof. The invention also relates to methods for treating diseases, disorders and/or conditions associated with KHK expression.

### BACKGROUND OF THE INVENTION

Ketohexokinase (KHK) is an important enzyme in fructose metabolism. KHK catalyzes the conversion of D-fructose to fructose-1-phosphate. Under conditions of elevated fructose consumption, a major part of fructose-1 phosphate contributes to fatty-acid and triglyceride synthesis among other things. In the liver, uncontrolled regulation of this process can lead to diseases such as non-alcoholic fatty liver disease (NAFLD) and non-alcoholic steatohepatitis (NASH). Similarly, fructose metabolism converts fructose to glucose in the liver. Increased levels of glucose can lead to glucose intolerance (i.e., pre-diabetes, type-2 diabetes, and impaired fasting glucose). Excess glucose is converted to fatty acids and triglycerides and heightens the risk of developing cardiovascular disease (e.g., hypertension). Decreasing the amount of KHK in the liver is likely to reduce the development of, or symptoms of these diseases. Strategies for targeting the KHK gene to prevent such diseases are needed. RNAi agents targeting the KHK gene have been disclosed e.g., in WO 2015/123264 and WO 2020/060986.

### SUMMARY OF THE INVENTION

The disclosure is based in part on the discovery that oligonucleotides (e.g., RNAi oligonucleotides) reduce KHK expression in the liver. Specifically, target sequences within KHK mRNA were identified and oligonucleotides that bind to these target sequences and inhibit KHK mRNA expression were generated. As demonstrated herein, the oligonucleotides inhibited murine KHK expression, and/or monkey and human KHK expression in the liver. Without being bound by theory, the oligonucleotides described herein are useful for treating a disease, disorder or condition associated with KHK expression (e.g., Non-alcoholic fatty liver disease (NAFLD), nonalcoholic steatohepatitis (NASH)). In some embodiments, the oligonucleotides described herein are useful for treating a disease, disorder or condition associated with mutations in the KHK gene.

Accordingly, in some aspects, the present disclosure provides a double stranded RNAi oligonucleotide for reducing KHK expression, the oligonucleotide comprising an antisense strand and a sense strand, wherein the antisense strand and the sense strand form a duplex region, wherein the antisense strand comprises a region of complementarity to a KHK mRNA target sequence of any one of SEQ ID NOs: 4-387 and wherein the region of complementarity is at least 15 contiguous nucleotides in length, or a pharmaceutically acceptable salt thereof. In some aspects, the present disclosure provides a double stranded RNAi oligonucleotide for reducing KHK expression, the oligonucleotide comprising an antisense strand and a sense strand, wherein the antisense strand and the sense strand form a duplex region, wherein the antisense strand comprises a region of complementarity to a KHK mRNA target sequence of any one of SEQ ID NOs: 4-387, wherein the region of complementarity is at least 15 contiguous nucleotides in length, and wherein KHK expression is reduced by at least 50%.

In any of the foregoing or related aspects, the sense strand comprises a sequence set forth in any one of SEQ ID NOs: 4-387.

In any of the foregoing or related aspects, the anti-sense strand comprises a sequence set forth in any one of SEQ ID NOs: 388-771.

In other aspects, the disclosure provides a double stranded RNAi oligonucleotide for inhibiting expression of KHK, wherein said double stranded RNAi agent comprises a sense strand and an antisense strand forming a duplex region, wherein said sense strand comprises at least 15 contiguous nucleotides differing by no more than 3 nucleotides from any one of the nucleotide sequences of SEQ ID NO: 4-387 and said antisense strand comprises at least 15 contiguous nucleotides differing by no more than 3 nucleotides from the nucleotide sequences of SEQ ID NO: 388-771, or a pharmaceutically acceptable salt thereof. In other aspects, the disclosure provides a double stranded RNAi oligonucleotide for inhibiting expression of KHK, wherein said double stranded RNAi agent comprises a sense strand and an antisense strand forming a duplex region, wherein said sense strand comprises at least 15 contiguous nucleotides differing by no more than 3 nucleotides from any one of the nucleotide sequences of SEQ ID NO:4-387 and said antisense strand comprises at least 15 contiguous nucleotides differing by no more than 3 nucleotides from the nucleotide sequences of SEQ ID NO: 388-771, and wherein KHK expression is reduced by at least 50%, or a pharmaceutically acceptable salt thereof.

In any of the foregoing or related aspects, the sense strand is 15 to 50 nucleotides in length. In some aspects, the sense strand is 18 to 36 nucleotides in length. In other aspects, the sense strand is 15 to 30 nucleotides in length. In some aspects, the antisense strand is 15-30 nucleotides in length. In some aspects, the antisense strand is 22 nucleotides in length.

In any of the foregoing or related aspects, the antisense strand and the sense strand form a duplex region of at least 19 nucleotides in length. In any of the foregoing or related aspects, the antisense strand and the sense strand form a duple region of at least 20 nucleotides in length. In any of the foregoing or related aspects, the antisense strand and the sense strand form a duplex region of 20 nucleotides in length. In some aspects, the antisense strand is 22 nucleotides in length and the antisense strand and the sense strand form a duplex region of at least 19 nucleotides in length. In some aspects, the antisense strand is 22 nucleotides in length and the antisense strand and the sense strand form a duplex region of at least 20 nucleotides in length. In some aspects, the antisense strand is 22 nucleotides in length and the antisense strand and the sense strand form a duplex region of 20 nucleotides in length.

In any of the foregoing or related aspects, the antisense strand comprises a region of complementarity of at least 19 contiguous nucleotides in length, optionally at least 20 nucleotides in length.

In any of the foregoing or related aspects, the sense strand comprises at its 3′ end a stem-loop set forth as: S1-L-S2, wherein S1 is complementary to S2, and wherein L forms a loop between S1 and S2 of 3 to 5 nucleotides in length.

In some aspects, the disclosure provides an RNAi oligonucleotide for reducing KHK expression, the oligonucleotide comprising a sense strand of 15 to 50 nucleotides in length and an antisense strand, wherein the sense strand and the antisense strand form a duplex region, wherein the antisense strand comprises a region of complementarity to a KHK mRNA target sequence of any one of SEQ ID NOs: 4-387, and wherein the region of complementarity is at least 15 contiguous nucleotides in length, or a pharmaceutically acceptable salt thereof.

In yet other aspects, the disclosure provides an RNAi oligonucleotide for reducing KHK expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a KHK mRNA target sequence of any one of SEQ ID NOs: 4-387, and wherein the region of complementarity is at least 15 contiguous nucleotides in length, or a pharmaceutically acceptable salt thereof.

In other aspects, the disclosure provides an RNAi oligonucleotide for reducing KHK expression, the oligonucleotide comprising a sense strand of 15 to 50 nucleotides in length and an antisense strand, wherein the sense strand and the antisense strand form a duplex region, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In other aspects, the disclosure provides an RNAi oligonucleotide for reducing KHK expression, the oligonucleotide comprising a sense strand of 18 to 36 nucleotides in length and an antisense strand, wherein the sense strand and the antisense strand form a duplex region, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In yet other aspects, the disclosure provides an RNAi oligonucleotide for reducing KHK expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In some aspects, the disclosure provides an RNAi oligonucleotide for reducing KHK expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In other aspects, the disclosure provides an RNAi oligonucleotide for reducing KHK expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In yet other aspects, the disclosure provides an RNAi oligonucleotide for reducing KHK expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length, or a pharmaceutically acceptable salt thereof.

In some aspects, the disclosure provides a RNAi oligonucleotide for reducing KHK expression, the oligonucleotide comprising:
(i) an antisense strand of 19-30 nucleotides in length, wherein the antisense strand comprises a nucleotide sequence comprising a region of complementarity to a KHK mRNA target sequence, wherein the region of complementarity is selected from SEQ ID NOs: 948-953; and
(ii) a sense strand of 19-50 nucleotides in length comprising a region of complementarity to the antisense strand,
wherein the antisense and sense strands are separate strands which form an asymmetric duplex region having an overhang of 1-4 nucleotides at the 3′ terminus of the antisense strand. In some aspects, the RNAi oligonucleotide comprises a stem-loop at the 3′ terminus, wherein the stem loop is set forth as S1-L-S2, wherein S1 is complementary to S2 and wherein L forms a loop between S1 and S2 of 3 to 5 nucleotides in length.

In any of the foregoing or related aspects, the disclosure provides an RNAi oligonucleotide comprising a stem loop set forth as S1-L-S2, wherein S1 is complementary to S2 and wherein L forms a loop between S1 and S2 of 3 to 5 nucleotides in length. In some aspects, L is a triloop or a tetraloop. In any of the foregoing or related aspects, L is a tetraloop. In some aspects, the tetraloop comprises the sequence 5′-GAAA-3′. In any of the foregoing or related aspects, S1 and S2 are 1-10 nucleotides in length and have the same length. In some aspects, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some aspects, S1 and S2 are 6 nucleotides in length. In any of the foregoing or related aspects, the stem-loop comprises the sequence 5′-GCAGCCGAAAGGCUGC-3′ (SEQ ID NO: 871).

In any of the foregoing or related aspects, the disclosure provides an RNAi oligonucleotide comprising a nicked tetraloop structure. In some aspects, the RNAi oligonucleotide comprises a nick between the 3′ terminus of the sense strand and the 5′ terminus of the antisense strand. In some aspects, the antisense and sense strands are not covalently linked.

In any of the foregoing or related aspects, the disclosure provides an RNAi oligonucleotide wherein the antisense strand comprises a 3′ overhang of one or more nucleotides in length. In some aspects, the 3′ overhang comprises purine nucleotides. In some aspects, the 3′ overhang is 2 nucleotides in length. In some aspects, the 3′ overhang is selected from AA, GG, AG and GA. In some aspects, the 3′ overhang is GG or AA. In some aspects, the 3′ overhang is GG.

In any of the foregoing or related aspects, the disclosure provides an RNAi oligonucleotide comprising at least one modified nucleotide. In some aspects, the modified nucleotide comprises a 2′-modification. In some aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some aspects, the 2′-modification is 2′-fluoro. In some aspects, the 2′-modification is 2′-O-methyl. In some aspects, the 2′-modification is 2′-fluoro and 2′-O-methyl.

In any of the foregoing or related aspects, the disclosure provides an RNAi oligonucleotide comprising at least one modified nucleotide, wherein about 10-15%, 10%, 11%, 12%, 13%, 14% or 15% of the nucleotides of the sense strand comprise a 2′-fluoro modification. In some aspects, about 25-35%, 25%, 26%, 27%, 28%, 29%, 30%, 31%, 32%, 33%, 34% or 35% of the nucleotides of the antisense strand comprise a 2′-fluoro modification.

In some aspects, about 25-35%, 25%, 26%, 27%, 28%, 29%, 30%, 31%, 32%, 33%, 34% or 35% of the nucleotides of the oligonucleotide comprise a 2′-fluoro modification. In some aspects, the sense strand comprises 36 nucleotides with positions 1-36 from 5′ to 3′, wherein positions 8-11 comprise a 2′-fluoro modification. In some aspects, the antisense strand comprises 22 nucleotides with positions 1-22 from 5′ to 3′, wherein positions 2, 3, 4, 5, 7, 10 and 14 comprise a 2′-fluoro modification. In some aspects, the remaining nucleotides of the sense and/or antisense strand comprise a 2′-O-methyl modification.

In any of the foregoing or related aspects, the disclosure provides an RNAi oligonucleotide wherein all of the nucleotides are modified. In some aspects, positions 8, 9, 10 and 11 (from 5′ to 3′) of the sense strand are modified. In some aspects, positions 3, 8, 9, 10, 12, 13 and 17 (from 5′ to 3′) of the sense strand are modified. In some aspects, positions 2, 3, 4, 5, 7, 10 and 14 (from 5′ to 3′) of the antisense strand are modified. In some aspects, positions 2-5, 7, 8, 10, 14, 16 and 19 (from 5′ to 3′) of the antisense strand are modified. In some aspects, positions 8, 9, 10 and 11 (from 5′ to 3′) of the sense strand and positions 2, 3, 4, 5, 7, 10 and 14 (from 5′ to 3′) of the antisense strand are modified. In some aspects, positions 3, 8, 9, 10, 12, 13 and 17 (from 5′ to 3′) of the sense strand and positions 2-5, 7, 8, 10, 14, 16 and 19 (from 5′ to 3′) of the antisense strand are modified. In some aspects, the modification is a 2′-fluoro modification.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length and comprises a phosphorothioate linkage between positions 20 and 21 and between positions 21 and 22, wherein positions are numbered 1-22 from 5′ to 3′.

In any of the foregoing or related aspects, the disclosure provides an RNAi oligonucleotide wherein the 4′-carbon of the sugar of the 5′-nucleotide of the antisense strand comprises a phosphate e analog. In some aspects, the phosphate analog oxymethylphosphonate, vinylphosphonate or malonyl phosphonate, optionally wherein the phosphate analog is a 4′-phosphate analog comprising 5′-methoxyphosphonate-4′-oxy.

In any of the foregoing or related aspects, the disclosure provides an RNAi oligonucleotide comprising an antisense strand comprising a phosphorylated nucleotide at the 5′ terminus, wherein the phosphorylated nucleotide is selected from uridine and adenosine. In some aspects, the phosphorylated nucleotide is uridine.

In any of the foregoing or related aspects, the oligonucleotide reduces or inhibits KHK expression in vivo. In any of the foregoing or related aspects, the oligonucleotide is a Dicer substrate. In some aspects, the oligonucleotide is a Dicer substrate that, upon endogenous Dicer processing, yields double-stranded nucleic acids of 19-23 nucleotides in length capable of reducing KHK expression in a mammalian cell.

In any of the foregoing or related aspects, the disclosure provides an RNAi oligonucleotide wherein at least one nucleotide of the oligonucleotide is conjugated to one or more targeting ligands. In some aspects, each targeting ligand comprises a carbohydrate, amino sugar, cholesterol, polypeptide, or lipid. In some aspects, the stem loop comprises one or more targeting ligands conjugated to one or more nucleotides of the stem loop. In some aspects, one or more targeting ligands is conjugated to one or more nucleotides of the loop. In some aspects, the loop comprises 4 nucleotides numbered 1-4 from 5′ to 3′, wherein nucleotides at positions 2, 3 and 4 each comprise one or more targeting ligands, wherein the targeting ligands are the same or different.

In any of the foregoing or related aspects, the disclosure provides an RNAi oligonucleotide wherein each targeting ligand comprises a N-acetylgalactosamine (GalNAc) moiety. In some aspects, the GalNAc moiety is a monovalent GalNAc moiety, a bivalent GalNAc moiety, a trivalent GalNAc moiety or a tetravalent GalNAc moiety. In some aspects, up to 4 nucleotides of L of the stem-loop are each conjugated to a monovalent GalNAc moiety.

In any of the foregoing or related aspects, the disclosure provides an RNAi oligonucleotide comprising an antisense strand comprising a region of complementarity, wherein the region of complementarity is fully complementary to the KHK mRNA target sequence at nucleotide positions 2-8 of the antisense strand, wherein nucleotide positions are numbered 5′ to 3′. In some aspects, the region of complementarity is fully complementary to the KHK mRNA target sequence at nucleotide positions 2-11 of the antisense strand, wherein nucleotide positions are numbered 5′ to 3′.

In any of the foregoing or related aspects, the disclosure provides an RNAi oligonucleotide wherein the sense strand comprises a nucleotide sequence of any one of SEQ ID NOs: 872-878 and 886-911.

In any of the foregoing or related aspects, the disclosure provides an RNAi oligonucleotide wherein the antisense strand comprises a nucleotide sequence of any one of SEQ ID NOs: 879-884 and 912-938.

In any of the foregoing or related aspects, the disclosure provides an RNAi oligonucleotide wherein the antisense strand comprises a nucleotide sequence selected from SEQ ID NOs: 913, 917, 918, 920, 923 and 936. In some aspects, the sense strand comprises a nucleotide sequence selected from SEQ ID NOs: 942-947. In some aspects, the sense strand comprises a nucleotide sequence selected from SEQ ID NOs: 887, 891, 892, 894, 897 and 909.

In any of the foregoing or related aspects, the disclosure provides an RNAi oligonucleotide wherein the sense and antisense strands comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(0) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd) SEQ ID NOs: 898 and 924, respectively;
(ee) SEQ ID NOs: 899 and 925, respectively;
(gg) SEQ ID NOs: 900 and 926, respectively; and
(hh) SEQ ID NOs: 909 and 936, respectively.

In some aspects, the disclosure provides an RNAi oligonucleotide wherein the sense and antisense strands comprise nucleotide sequences selected from the group consisting of:
(a) SEQ ID NOs: 887 and 913, respectively;
(b) SEQ ID NOs: 891 and 917, respectively;
(c) SEQ ID NOs: 892 and 918, respectively;
(d) SEQ ID NOs: 894 and 920, respectively;
(e) SEQ ID NOs: 897 and 923, respectively; and
(f) SEQ ID NOs: 909 and 936, respectively.

In some aspects, the disclosure provides an RNAi oligonucleotide wherein the sense and antisense strands comprise the nucleotide sequences set forth in SEQ ID NOs: 909 and 936, respectively. In other aspects, the disclosure provides an RNAi oligonucleotide wherein the sense and antisense strands comprise the nucleotide sequences set forth in SEQ ID NOs: 894 and 920, respectively. In yet other aspects, the disclosure provides an RNAi oligonucleotide wherein the sense and antisense strands comprise the nucleotide sequences set forth in SEQ ID NOs: 897 and 923, respectively. In some aspects, the disclosure provides an RNAi oligonucleotide wherein the sense and antisense strands comprise the nucleotide sequences set forth in SEQ ID NOs: 892 and 918, respectively. In other aspects, the disclosure provides an RNAi oligonucleotide wherein the sense and antisense strands comprise the nucleotide sequences set forth in SEQ ID NOs: 891 and 917, respectively. In yet further aspects, the disclosure provides an RNAi oligonucleotide wherein the sense and antisense strands comprise the nucleotide sequences set forth in SEQ ID NOs: 887 and 913, respectively.

In any of the foregoing or related aspects, the disclosure provides an RNAi oligonucleotide wherein the oligonucleotide as described herein achieves at least 50% knockdown of KHK mRNA. In some aspects, an oligonucleotide described herein achieves at least 50% knockdown of KHK mRNA in vitro. In some aspects, an oligonucleotide described herein achieves at least 50% knockdown of KHK mRNA in vivo. In some aspects, an oligonucleotide described herein achieves at least 50% knockdown of KHK mRNA in vitro and in vivo. In some aspects, an oligonucleotide described herein that achieves at least 50% knockdown of KHK mRNA comprises a sense strand and an antisense strand, wherein the sense and antisense strands comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 890 and 916, respectively;
(e) SEQ ID NOs: 891 and 917, respectively;
(f) SEQ ID NOs: 892 and 918, respectively;
(g) SEQ ID NOs: 893 and 919, respectively;
(h) SEQ ID NOs: 894 and 920, respectively;
(i) SEQ ID NOs: 911 and 938, respectively;
(j) SEQ ID NOs: 899 and 925, respectively;
(k) SEQ ID NOs: 900 and 926, respectively;
(l) SEQ ID NOs: 909 and 936, respectively; and
(m) SEQ ID NOs: 897 and 923, respectively.

In any of the foregoing or related aspects, the disclosure provides an RNAi oligonucleotide wherein the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog.

In any of the foregoing or related aspects, the disclosure provides an RNAi oligonucleotide wherein the sense strand comprises a nucleotide sequence of any one of SEQ ID NOs: 774-804.

In any of the foregoing or related aspects, the disclosure provides an RNAi oligonucleotide wherein the antisense strand comprises a nucleotide sequence of any one of SEQ ID NOs: 819-849.

In any of the foregoing or related aspects, the disclosure provides an RNAi oligonucleotide wherein the sense and antisense strands comprise nucleotide sequences selected from the group consisting of:
(a) SEQ ID NOs: 774 and 819, respectively;
(b) SEQ ID NOs: 775 and 820, respectively;
(c) SEQ ID NOs: 776 and 821, respectively;
(d) SEQ ID NOs: 777 and 822, respectively;
(e) SEQ ID NOs: 778 and 823, respectively;
(f) SEQ ID NOs: 779 and 824, respectively;
(g) SEQ ID NOs: 780 and 825, respectively;
(h) SEQ ID NOs: 781 and 826, respectively;
(i) SEQ ID NOs: 782 and 827, respectively;
(j) SEQ ID NOs: 783 and 828, respectively;
(k) SEQ ID NOs: 784 and 829, respectively;
(l) SEQ ID NOs: 785 and 830, respectively;
(m) SEQ ID NOs: 786 and 831, respectively;
(n) SEQ ID NOs: 787 and 832, respectively;
(o) SEQ ID NOs: 788 and 833, respectively;
(p) SEQ ID NOs: 789 and 834, respectively;
(q) SEQ ID NOs: 790 and 835, respectively;
(r) SEQ ID NOs: 791 and 836, respectively;
(s) SEQ ID NOs: 792 and 837, respectively;
(t) SEQ ID NOs: 793 and 838, respectively;
(u) SEQ ID NOs: 794 and 839, respectively;
(v) SEQ ID NOs: 795 and 840, respectively;
(w) SEQ ID NOs: 796 and 841, respectively;
(x) SEQ ID NOs: 797 and 842, respectively;
(y) SEQ ID NOs: 798 and 843, respectively;
(z) SEQ ID NOs: 799 and 844, respectively;
(aa) SEQ ID NOs: 800 and 845, respectively;
(bb) SEQ ID NOs: 801 and 846, respectively;
(cc) SEQ ID NOs: 802 and 847, respectively;
(dd) SEQ ID NOs: 803 and 848, respectively; and
(ee) SEQ ID NOs: 804 and 849, respectively.

In some aspects, the disclosure provides an RNAi oligonucleotide wherein the sense and antisense strands comprise nucleotide sequences selected from the group consisting of:
(a) SEQ ID NOs: 775 and 820, respectively;
(b) SEQ ID NOs: 779 and 824, respectively;
(c) SEQ ID NOs: 780 and 825, respectively;
(d) SEQ ID NOs: 782 and 827, respectively;
(e) SEQ ID NOs: 785 and 830, respectively; and
(f) SEQ ID NOs: 804 and 849, respectively.

In some aspects, the disclosure provides an RNAi oligonucleotide wherein the sense and antisense strands comprise the nucleotide sequences set forth in SEQ ID NOs: 804 and 849, respectively. In other aspects, the disclosure provides an RNAi oligonucleotide wherein the sense and antisense strands comprise the nucleotide sequences set forth in SEQ ID NOs: 782 and 827, respectively. In some aspects, the disclosure provides an RNAi oligonucleotide wherein the sense and antisense strands comprise the nucleotide sequences set forth in SEQ ID NOs: 775 and 820, respectively. In yet other aspects, the disclosure provides an RNAi oligonucleotide wherein the sense and antisense strands comprise the nucleotide sequences set forth in SEQ ID NOs: 779 and 824, respectively. In some aspects, the disclosure provides an RNAi oligonucleotide wherein the sense and antisense strands comprise the nucleotide sequences set forth in SEQ ID NOs: 780 and 825, respectively. In other aspects, the disclosure provides an RNAi oligonucleotide wherein the sense and antisense strands comprise the nucleotide sequences set forth in SEQ ID NOs: 785 and 830, respectively.

In any of the foregoing or related aspects, the disclosure provides an RNAi oligonucleotide wherein the sense strand comprises a nucleotide sequence of any one of SEQ ID NOs: 805-818.

In any of the foregoing or related aspects, the disclosure provides an RNAi oligonucleotide wherein the antisense strand comprises a nucleotide sequence of any one of SEQ ID NOs: 850-863.

In any of the foregoing or related aspects, the disclosure provides an RNAi oligonucleotide wherein the sense and antisense strands comprise nucleotide sequences selected from the group consisting of:
(a) SEQ ID NOs: 805 and 850, respectively;
(b) SEQ ID NOs: 806 and 851, respectively;
(c) SEQ ID NOs: 807 and 852, respectively;
(d) SEQ ID NOs: 808 and 853, respectively;
(e) SEQ ID NOs: 809 and 854, respectively;
(f) SEQ ID NOs: 810 and 855, respectively;
(g) SEQ ID NOs: 811 and 856, respectively;
(h) SEQ ID NOs: 812 and 857, respectively;
(i) SEQ ID NOs: 813 and 858, respectively;
(j) SEQ ID NOs: 814 and 859, respectively;
(k) SEQ ID NOs: 815 and 860, respectively;
(l) SEQ ID NOs: 816 and 861, respectively;
(m) SEQ ID NOs: 817 and 862, respectively and;
(n) SEQ ID NOs: 818 and 863, respectively.

In some aspects, the disclosure provides an RNAi oligonucleotide wherein the sense and antisense strands comprise the nucleotide sequences set forth in SEQ ID NOs: 805 and 850, respectively. In other aspects, the disclosure provides an RNAi oligonucleotide wherein the sense and antisense strands comprise the nucleotide sequences set forth in SEQ ID NOs: 809 and 854, respectively. In yet other aspects, the disclosure provides an RNAi oligonucleotide wherein the sense and antisense strands comprise the nucleotide sequences set forth in SEQ ID NOs: 810 and 855, respectively. In some aspects, the disclosure provides an RNAi oligonucleotide wherein the sense and antisense strands comprise the nucleotide sequences set forth in SEQ ID NOs: 812 and 857, respectively. In other aspects, the disclosure provides an RNAi oligonucleotide wherein the sense and antisense strands comprise the nucleotide sequences set forth in SEQ ID NOs: 815 and 860, respectively. In yet other aspects, the disclosure provides an RNAi oligonucleotide wherein the sense and antisense strands comprise the nucleotide sequences set forth in SEQ ID NOs: 818 and 863, respectively.

In some aspects, the present disclosure provides a double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mG-S-mA-mA-mG-mA-mG-mA-fA-fG-fC-fA-mG-mA-mU-mC-mC-mU-mG-mU-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 775), and wherein the antisense strand comprises the sequence and all of the modifications of 5′-[MePhosphonate-40-mU]-S-A-S-fC-fA-fG-mG-fA-mU-mC-fU-mG-mC-mU-fU-mC-mU-mC-mU-mU-mC-S-mG-S-mG-3′ (SEQ ID NO: 820), wherein mC, mA, mG, mU=2′-OMe ribonucleosides; fA, fC, fG, fU=2′F ribonucleosides; “-”-phosphodiester linkage, “—S-”-phosphorothioate linkage, and wherein ademA-GalNAc=

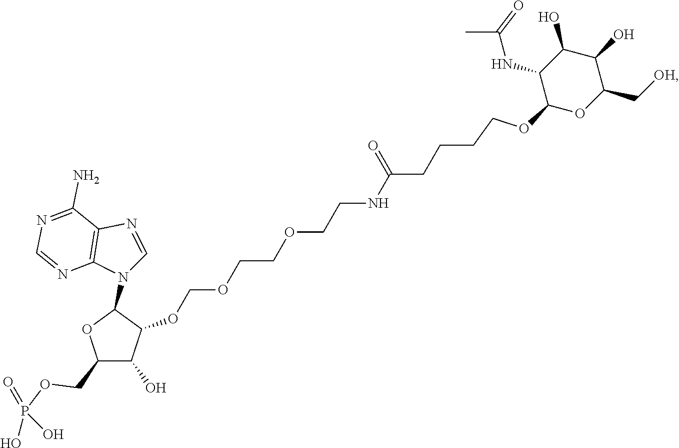 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In some aspects, the present disclosure provides a double stranded RNAi oligonucleotide (dsRNAi) for inhibiting expression of KHK, wherein said dsRNA comprises a sense strand and an antisense strand, the antisense strand comprising a region of complementarity to a KHK RNA transcript, e.g. KHK mRNA, wherein the sense strand comprises the sequence and all of the modifications of 5′-mC-S-mA-mG-mA-mU-mG-mU-fG-fU-fC-fU-mG-mC-mU-mA-mC-mA-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 779), and wherein the antisense strand comprises the sequence and all of the modifications of 5′-[MePhosphonate-40-mU]-S-fU-S-fC-S-fU-fG-mU-fA-mG-mC-fA-mG-mA-mC-fA-mC-mA-mU-mC-mU-mG-S-mG-S-mG-3′ (SEQ ID NO: 824), wherein mC, mA, mG, mU=2′-OMe ribonucleosides; fA, fC, fG, fU=2′F ribonucleosides; “-”=phosphodiester linkage, “—S-”=phosphorothioate linkage, and wherein ademA-GalNAc=

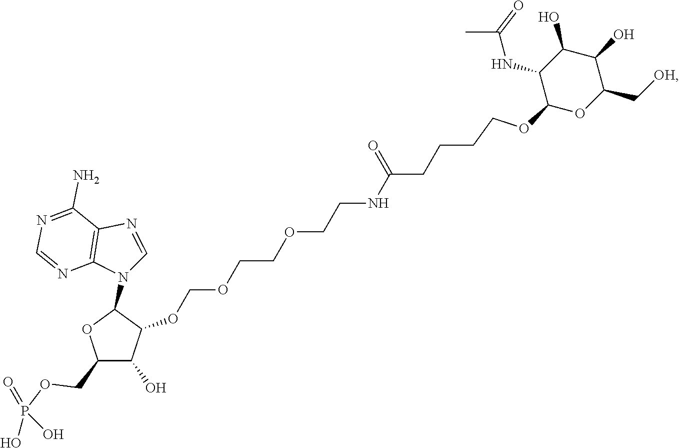 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In some aspects, the present disclosure provides a double stranded RNAi oligonucleotide (dsRNAi) for inhibiting expression of KHK, wherein said dsRNA comprises a sense strand and an antisense strand, the antisense strand comprising a region of complementarity to a KHK RNA transcript, e.g. KHK mRNA, wherein the sense strand comprises the sequence and all of the modifications of 5′-mG-S-mA-mC-mU-mU-mU-mG-fA-fG-fA-fA-mG-mG-mU-mU-mG-mA-mU-mC-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 780), and wherein the antisense strand comprises the sequence and all of the modifications of 5′-[MePhosphonate-40-mU]-S-fG-S-fA-S-fU-fC-mA-fA-mC-mC-fU-mU-mC-mU-fC-mA-mA-mA-mG-mU-mC-S-mG-S-mG-3′ (SEQ ID NO: 825), wherein mC, mA, mG, mU=2′-OMe ribonucleosides; fA, fC, fG, fU=2′F ribonucleosides; “-”=phosphodiester linkage, “—S-”-phosphorothioate linkage, and wherein ademA-GalNAc=

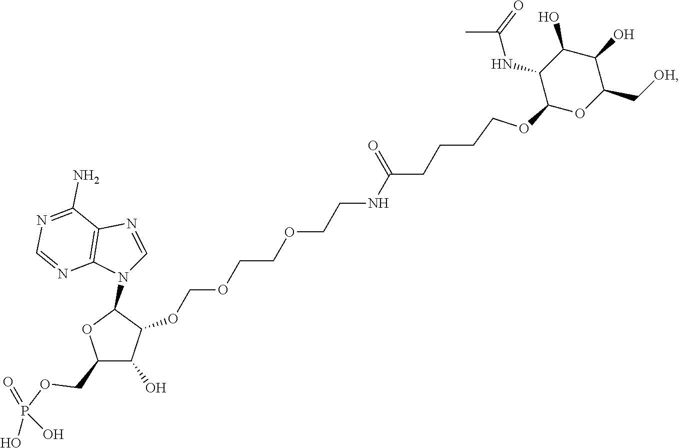 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In some aspects, the present disclosure provides a double stranded RNAi oligonucleotide (dsRNAi) for inhibiting expression of KHK, wherein said dsRNA comprises a sense strand and an antisense strand, the antisense strand comprising a region of complementarity to a KHK RNA transcript, e.g. KHK mRNA, wherein the sense strand comprises the sequence and all of the modifications of 5′-mU-S-mG-mU-mU-mU-mG-mU-fC-fA-fG-fC-mA-mA-mA-mG-mA-mU-mG-mU-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 785), and wherein the antisense strand comprises the sequence and all of the modifications of 5′-[MePhosphonate-40-mU]-S-fA-S-fC-fA-fU-mC-fU-mU-mU-fG-mC-mU-mG-fA-mC-mA-mA-mA-mC-mA-S-mG-S-mG-3′ (SEQ ID NO: 830), wherein mC, mA, mG, mU=2′-OMe ribonucleosides; fA, fC, fG, fU=2′F ribonucleosides; “-”=phosphodiester linkage, “—S-”=phosphorothioate linkage, and wherein ademA-GalNAc=

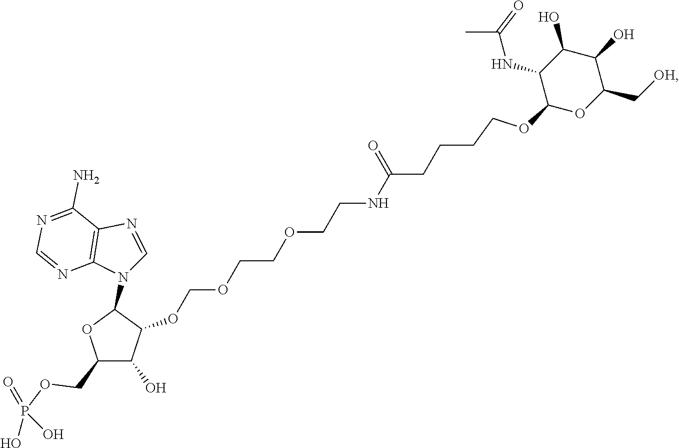 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In some aspects, the present disclosure provides a double stranded RNAi oligonucleotide (dsRNAi) for inhibiting expression of KHK, wherein said dsRNA comprises a sense strand and an antisense strand, the antisense strand comprising a region of complementarity to a KHK RNA transcript, e.g. KHK mRNA, wherein the sense strand comprises the sequence and all of the modifications of 5′-mG-S-mC-mA-mG-mG-mA-mA-fG-fC-fA-fC-mU-mG-mA-mG-mA-mU-mU-mC-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 804), and wherein the antisense strand comprises the sequence and all of the modifications of 5′-[MePhosphonate-40-mU]-S-fG-S-fA-S-fA-fU-mC-fU-mC-mA-fG-mU-mG-mC-fU-mU-mC-mC-mU-mG-mC-S-mG-S-mG-3′ (SEQ ID NO: 849), wherein mC, mA, mG, mU=2′-OMe ribonucleosides; fA, fC, fG, fU-2′F ribonucleosides; “-”-phosphodiester linkage, “—S-”=phosphorothioate linkage, and wherein ademA-GalNAc=

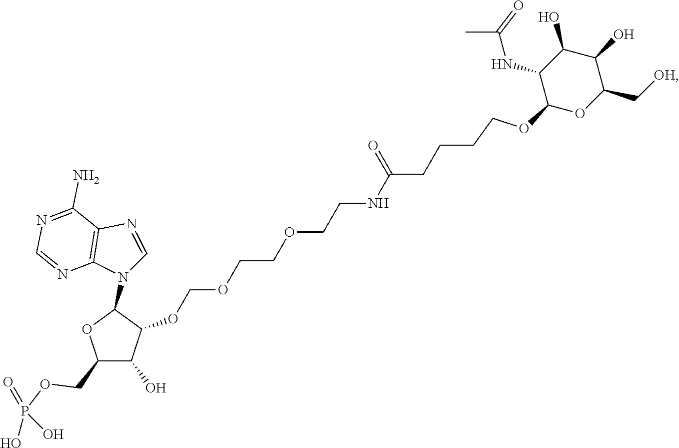 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In some aspects, the present disclosure provides a double stranded RNAi oligonucleotide (dsRNAi) for inhibiting expression of KHK, wherein said dsRNA comprises a sense strand and an antisense strand, the antisense strand comprising a region of complementarity to a KHK RNA transcript, e.g. KHK mRNA, wherein the sense strand comprises the sequence and all of the modifications of 5′-mU-S-mU-mU-mG-mA-mG-mA-fA-fG-fG-fU-mU-mG-mA-mU-mC-mU-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 782), and wherein the antisense strand comprises the sequence and all of the modifications of 5′ [MePhosphonate-40-mU]-S-fU-S-fC-S-fA-fG-mA-fU-mC-mA-fA-mC-mC-mU-fU-mC-mU-mC-mA-mA-mA-S-mG-S-mG-3′ (SEQ ID NO: 827), wherein mC, mA, mG, mU=2′-OMe ribonucleosides; fA, fC, fG, fU=2′F ribonucleosides; “-”-phosphodiester linkage, “—S-”=phosphorothioate linkage, and wherein ademA-GalNAc=

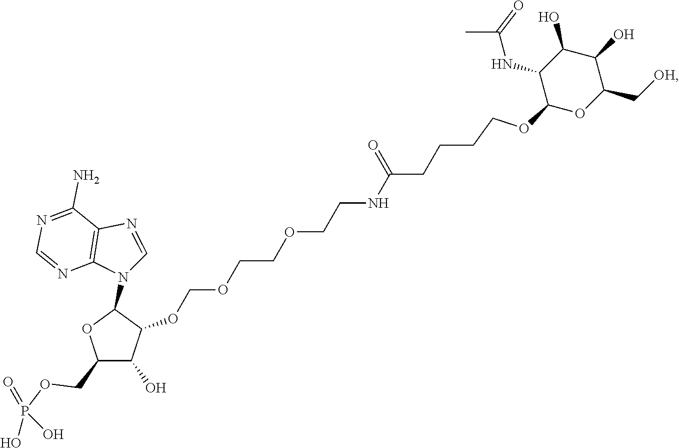 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

In yet other aspects, the present disclosure provides a double stranded RNAi oligonucleotide (dsRNAi) for inhibiting expression of KHK, wherein said dsRNA comprises a sense strand comprising SEQ ID NO: 775 and an antisense strand comprising SEQ ID NO: 820, the antisense strand comprising a region of complementarity to a KHK RNA transcript, e.g. KHK mRNA, wherein said dsRNA is in the form of a conjugate having the structure depicted in FIG. 10A continuing to FIG. 10B, or pharmaceutically acceptable salts thereof.

In another aspect, the present disclosure provides a double stranded RNAi oligonucleotide (dsRNAi) for inhibiting expression of KHK, wherein said dsRNA comprises a sense strand comprising SEQ ID NO: 779 and an antisense strand comprising SEQ ID NO: 824, the antisense strand comprising a region of complementarity to a KHK RNA transcript, e.g. KHK mRNA, wherein said dsRNA is in the form of a conjugate having the structure depicted in FIG. 11A continuing to FIG. 11B, or pharmaceutically acceptable salts thereof.

In another aspect, the present disclosure provides a double stranded RNAi oligonucleotide (dsRNAi) for inhibiting expression of KHK, wherein said dsRNA comprises a sense strand comprising SEQ ID NO: 780 and an antisense strand comprising SEQ ID NO: 825, the antisense strand comprising a region of complementarity to a KHK RNA transcript, e.g. KHK mRNA, wherein said dsRNA is in the form of a conjugate having the structure depicted in FIG. 12A continuing to FIG. 12B, or pharmaceutically acceptable salts thereof.

In another aspect, the present disclosure provides a double stranded RNAi oligonucleotide (dsRNAi) for inhibiting expression of KHK, wherein said dsRNA comprises a sense strand comprising SEQ ID NO: 782 and an antisense strand comprising SEQ ID NO: 827, the antisense strand comprising a region of complementarity to a KHK RNA transcript, e.g. KHK mRNA, wherein said dsRNA is in the form of a conjugate having the structure depicted in FIG. 13A continuing to FIG. 13B, or pharmaceutically acceptable salts thereof.

In another aspect, the present disclosure provides a double stranded RNAi oligonucleotide (dsRNAi) for inhibiting expression of KHK, wherein said dsRNA comprises a sense strand comprising SEQ ID NO: 785 and an antisense strand comprising SEQ ID NO: 830, the antisense strand comprising a region of complementarity to a KHK RNA transcript, e.g. KHK mRNA, wherein said dsRNA is in the form of a conjugate having the structure depicted in FIG. 14A continuing to FIG. 14B, or pharmaceutically acceptable salts thereof.

In another aspect, the present disclosure provides a double stranded RNAi oligonucleotide (dsRNAi) for inhibiting expression of KHK, wherein said dsRNA comprises a sense strand comprising SEQ ID NO: 804 and an antisense strand comprising SEQ ID NO: 849, the antisense strand comprising a region of complementarity to a KHK RNA transcript, e.g. KHK mRNA, wherein said dsRNA is in the form of a conjugate having the structure depicted in FIG. 15A continuing to FIG. 15B, or pharmaceutically acceptable salts thereof.

In some aspects, the present disclosure provides a method for treating a subject having a disease, disorder or condition associated with KHK expression, the method comprising administering to the subject a therapeutically effective amount of any RNAi oligonucleotide or pharmaceutical composition described herein, thereby treating the subject.

In some aspects, the disclosure provides a pharmaceutically acceptable salt of any of the oligonucleotides described herein. In some aspects, the present disclosure provides a pharmaceutical composition comprising any RNAi oligonucleotide described herein, and a pharmaceutically acceptable carrier, salt, delivery agent or excipient. In some aspects, the present disclosure provides a pharmaceutical composition comprising any RNAi oligonucleotide described herein, and a pharmaceutically acceptable diluent, solvent, carrier, salt and/or adjuvant. Likewise, the oligonucleotides herein may be provided in the form of their free acids.

In some aspects, the disclosure provides a method for modulating KHK expression in a target cell expressing KHK, the method comprising administering an RNAi oligonucleotide or pharmaceutical composition described herein in an effective amount to the target cell.

In some aspects, the present disclosure provides a method of delivering an oligonucleotide to a subject, the method comprising administering a pharmaceutical composition described herein.

In some aspects, the present disclosure provides a method for reducing KHK expression in a cell, a population of cells or a subject, the method comprising the step of:
i. contacting the cell or the population of cells with any RNAi oligonucleotide or pharmaceutical composition described herein; or
ii. administering to the subject any RNAi oligonucleotide or pharmaceutical composition described herein.

In any of the foregoing or related aspects, the method of reducing KHK expression comprises reducing an amount or level of KHK mRNA, an amount or level of KHK protein, or both.

In any of the foregoing or related aspects, the subject has a disease, disorder or condition associated with KHK expression. In some aspects, the disease, disorder, or condition associated with KHK expression is non-alcoholic fatty liver disease (NAFLD) and non-alcoholic steatohepatitis (NASH).

In any of the foregoing or related aspects, the RNAi oligonucleotide, or pharmaceutical composition, is administered in combination with a second composition or therapeutic agent.

In some aspects, the present disclosure provides a method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strand comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(o) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd) SEQ ID NOs: 898 and 924, respectively;
(ee) SEQ ID NOs: 899 and 925, respectively
(ff) SEQ ID NOs: 900 and 926, respectively; and
(gg) SEQ ID NOs: 909 and 936, respectively.

In some aspects, the disclosure provides an RNAi oligonucleotide wherein the sense and antisense strands comprise nucleotide sequences selected from the group consisting of:
(a) SEQ ID NOs: 887 and 913, respectively;
(b) SEQ ID NOs: 891 and 917, respectively;
(c) SEQ ID NOs: 892 and 918, respectively;
(d) SEQ ID NOs: 894 and 920, respectively;
(e) SEQ ID NOs: 897 and 923, respectively; and
(f) SEQ ID NOs: 909 and 936, respectively.

In some aspects, the disclosure provides an RNAi oligonucleotide wherein the sense and antisense strands comprise the nucleotide sequences set forth in SEQ ID NOs: 887 and 913, respectively. In other aspects, the disclosure provides an RNAi oligonucleotide wherein the sense and antisense strands comprise the nucleotide sequences set forth in SEQ ID NOs: 891 and 917, respectively. In other aspects, the disclosure provides an RNAi oligonucleotide wherein the sense and antisense strands comprise the nucleotide sequences set forth in SEQ ID NOs: 892 and 918, respectively. In other aspects, the disclosure provides an RNAi oligonucleotide wherein the sense and antisense strands comprise the nucleotide sequences set forth in SEQ ID NOs: 894 and 920, respectively. In other aspects, the disclosure provides an RNAi oligonucleotide wherein the sense and antisense strands comprise the nucleotide sequences set forth in SEQ ID NOs: 897 and 923, respectively. In other aspects, the disclosure provides an RNAi oligonucleotide wherein the sense and antisense strands comprise the nucleotide sequences set forth in SEQ ID NOs: 909 and 936, respectively.

In some aspects, the present disclosure provides a method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strands are selected from the group consisting of:
(a) SEQ ID NOs: 774 and 819, respectively;
(b) SEQ ID NOs: 775 and 820, respectively;
(c) SEQ ID NOs: 776 and 821, respectively;
(d) SEQ ID NOs: 777 and 822, respectively;
(e) SEQ ID NOs: 778 and 823, respectively;
(f) SEQ ID NOs: 779 and 824, respectively;
(g) SEQ ID NOs: 780 and 825, respectively;
(h) SEQ ID NOs: 781 and 826, respectively;
(i) SEQ ID NOs: 782 and 827, respectively;
(j) SEQ ID NOs: 783 and 828, respectively;
(k) SEQ ID NOs: 784 and 829, respectively;
(l) SEQ ID NOs: 785 and 830, respectively;
(m) SEQ ID NOs: 786 and 831, respectively;
(n) SEQ ID NOs: 787 and 832, respectively;
(o) SEQ ID NOs: 788 and 833, respectively;
(p) SEQ ID NOs: 789 and 834, respectively;
(q) SEQ ID NOs: 790 and 835, respectively;
(r) SEQ ID NOs: 791 and 836, respectively;
(s) SEQ ID NOs: 792 and 837, respectively;
(t) SEQ ID NOs: 793 and 838, respectively;
(u) SEQ ID NOs: 794 and 839, respectively;
(v) SEQ ID NOs: 795 and 840, respectively;
(w) SEQ ID NOs: 796 and 841, respectively;
(x) SEQ ID NOs: 797 and 842, respectively;
(y) SEQ ID NOs: 798 and 843, respectively;
(z) SEQ ID NOs: 799 and 844, respectively;
(aa) SEQ ID NOs: 800 and 845, respectively;
(bb) SEQ ID NOs: 801 and 846, respectively;
(cc) SEQ ID NOs: 802 and 847, respectively;
(dd) SEQ ID NOs: 803 and 848, respectively; and
(ee) SEQ ID NOs: 804 and 849, respectively.

In any of the foregoing or related aspects, the disclosure provides an RNAi oligonucleotide wherein the sense and antisense strands comprise nucleotide sequences selected from the group consisting of:
(a) SEQ ID NOs: 775 and 820, respectively;
(b) SEQ ID NOs: 779 and 824, respectively;
(c) SEQ ID NOs: 780 and 825, respectively;
(d) SEQ ID NOs: 782 and 827, respectively;
(e) SEQ ID NOs: 785 and 830, respectively; and
(f) SEQ ID NOs: 804 and 849, respectively.

In some aspects, the disclosure provides an RNAi oligonucleotide wherein the sense and antisense strands comprise the nucleotide sequences set forth in SEQ ID NOs: 775 and 820, respectively. In some aspects, the disclosure provides an RNAi oligonucleotide wherein the sense and antisense strands comprise the nucleotide sequences set forth in SEQ ID NOS: 779 and 824, respectively. In some aspects, the disclosure provides an RNAi oligonucleotide wherein the sense and antisense strands comprise the nucleotide sequences set forth in SEQ ID NOs: 780 and 825, respectively. In other aspects, the disclosure provides an RNAi oligonucleotide wherein the sense and antisense strands comprise the nucleotide sequences set forth in SEQ ID NOs: 782 and 827, respectively. In some aspects, the disclosure provides an RNAi oligonucleotide wherein the sense and antisense strands comprise the nucleotide sequences set forth in SEQ ID NOs: 785 and 830, respectively. In some aspects, the disclosure provides an RNAi oligonucleotide wherein the sense and antisense strands comprise the nucleotide sequences set forth in SEQ ID NOs: 804 and 849, respectively.

In some aspects, the present disclosure provides a method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strands are selected from the group consisting of:
(a) SEQ ID NOs: 805 and 850, respectively;
(b) SEQ ID NOs: 806 and 851, respectively;
(c) SEQ ID NOs: 807 and 852, respectively;
(d) SEQ ID NOs: 808 and 853, respectively;
(e) SEQ ID NOs: 809 and 854, respectively;
(f) SEQ ID NOs: 810 and 855, respectively;
(g) SEQ ID NOs: 811 and 856, respectively;
(h) SEQ ID NOs: 812 and 857, respectively;
(i) SEQ ID NOs: 813 and 858, respectively;
(j) SEQ ID NOs: 814 and 859, respectively;
(k) SEQ ID NOs: 815 and 860, respectively;
(l) SEQ ID NOs: 816 and 861, respectively;
(m) SEQ ID NOs: 817 and 862, respectively and;
(n) SEQ ID NOs: 818 and 863, respectively.

In some aspects, the disclosure provides an RNAi oligonucleotide wherein the sense and antisense strands comprise the nucleotide sequences set forth in SEQ ID NOs: 805 and 850, respectively. In other aspects, the disclosure provides an RNAi oligonucleotide wherein the sense and antisense strands comprise the nucleotide sequences set forth in SEQ ID NOS: 809 and 854, respectively. In yet other aspects, the disclosure provides an RNAi oligonucleotide wherein the sense and antisense strands comprise the nucleotide sequences set forth in SEQ ID NOs: 810 and 855, respectively. In further aspects, the disclosure provides an RNAi oligonucleotide wherein the sense and antisense strands comprise the nucleotide sequences set forth in SEQ ID NOs: 812 and 857, respectively. In other aspects, the disclosure provides an RNAi oligonucleotide wherein the sense and antisense strands comprise the nucleotide sequences set forth in SEQ ID NOs: 815 and 860, respectively. In yet other aspects, the disclosure provides an RNAi oligonucleotide wherein the sense and antisense strands comprise the nucleotide sequences set forth in SEQ ID NOs: 818 and 863, respectively.

In any of the foregoing or related aspects, the disease, disorder, or condition associated with KHK expression is non-alcoholic fatty liver disease (NAFLD) and non-alcoholic steatohepatitis (NASH).

In any of the foregoing or related aspects, a RNAi oligonucleotide described herein is administered at a concentration of 0.01 mg/kg-5 mg/kg.

In some aspects, the disclosure provides use of any RNAi oligonucleotide or pharmaceutical composition described herein, in the manufacture of a medicament for the treatment of a disease, disorder or condition associated with KHK expression, optionally for the treatment of non-alcoholic fatty liver disease (NAFLD) and non-alcoholic steatohepatitis (NASH).

In some aspects, the disclosure provides any RNAi oligonucleotide or pharmaceutical composition described herein, for use, or adaptable for use, in the treatment of a disease, disorder or condition associated with KHK expression, optionally for the treatment of non-alcoholic fatty liver disease (NAFLD) and non-alcoholic steatohepatitis (NASH).

In some aspects, the disclosure provides a kit comprising any RNAi oligonucleotide described herein, an optional pharmaceutically acceptable carrier, and a package insert comprising instructions for administration to a subject having a disease, disorder or condition associated with KHK expression.

In some aspects, the disease, disorder, or condition associated with KHK expression is non-alcoholic fatty liver disease (NAFLD) and non-alcoholic steatohepatitis (NASH).

In some aspects, the disclosure provides an oligonucleotide for reducing KHK expression, the oligonucleotide comprising a nucleotide sequence of 15-50 nucleotides in length, wherein the nucleotide sequence comprises a region of complementarity to a KHK mRNA target sequence of any one of SEQ ID NOs: 4-387, and wherein the region of complementarity is at least 15 contiguous nucleotides in length, or a pharmaceutically acceptable salt thereof. In some aspects, the oligonucleotide is single stranded. In some aspects, the oligonucleotide is an antisense oligonucleotide. In some aspects, the nucleotide sequence is 15-30 nucleotides in length. In some aspects, the nucleotide sequence is 20-25 nucleotides in length. In some aspects, the nucleotide sequence is 22 nucleotides in length. In some aspects, the region of complementarity is 19 contiguous nucleotides in length. In some aspects, the region of complementarity is 20 contiguous nucleotides in length. In some aspects, the nucleotide sequence comprises at least one modification. In some aspects, the nucleotide sequence comprises a nucleotide sequence selected from the group consisting of SEQ ID NOs: 879-885 and 912-938. In some aspects, the nucleotide sequence comprises the nucleotide sequence set forth in SEQ ID NO: 920.

In other aspects, the nucleotide sequence comprises the nucleotide sequence set forth in SEQ ID NO: 923. In yet other aspects, the nucleotide sequence comprises the nucleotide sequence set forth in SEQ ID NO: 918. In further aspects, the nucleotide sequence comprises the nucleotide sequence set forth in SEQ ID NO: 917. In yet further aspects, the nucleotide sequence comprises the nucleotide sequence set forth in SEQ ID NO: 913. In yet further aspects, the nucleotide sequence comprises the nucleotide sequence set forth in SEQ ID NO: 936. In some aspects, the nucleotide sequence comprises the nucleotide sequence set forth in SEQ ID NO: 894. In other aspects, the nucleotide sequence comprises the nucleotide sequence set forth in SEQ ID NO: 897. In yet other aspects, the nucleotide sequence comprises the nucleotide sequence set forth in SEQ ID NO: 892. In further aspects, the nucleotide sequence comprises the nucleotide sequence set forth in SEQ ID NO: 891. In yet further aspects, the nucleotide sequence comprises the nucleotide sequence set forth in SEQ ID NO: 887. In yet further aspects, the nucleotide sequence comprises the nucleotide sequence set forth in SEQ ID NO: 909.

In some aspects, the disclosure provides a cell comprising an oligonucleotide described herein.

In some aspects, the disclosure provides a pharmaceutical composition comprising an oligonucleotide disclosed herein, or a pharmaceutically acceptable salt thereof, and at least one pharmaceutically acceptable carrier, delivery agent or excipient.

In some aspects, the disclosure provides a method for treating a subject having a disease, disorder or condition associated with KHK expression, the method comprising administering to the subject a therapeutically effective amount of an oligonucleotide or pharmaceutical composition described herein.

In some aspects, the disclosure provides a method of delivering an oligonucleotide to a subject, the method comprising administering a pharmaceutical composition described herein to the subject.

In some aspects, the disclosure provides a method for reducing KHK expression in a cell, a population of cells or a subject, the method comprising the step of:
i. contacting the cell or the population of cells with an oligonucleotide or a pharmaceutical composition described herein; or
ii. administering to the subject an oligonucleotide or a pharmaceutical composition described herein. In some aspects, reducing KHK expression comprises reducing an amount or level of KHK mRNA, an amount or level of KHK protein, or both.

In any of the foregoing or related aspects, the subject has a disease, disorder or condition associated with KHK expression. In some aspects, the disease, disorder, or condition associated with KHK expression is non-alcoholic fatty liver disease (NAFLD) and non-alcoholic steatohepatitis (NASH).

In any of the foregoing or related aspects, the oligonucleotide, or pharmaceutical composition, is administered in combination with a second composition or therapeutic agent.

In some aspects, the disclosure provides use of an oligonucleotide or pharmaceutical composition described herein, in the manufacture of a medicament for the treatment of a disease, disorder or condition associated with KHK expression, optionally for the treatment of non-alcoholic fatty liver disease (NAFLD) and non-alcoholic steatohepatitis (NASH). In other aspects, the disclosure provides an oligonucleotide or pharmaceutical composition described herein for use, or adaptable for use, in the treatment of a disease, disorder or condition associated with KHK expression, optionally for the treatment of non-alcoholic fatty liver disease (NAFLD) and non-alcoholic steatohepatitis (NASH).

In some aspects, the disclosure provides a kit comprising an oligonucleotide described herein, an optional pharmaceutically acceptable carrier, and a package insert comprising instructions for administration to a subject having a disease, disorder or condition associated with KHK expression.

In any of the foregoing or related aspects, the disease, disorder, or condition associated with KHK expression is non-alcoholic fatty liver disease (NAFLD) and non-alcoholic steatohepatitis (NASH).

In some aspects, the disclosure provides a double stranded ribonucleic acid (dsRNA) agent for inhibiting expression of KHK, wherein the dsRNA agent comprises a sense strand and an antisense strand forming a duplex region, wherein the sense strand comprises at least 15 contiguous nucleotides differing by no more than 3 nucleotides from a nucleotide sequence selected from SEQ ID NOs: 4-387, and the antisense strand comprises at least 15 contiguous nucleotides differing by no more than 3 nucleotides from a nucleotide sequence selected from SEQ ID NOs: 388-771.

In some aspects, the disclosure provides a double stranded ribonucleic acid (dsRNA) agent for inhibiting expression of KHK, wherein the dsRNA agent comprises a sense strand and an antisense strand forming a duplex region, wherein the sense strand comprises at least 15 contiguous nucleotides differing by no more than 3 nucleotides from a nucleotide sequence selected from SEQ ID NOs: 872-878 and 886-911, and the antisense strand comprises at least 15 contiguous nucleotides differing by no more than 3 nucleotides from a nucleotide sequence selected from SEQ ID NOs: 879-885 and 912-938.

### BRIEF DESCRIPTION OF THE DRAWINGS

FIG. 1 provides a graph depicting the percent (%) mRNA remaining in Hep3B cells (expressing endogenous human KHK) after 24-hour treatment with 1 nM of DsiRNA targeting various regions of the KHK gene. 384 DsiRNAs were designed and screened. Three primer pairs were used that recognized the KHK-A isoform (KHK-F763, NM_000221.2), KHK-C (KHK-825, NM_006488.3) and KHK-All (both isoforms) (KHK-F495, KHK-F1026, NM_006488.3). Expression was normalized between samples using HPRT and SFRS9 housekeeping genes.

FIG. 2A and FIG. 2B provide schematics of the Low-2′-Fluoro modification pattern (Low-2′-Fluoro (3PS) and Low-2′-Fluoro (2PS), respectively) applied to KHK mRNA targeting sequences to generate GalNAc-KHK constructs. The sense strand includes a tetraloop structure of nucleotides 26-31 of the 36-nucleotide strand. The anti-sense strand is complementary and includes a 2-nucleotide overhang.

FIG. 3 provides a graph depicting the percent (%) remaining KHK mRNA in KHK-A and KHK-C HDI (hydrodynamic injection) mice treated with human/non-human primate (NHP)-conserved GalNAc-KHK constructs. 3 days after subcutaneous dosing of 2 mg/kg of GalNAc-KHK constructs formulated in PBS, plasmids encoding either KHK-A and KHK-C were injected into mice via HDI and the percent (%) of KHK mRNA was measured 1 day later in liver samples relative to mice treated with PBS. mRNA was measured from livers using primers recognizing KHK-All (up-right triangle), KHK-C(upside-down triangle), and KHK-A (hexagon). The notation “Hs, 1 mm Mf” represents a human specific sequence that is one base mismatch different from monkey sequence.

FIG. 4A provides a schematic of the Med-2′-Fluoro modification pattern applied to KHK targeting sequence to generate GalNAc-KHK constructs. The sense strand includes a tetraloop structure of nucleotides 26-31 of the 36-nucleotide strand. The anti-sense strand is complementary and includes a 2-nucleotide overhang.

FIGS. 4B-4C provide graphs depicting the percent (%) KHK mRNA remaining after treating mice with GalNAc-KHK constructs having the Med-2′-Fluoro modification pattern. 3 days after subcutaneous dosing of 2 mg/kg of GalNAc-KHK constructs formulated in PBS, plasmid encoding KHK-C was injected into mice via HDI and the percent (%) of KHK mRNA was measured 1 day later in liver samples relative to mice treated with PBS. mRNA was measured using primers identifying both KHK-A and KHK—C isoform s (i.e., KHK-All) (FIG. 4B) and primers identifying only the KHK-C isoform (FIG. 4C). Multiple GalNAc-KHK-constructs were combined in a “mixed” group at 2 mg/kg for a total 10 mg/kg treatment (KHK-0861,-0865,-0882,-0883,-0885) as a positive knock-down control. The notation “Hs, 1 mm Mf” and the like represents a human specific sequence that is one base mismatch different from monkey sequence.

FIG. 4D provides a graph depicting the percent (%) KHK mRNA remaining after treating mice with different GalNAc-KHK constructs having the Med-2′-Fluoro modification pattern. 3 days after subcutaneous dosing of 2 mg/kg of GalNAc-KHK constructs formulated in PBS, plasmids encoding KHK-C were injected into mice via HDI and the percent (%) of KHK mRNA was measured 1 day later in liver samples relative to mice treated with PBS. mRNA was measured using primers (MmKHK-ALL-5-6, Forward: GCTCTTCCAGTTGTTTAGCTATGGT (SEQ ID NO: 939), Reverse: CAGGTGCTTGGCCACATCTT (SEQ ID NO:940), Probe: AGGTGGTGTTTGTCAGC (SEQ ID NO: 941)) identifying only mouse KHK. Remaining mRNA was normalized to a PBS control. Multiple GalNAc-KHK constructs were combined in a “mixed” group as a positive knock-down control.

FIG. 4E provides a graph depicting the difference in percent (%) KHK mRNA remaining after treatment with GalNAc-KHK constructs with different modification patterns (Low-2′F (FIG. 2A and FIG. 2B) and Med-2′F (FIG. 4A)). Remaining mRNA was normalized to a PBS control. Multiple GalNAc-KHK constructs were combined in a “mixed” group as a positive knock-down control.

FIG. 5 provides a graph depicting the difference in percent (%) KHK mRNA remaining after treating mice with GalNAc-KHK constructs. 3 days after subcutaneous dosing of 2 mg/kg of GalNAc-KHK constructs formulated in PBS, plasmid encoding KHK-C(NM_006488) (pCMV6-KHK-C, Cat #: RC223488, OriGene) was injected into mice via HDI and the percent (%) of KHK mRNA remaining was measured 1 day later in liver samples relative to mice treated with PBS. Results include mRNA measured from primers for KHK-All (up-right triangle), and KHK-C(upside-down triangle). Grey arrow shows 30 mg/kg treatment of KHK-885 has more than 98% knockdown.

FIGS. 6A-6B provide graphs depicting the percent (%) KHK mRNA remaining after treating KHK-C plasmid HDI mice (as described in FIG. 5) with different GalNAc-KHK constructs. mRNA was measured using primers identifying both KHK-A and KHK—C isoforms (KHK-AII; FIG. 6A) and primers identifying only the KHK-C isoform (FIG. 6B).

FIG. 6C provides a graph depicting the percent (%) KHK mRNA remaining in the liver after treating KHK-C plasmid HDI mice (as described in FIG. 5) with different GalNAc-KHK constructs. mRNA was measured using primers identifying only mouse KHK.

FIGS. 7A-7C provide graphs depicting the percent (%) KHK mRNA remaining in liver biopsies from non-human primates (NHP) 28 days (FIG. 7A), 56 days (FIG. 7B), and 84 days (FIG. 7C) after a single dose of specified GalNAc-constructs. NHP were subcutaneously injected with 6 mg/kg of GalNAc-KHK on Study Day 0. The percent indicated is the average reduction in KHK-mRNA compared to a PBS control.

FIG. 7D provides a line graph demonstrating the changes in KHK mRNA in liver biopsies taken at various time points from NHP (as treated in FIGS. 7A-7C) after a single dose of GalNAc-KHK constructs.

FIGS. 8A-8C provide graphs depicting the percent (%) KHK protein remaining in liver biopsies from non-human primates (NHP) 28 days (FIG. 8A), 56 days (FIG. 8B), and 84 days (FIG. 8C) after treatment. NHP were treated as in FIGS. 7A-7C. The percent indicated is the average reduction in KHK-protein compared to a PBS control.

FIG. 8D provides a line graph demonstrating the changes in KHK protein in liver biopsies taken at various time points from NHP (as treated in FIGS. 7A-7C) after a single dose of GalNAc-KHK constructs.

FIGS. 9A-9C provide correlation graphs demonstrating the relationship between remaining KHK mRNA expression and remaining KHK protein expression in liver biopsies from NHP treated with a single dose of GalNAc-KHK constructs. Correlation among all constructs is compared at days 28 (FIG. 9A), 56 (FIG. 9B), and 84 (FIG. 9C) after dosing. Individual dots represent individual biopsies.

FIG. 10A continuing to FIG. 10B depict a dsRNAi oligonucleotide of the invention comprising a sense strand comprising SEQ ID NO: 775 and an antisense strand comprising SEQ ID NO: 820, wherein said dsRNA is in the form of a conjugate.

FIG. 11A continuing to FIG. 11B depict a dsRNAi oligonucleotide of the invention comprising a sense strand comprising SEQ ID NO: 779 and an antisense strand comprising SEQ ID NO: 824, wherein said dsRNA is in the form of a conjugate.

FIG. 12A continuing to FIG. 12B depict a dsRNAi oligonucleotide of the invention comprising a sense strand comprising SEQ ID NO: 780 and an antisense strand comprising SEQ ID NO: 825, wherein said dsRNA is in the form of a conjugate.

FIG. 13A continuing to FIG. 13B depict a dsRNAi oligonucleotide of the invention comprising a sense strand comprising SEQ ID NO: 782 and an antisense strand comprising SEQ ID NO: 827, wherein said dsRNA is in the form of a conjugate.

FIG. 14A continuing to FIG. 14B depict a dsRNAi oligonucleotide of the invention comprising a sense strand comprising SEQ ID NO: 785 and an antisense strand comprising SEQ ID NO: 830, wherein said dsRNA is in the form of a conjugate.

FIG. 15A continuing to FIG. 15B depict a dsRNAi oligonucleotide of the invention comprising a sense strand comprising SEQ ID NO: 804 and an antisense strand comprising SEQ ID NO: 849, wherein said dsRNA is in the form of a conjugate.

### DETAILED DESCRIPTION OF THE INVENTION

According to some aspects, the disclosure provides oligonucleotides that reduce KHK expression in the liver. In some embodiments, the oligonucleotides provided herein are useful to treat diseases associated with KHK expression in the liver. In some respects, the disclosure provides methods of treating a disease associated with KHK expression by reducing KHK gene expression in cells (e.g., cells of the liver).

### Oligonucleotide Inhibitors of KHK Expression

### Ketohexokinase (KHK) Target Sequences

In some embodiments, the disclosure provides an oligonucleotide which is targeted to a target sequence comprising a ketohexokinase (KHK) mRNA. In some embodiments, the oligonucleotide, or a portion, fragment, or strand thereof (e.g., an antisense strand or a guide strand of a dsRNA) binds or anneals to a target sequence comprising a KHK mRNA, thereby inhibiting KHK expression. In some embodiments, the oligonucleotide is targeted to a target sequence comprising a KHK-A isoform mRNA. In some embodiments, the oligonucleotide is targeted to a target sequence comprising a KHK-C isoform mRNA. In some embodiments, the oligonucleotide is targeted to a KHK target sequence for the purpose of inhibiting KHK expression in vivo. In some embodiments, the amount or extent of inhibition of KHK expression by an oligonucleotide targeted to a KHK target sequence correlates with the potency of the oligonucleotide. In some embodiments, the amount or extent of inhibition of KHK expression by an oligonucleotide targeted to a KHK target sequence correlates with the amount or extent of therapeutic benefit in a subject or patient having a disease, disorder or condition associated with the expression of KHK treated with the oligonucleotide.

Through examination of the nucleotide sequence of mRNAs encoding KHK, including mRNAs of multiple different species (e.g., human, cynomolgus monkey, mouse, and rat; see, e.g., Example 2) and as a result of in vitro and in vivo testing (see, e.g., Examples 2-6), it has been discovered that certain nucleotide sequences of KHK mRNA are more amenable than others to oligonucleotide-based inhibition and are thus useful as target sequences for the oligonucleotides herein. In some embodiments, a sense strand of an oligonucleotide (e.g., a dsRNA) described herein comprises a KHK target sequence. In some embodiments, a portion or region of the sense strand of a dsRNA described herein comprises a KHK target sequence. In some embodiments, a KHK target sequence comprises, or consists of, a sequence of any one of SEQ ID Nos: 4-387. In some embodiments, a KHK target sequence comprises, or consists of, nucleotides 1-19 of any one of SEQ ID Nos: 4-387. In some embodiments, a KHK target sequence comprises, or consists of, the sequence set forth in SEQ ID No: 39. In some embodiments, a KHK target sequence comprises, or consists of, nucleotides 1-19 of the sequence set forth in SEQ ID No: 39. In some embodiments, a KHK target sequence comprises, or consists of, the sequence set forth in SEQ ID No: 102. In some embodiments, a KHK target sequence comprises, or consists of, nucleotides 1-19 of the sequence set forth in SEQ ID No: 102. In some embodiments, a KHK target sequence comprises, or consists of, the sequence set forth in SEQ ID No: 104. In some embodiments, a KHK target sequence comprises, or consists of, nucleotides 1-19 of the sequence set forth in SEQ ID No: 104. In some embodiments, a KHK target sequence comprises, or consists of, the sequence set forth in SEQ ID No: 107. In some embodiments, a KHK target sequence comprises, or consists of, nucleotides 1-19 of the sequence set forth in SEQ ID No: 107. In some embodiments, a KHK target sequence comprises, or consists of, the sequence set forth in SEQ ID No: 191. In some embodiments, a KHK target sequence comprises, or consists of, nucleotides 1-19 of the sequence set forth in SEQ ID No: 191. In some embodiments, a KHK target sequence comprises, or consists of, the sequence set forth in SEQ ID No: 269. In some embodiments, a KHK target sequence comprises, or consists of, nucleotides 1-19 of the sequence set forth in SEQ ID No: 269.

### KHK Targeting Sequences

In some embodiments, the oligonucleotides herein have regions of complementarity to KHK mRNA (e.g., within a target sequence of KHK mRNA) for purposes of targeting the mRNA in cells and inhibiting its expression. In some embodiments, the oligonucleotides herein comprise a KHK targeting sequence (e.g., an antisense strand or a guide strand of a dsRNA) having a region of complementarity that binds or anneals to a KHK target sequence by complementary (Watson-Crick) base pairing. The targeting sequence or region of complementarity is generally of a suitable length and base content to enable binding or annealing of the oligonucleotide (or a strand thereof) to a KHK mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a target sequence or region of complementarity complementary to a sequence of any one of SEQ ID NOs: 4-387, and the targeting sequence or region of complementarity is 18 nucleotides in length. In some embodiments, an oligonucleotide comprises a target sequence or region of complementarity complementary to a sequence of any one of SEQ ID NOs: 4-387, and the targeting sequence or region of complementarity is 19 nucleotides in length. In some embodiments, an oligonucleotide comprises a target sequence or region of complementarity complementary to a sequence of any one of SEQ ID NOs: 4-387, and the targeting sequence or region of complementarity is 20 nucleotides in length. In some embodiments, an oligonucleotide comprises a target sequence or region of complementarity complementary to a sequence of any one of SEQ ID NOs: 4-387, and the targeting sequence or region of complementarity is 21 nucleotides in length. In some embodiments, an oligonucleotide comprises a target sequence or region of complementarity complementary to a sequence of any one of SEQ ID NOs: 4-387, and the targeting sequence or region of complementarity is 22 nucleotides in length. In some embodiments, an oligonucleotide comprises a target sequence or region of complementarity complementary to a sequence of any one of SEQ ID NOs: 4-387, and the targeting sequence or region of complementarity is 23 nucleotides in length. In some embodiments, an oligonucleotide comprises a target sequence or region of complementarity complementary to a sequence of any one of SEQ ID NOs: 4-387, and the targeting sequence or region of complementarity is 24 nucleotides in length.

In some embodiments, an oligonucleotide herein comprises a targeting sequence or a region of complementarity (e.g., an antisense strand or a guide strand of a double-stranded oligonucleotide) that is fully complementary to a KHK target sequence. In some embodiments, the targeting sequence or region of complementarity is partially complementary to a KHK target sequence. In some embodiments, the targeting sequence or region of complementarity has up to 3 nucleotide mismatches to a KHK target sequence. In some embodiments, the oligonucleotide comprises a targeting sequence or region of complementarity that is fully complementary to a sequence of KHK. In some embodiments, the oligonucleotide comprises a targeting sequence or region of complementarity that is partially complementary to a sequence of KHK. In some embodiments, the oligonucleotide comprises a targeting sequence or region of complementarity that is fully complementary to a sequence of any one of SEQ ID NOs: 4-387. In some embodiments, the oligonucleotide comprises a targeting sequence or region of complementarity that is partially complementary to a sequence of KHK. In some embodiments, the oligonucleotide comprises a targeting sequence or region of complementarity that is fully complementary to nucleotides 1-19 of a sequence of any one of SEQ ID NOs: 4-387. In some embodiments, the oligonucleotide comprises a targeting sequence or region of complementarity that is fully complementary to the sequence set forth in SEQ ID NO: 39. In some embodiments, the oligonucleotide comprises a targeting sequence or region of complementarity that is fully complementary to nucleotides 1-19 of the sequence set forth in SEQ ID NO: 39. In some embodiments, the oligonucleotide comprises a targeting sequence or region of complementarity that is fully complementary to the sequence set forth in SEQ ID NO: 102. In some embodiments, the oligonucleotide comprises a targeting sequence or region of complementarity that is fully complementary to nucleotides 1-19 of the sequence set forth in SEQ ID NO: 102. In some embodiments, the oligonucleotide comprises a targeting sequence or region of complementarity that is fully complementary to the sequence set forth in SEQ ID NO: 104. In some embodiments, the oligonucleotide comprises a targeting sequence or region of complementarity that is fully complementary to nucleotides 1-19 of the sequence set forth in SEQ ID NO: 104. In some embodiments, the oligonucleotide comprises a targeting sequence or region of complementarity that is fully complementary to the sequence set forth in SEQ ID NO: 107. In some embodiments, the oligonucleotide comprises a targeting sequence or region of complementarity that is fully complementary to nucleotides 1-19 of the sequence set forth in SEQ ID NO: 107. In some embodiments, the oligonucleotide comprises a targeting sequence or region of complementarity that is fully complementary to the sequence set forth in SEQ ID NO: 191. In some embodiments, the oligonucleotide comprises a targeting sequence or region of complementarity that is fully complementary to nucleotides 1-19 of the sequence set forth in SEQ ID NO: 191. In some embodiments, the oligonucleotide comprises a targeting sequence or region of complementarity that is fully complementary to the sequence set forth in SEQ ID NO: 269. In some embodiments, the oligonucleotide comprises a targeting sequence or region of complementarity that is fully complementary to nucleotides 1-19 of the sequence set forth in SEQ ID NO: 269. In some embodiments, the oligonucleotide comprises a targeting sequence or region of complementarity that is partially complementary to a sequence of any one of SEQ ID NOs: 4-387. In some embodiments, the oligonucleotide comprises a targeting sequence or region of complementarity that is partially complementary to nucleotides 1-19 of a sequence of any one of SEQ ID NOs: 4-387. In some embodiments, the oligonucleotide comprises a targeting sequence or region of complementarity that is partially complementary to the sequence set forth in SEQ ID NO: 39. In some embodiments, the oligonucleotide comprises a targeting sequence or region of complementarity that is partially complementary to nucleotides 1-19 of the sequence set forth in SEQ ID NO: 39. In some embodiments, the oligonucleotide comprises a targeting sequence or region of complementarity that is partially complementary to the sequence set forth in SEQ ID NO: 102. In some embodiments, the oligonucleotide comprises a targeting sequence or region of complementarity that is partially complementary to nucleotides 1-19 of the sequence set forth in SEQ ID NO: 102. In some embodiments, the oligonucleotide comprises a targeting sequence or region of complementarity that is partially complementary to the sequence set forth in SEQ ID NO: 104. In some embodiments, the oligonucleotide comprises a targeting sequence or region of complementarity that is partially complementary to nucleotides 1-19 of the sequence set forth in SEQ ID NO: 104. In some embodiments, the oligonucleotide comprises a targeting sequence or region of complementarity that is partially complementary to the sequence set forth in SEQ ID NO: 107. In some embodiments, the oligonucleotide comprises a targeting sequence or region of complementarity that is partially complementary to nucleotides 1-19 of the sequence set forth in SEQ ID NO: 107. In some embodiments, the oligonucleotide comprises a targeting sequence or region of complementarity that is partially complementary to the sequence set forth in SEQ ID NO: 191. In some embodiments, the oligonucleotide comprises a targeting sequence or region of complementarity that is partially complementary to nucleotides 1-19 of the sequence set forth in SEQ ID NO: 191. In some embodiments, the oligonucleotide comprises a targeting sequence or region of complementarity that is partially complementary to the sequence set forth in SEQ ID NO: 269. In some embodiments, the oligonucleotide comprises a targeting sequence or region of complementarity that is partially complementary to nucleotides 1-19 of the sequence set forth in SEQ ID NO: 269. In some embodiments, the oligonucleotide comprises a targeting sequence or region of complementarity that is fully complementary to a sequence of any one of SEQ ID NOs: 872-878 and 886-911. In some embodiments, the oligonucleotide comprises a targeting sequence or region of complementarity that is partially complementary to a sequence of any one of SEQ ID NOs: 872-878 and 886-911. In some embodiments, the oligonucleotide comprises a targeting sequence or region of complementarity that is fully complementary to a sequence of any one of SEQ ID NOs: 887, 891, 892, 894, 897 and 909. In some embodiments, the oligonucleotide comprises a targeting sequence or region of complementarity that is partially complementary to a sequence of any one of SEQ ID NOs: 887, 891, 892, 894, 897 and 909.

In some embodiments, the oligonucleotide herein comprises a targeting sequence or region of complementarity that is complementary to a contiguous sequence of nucleotides comprising a KHK mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising a KHK mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising a KHK mRNA, wherein the contiguous sequence of nucleotides is 19 nucleotides in length. In some embodiments, the oligonucleotide comprises a targeting sequence or region of complementarity that is complementary to a contiguous sequence of nucleotides comprising a KHK mRNA, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 4-387, optionally wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 4-387, wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 4-387, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 872-878 and 886-911, optionally wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 872-878 and 886-911, wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 872-878 and 886-911, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 887, 891, 892, 894, 897, and 909, optionally wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 887, 891, 892, 894, 897, and 909, wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 887, 891, 892, 894, 897, and 909, wherein the contiguous sequence of nucleotides is 20 nucleotides in length.

In some embodiments, a targeting sequence or region of complementarity of an oligonucleotide is provided that is complementary to contiguous nucleotides of a sequence as set forth in any one of SEQ ID NOs: 4-387 and spans the entire length of an antisense strand. In some embodiments, a targeting sequence or region of complementarity of an oligonucleotide is provided that is complementary to contiguous nucleotides of nucleotides 1-19 a sequence as set forth in any one of SEQ ID NOs: 4-387 and spans the entire length of an antisense strand. In some embodiments, a region of complementarity of an oligonucleotide is provided that is complementary to contiguous nucleotides of a sequence as set forth in any one of SEQ ID NOs: 4-387 and spans a portion of the entire length of an antisense strand. In some embodiments, a region of complementarity of an oligonucleotide is provided that is complementary to contiguous nucleotides of nucleotides 1-19 a sequence as set forth in any one of SEQ ID NOs: 4-387 and spans a portion of the entire length of an antisense strand. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-19 of a sequence as set forth in any one of SEQ ID NOs: 4-387. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-20 of a sequence as set forth in any one of SEQ ID NOs: 4-387. In some embodiments, a targeting sequence or region of complementarity of an oligonucleotide is provided that is complementary to contiguous nucleotides of a sequence as set forth in any one of SEQ ID NOs: 872-878 and 886-911 and spans the entire length of an antisense strand. In some embodiments, a region of complementarity of an oligonucleotide that is complementary to contiguous nucleotides of a sequence as set forth in any one of SEQ ID NOs: 872-878 and 886-911 and spans a portion of the entire length of an antisense strand. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-19 of a sequence as set forth in any one of SEQ ID NOs: 872-878 and 886-911. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-20 of a sequence as set forth in any one of SEQ ID NOs: 872-878 and 886-911. In some embodiments, a targeting sequence or region of complementarity of an oligonucleotide is provided that is complementary to contiguous nucleotides of a sequence as set forth in any one of SEQ ID NOs: 887, 891, 892, 894, 897, and 909 and spans the entire length of an antisense strand. In some embodiments, a region of complementarity of an oligonucleotide is provided that is complementary to contiguous nucleotides of a sequence as set forth in any one of SEQ ID NOs: 887, 891, 892, 894, 897, and 909 and spans a portion of the entire length of an antisense strand. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-19 of a sequence as set forth in any one of SEQ ID NOs: 887, 891, 892, 894, 897, and 909. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-20 of a sequence as set forth in any one of SEQ ID NOs: 887, 891, 892, 894, 897, and 909.

In some embodiments, an oligonucleotide herein comprises a targeting sequence or region of complementarity having one or more base pair (bp) mismatches with the corresponding KHK target sequence. In some embodiments, the targeting sequence or region of complementarity may have up to about 1, up to about 2, up to about 3, up to about 4, up to about 5, etc. mismatches with the corresponding KHK target sequence provided that the ability of the targeting sequence or region of complementarity to bind or anneal to the KHK mRNA under appropriate hybridization conditions and/or the ability of the oligonucleotide to inhibit KHK expression is maintained. Alternatively, the targeting sequence or region of complementarity may have no more than 1, no more than 2, no more than 3, no more than 4, or no more than 5 mismatches with the corresponding KHK target sequence provided that the ability of the targeting sequence or region of complementarity to bind or anneal to the KHK mRNA under appropriate hybridization conditions and/or the ability of the oligonucleotide to inhibit KHK expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed throughout the targeting sequence or region of complementarity. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof. In some embodiments, the oligonucleotide comprises a targeting sequence or a region of complementarity that is complementary to a contiguous sequence of nucleotides of any one of SEQ ID NOs: 4-387, wherein the targeting sequence or region of complementarity may have up to about 1, up to about 2, up to about 3, up to about 4, up to about 5, etc. mismatches with the corresponding KHK target sequence. In some embodiments, the oligonucleotide comprises a targeting sequence or a region of complementarity that is complementary to a contiguous sequence of nucleotides of nucleotides 1-19 of any one of SEQ ID NOs: 4-387, wherein the targeting sequence or region of complementarity may have up to about 1, up to about 2, up to about 3, up to about 4, up to about 5, etc. mismatches with the corresponding KHK target sequence. In some embodiments, the oligonucleotide comprises a targeting sequence or a region of complementarity that is complementary to a contiguous sequence of nucleotides of any one of SEQ ID NOs: 4-387, wherein the targeting sequence or region of complementarity may have no more than 1, no more than 2, no more than 3, no more than 4, or no more than 5 mismatches with the corresponding KHK target sequence. In some embodiments, the oligonucleotide comprises a targeting sequence or a region of complementarity that is complementary to a contiguous sequence of nucleotides of nucleotides 1-19 of any one of SEQ ID NOs: 4-387, wherein the targeting sequence or region of complementarity may have no more than 1, no more than 2, no more than 3, no more than 4, or no more than 5 mismatches with the corresponding KHK target sequence. In some embodiments, the oligonucleotide comprises a targeting sequence or a region of complementarity that is complementary to a contiguous sequence of nucleotides of any one of SEQ ID NOs: 872-878 and 886-911, wherein the targeting sequence or region of complementarity may have up to about 1, up to about 2, up to about 3, up to about 4, up to about 5, etc. mismatches with the corresponding KHK target sequence. In some embodiments, the oligonucleotide comprises a targeting sequence or a region of complementarity that is complementary to a contiguous sequence of nucleotides of any one of SEQ ID NOs: 872-878 and 886-911, wherein the targeting sequence or region of complementarity may have no more than 1, no more than 2, no more than 3, no more than 4, or no more than 5 mismatches with the corresponding KHK target sequence. In some embodiments, the oligonucleotide comprises a targeting sequence or a region of complementarity that is complementary to a contiguous sequence of nucleotides of any one of SEQ ID NOs: 887, 891, 892, 894, 897, and 909, wherein the targeting sequence or region of complementarity may have up to about 1, up to about 2, up to about 3, up to about 4, up to about 5, etc. mismatches with the corresponding KHK target sequence. In some embodiments, the oligonucleotide comprises a targeting sequence or a region of complementarity that is complementary to a contiguous sequence of nucleotides of any one of SEQ ID NOs: 887, 891, 892, 894, 897, and 909, wherein the targeting sequence or region of complementarity may have no more than 1, no more than 2, no more than 3, no more than 4, or no more than 5 mismatches with the corresponding KHK target sequence.

### Types of Oligonucleotides

A variety of oligonucleotide types and/or structures are useful for targeting KHK in the methods herein including, but not limited to, RNAi oligonucleotides, antisense oligonucleotides, miRNAs, etc. Any of the oligonucleotide types described herein or elsewhere are contemplated for use as a framework to incorporate a KHK targeting sequence herein for the purposes of inhibiting KHK expression.

In some embodiments, the oligonucleotides herein inhibit KHK expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically stabilizing tetraloop structure (see, e.g., U.S. Pat. Nos. 8,513,207 and 8,927,705, as well as Intl. Patent Application Publication No. WO 2010/033225). Such structures may include single-stranded (ss) extensions (on one or both sides of the molecule) as well as double-stranded (ds) extensions.

In some embodiments, the oligonucleotides herein engage with the RNAi pathway downstream of the involvement of Dicer (e.g., Dicer cleavage). In some embodiments, the oligonucleotides described herein are Dicer substrates. In some embodiments, upon endogenous Dicer processing, double-stranded nucleic acids of 19-23 nucleotides in length capable of reducing KHK expression are produced. In some embodiments, the oligonucleotide has an overhang (e.g., of 1, 2, or 3 nucleotides in length) in the 3′ end of the sense strand. In some embodiments, the oligonucleotide has an overhang (e.g., of 1, 2, or 3 nucleotides in length) in the 3′ end of the antisense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 36 (e.g., 17 to 36, 20 to 25 or 21-23) nucleotides in length. In some embodiments, the oligonucleotides described herein comprise an antisense strand of 19-30 nucleotides in length and a sense strand of 19-50 nucleotides in length, wherein the antisense and sense strands are separate strands which form an asymmetric duplex region having an overhang of 1-4 nucleotides at the 3′ terminus of the antisense strand. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY. Blackburn (ed.), Royal Society of Chemistry, 2006), shRNAs (e.g., having 19 bp or shorter stems; see, e.g., Moore et al. (2010) METHODS MOL. BIOL. 629:139-156), blunt siRNAs (e.g., of 19 bps in length; see, e.g., Kraynack & Baker (2006) RNA 12:163-176), asymmetrical siRNAs (aiRNA; see, e.g., Sun et al. (2008) NAT. BIOTECHNOL. 26:1379-1382), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structures that may be used in some embodiments to reduce or inhibit the expression of KHK are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting KHK expression herein is single-stranded (ss). Such structures may include but are not limited to single-stranded RNAi molecules. Recent efforts have demonstrated the activity of ss RNAi molecules (see, e.g., Matsui et al. (2016) MOL. THER. 24:946-955). However, in some embodiments, oligonucleotides herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

In some embodiments, the antisense oligonucleotide shares a region of complementarity with KHK mRNA. In some embodiments, the antisense oligonucleotide targets SEQ ID NO: 1. In some embodiments, the antisense oligonucleotide targets SEQ ID NO: 2. In some embodiments, the antisense oligonucleotide targets SEQ ID NO: 3. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is complementary to any one of SEQ ID NOs: 4-387. In some embodiments, the antisense oligonucleotide is complementary to nucleotides 1-19 of any one of SEQ ID NOs: 4-387. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### Double-Stranded Oligonucleotides

In some aspects, the disclosure provides double-stranded (ds) RNAi oligonucleotides for targeting KHK mRNA and inhibiting KHK expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a tetraloop (L) or triloop (triL), and a second subregion (S2), wherein L or triL is located between S1 and S2, and wherein S1 and S2 form a second duplex (D2). D2 may have various length. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R¹ of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, a dsRNAi provided herein comprises a sense strand having a sequence of any one of SEQ ID NOs: 4-387; and an antisense strand comprising a complementary sequence selected from SEQ ID NOs: 388-771 as is arranged Table 2.

In some embodiments, a dsRNAi oligonucleotide comprises a sense strand and an antisense strand comprising nucleotide sequences selected from:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(o) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd) SEQ ID NOs: 898 and 924, respectively;
(ee) SEQ ID NOs: 899 and 925, respectively
(ff) SEQ ID NOs: 900 and 926, respectively; and
(gg) SEQ ID NOs: 909 and 936, respectively.

In some embodiments, a dsRNAi oligonucleotide comprises a sense strand and an antisense strand comprising nucleotide sequences selected from:
(a) SEQ ID NOs: 887 and 913, respectively;
(b) SEQ ID NOs: 891 and 917, respectively;
(c) SEQ ID NOs: 892 and 918, respectively;
(d) SEQ ID NOs: 894 and 920, respectively;
(e) SEQ ID NOs: 897 and 923, respectively; and
(f) SEQ ID NOs: 909 and 936, respectively.

In some embodiments, the sense strand comprises the sequence of SEQ ID NO: 887 and the antisense strand comprises the sequence of SEQ ID NO: 913. In some embodiments, the sense strand comprises the sequence of SEQ ID NO: 891 and the antisense strand comprises the sequence of SEQ ID NO: 917. In some embodiments, the sense strand comprises the sequence of SEQ ID NO: 892 and the antisense strand comprises the sequence of SEQ ID NO: 918. In some embodiments, the sense strand comprises the sequence of SEQ ID NO: 894 and the antisense strand comprises the sequence of SEQ ID NO: 920. In some embodiments, the sense strand comprises the sequence of SEQ ID NO: 897 and the antisense strand comprises the sequence of SEQ ID NO: 923. In some embodiments, the sense strand comprises the sequence of SEQ ID NO: 909 and the antisense strand comprises the sequence of SEQ ID NO: 936.

It should be appreciated that, in some embodiments, sequences presented in the Sequence Listing may be referred to in describing the structure of an oligonucleotide (e.g., a dsRNAi oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a dsRNAi oligonucleotide herein comprises a 25-nucleotide sense strand and a 27-nucleotide antisense strand that when acted upon by a Dicer enzyme results in an antisense strand that is incorporated into the mature RISC. In some embodiments, the sense strand of the dsRNA is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the dsRNA is longer than 25 nucleotides (e.g., 26, 27, 28, 29 or 30 nucleotides). In some embodiments, the sense strand of the dsRNA comprises a nucleotide sequence selected from SEQ ID NOs: 4-387, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the dsRNA comprises a nucleotide sequence selected from SEQ ID NOs: 4-387, wherein the nucleotide sequence is longer than 25 nucleotides (e.g., 26, 27, 28, 29 or 30 nucleotides).

In some embodiments, oligonucleotides herein have one 5′ end that is thermodynamically less stable when compared to the other 5′ end. In some embodiments, an asymmetric oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, a dsRNAi oligonucleotide has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. In some embodiments, the oligonucleotide comprises a targeting sequence or a region of complementarity that is complementary to a contiguous sequence of nucleotides of any one of SEQ ID NOs: 4-387, and a 5′-overhang comprising a length of between 1 and 6 nucleotides. In some embodiments, the oligonucleotide comprises a sense strand comprising a nucleotide sequence selected from SEQ ID NOs: 4-387, wherein the oligonucleotide comprises a 5′-overhang comprising a length of between 1 and 6 nucleotides. In some embodiments, the oligonucleotide comprises an antisense strand comprising a nucleotide sequence selected from SEQ ID NOs: 388-771, wherein the oligonucleotide comprises a 5′-overhang comprising a length of between 1 and 6 nucleotides. In some embodiments, the oligonucleotide comprises a sense strand comprising a nucleotide sequence selected from SEQ ID NOs: 4-387 and antisense strand comprising a nucleotide sequence selected from SEQ ID NOs: 388-771, wherein the oligonucleotide comprises a 5′-overhang comprising a length of between 1 and 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., KHK mRNA). In some embodiments, the two terminal nucleotides on the 3′ end of the antisense strand are not complementary with the target mRNA. In some embodiments, the two terminal nucleotides on the 3′ end of the antisense strand of a dsRNAi oligonucleotide herein are unpaired. In some embodiments, the two terminal nucleotides on the 3′ end of the antisense strand of a dsRNAi oligonucleotide herein comprise an unpaired GG. In some embodiments, the two terminal nucleotides on the 3′ end of an antisense strand of a dsRNAi oligonucleotide herein are not complementary to the target mRNA. In some embodiments, two terminal nucleotides on each 3′ end of a dsRNAi oligonucleotide are GG. Typically, one or both of the two terminal GG nucleotides on each 3′ end of a double-stranded oligonucleotide is not complementary with the target mRNA. In some embodiments, the oligonucleotide comprises a targeting sequence or a region of complementarity that is complementary to a contiguous sequence of nucleotides of any one of SEQ ID NOs: 4-387, wherein the two terminal nucleotides on the 3′ end of the antisense strand of a dsRNAi oligonucleotide herein comprise an unpaired GG. In some embodiments, the oligonucleotide comprises a targeting sequence or a region of complementarity that is complementary to a contiguous sequence of nucleotides of nucleotides 1-19 of any one of SEQ ID NOs: 4-387, wherein the two terminal nucleotides on the 3′ end of the antisense strand of a dsRNAi oligonucleotide herein comprise an unpaired GG. In some embodiments, the oligonucleotide comprises an antisense strand comprising a nucleotide sequence selected from SEQ ID NOs: 388-771, wherein the two terminal nucleotides on the 3′ end of the antisense strand of a dsRNAi oligonucleotide herein comprise an unpaired GG. In some embodiments, the oligonucleotide comprises a sense strand comprising a nucleotide sequence selected from SEQ ID NOs: 4-387 and antisense strand comprising a nucleotide sequence selected from SEQ ID NOs: 388-771, wherein the two terminal nucleotides on the 3′ end of the antisense strand of a dsRNAi oligonucleotide herein comprise an unpaired GG.

In some embodiments, there is one or more (e.g., 1, 2, 3, 4 or 5) mismatch(es) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In some embodiments, two mismatches are incorporated at the 3′ end of the sense strand. In some embodiments, base mismatches, or destabilization of segments at the 3′ end of the sense strand of the dsRNAi oligonucleotide improves or increases the potency of the dsRNAi oligonucleotide. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein there is one or more (e.g., 1, 2, 3, 4 or 5) mismatch(es) between the sense and antisense strands.

### Antisense Strands

In some embodiments, an antisense strand of a dsRNAi oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, as the antisense strand is referred to as a guide strand. In some embodiments, a sense strand complementary to a guide strand may be referred to as a “passenger strand.”

In some embodiments, a dsRNAi oligonucleotide herein comprises an antisense strand of up to about 50 nucleotides in length (e.g., up to 50, up to 40, up to 35, up to 30, up to 27, up to 25, up to 21, up to 19, up to 17 or up to 12 nucleotides in length). In some embodiments, a dsRNAi oligonucleotide comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, a dsRNAi oligonucleotide comprises an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, a dsRNAi oligonucleotide comprises antisense strand of 15 to 30 nucleotides in length. In some embodiments, an antisense strand of any one of the dsRNAi oligonucleotides disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, a dsRNAi oligonucleotide comprises an antisense strand of 22 nucleotides in length.

In some embodiments, a dsRNAi oligonucleotide disclosed herein for targeting KHK comprises an antisense strand comprising or consisting of a sequence as set forth in any one of SEQ ID NOs: 388-771. In some embodiments, a dsRNAi oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 388-771. In some embodiments, a dsRNAi oligonucleotide disclosed herein for targeting KHK comprises an antisense strand comprising or consisting of a sequence as set forth in any one of SEQ ID NOs: 879-885 and 912-938. In some embodiments, a dsRNAi oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 879-885 and 912-938. In some embodiments, a dsRNAi oligonucleotide disclosed herein for targeting KHK comprises an antisense strand comprising or consisting of a sequence as set forth in any one of SEQ ID NOs: 913, 917, 918, 920, 923 and 936. In some embodiments, a dsRNAi oligonucleotide herein comprises an antisense strand comprising at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 913, 917, 918, 920, 923 and 936.

In some embodiments, a dsRNAi oligonucleotide herein comprises an antisense strand comprising a nucleotide sequence selected from SEQ ID NOs: 948-953.

### Sense Strands

In some embodiments, a dsRNAi oligonucleotide disclosed herein for targeting KHK mRNA and inhibiting KHK expression comprises a sense strand sequence as set forth in any one of SEQ ID NOs: 4-387. In some embodiments, a dsRNAi oligonucleotide has a sense strand that comprises at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 4-387. In some embodiments, a dsRNAi oligonucleotide disclosed herein for targeting KHK mRNA and inhibiting KHK expression comprises a sense strand sequence as set forth in any one of SEQ ID NOs: 872-878 and 886-911. In some embodiments, a dsRNAi oligonucleotide has a sense strand that comprises at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 872-878 and 886-911. In some embodiments, a dsRNAi oligonucleotide disclosed herein for targeting KHK mRNA and inhibiting KHK expression comprises a sense strand sequence as set forth in any one of SEQ ID NOs: 887, 891, 892, 894, 897 and 909. In some embodiments, a dsRNAi oligonucleotide has a sense strand that comprises at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 887, 891, 892, 894, 897 and 909.

In some embodiments, a dsRNAi oligonucleotide herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, a dsRNAi oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, a dsRNAi oligonucleotide comprises a sense strand of 15 to 50 nucleotides in length. In some embodiments, a dsRNAi oligonucleotide comprises a sense strand of 18 to 36 nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a dsRNAi oligonucleotide comprises a sense strand of 36 nucleotides in length.

In some embodiments, a sense strand comprises a stem-loop structure at its 3′ end. In some embodiments, the stem-loop is formed by intrastrand base pairing. In some embodiments, a sense strand comprises a stem-loop structure at its 5′ end. In some embodiments, a stem is a duplex of 2, 3, 4, 5, 6, 7, 8, 9, 10, 11, 12, 13 or 14 nucleotides in length. In some embodiments, a stem-loop provides the dsRNAi oligonucleotide protection against degradation (e.g., enzymatic degradation), facilitates or improves targeting and/or delivery to a target cell, tissue, or organ (e.g., the liver), or both. For example, in some embodiments, the loop of a stem-loop provides nucleotides comprising one or more modifications that facilitate, improve, or increase targeting to a target mRNA (e.g., a KHK mRNA), inhibition of target gene expression (e.g., KHK expression), and/or delivery to a target cell, tissue, or organ (e.g., the liver), or a combination thereof. In some embodiments, the stem-loop itself or modification(s) to the stem-loop do not substantially affect the inherent gene expression inhibition activity of the dsRNAi oligonucleotide, but facilitates, improves, or increases stability (e.g., provides protection against degradation) and/or delivery of the oligonucleotide to a target cell, tissue, or organ (e.g., the liver). In certain embodiments, a dsRNAi oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length. In some embodiments, an oligonucleotide comprises a targeting sequence or a region of complementarity that is complementary to a contiguous sequence of nucleotides of any one of SEQ ID NOs: 4-387, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, an oligonucleotide comprises a targeting sequence or a region of complementarity that is complementary to a contiguous sequence of nucleotides 1-19 of any one of SEQ ID NOs: 4-387, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementarity that is complementary to a contiguous sequence of nucleotides of any one of SEQ ID NOs: 872-878 and 886-911, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementarity that is complementary to a contiguous sequence of nucleotides of any one of SEQ ID NOs: 887, 891, 892, 894, 897, and 909, and the oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length).

In some embodiments, a loop (L) of a stem-loop having the structure S1-L-S2 as described above is a triloop. In some embodiments, the oligonucleotide comprises a targeting sequence or a region of complementarity that is complementary to a contiguous sequence of nucleotides of any one of SEQ ID NOs: 4-387 and a triloop. In some embodiments, the oligonucleotide comprises a targeting sequence or a region of complementarity that is complementary to a contiguous sequence of nucleotides 1-19 of any one of SEQ ID NOs: 4-387 and a triloop. In some embodiments, the oligonucleotide comprises a targeting sequence or a region of complementarity that is complementary to a contiguous sequence of nucleotides of any one of SEQ ID NOs: 872-878 and 886-911, and a triloop. In some embodiments, the oligonucleotide comprises a targeting sequence or a region of complementarity that is complementary to a contiguous sequence of nucleotides of any one of SEQ ID NOs: 887, 891, 892, 894, 897, and 909, and a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure) comprises a targeting sequence or a region of complementarity that is complementary to a contiguous sequence of nucleotides of any one of SEQ ID NOs: 4-387 and a tetraloop. In some embodiments, a loop (L) of a stem-loop having the structure S1-L-S2 as described above is a tetraloop (e.g., within a nicked tetraloop structure) comprises a targeting sequence or a region of complementarity that is complementary to a contiguous sequence of nucleotides 1-19 of any one of SEQ ID NOs: 4-387 and a tetraloop. In some embodiments, the oligonucleotide comprises a targeting sequence or a region of complementarity that is complementary to a contiguous sequence of nucleotides of any one of SEQ ID NOs: 872-878 and 886-911, and a tetraloop. In some embodiments, the oligonucleotide comprises a targeting sequence or a region of complementarity that is complementary to a contiguous sequence of nucleotides of any one of SEQ ID NOs: 887, 891, 892, 894, 897, and 909, and a tetraloop. In some embodiments, the tetraloop comprises ribonucleotides, deoxyribonucleotides, modified nucleotides, delivery ligands, and combinations thereof.

In some embodiments, a dsRNAi oligonucleotide herein comprises a sense strand comprising a nucleotide sequence selected from SEQ ID NOs: 942-947.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

### Oligonucleotide Ends

In some embodiments, a dsRNAi oligonucleotide herein comprises sense and antisense strands, such that there is a 3′-overhang on either the sense strand or the antisense strand, or both the sense and antisense strand. In some embodiments, a dsRNAi oligonucleotide herein comprises sense and antisense strands that are separate strands which form an asymmetric duplex region having an overhang at the 3′ terminus of the antisense strand. In some embodiments, a dsRNAi oligonucleotide provided herein has one 5′end that is thermodynamically less stable compared to the other 5′ end. In some embodiments, an asymmetric dsRNAi oligonucleotide is provided that includes a blunt end at the 3′end of a sense strand and overhang at the 3′ end of the antisense strand. In some embodiments, a 3′ overhang on an antisense strand is 1-8 nucleotides in length (e.g., 1, 2, 3, 4, 5, 6, 7 or 8 nucleotides in length). In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the antisense strand comprises a 3′ overhang of 1-8 nucleotides in length (e.g., 1, 2, 3, 4, 5, 6, 7 or 8 nucleotides in length).

Typically, an oligonucleotide for RNAi has a two (2) nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides. In some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the antisense strand comprises a 5′ overhang of 1-6 nucleotides in length.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, a dsRNAi oligonucleotide herein comprises a stem-loop structure at the 3′ end of the sense strand and comprises two terminal overhang nucleotides at the 3′ end of the antisense strand. In some embodiments, a dsRNAi oligonucleotide herein comprises a nicked tetraloop structure, wherein the 3′ end of the sense strand comprises a stem-tetraloop structure and comprises two terminal overhang nucleotides at the 3′ end of the antisense strand. In some embodiments, the two terminal overhang nucleotides are GG. Typically, one or both of the two terminal GG nucleotides of the antisense strand are not complementary with the target.

In some embodiments, the 5′ end and/or the 3′end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

### Oligonucleotide Modifications

In some embodiments, a dsRNAi oligonucleotide described herein comprises a modification. Oligonucleotides (e.g., dsRNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic or research use.

In some embodiments, the modification is a modified sugar. In some embodiments, the modification is a 5′-terminal phosphate group. In some embodiments, the modification is a modified internucleotide linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleotide linkage, and at least one modified base. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises at least one modified sugar, a 5′-terminal phosphate group, at least one modified internucleotide linkage, and at least one modified base.

The number of modifications on an oligonucleotide (e.g., a dsRNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all the nucleotides of an oligonucleotide are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, a dsRNAi oligonucleotide described herein comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2: e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a dsRNAi oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the dsRNAi oligonucleotide comprises at least about 1 modified nucleotide (e.g., at least 1, at least 5, at least 10, at least 15, at least 20, at least 25, at least 30, at least 35, or more). In some embodiments, the antisense strand of the dsRNAi oligonucleotide comprises at least about 1 modified nucleotide (e.g., at least 1, at least 5, at least 10, at least 15, at least 20, or more).

In some embodiments, all the nucleotides of the sense strand of the dsRNAi oligonucleotide are modified. In some embodiments, all the nucleotides of the antisense strand of the dsRNAi oligonucleotide are modified. In some embodiments, all the nucleotides of the dsRNAi oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides dsRNAi oligonucleotides having different modification patterns. Exemplary modification patterns are set forth in U.S. Provisional Application No. 62/909,278 and in WO 2021/067744, both incorporated herein by this reference. In some embodiments, the modified dsRNAi oligonucleotides comprise a sense strand sequence having a modification pattern as set forth in the Examples and Sequence Listing and an antisense strand having a modification pattern as set forth in the Examples and

### SEQUENCE LISTING

In some embodiments, a dsRNAi oligonucleotide disclosed herein comprises an antisense strand having nucleotides that are modified with 2′-F. In some embodiments, a dsRNAi oligonucleotide disclosed herein comprises an antisense strand comprising nucleotides that are modified with 2′-F and 2′-OMe. In some embodiments, a dsRNAi oligonucleotide disclosed herein comprises a sense strand having nucleotides that are modified with 2′-F. In some embodiments, a dsRNAi oligonucleotide disclosed herein comprises a sense strand comprising nucleotides that are modified with 2′-F and 2′-OMe.

In some embodiments, a dsRNAi oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2-fluoro modification. In some embodiments, a dsRNAi oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the dsRNAi oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the dsRNAi oligonucleotide comprise a 2′-fluoro modification.

In some embodiments, one or more of positions 8, 9, 10 or 11 of the sense strand is modified with a 2′—F group. In some embodiments, one or more of positions 3, 8, 9, 10, 12, 13 and 17 of the sense strand is modified with a 2′—F group. In some embodiments, one or more of positions 2, 3, 4, 5, 7, 10 and 14 of the antisense strand is modified with a 2′—F group. In some embodiments, one or more of positions 2, 3, 4, 5, 7, 8, 10, 14, 16 and 19 of the antisense strand is modified with a 2′—F group. In some embodiments, the sugar moiety at each of nucleotides at positions 1-7 and 12-20 in the sense strand is modified with a 2′-OMe. In some embodiments, the sugar moiety at each of nucleotides at positions 1-7, 12-27 and 31-36 in the sense strand is modified with a 2′-OMe. In some embodiments, the sugar moiety at each of nucleotides at positions 1-2, 4-7, 11, 14-16 and 18-20 in the sense strand is modified with a 2′-OMe. In some embodiments, the sugar moiety at each of nucleotides at positions 1-2, 4-7, 11, 14-16, 18-27 and 31-36 in the sense strand is modified with a 2′-OMe. In some embodiments, the sugar moiety at each of nucleotides at positions 1, 6, 8-9, 11-13, and 15-22 in the antisense strand is modified with a 2′-OMe. In some embodiments, the sugar moiety at each of nucleotides at positions 6, 9, 11-13, 15, 17, 18 and 20-22 in the antisense strand is modified with a 2′-OMe. In some embodiments, the sugar moiety at each of nucleotides at positions 1, 6, 9, 11-13, 15, 17, 18 and 20-22 in the antisense strand is modified with a 2′-OMe.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein one or more of positions 8, 9, 10 or 11 of the sense strand is modified with a 2′—F group.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein one or more of positions 3, 8, 9, 10, 12, 13 and 17 of the sense strand is modified with a 2′—F group.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In other embodiments, the sugar moiety at each of the positions 2, 5 and 14 of the antisense strand is modified with the 2′-F. In other embodiments, the sugar moiety at each of the positions 1, 2, 5 and 14 of the antisense strand is modified with the 2′-F. In still other embodiments, the sugar moiety at each of the positions 1, 2, 3, 5, 7 and 14 of the antisense strand is modified with the 2′-F. In yet another embodiment, the sugar moiety at each of the positions 1, 2, 3, 5, 10 and 14 of the antisense strand is modified with the 2′-F. In another embodiment, the sugar moiety at each of the positions 2, 3, 5, 7, 10 and 14 of the antisense strand is modified with the 2′-F.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 3, 4, 7 and 10 of the antisense strand are modified with a 2′-F. In other embodiments, the sugar moiety at each of positions 2, 5 and 14 of the antisense strand is modified with the 2′-F. In other embodiments, the sugar moiety at each of positions 2, 4, 5 and 14 of the antisense strand is modified with the 2′-F. In still other embodiments, the sugar moiety at each of positions 2, 3, 4, 5, 7 and 14 of the antisense strand is modified with the 2′-F. In yet another embodiment, the sugar moiety at each of positions 2, 3, 4, 5, 10 and 14 of the antisense strand is modified with the 2′-F. In another embodiment, the sugar moiety at each of positions 2, 3, 4, 5, 7, 10 and 14 of the antisense strand is modified with the 2′-F. In some embodiments, the sugar moiety at each of positions 2, 3, 4, 5, 7, 8, 10, 14, 16 and 19 is modified with the 2′-F.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the sugar moiety at one or more positions 2, 3, 4, 5, 7, 10 and 14 of the antisense strand is modified with the 2′-F.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the sugar moiety at one or more positions 2, 3, 4, 5, 7, 8, 10, 14, 16 and 19 of the antisense strand is modified with the 2′-F.

In some embodiments, an oligonucleotide provided herein comprises an antisense strand having the sugar moiety at positions 2 and 14 modified with 2′-F. In some embodiments, an oligonucleotide provided herein comprises an antisense strand having the sugar moiety at positions 2, 5, and 14 modified with 2′-F. In some embodiments, an oligonucleotide provided herein comprises an antisense strand having the sugar moiety at positions 1, 2, 5, and 14 modified with 2′-F. In some embodiments, an oligonucleotide provided herein comprises an antisense strand having the sugar moiety at positions 1, 2, 3, 5, 7, and 14 modified with 2′-F. In some embodiments, an oligonucleotide provided herein comprises an antisense strand having the sugar moiety at positions 1, 2, 3, 5, 10, and 14 modified with 2′-F.

In some embodiments, an oligonucleotide provided herein comprises an antisense strand having the sugar moiety at positions 2 and 14 modified with 2′-F. In some embodiments, an oligonucleotide provided herein comprises an antisense strand having the sugar moiety at positions 2, 5, and 14 modified with 2′-F. In some embodiments, an oligonucleotide provided herein comprises an antisense strand having the sugar moiety at positions 2, 4, 5, and 14 modified with 2′-F. In some embodiments, an oligonucleotide provided herein comprises an antisense strand having the sugar moiety at positions 2, 3, 4, 5, 7, and 14 modified with 2′-F. In some embodiments, an oligonucleotide provided herein comprises an antisense strand having the sugar moiety at positions 2, 3, 4, 5, 10, and 14 modified with 2′-F. In some embodiments, an oligonucleotide provided herein comprises an antisense strand having the sugar moiety at positions 2, 3, 4, 5, 7, 10 and 14 modified with 2′-F. In some embodiments, an oligonucleotide provided herein comprises an antisense strand having the sugar moiety at positions 2, 3, 4, 5, 7, 8, 10, 14, 16 and 19 modified with 2′-F.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein one or more of positions 8, 9, 10 or 11 of the sense strand and one or more positions 2, 3, 4, 5, 7, 10 and 14 of the antisense strand is modified with the 2′-F.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein one or more of positions 3, 8, 9, 10, 12, 13 and 17 of the sense strand and one or more positions 2, 3, 4, 5, 7, 8, 10, 14, 16 and 19 of the antisense strand is modified with the 2′-F.

In some embodiments, an oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 4, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 8, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 3, 8, 9, 10, 12, 13 and 17 modified with 2′-F. In some embodiments, an oligonucleotide provided herein comprises a sense strand having the sugar moiety at positions 1-7 and 12-17 or 12-20 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, an oligonucleotide provided herein comprises a sense strand having the sugar moiety at positions 1-2, 4-7, 11, 14-16 and 18-20 modified with 2′OMe. In some embodiments, an oligonucleotide provided herein comprises a sense strand having the sugar moiety of each of the nucleotides at positions 1-2, 4-7, 11, 14-16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, an oligonucleotide described herein comprises a 5′-terminal phosphate. In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, an oligonucleotide (e.g., a double-stranded oligonucleotide) herein includes analogs of 5′ phosphates that are resistant to such degradation. In some embodiments, the phosphate analog is oxymethylphosphonate, vinylphosphonate or malonylphosphonate, or a combination thereof. In certain embodiments, the 5′ end of an oligonucleotide strand is attached to chemical moiety that mimics the electrostatic and steric properties of a natural 5′-phosphate group (“phosphate mimic”). In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises a 5′-terminal phosphate.

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy.

In some embodiments, a dsRNAi oligonucleotide provided herein comprises an antisense strand comprising a 4′-phosphate analog at the 5′-terminal nucleotide, wherein 5′-terminal nucleotide comprises the following structure:

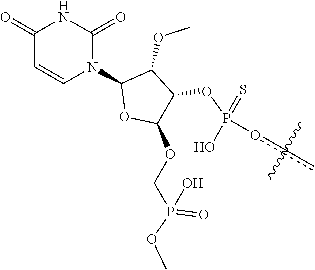

### 5′-methoxyphosphonate-4′-oxy-2′-O-methyluridine [MePhosphonate-40-mUs] phosphorothioate

### Modified Internucleotide Linkage

In some embodiments, an oligonucleotide (e.g., a dsRNAi oligonucleotide) herein comprises a modified internucleotide linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an oligonucleotide provided herein (e.g., a dsRNAi oligonucleotide) has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises a modified internucleotide linkage. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and,
(f) SEQ ID NOs: 887 and 913, respectively;
   wherein the oligonucleotide comprises a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

### Base Modifications

In some embodiments, oligonucleotides herein (e.g., dsRNAi oligonucleotides) have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic). In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target the oligonucleotides of the disclosure (e.g., dsRNAi oligonucleotides) to one or more cells or one or more organs. Such a strategy can help to avoid undesirable effects in other organs or avoid undue loss of the oligonucleotide to cells, tissue or organs that would not benefit from the oligonucleotide. Accordingly, in some embodiments, oligonucleotides disclosed herein (e.g., dsRNAi oligonucleotides) are modified to facilitate targeting and/or delivery to a particular tissue, cell, or organ (e.g., to facilitate delivery of the oligonucleotide to the liver). In some embodiments, an oligonucleotide comprises at least one nucleotide (e.g., 1, 2, 3, 4, 5, 6 or more nucleotides) conjugated to one or more targeting ligand(s). In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises a targeting ligand conjugated to at least once nucleotide,

In some embodiments, the targeting ligand comprises a carbohydrate, amino sugar, cholesterol, peptide, polypeptide, protein, or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a dsRNAi oligonucleotide) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectively, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety targets the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, three (3) or four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (L) has a monovalent GalNAc moiety attached to any one or more guanine nucleotides of the tetraloop via any linker described herein, as depicted below (X=heteroatom):

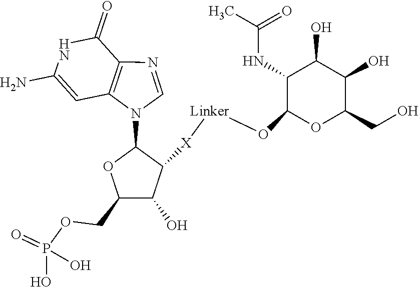

In some embodiments, the tetraloop (L) has a monovalent GalNAc attached to any one or more adenine nucleotides of the tetraloop via any linker described herein, as depicted 5 below (X=heteroatom):

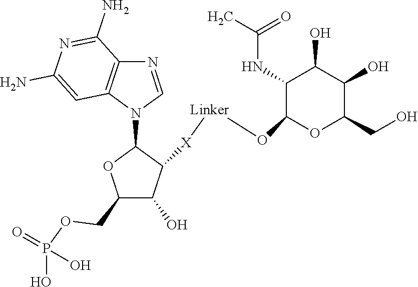

In some embodiments, an oligonucleotide herein comprises a monovalent GalNAc 10 attached to a guanine nucleotide referred to as [ademG-GalNAc] or 2′-aminodiethoxymethanol-Guanine-GalNAc, as depicted below:

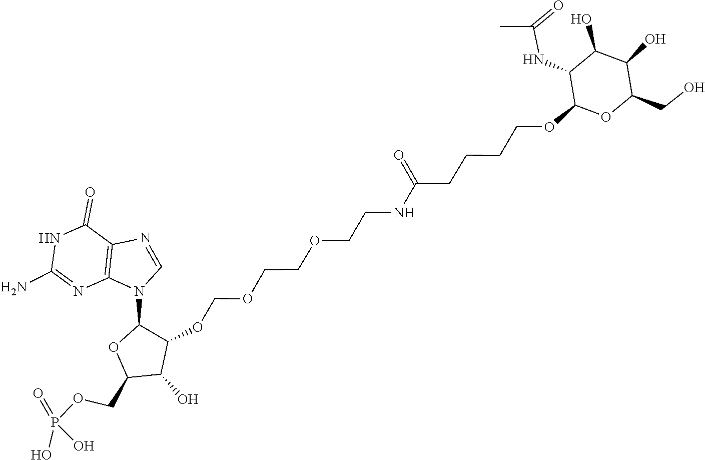 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

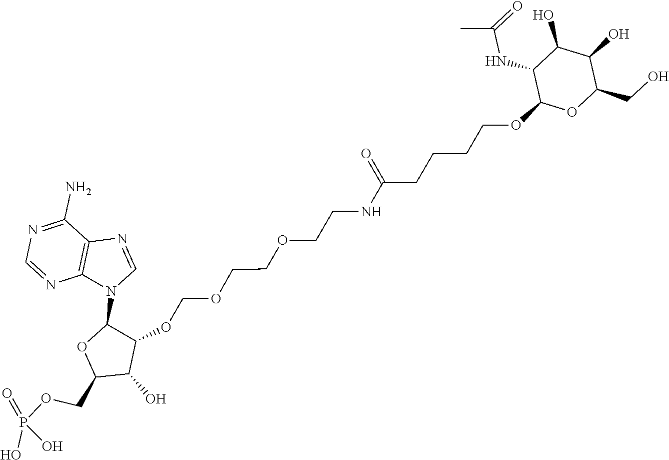 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

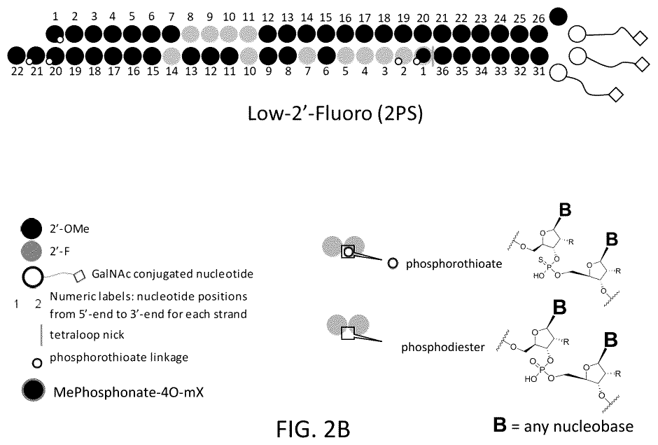

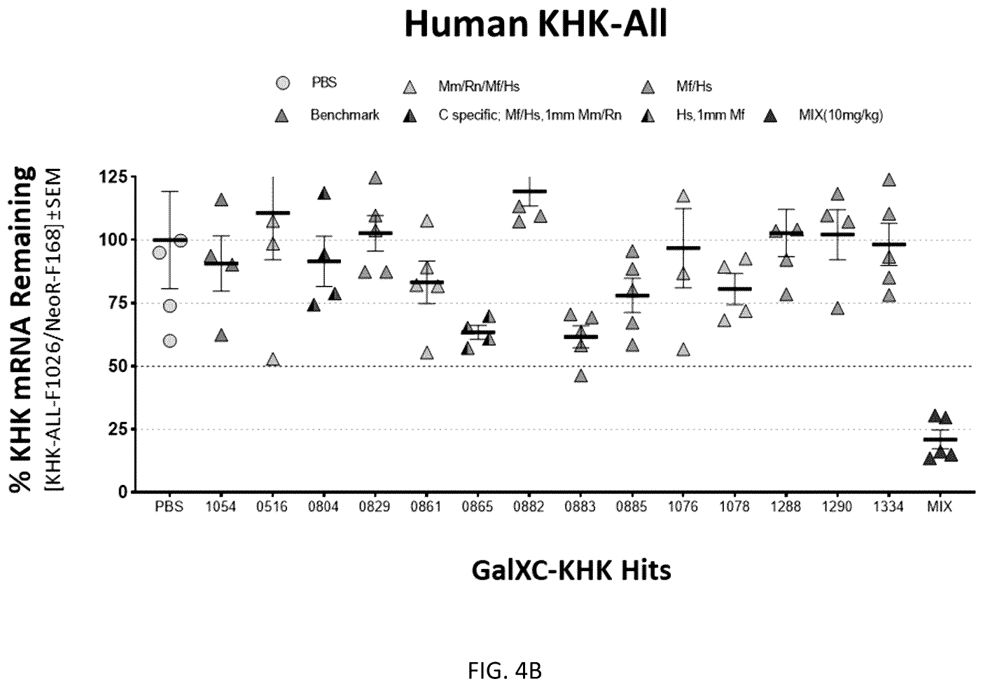

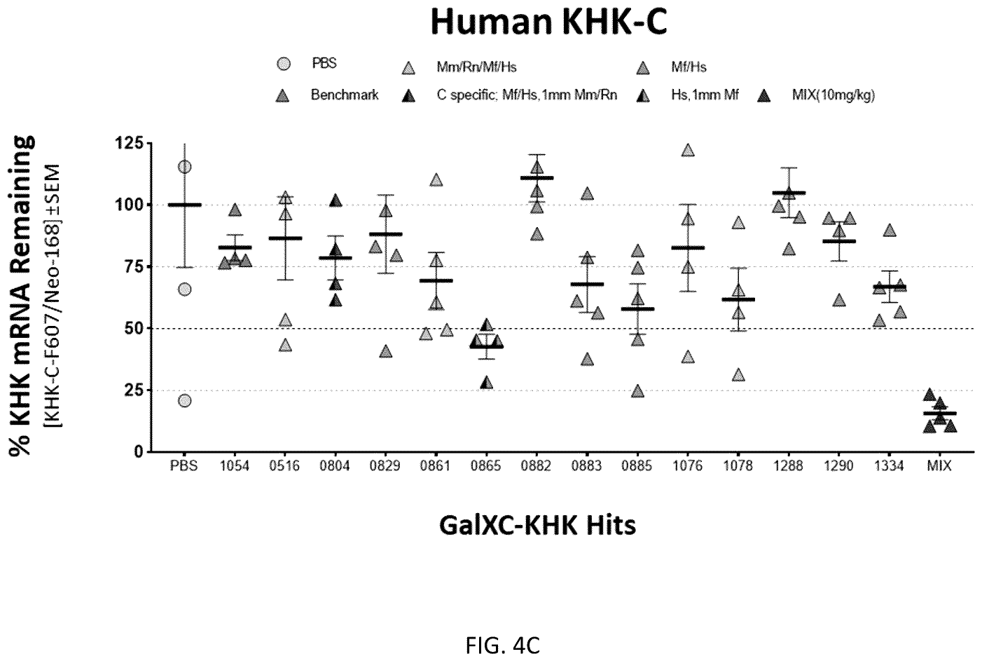

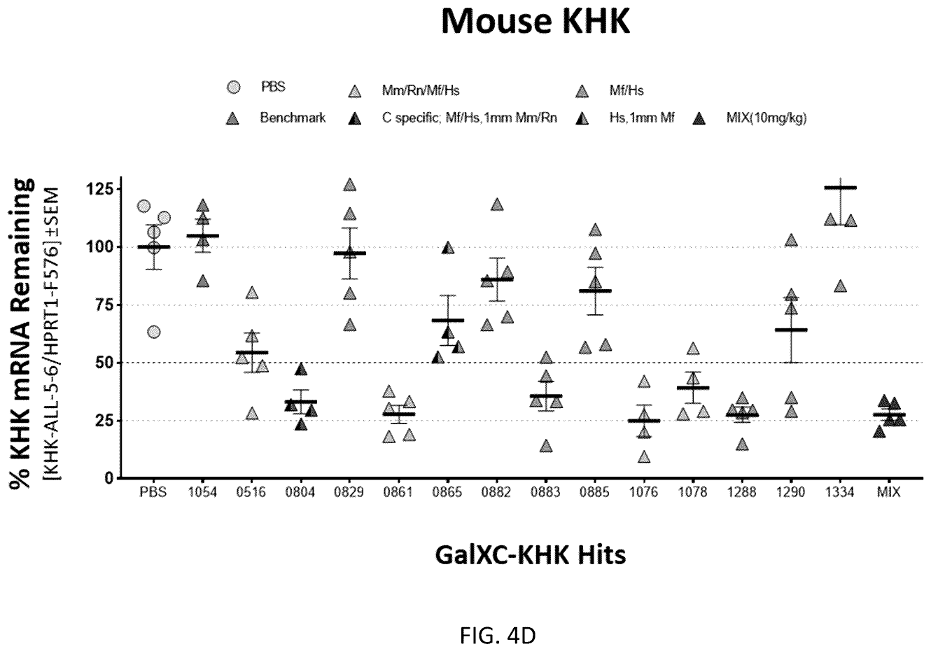

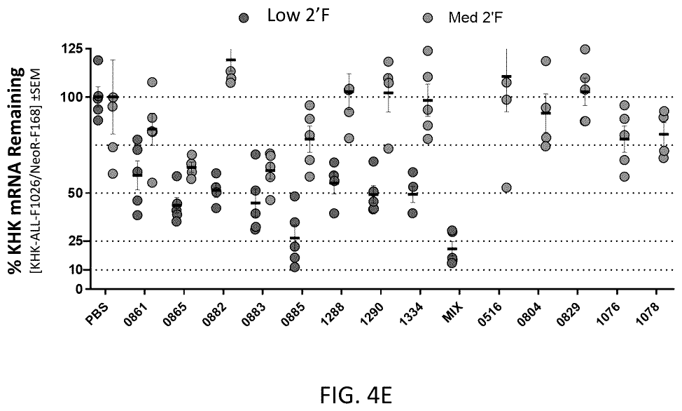

An example of such conjugation is shown below for a loop comprising from 5′ to 3′ the nucleotide sequence GAAA (L=linker, X=heteroatom). Such a loop may be present, for example, at positions 27-30 of a sense strand provided herein, as shown in FIGS. 2 and 4A.

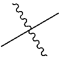

In the chemical formula, is used to describe an attachment point to the oligonucleotide strand.

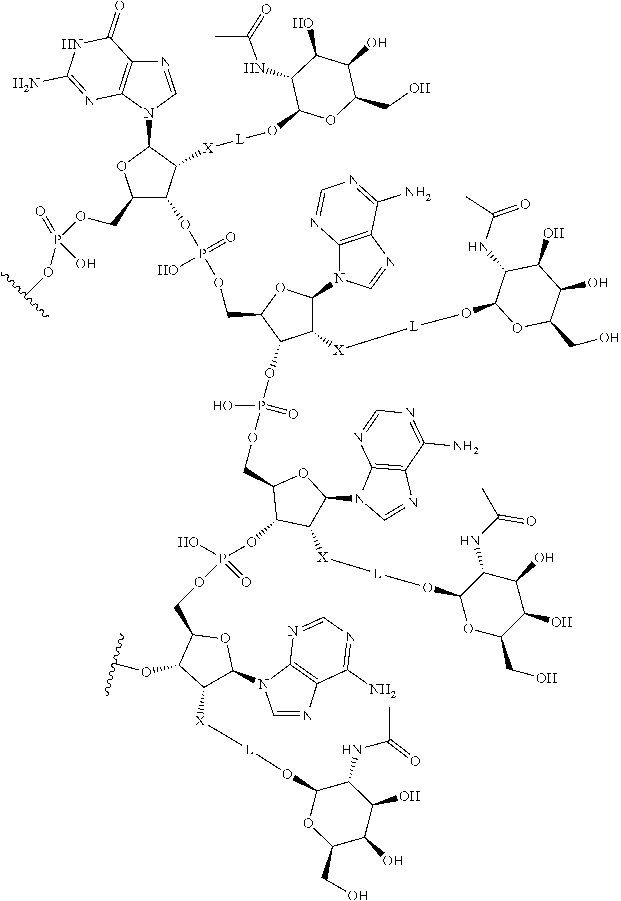

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to nucleotides of the loop using an acetal linker. Such a loop may be present, for example, at positions 27-30 of the sense strand as shown in FIGS. 2 and 4A. In the chemical formula,

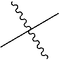

is an attachment point to the oligonucleotide strand.

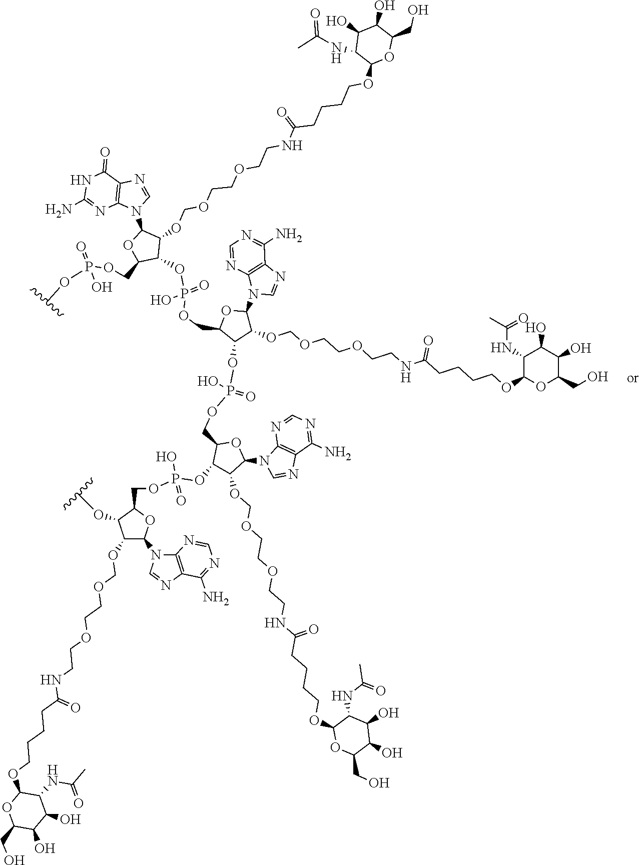 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

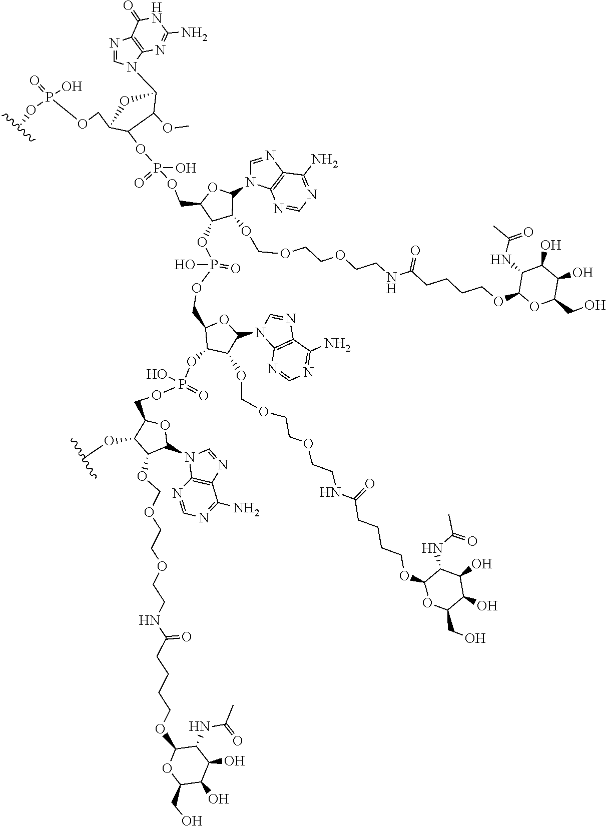

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a dsRNA. In some embodiments, the oligonucleotides herein (e.g., dsRNAi oligonucleotides) do not have a GalNAc conjugated thereto.

In some embodiments, the sense and antisense strands of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide comprises at least one GalNAc moiety conjugated to a nucleotide.
   Exemplary KHK-Targeting dsRNAi Oligonucleotides

In some embodiments, the disclosure provides dsRNAi oligonucleotides that target KHK mRNA and reduce KHK expression (referred to herein as KHK-targeting dsRNAi oligonucleotides), wherein the oligonucleotides comprise a sense strand and an antisense strand that form a duplex region, and wherein the antisense strand comprises a region of complementarity to KHK mRNA target sequence of any one of SEQ ID NOs: 4-387, and wherein the region of complementarity is at least 15 contiguous nucleotides in length. In some embodiments, the disclosure provides dsRNAi oligonucleotides that target KHK mRNA and reduce KHK expression (referred to herein as KHK-targeting dsRNAi oligonucleotides), wherein the oligonucleotides comprise a sense strand and an antisense strand that form a duplex region, and wherein the antisense strand comprises a region of complementarity to KHK mRNA target sequence of nucleotides 1-19 of any one of SEQ ID NOs: 4-387, and wherein the region of complementarity is at least 15 contiguous nucleotides in length. In some embodiments, the region of complementarity is 15-20 nucleotides in length. In some embodiments, the region of complementarity is 15 nucleotides, 16 nucleotides, 17 nucleotides, 18 nucleotides, 19 nucleotides, or 20 nucleotides in length. In some embodiments, the region of complementarity is at least 19 contiguous nucleotides in length. In some embodiments, the region of complementarity is at least 20 nucleotides in length. In some embodiments, the region of complementarity is 19 nucleotides in length. In some embodiments, the region of complementarity is 20 nucleotides in length.

In some embodiments, the sense strand is 15 to 50 nucleotides in length. In some embodiments, the sense strand is 18 to 36 nucleotides in length. In some embodiments, the sense strand comprises a nucleotide sequence selected from SEQ ID NOs: 909, 894, 897, 892, 891 and 887, and is 15 to 50 nucleotides in length. In some embodiments, the sense strand is 36 nucleotides in length. In some embodiments, the antisense strand is 15 to 30 nucleotides in length. In some embodiments, the antisense strand comprises a nucleotide sequence selected from SEQ ID NOs: 936, 920, 923, 917, 918 and 913, and is 15 to 50 nucleotides in length. In some embodiments, the antisense strand is 22 nucleotides in length. In some embodiments, the sense strand is 36 nucleotides in length and the antisense strand is 22 nucleotides in length and the sense and antisense strand form a duplex region that is at least 19 nucleotides in length. In some embodiments, the duplex region is 20 nucleotides in length.

In some embodiments, the KHK-targeting dsRNAi oligonucleotides for reducing KHK expression provided by the disclosure comprise a stem-loop set forth as S1-L-S2, wherein S1 is complementary to S2, and wherein L forms a loop between S1 and S2 of 3-5 nucleotides in length. In some embodiments, S1 and S2 are 1-10 nucleotides in length and are the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments the loop is 3 nucleotides in length. In some embodiments, the loop is 4 nucleotides in length. In some embodiments, the loop is 5 nucleotides in length. In some embodiments, L is a triloop or a tetraloop. In some embodiments, L is a triloop. In some embodiments, L is a tetraloop. In some embodiments, the tetraloop comprises the sequence 5′-GAAA-3′. In some embodiments, the stem loop comprises the sequence 5′-GCAGCCGAAAGGCUGC-3′ (SEQ ID NO: 871). In some embodiments, up to 4 nucleotides comprising L are each conjugated to a targeting ligand. In some embodiments, 1 nucleotide, 2 nucleotides, 3 nucleotides, or 4 nucleotides comprising L are each conjugated to a targeting ligand. In some embodiments, 3 nucleotides comprising L are each conjugated to a targeting ligand. In some embodiments, L is a tetraloop comprising the sequence 5′-GAAA-3′, wherein each adenosine (A) nucleoside comprising the tetraloop is conjugated to a targeting ligand comprising a monovalent N-acetylgalactosamine (GalNAc) moiety.

In some embodiments, the antisense strand comprises a 3′ overhang of one or more nucleotides in length. In some embodiments, the 3′ overhang is two (2) nucleotides in length. In some embodiments, the sequence of the 3′ overhang is 5′-GG-3′.

In some embodiments, the KHK-targeting dsRNAi oligonucleotides for reducing KHK expression provided by the disclosure comprise a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a KHK mRNA target sequence of any one of SEQ ID NOs: 4-387, and wherein the region of complementarity is 19 contiguous nucleotides in length, optionally 20 nucleotides in length. In some embodiments, the KHK-targeting dsRNAi oligonucleotides for reducing KHK expression provided by the disclosure comprise a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a KHK mRNA target sequence of nucleotides 1-19 of any one of SEQ ID NOs: 4-387, and wherein the region of complementarity is 19 contiguous nucleotides in length, optionally 20 nucleotides in length.

In some embodiments, the KHK-targeting dsRNAi oligonucleotides for reducing KHK expression provided by the disclosure comprises at least one modified nucleotide. In some embodiments, the modified nucleotide comprises a five (5) carbon sugar (e.g., ribose) with a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the 2′-modification is 2′-fluoro or 2′-O-methyl. In some embodiments, all nucleotides comprising the KHK-targeting dsRNAi oligonucleotides are modified. In some embodiments, all nucleotides comprising the KHK-targeting dsRNAi oligonucleotides are modified with a 2′-modification selected from 2′-fluoro and 2′-O-methyl. In some embodiments, all nucleotides comprising the KHK-targeting dsRNAi oligonucleotides are modified with a combination of 2′-fluoro and 2′-O-methyl. In some embodiments, the sense and antisense strand of an oligonucleotide comprise nucleotide sequences selected from the group consisting of:
(a) SEQ ID NOs: 909 and 936, respectively;
(b) SEQ ID NOs: 894 and 920, respectively;
(c) SEQ ID NOs: 897 and 923, respectively;
(d) SEQ ID NOs: 892 and 918, respectively;
(e) SEQ ID NOs: 891 and 917, respectively; and
(f) SEQ ID NOs: 887 and 913, respectively;
   wherein the oligonucleotide is modified with a combination of 2′-fluoro and 2′-O-methyl.

In some embodiments, the KHK-targeting dsRNAi oligonucleotides comprises at least one modified internucleotide linkage. In some embodiments, the at least one modified internucleotide linkage is a phosphorothioate linkage.

In some embodiments, the KHK-targeting dsRNAi oligonucleotides comprise an antisense strand wherein the 4′-carbon of the sugar of the 5′-terminal nucleotide of the antisense strand comprises a phosphate analog. In some embodiments, the phosphate analog is oxymethylphosphonate, vinylphosphonate or malonylphosphonate. In some embodiments, the phosphate analog is a 4′-phosphate analog comprising 5′-methoxyphosphonate-4′-oxy.

In some embodiments, the KHK-targeting dsRNAi oligonucleotides for reducing KHK expression provided by the disclosure comprise a sense strand and an antisense strand, wherein all nucleotides comprising the sense strand and antisense strand are modified, wherein the antisense strand comprises a region of complementarity to a KHK mRNA target sequence of any one of SEQ ID NOs: 4-387, and wherein the region of complementarity is at least 15 contiguous nucleotides in length. In some embodiments, the KHK-targeting dsRNAi oligonucleotides for reducing KHK expression provided by the disclosure comprise a sense strand and an antisense strand, wherein all nucleotides comprising the sense strand and antisense strand are modified, wherein the antisense strand comprises a region of complementarity to a KHK mRNA target sequence of nucleotides 1-19 of any one of SEQ ID NOs: 4-387, and wherein the region of complementarity is at least 15 contiguous nucleotides in length. In some embodiments, the 5′-terminal nucleotide of the antisense strand comprises 5′-methoxyphosphonate-4′-oxy-2′-O-methyluridine [MePhosphonate-40-mU], as described herein. In some embodiments, the 5′-terminal nucleotide of the antisense strand comprises a phosphorothioate linkage. In some embodiments, the antisense strand and the sense strand comprise one or more 2′-fluoro (2′-F) and 2′-O-methyl (2′-OMe) modified nucleotides and at least one phosphorothioate linkage. In some embodiments, the antisense strand comprises four (4) phosphorothioate linkages and the sense strand comprises one (1) phosphorothioate linkage. In some embodiments, the antisense strand comprises five (5) phosphorothioate linkages and the sense strand comprises one (1) phosphorothioate linkage.

In some embodiments, the KHK-targeting dsRNAi oligonucleotides for reducing KHK expression comprise:
a sense strand comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 5′-methoxyphosphonate-4′-oxy-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(o) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd SEQ ID NOs: 898 and 924, respectively;
(ee) SEQ ID NOs: 899 and 925, respectively
(ff) SEQ ID NOs: 900 and 926, respectively; and
(gg) SEQ ID NOs: 909 and 936, respectively.

In some embodiments, the KHK-targeting dsRNAi oligonucleotides for reducing KHK expression comprise:
a sense strand comprising a 2′-F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′-F modified nucleotide at positions 2, 3, 4, 5, 7, 10 and 14, a 2′-OMe at positions 1, 6, 8, 9, 11-13, and 15-22, a phosphorothioate linkage between positions 1 and 2, positions 2 and 3, positions 20 and 21, and positions 21 and 22, and a 5′-terminal nucleotide at position 1 comprising a 4′-phosphate analog, optionally wherein the 5′-terminal nucleotide comprises 5′-methoxyphosphonate-4′-oxy-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(o) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd) SEQ ID NOs: 898 and 924, respectively;
(ee) SEQ ID NOs: 899 and 925, respectively
(ff) SEQ ID NOs: 900 and 926, respectively; and
(gg) SEQ ID NOs: 909 and 936, respectively

In some embodiments, a KHK-targeting dsRNAi oligonucleotide for reducing KHK expression provided by the disclosure comprises a sense strand comprising the nucleotide sequence as set forth in SEQ ID NO: 887 and an antisense strand comprising the nucleotide sequence as set forth in SEQ ID NO: 913. In some embodiments, a KHK-targeting dsRNAi oligonucleotide for reducing KHK expression provided by the disclosure comprises a sense strand comprising the nucleotide sequence as set forth in SEQ ID NO: 891 and an antisense strand comprising the nucleotide sequence as set forth in SEQ ID NO: 917. In some embodiments, a KHK-targeting dsRNAi oligonucleotide for reducing KHK expression provided by the disclosure comprises a sense strand comprising the nucleotide sequence as set forth in SEQ ID NO: 892 and an antisense strand comprising the nucleotide sequence as set forth in SEQ ID NO: 918. In some embodiments, a KHK-targeting dsRNAi oligonucleotide for reducing KHK expression provided by the disclosure comprises a sense strand comprising the nucleotide sequence as set forth in SEQ ID NO: 894 and an antisense strand comprising the nucleotide sequence as set forth in SEQ ID NO: 920. In some embodiments, a KHK-targeting dsRNAi oligonucleotide for reducing KHK expression provided by the disclosure comprises a sense strand comprising the nucleotide sequence as set forth in SEQ ID NO: 897 and an antisense strand comprising the nucleotide sequence as set forth in SEQ ID NO: 923. In some embodiments, a KHK-targeting dsRNAi oligonucleotide for reducing KHK expression provided by the disclosure comprises a sense strand comprising the nucleotide sequence as set forth in SEQ ID NO: 909 and an antisense strand comprising the nucleotide sequence as set forth in SEQ ID NO: 936.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 948; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 949; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 950; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 951; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 952; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 953; and (ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 948; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 949; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 950; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 951; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 952; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 953; and (ii) a sense strand of 19-50 nucleotides in length comprising a region of complementarity to the antisense strand and a stem-loop at the 3′terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 948; and (ii) a sense strand of 19-50 nucleotides in length comprising a region of complementarity to the antisense strand, wherein the region of complementarity to the antisense strand is set forth in SEQ ID NO: 942, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 949; and (ii) a sense strand of 19-50 nucleotides in length comprising a region of complementarity to the antisense strand, wherein the region of complementarity to the antisense strand is set forth in SEQ ID NO: 943, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 950; and (ii) a sense strand of 19-50 nucleotides in length comprising a region of complementarity to the antisense strand, wherein the region of complementarity to the antisense strand is set forth in SEQ ID NO: 944, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 951; and (ii) a sense strand of 19-50 nucleotides in length comprising a region of complementarity to the antisense strand, wherein the region of complementarity to the antisense strand is set forth in SEQ ID NO: 945, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 952; and (ii) a sense strand of 19-50 nucleotides in length comprising a region of complementarity to the antisense strand, wherein the region of complementarity to the antisense strand is set forth in SEQ ID NO: 946, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 953; and (ii) a sense strand of 19-50 nucleotides in length comprising a region of complementarity to the antisense strand, wherein the region of complementarity to the antisense strand is set forth in SEQ ID NO: 947, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 948; and (ii) a sense strand of 19-50 nucleotides in length comprising a region of complementarity to the antisense strand and a stem-loop at the 3′terminus, wherein the region of complementarity to the antisense strand is set forth in SEQ ID NO: 942, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 949; and (ii) a sense strand of 19-50 nucleotides in length comprising a region of complementarity to the antisense strand and a stem-loop at the 3′terminus, wherein the region of complementarity to the antisense strand is set forth in SEQ ID NO: 943, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 950; and (ii) a sense strand of 19-50 nucleotides in length comprising a region of complementarity to the antisense strand and a stem-loop at the 3′terminus, wherein the region of complementarity to the antisense strand is set forth in SEQ ID NO: 944, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 951; and (ii) a sense strand of 19-50 nucleotides in length comprising a region of complementarity to the antisense strand and a stem-loop at the 3′terminus, wherein the region of complementarity to the antisense strand is set forth in SEQ ID NO: 945, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 952; and (ii) a sense strand of 19-50 nucleotides in length comprising a region of complementarity to the antisense strand and a stem-loop at the 3′terminus, wherein the region of complementarity to the antisense strand is set forth in SEQ ID NO: 946, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises (i) an antisense strand of 19-30 nucleotides in length, wherein the antisense strand comprises a nucleotide sequence comprising a region of complementarity to a KHK mRNA target sequence, wherein the region of complementarity is set forth in SEQ ID NO: 953; and (ii) a sense strand of 19-50 nucleotides in length comprising a region of complementarity to the antisense strand and a stem-loop at the 3′terminus, wherein the region of complementarity to the antisense strand is set forth in SEQ ID NO: 947, wherein the stem-loop is set forth as S1-L-S2, wherein S1 is complementary to S2 and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, a KHK-targeting dsRNAi oligonucleotide for reducing KHK expression comprises the modification pattern of:

Sense Strand:
5′-mX-S-mX-mX-mX-mX-mX-mX-fX-fX-fX-fX-mX-mX-mX-
mX-mX-mX-mX-mX-mX-mX-mX-mX-mX-mX-mX-mX-[ademA-
GalNAc]-[ademA-GalNAc]-[ademA-GalNAc]-mX-mX-mX-
mX-mX-mX-3′.
Hybridized to:
Antisense Strand:
5′-[MePhosphonate-4O-mX]-S-fX-S-fX-S-fX-fX-mX-
fX-mX-mX-fX-mX-mX-mX-fX-mX-mX-mX-mX-mX-mX-S-mX-
S-mX-3′;

wherein mX=2′-O-methyl modified nucleotide, fX=2′-fluoro modified nucleotide, —S—=phosphorothioate linkage, =phosphodiester linkage, [MePhosphonate-4O-mX]=5′-methoxyphosphonate-4-oxy modified nucleotide, and ademA-GalNAc=GalNAc attached to an adenine nucleotide

In some embodiments, a KHK-targeting dsRNAi oligonucleotide for reducing KHK expression comprises the modification pattern of

Sense Strand:
5′-mX-S-mX-fX-mX-mX-mX-mX-fX-fX-fX-mX-fX-fX-mX-
mX-mX-fX-mX-mX-mX-mX-mX-mX-mX-mX-mX-mX-[ademA-
GalNAc]-[ademA-GalNAc]-[ademA-GalNAc]-mX-mX-mX-
mX-mX-mX-3′.
Hybridized to:
Antisense Strand:
5′-[MePhosphonate-40-mX]-S-fX-S-fX-S-fX-fX-mX-
fX-fX-mX-fX-mX-mX-mX-fX-mX-fX-mX-mX-fX-mX-S-mX-
S-mX-3′;

wherein mX=2′-O-methyl modified nucleotide, fX=2′-fluoro modified nucleotide,-S-=phosphorothioate linkage,-=phosphodiester linkage, [MePhosphonate-40-mX]=5′-methoxyphosphonate-4-oxy modified nucleotide, and ademA-GalNAc=GalNAc attached to an adenine nucleotide

In some embodiments, a KHK-targeting dsRNAi oligonucleotide for reducing KHK expression provided by the disclosure comprising a sense strand selected from SEQ ID NOs: 774-804 and antisense strand selected from SEQ ID NOs: 819-849. In some embodiments, a KHK-targeting dsRNAi oligonucleotide for reducing KHK expression provided by the disclosure comprises a sense strand comprising the nucleotide sequence as set forth in SEQ ID NO: 775 and an antisense strand comprising the nucleotide sequence as set forth in SEQ ID NO: 820. In some embodiments, a KHK-targeting dsRNAi oligonucleotide for reducing KHK expression provided by the disclosure comprises a sense strand comprising the nucleotide sequence as set forth in SEQ ID NO: 779 and an antisense strand comprising the nucleotide sequence as set forth in SEQ ID NO: 824. In some embodiments, a KHK-targeting dsRNAi oligonucleotide for reducing KHK expression provided by the disclosure comprises a sense strand comprising the nucleotide sequence as set forth in SEQ ID NO: 780 and an antisense strand comprising the nucleotide sequence as set forth in SEQ ID NO: 825. In some embodiments, a KHK-targeting dsRNAi oligonucleotide for reducing KHK expression provided by the disclosure comprises a sense strand comprising the nucleotide sequence as set forth in SEQ ID NO: 782 and an antisense strand comprising the nucleotide sequence as set forth in SEQ ID NO: 827. In some embodiments, a KHK-targeting dsRNAi oligonucleotide for reducing KHK expression provided by the disclosure comprises a sense strand comprising the nucleotide sequence as set forth in SEQ ID NO: 785 and an antisense strand comprising the nucleotide sequence as set forth in SEQ ID NO: 830. In some embodiments, a KHK-targeting dsRNAi oligonucleotide for reducing KHK expression provided by the disclosure comprises a sense strand comprising the nucleotide sequence as set forth in SEQ ID NO: 804 and an antisense strand comprising the nucleotide sequence as set forth in SEQ ID NO: 849.

In some embodiments, the KHK-targeting dsRNAi oligonucleotides sense and antisense strands comprise nucleotide sequences selected from the group consisting of:
(a) SEQ ID NOs: 774 and 819, respectively;
(b) SEQ ID NOs: 775 and 820, respectively;
(c) SEQ ID NOs: 776 and 821, respectively;
(d) SEQ ID NOs: 777 and 822, respectively;
(e) SEQ ID NOs: 778 and 823, respectively;
(f) SEQ ID NOs: 779 and 824, respectively;
(g) SEQ ID NOs: 780 and 825, respectively;
(h) SEQ ID NOs: 781 and 826, respectively;
(i) SEQ ID NOs: 782 and 827, respectively;
(j) SEQ ID NOs: 783 and 828, respectively;
(k) SEQ ID NOs: 784 and 829, respectively;
(l) SEQ ID NOs: 785 and 830, respectively;
(m) SEQ ID NOs: 786 and 831, respectively;
(n) SEQ ID NOs: 787 and 832, respectively;
(0) SEQ ID NOs: 788 and 833, respectively;
(p) SEQ ID NOs: 789 and 834, respectively;
(q) SEQ ID NOs: 790 and 835, respectively;
(r) SEQ ID NOs: 791 and 836, respectively;
(s) SEQ ID NOs: 792 and 837, respectively;
(t) SEQ ID NOs: 793 and 838, respectively;
(u) SEQ ID NOs: 794 and 839, respectively;
(v) SEQ ID NOs: 795 and 840, respectively;
(w) SEQ ID NOs: 796 and 841, respectively;
(x) SEQ ID NOs: 797 and 842, respectively;
(y) SEQ ID NOs: 798 and 843, respectively;
(z) SEQ ID NOs: 799 and 844, respectively;
(aa) SEQ ID NOs: 800 and 845, respectively;
(bb) SEQ ID NOs: 801 and 846, respectively;
(cc) SEQ ID NOs: 802 and 847, respectively;
(dd) SEQ ID NOs: 803 and 848, respectively; and
(ee) SEQ ID NOs: 804 and 849, respectively.

In some embodiments, a KHK-targeting dsRNAi oligonucleotide for reducing KHK expression provided by the disclosure comprising a sense strand selected from SEQ ID NOs: 805-818 and an antisense strand selected from SEQ ID NOs: 850-863.

In some embodiments, the KHK-targeting dsRNAi oligonucleotides sense and antisense strands comprise nucleotide sequences selected from the group consisting of:
(a) SEQ ID NOs: 805 and 850, respectively;
(b) SEQ ID NOs: 806 and 851, respectively;
(c) SEQ ID NOs: 807 and 852, respectively;
(d) SEQ ID NOs: 808 and 853, respectively;
(e) SEQ ID NOs: 809 and 854, respectively;
(f) SEQ ID NOs: 810 and 855, respectively;
(g) SEQ ID NOs: 811 and 856, respectively;
(h) SEQ ID NOs: 812 and 857, respectively;
(i) SEQ ID NOs: 813 and 858, respectively;
(j) SEQ ID NOs: 814 and 859, respectively;
(k) SEQ ID NOs: 815 and 860, respectively;
(l) SEQ ID NOs: 816 and 861, respectively;
(m) SEQ ID NOs: 817 and 862, respectively and;
(n) SEQ ID NOs: 818 and 863, respectively.

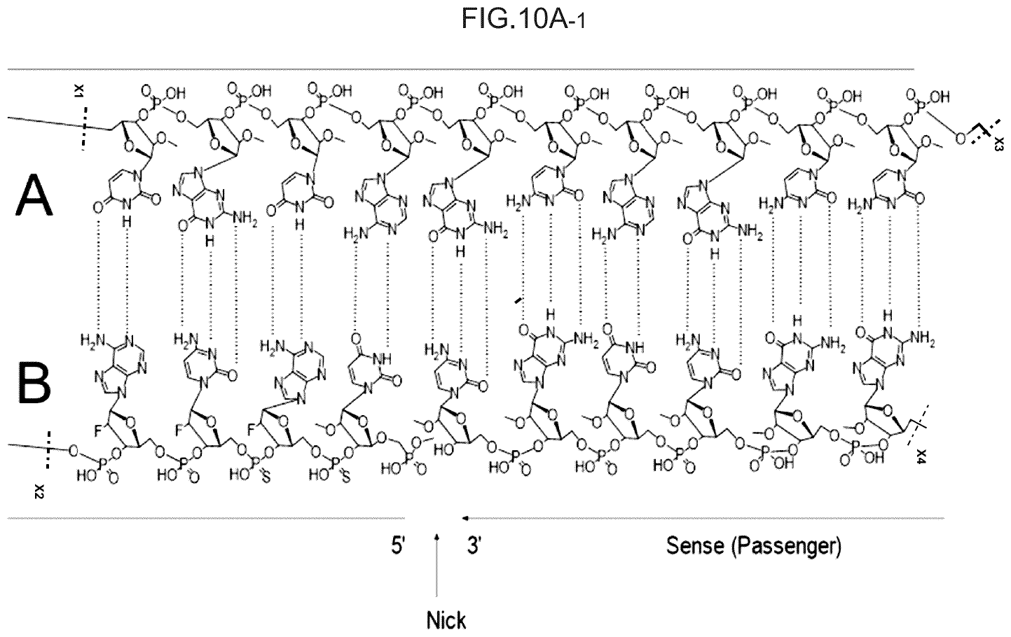

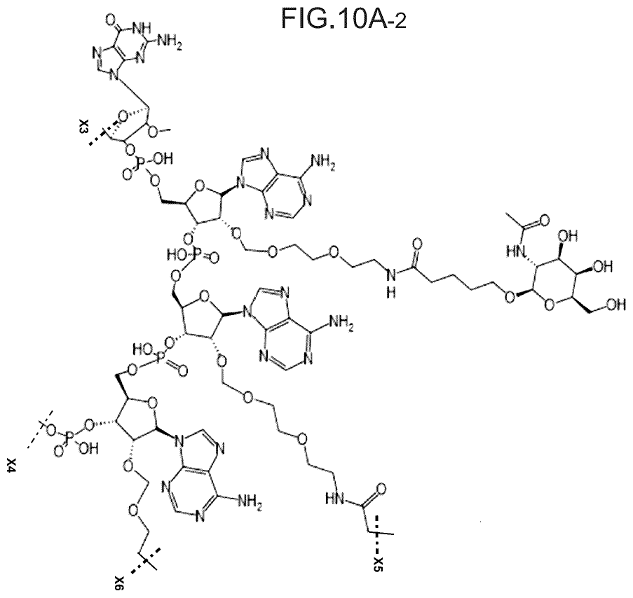

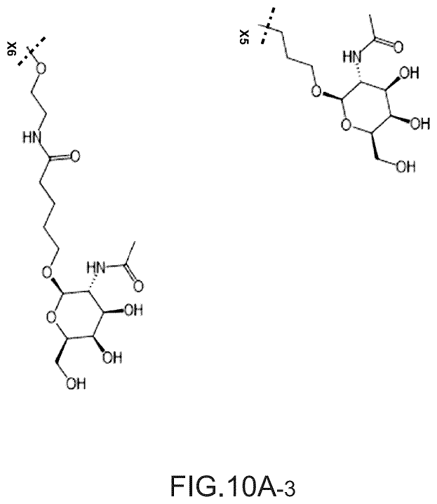

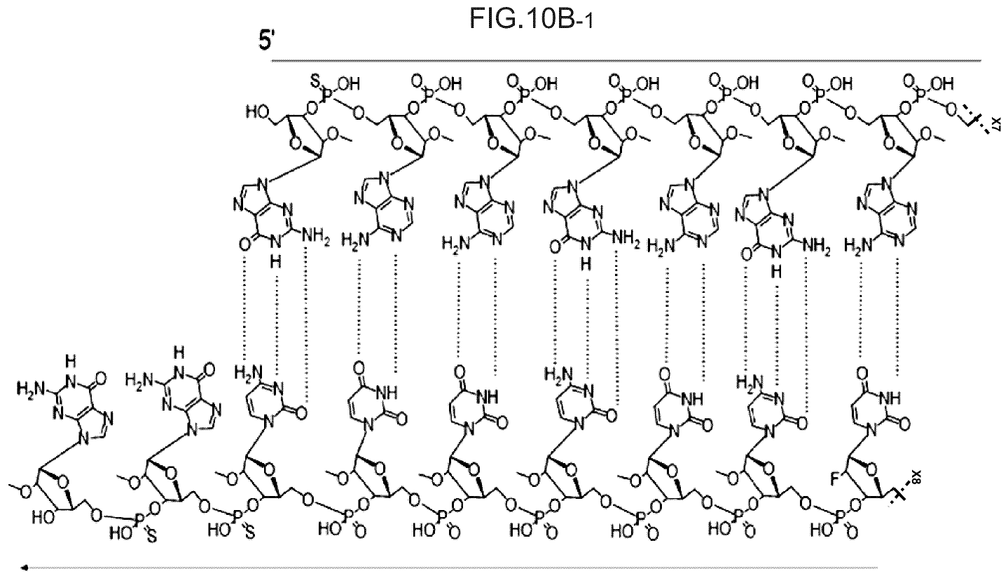

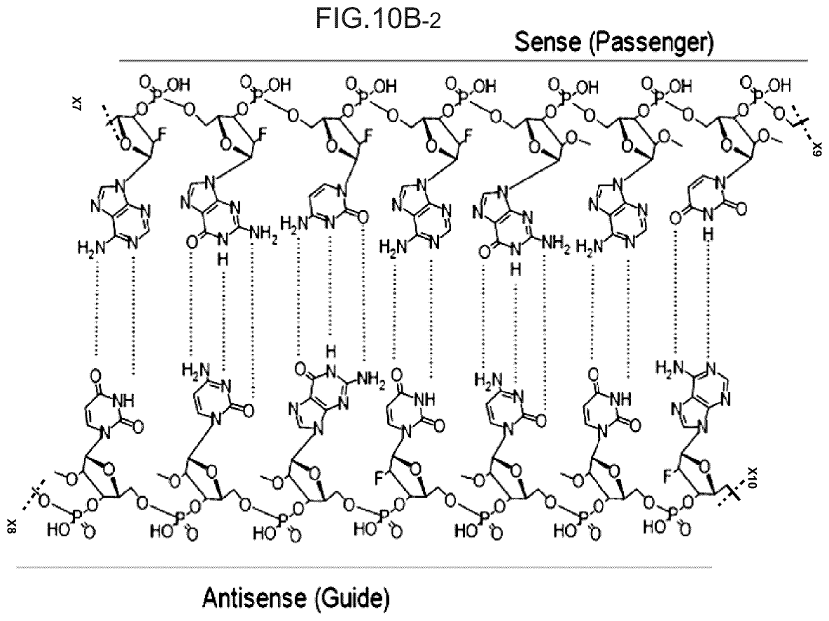

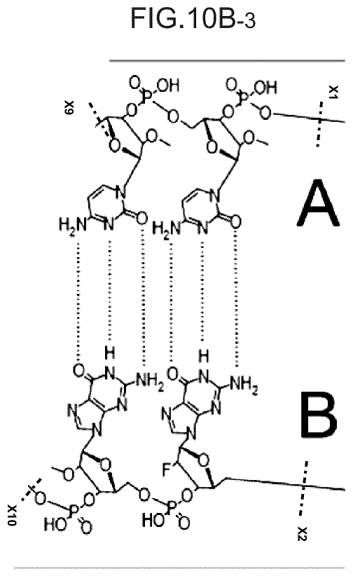

In some embodiments, the KHK-targeting dsRNAi oligonucleotides comprise a sense strand comprising SEQ ID NO: 775 and an antisense strand comprising SEQ ID NO: 820, wherein said dsRNA is in the form of a conjugate having the structure as shown in FIG. 10A continuing to FIG. 10B, or pharmaceutically acceptable salts thereof.

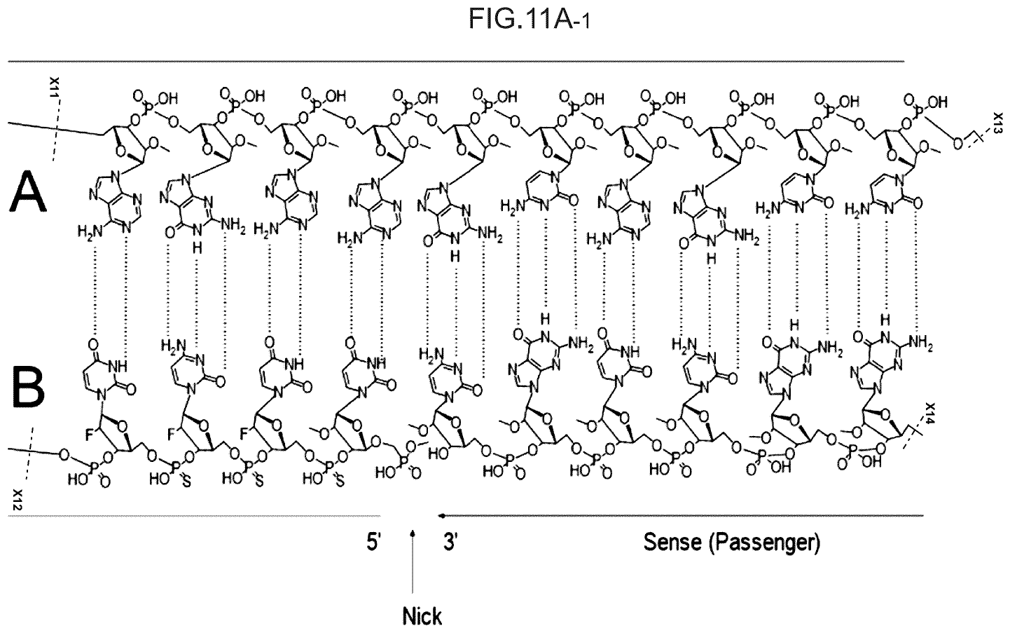

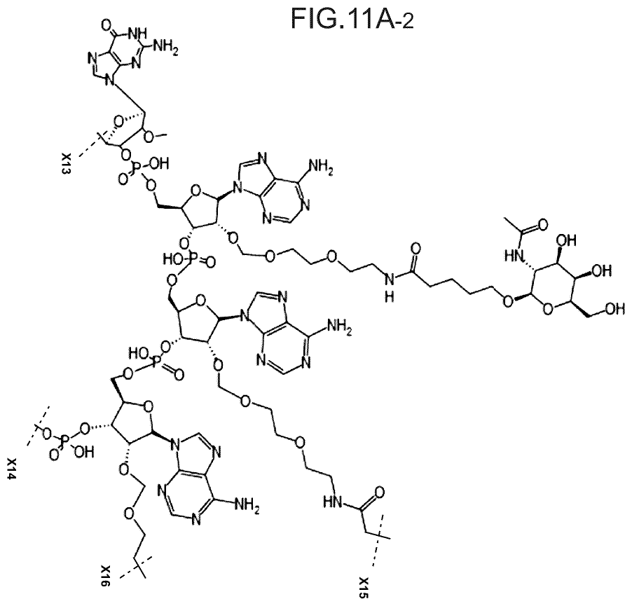

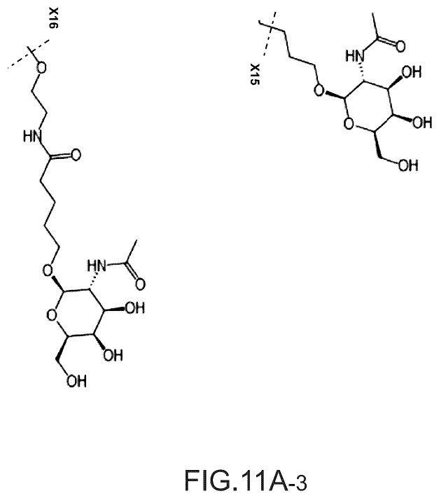

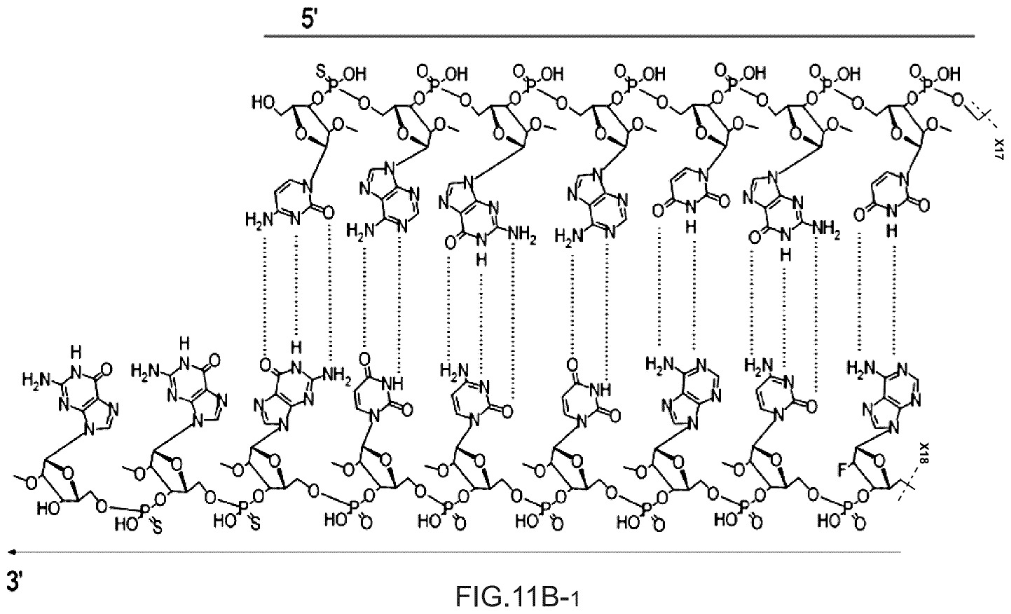

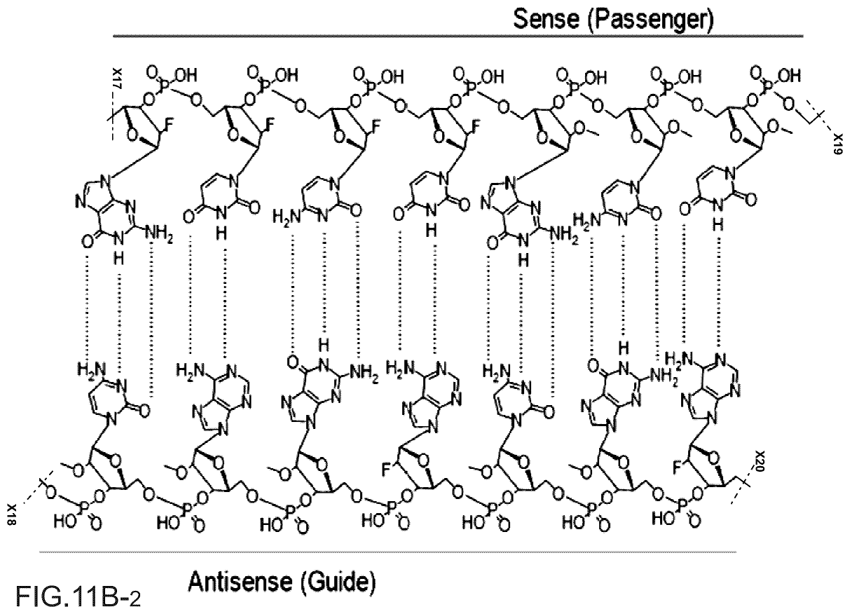

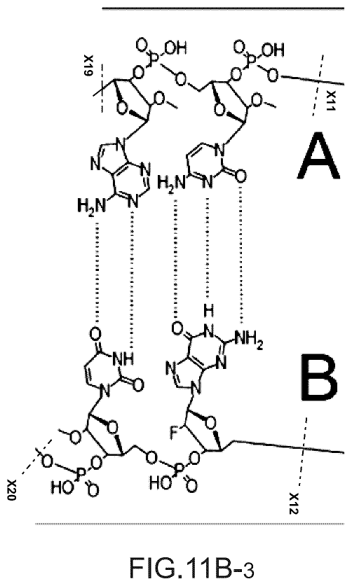

In some embodiments, the KHK-targeting dsRNAi oligonucleotides comprise a sense strand comprising SEQ ID NO: 779 and an antisense strand comprising SEQ ID NO: 824, wherein said dsRNA is in the form of a conjugate having as shown in FIG. 11A continuing to FIG. 11B, or pharmaceutically acceptable salts thereof.

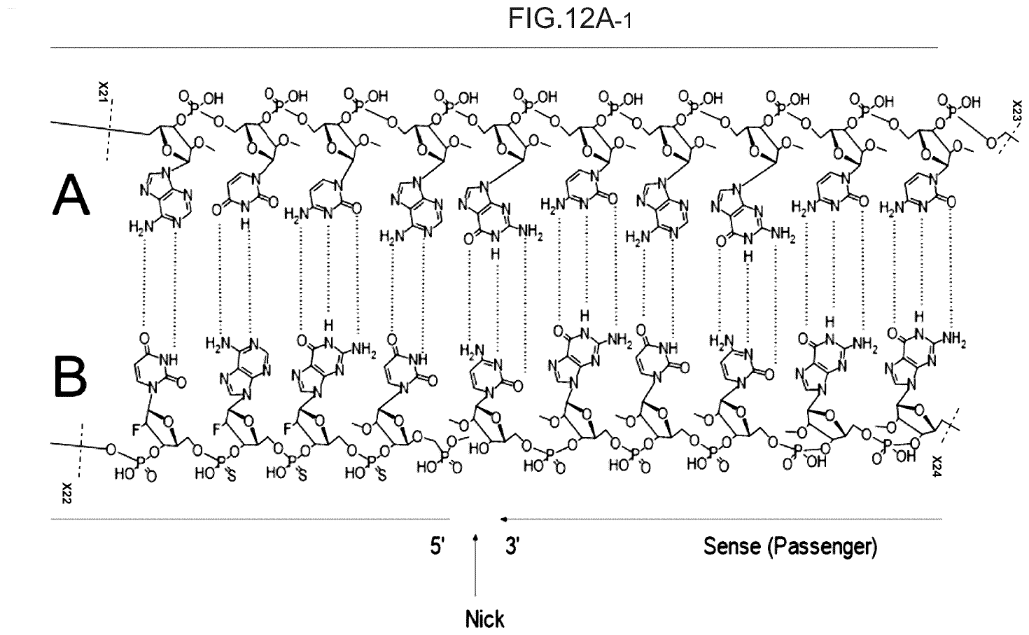

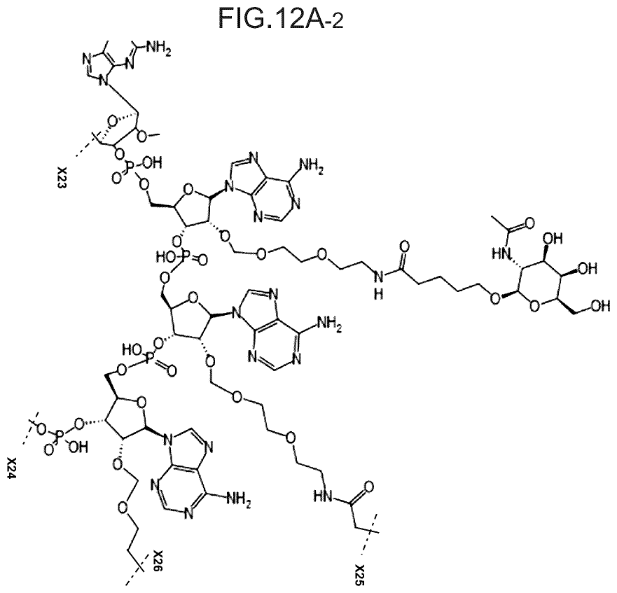

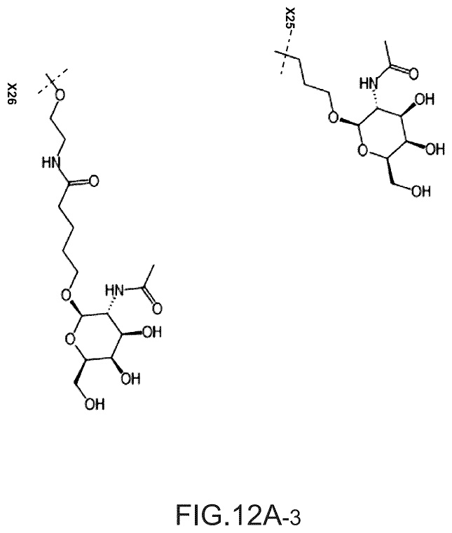

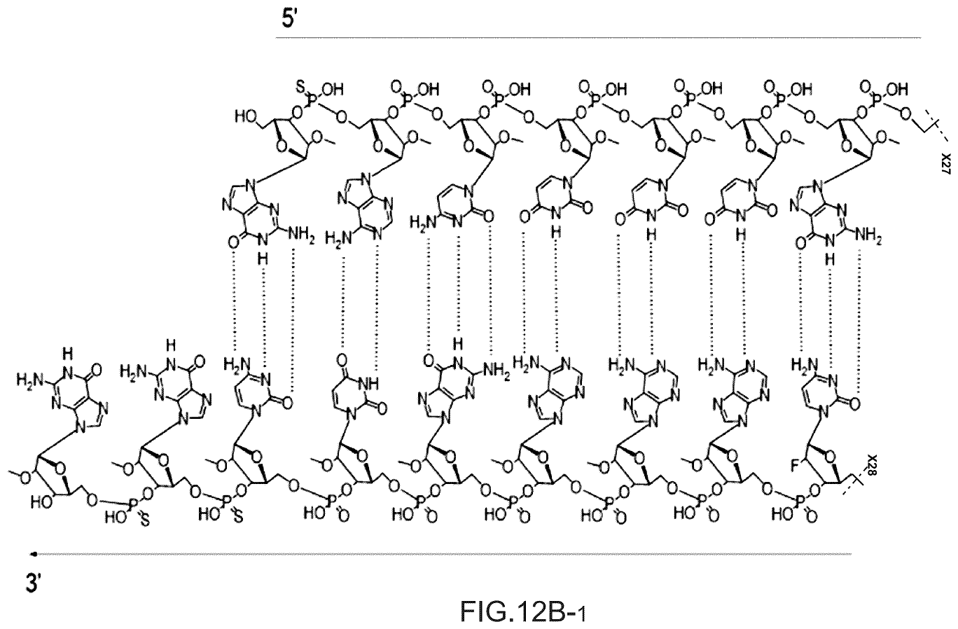

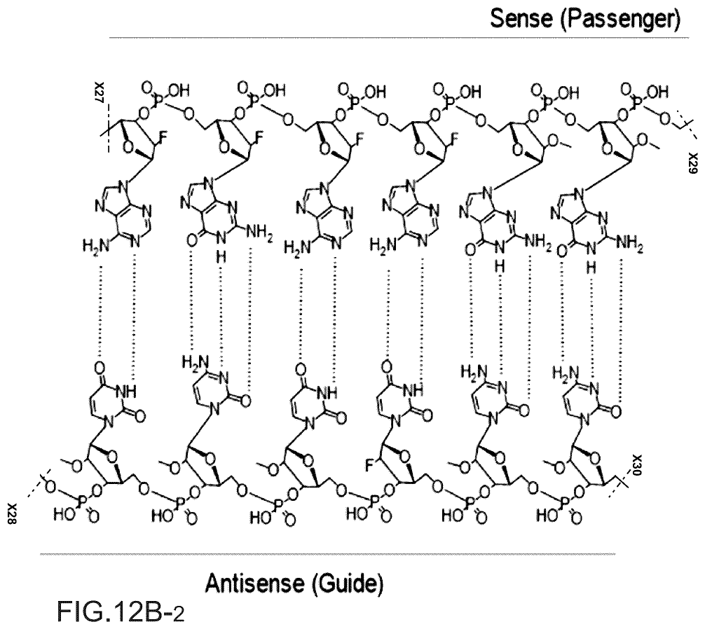

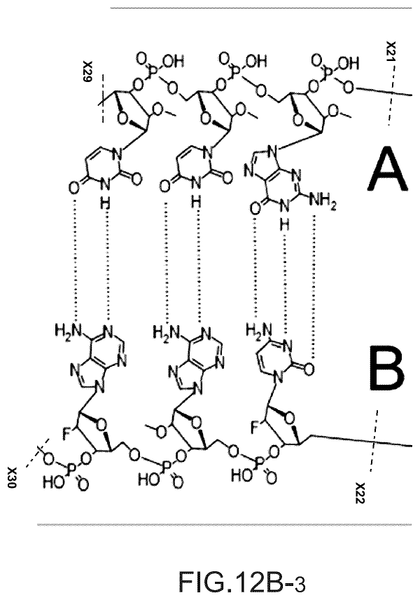

In some embodiments, the KHK-targeting dsRNAi oligonucleotides comprise a sense strand comprising SEQ ID NO: 780 and an antisense strand comprising SEQ ID NO: 825, wherein said dsRNA is in the form of a conjugate as depicted in FIG. 12A continuing to FIG. 12B, or pharmaceutically acceptable salts thereof.

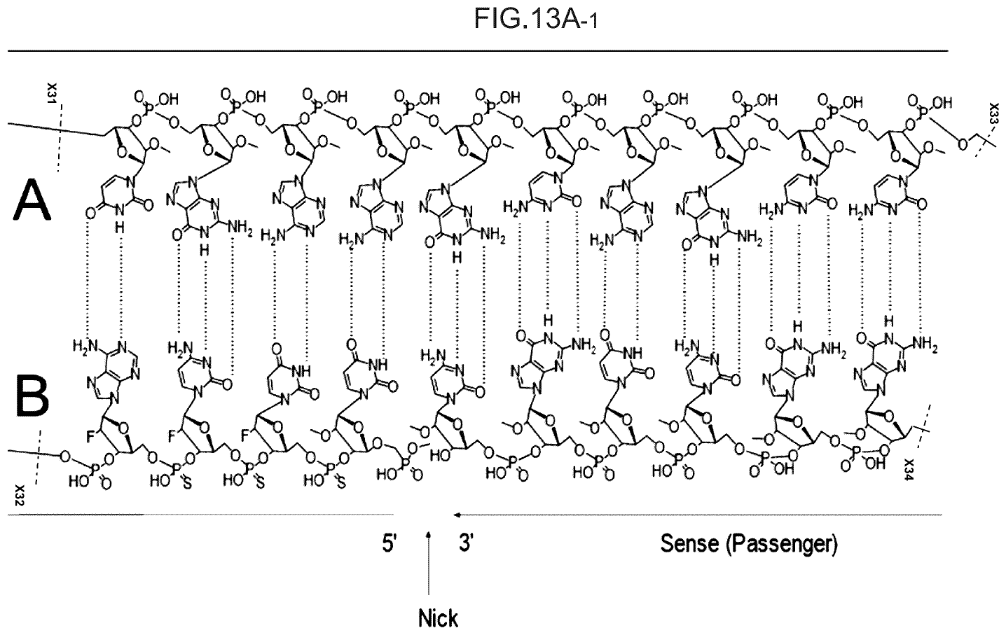

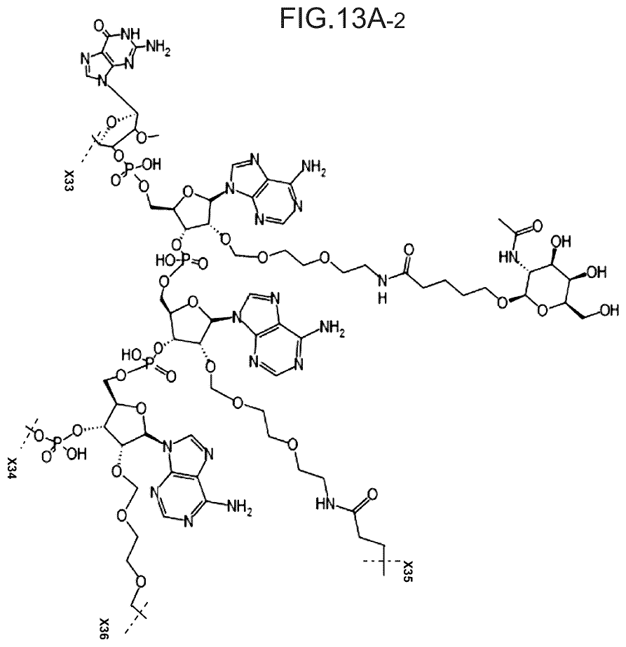

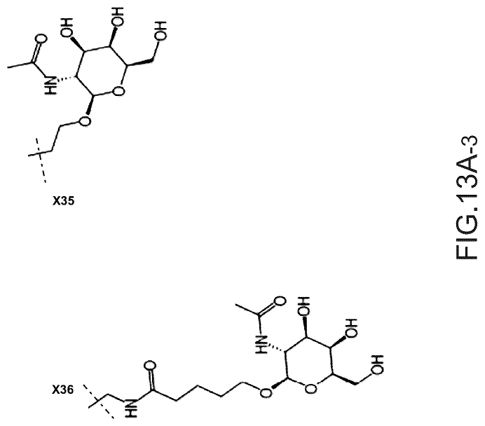

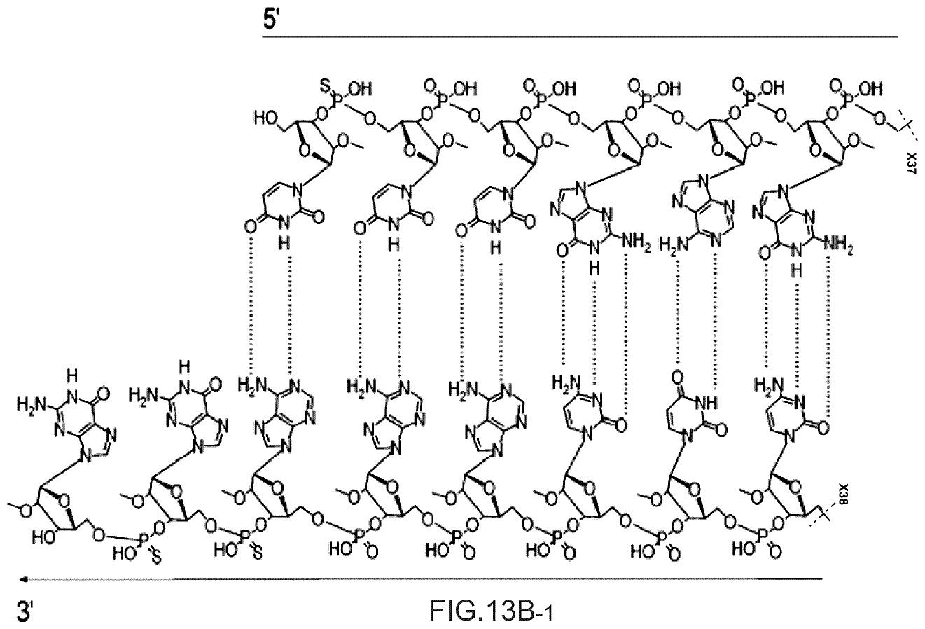

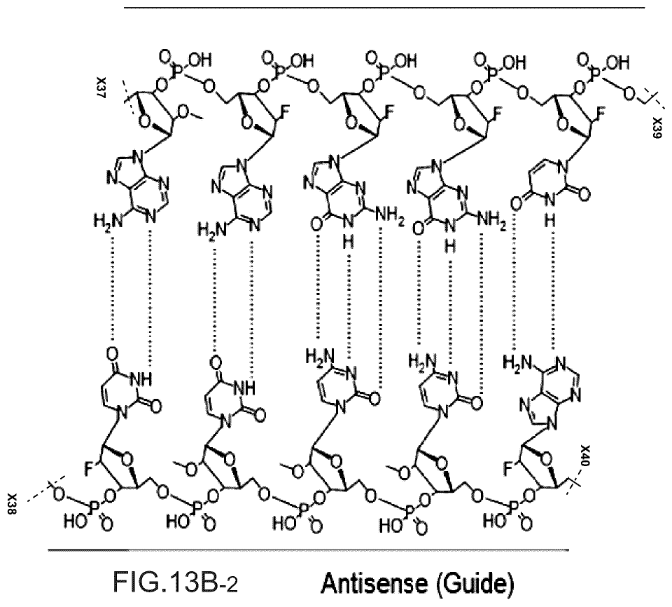

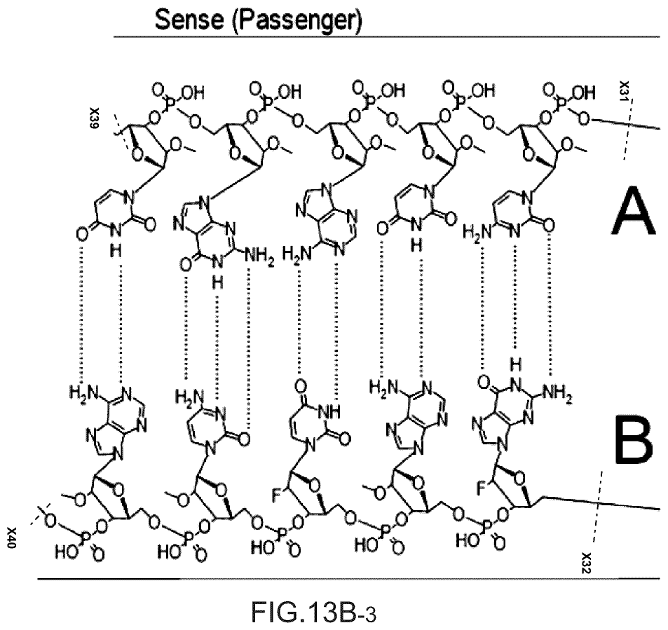

In some embodiments, the KHK-targeting dsRNAi oligonucleotides comprise a sense strand comprising SEQ ID NO: 782 and an antisense strand comprising SEQ ID NO: 827, wherein said dsRNA is in the form of a conjugate having the structures depicted in FIG. 13A continuing to FIG. 13B, or pharmaceutically acceptable salts thereof.

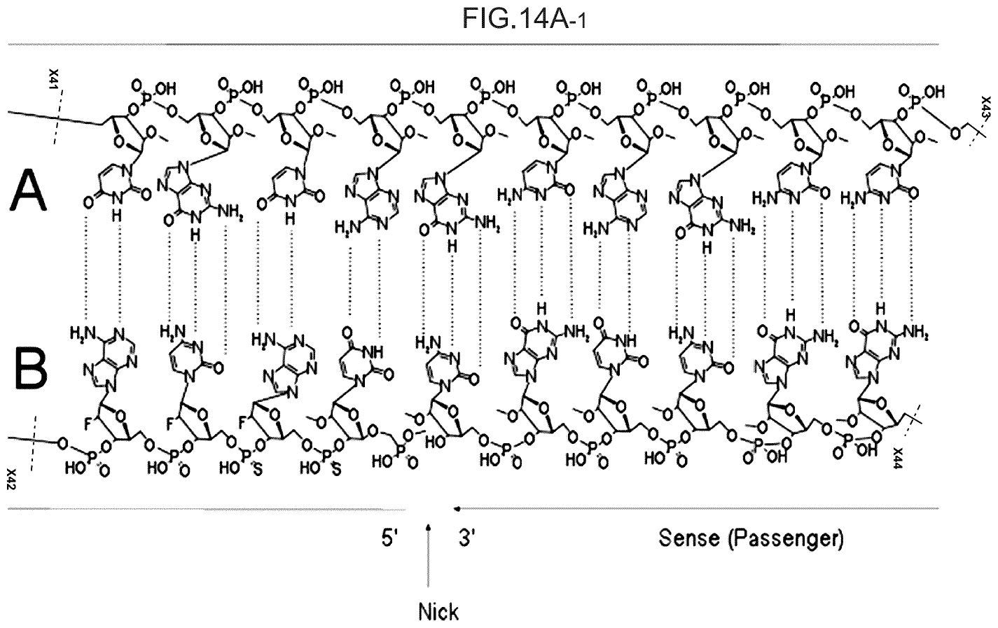

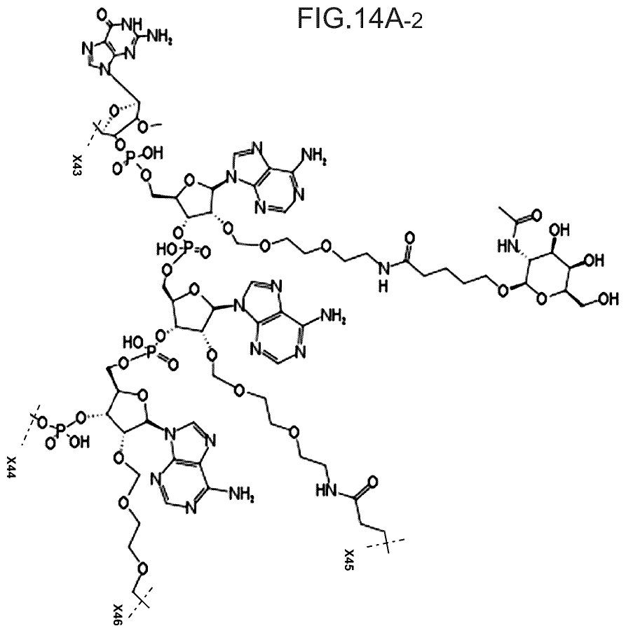

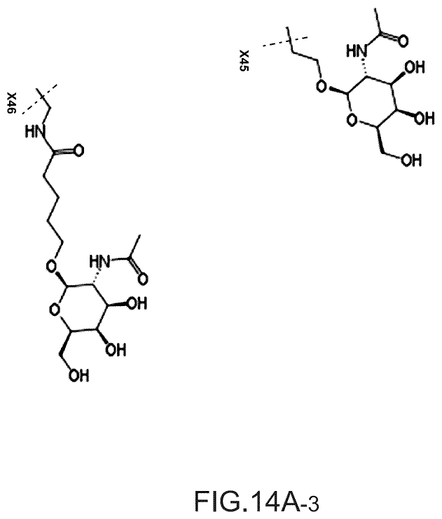

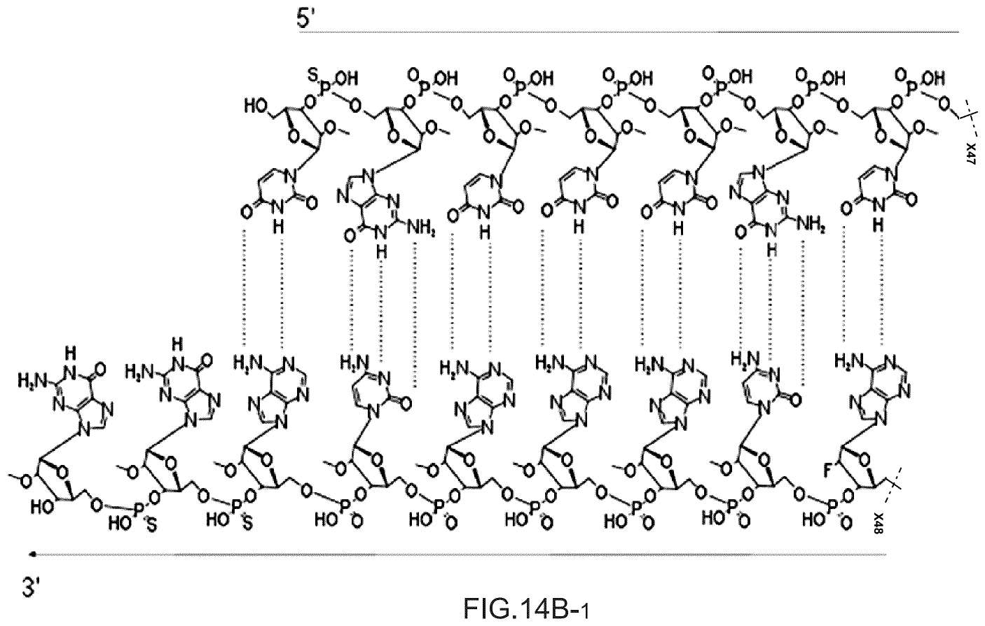

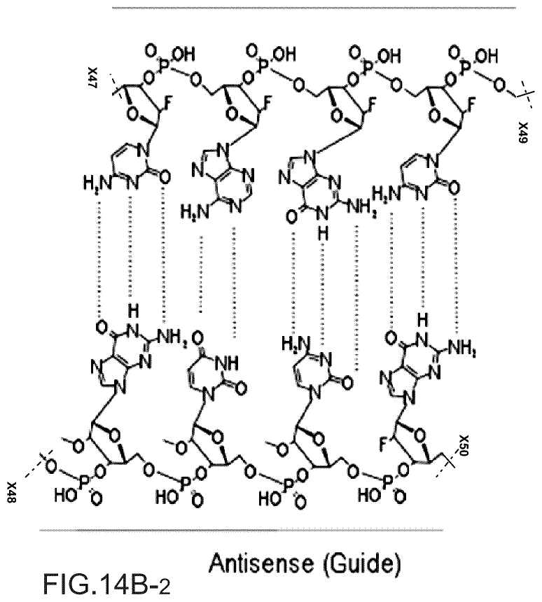

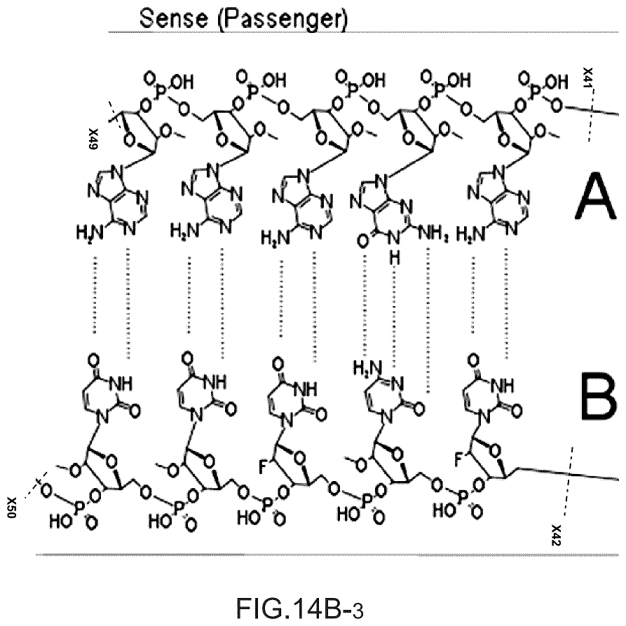

In some embodiments, the KHK-targeting dsRNAi oligonucleotides comprise a sense strand comprising SEQ ID NO: 785 and an antisense strand comprising SEQ ID NO: 830, wherein said dsRNA is in the form of a conjugate having the structures depicted in FIG. 14A continuing to FIG. 14B, or pharmaceutically acceptable salts thereof.

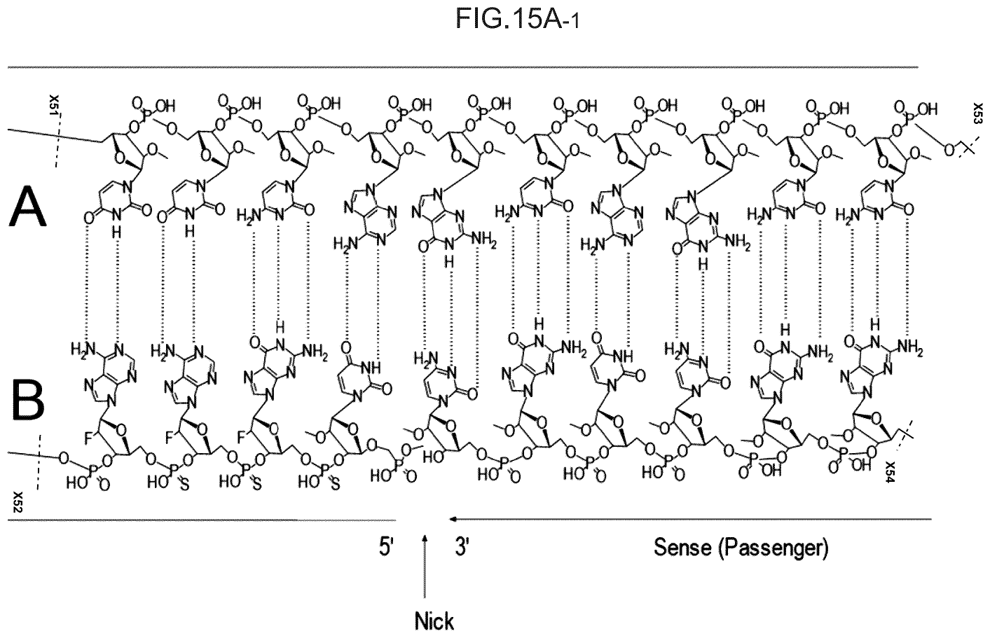

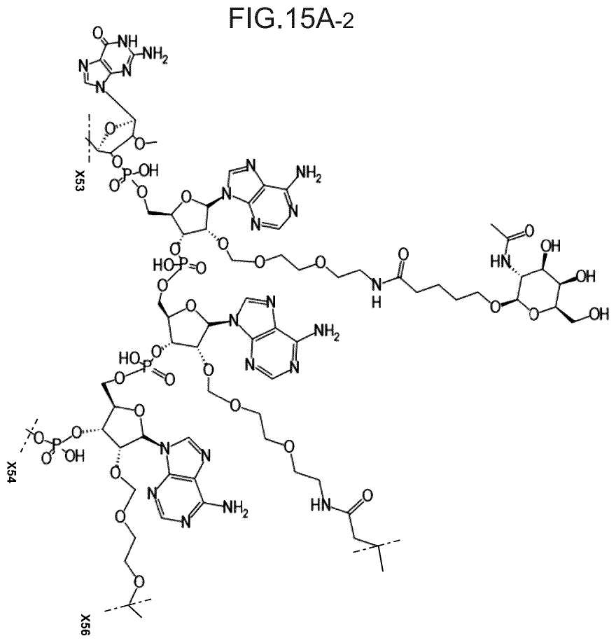

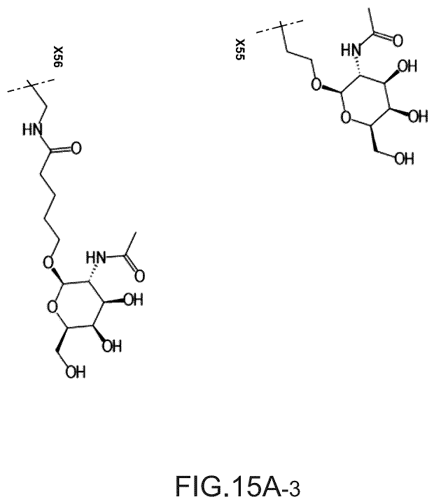

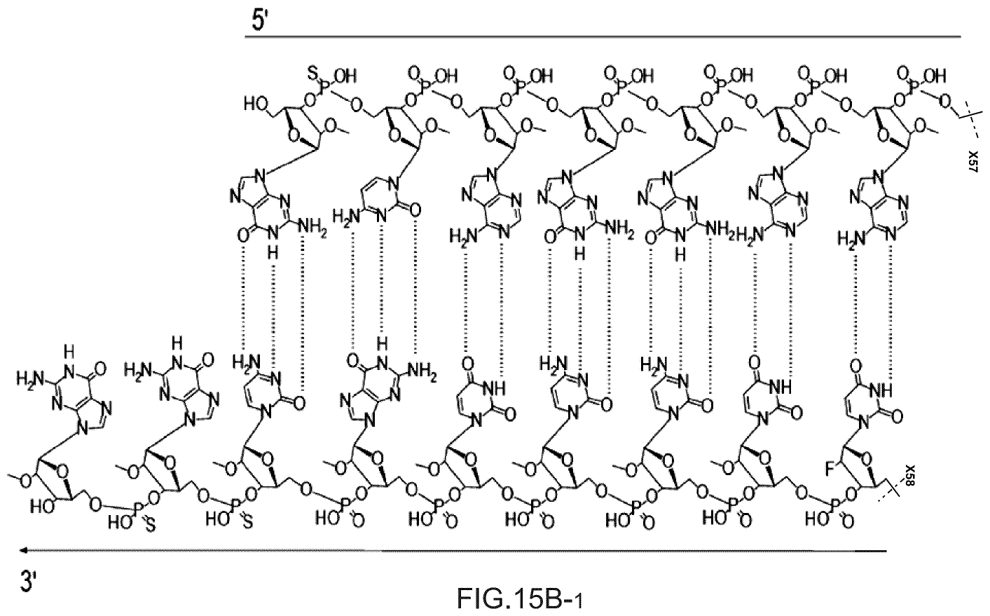

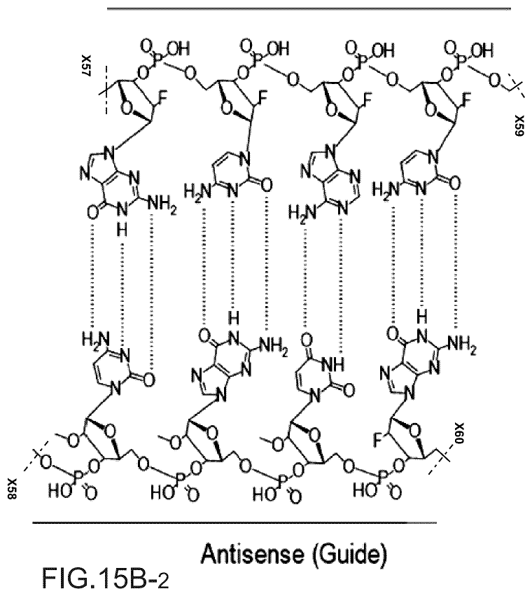

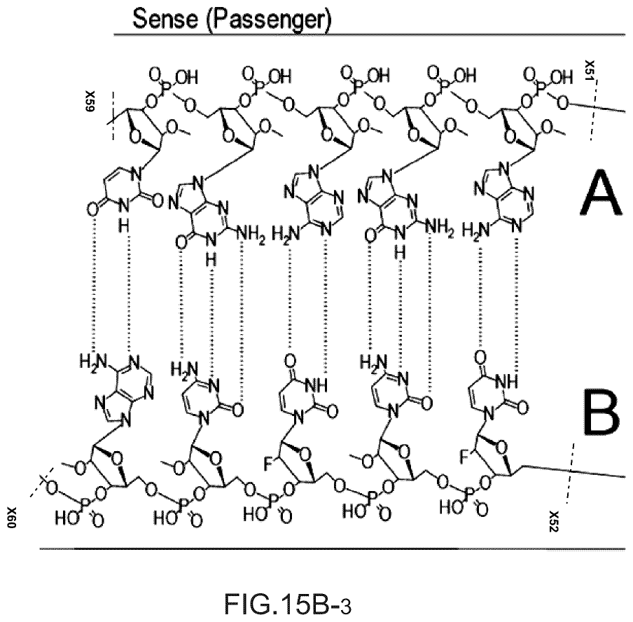

In some embodiments, the KHK-targeting dsRNAi oligonucleotides comprise a sense strand comprising SEQ ID NO: 804 and an antisense strand comprising SEQ ID NO: 849, wherein said dsRNA is in the form of a conjugate having the structures depicted in FIG. 15A continuing to FIG. 15B, or pharmaceutically acceptable salts thereof.

### Formulations

Various formulations have been developed to facilitate oligonucleotide use. For example, oligonucleotides (e.g., dsRNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., dsRNAi oligonucleotides) reduce the expression of KHK. Such compositions can be suitably formulated such that when administered to a subject, either into the immediate environment of a target cell or systemically, a sufficient portion of the oligonucleotides enter the cell to reduce KHK expression. Any variety of suitable oligonucleotide formulations can be used to deliver oligonucleotides for the reduction of KHK as disclosed herein. In some embodiments, an oligonucleotide is formulated in buffer solutions such as phosphate buffered saline solutions, liposomes, micellar structures, and capsids. Any of the oligonucleotides described herein may be provided not only as nucleic acids, but also in the form of a pharmaceutically acceptable salt.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a dsRNAi oligonucleotide for reducing KHK expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing KHK Expression

In some embodiments, the disclosure provides methods for contacting or delivering to a cell or population of cells an effective amount of oligonucleotides (e.g., dsRNAi oligonucleotides) herein to reduce KHK expression. In some embodiments, a reduction of KHK expression is determined by measuring a reduction in the amount or level of KHK mRNA, KHK protein, or KHK activity in a cell. The methods include those described herein and known to one of ordinary skill in the art.

Methods provided herein are useful in any appropriate cell type. In some embodiments, a cell is any cell that expresses KHK mRNA (e.g., hepatocytes). In some embodiments, the cell is a primary cell obtained from a subject. In some embodiments, the primary cell has undergone a limited number of passages such that the cell substantially maintains its natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein are delivered to a cell or population of cells using a nucleic acid delivery method known in the art including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other methods known in the art for delivering oligonucleotides to cells may be used, such as lipid-mediated carrier transport, chemical-mediated transport, and cationic liposome transfection such as calcium phosphate, and others.

In some embodiments, reduction of KHK expression is determined by an assay or technique that evaluates one or more molecules, properties, or characteristics of a cell or population of cells associated with KHK expression, or by an assay or technique that evaluates molecules that are directly indicative of KHK expression in a cell or population of cells (e.g., KHK mRNA or KHK protein). In some embodiments, the extent to which an oligonucleotide provided herein reduces KHK expression is evaluated by comparing KHK expression in a cell or population of cells contacted with the oligonucleotide to an appropriate control (e.g., an appropriate cell or population of cells not contacted with the oligonucleotide or contacted with a control oligonucleotide). In some embodiments, a control amount or level of KHK expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide (e.g., dsRNAi oligonucleotides) described herein to a cell or a population of cells results in a reduction in KHK expression in a cell or population of cells not contacted with the oligonucleotide or contacted with a control oligonucleotide. In some embodiments, the reduction in KHK expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of KHK expression. In some embodiments, the control amount or level of KHK expression is an amount or level of KHK mRNA and/or KHK protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, KHK expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, KHK expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

### Treatment Methods

The disclosure provides oligonucleotides for use as a medicament, in particular for use in a method for the treatment of diseases, disorders, and conditions associated with expression of KHK. The disclosure also provides oligonucleotides for use, or adaptable for use, to treat a subject (e.g., a human having a disease, disorder or condition associated with KHK expression) that would benefit from reducing KHK expression. In some respects, the disclosure provides oligonucleotides for use, or adapted for use, to treat a subject having a disease, disorder or condition associated with expression of KHK. The disclosure also provides oligonucleotides for use, or adaptable for use, in the manufacture of a medicament or pharmaceutical composition for treating a disease, disorder or condition associated with KHK expression. In some embodiments, the oligonucleotides for use, or adaptable for use, target KHK mRNA and reduce KHK expression (e.g., via the RNAi pathway). In some embodiments, the oligonucleotides for use, or adaptable for use, target KHK mRNA and reduce the amount or level of KHK mRNA, KHK protein and/or KHK activity.

In addition, in some embodiments of the methods herein, a subject having a disease, disorder, or condition associated with KHK expression or is predisposed to the same is selected for treatment with an oligonucleotide (e.g., a double-stranded oligonucleotide) herein. In some embodiments, the method comprises selecting an individual having a marker (e.g., a biomarker) for a disease, disorder, or condition associated with KHK expression or predisposed to the same, such as, but not limited to, KHK mRNA, KHK protein, or a combination thereof. Likewise, and as detailed below, some embodiments of the methods provided by the disclosure include steps such as measuring or obtaining a baseline value for a marker of KHK expression (e.g., KHK), and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with a KHK expression with an oligonucleotide provided herein. In some aspects, the disclosure provides methods of treating or attenuating the onset or progression of a disease, disorder or condition associated with KHK expression using the oligonucleotides herein. In other aspects, the disclosure provides methods to achieve one or more therapeutic benefits in a subject having a disease, disorder, or condition associated with KHK expression using the oligonucleotides provided herein. In some embodiments of the methods herein, the subject is treated by administering a therapeutically effective amount of any one or more of the oligonucleotides provided herein. In some embodiments, treatment comprises reducing KHK expression. In some embodiments, the subject is treated therapeutically. In some embodiments, the subject is treated prophylactically.

In some embodiments of the methods herein, one or more oligonucleotides (e.g., dsRNAi oligonucleotides) herein, or a pharmaceutical composition comprising one or more oligonucleotides, is administered to a subject having a disease, disorder or condition associated with KHK expression such that KHK expression is reduced in the subject, thereby treating the subject. In some embodiments, an amount or level of KHK mRNA is reduced in the subject. In some embodiments, an amount or level of KHK protein is reduced in the subject. In some embodiments, an amount or level of KHK activity is reduced in the subject. In some embodiments, an amount or level of triglyceride (TG) (e.g., one or more TG(s) or total TGs) is reduced in the subject. In some embodiments, an amount or level of plasma glucose is reduced in the subject. In some embodiments, an amount or level of blood pressure (e.g., systolic pressure, diastolic pressure, or both) is reduced in the subject. In some embodiments, an amount or level of abdominal fat is reduced in the subject. In some embodiments, an amount or level of cholesterol (e.g., total cholesterol, LDL cholesterol, and/or HDL cholesterol) is reduced in the subject. In some embodiments, an amount or level of liver steatosis is reduced in the subject. In some embodiments, an amount or level of liver fibrosis is reduced in the subject. In some embodiments, the ratio of total cholesterol to HDL cholesterol is altered in the subject. In some embodiments, any combination of the following is reduced or altered in the subject: KHK expression, an amount or level of KHK mRNA, an amount or level of KHK protein, an amount or level of KHK activity, an amount or level of blood glucose, an amount or level of abdominal fat, an amount or level of blood pressure, an amount or level of TG, an amount or level of cholesterol and/or the ratio of total cholesterol to HDL cholesterol, an amount or level of liver steatosis, and amount or level of liver fibrosis.

In some embodiments of the methods herein, an oligonucleotide (e.g., dsRNAi oligonucleotides) herein, or a pharmaceutical composition comprising the oligonucleotide, is administered to a subject having a disease, disorder or condition associated with KHK such that KHK expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to KHK expression prior to administration of one or more oligonucleotides or pharmaceutical composition. In some embodiments, KHK expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to KHK expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with KHK expression such that an amount or level of KHK mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of KHK mRNA prior to administration of the oligonucleotide or pharmaceutical composition. In some embodiments, an amount or level of KHK mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of KHK mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with KHK expression such that an amount or level of KHK protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of KHK protein prior to administration of the oligonucleotide or pharmaceutical composition. In some embodiments, an amount or level of KHK protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of KHK protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides (e.g., dsRNAi oligonucleotides) herein, or a pharmaceutical composition comprising the oligonucleotide or oligonucleotides, is administered to a subject having a disease, disorder or condition associated with KHK such that an amount or level of KHK activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of KHK activity prior to administration of the oligonucleotide or pharmaceutical composition. In some embodiments, an amount or level of KHK activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of KHK activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with KHK expression such that an amount or level of TG (e.g., one or more TGs or total TGs) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of TG prior to administration of the oligonucleotide or pharmaceutical composition. In some embodiments, an amount or level of TG is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of TG in a subject (e.g., a reference or control subject) not receiving the oligonucleotide or pharmaceutical composition or receiving a control oligonucleotide, pharmaceutical composition or treatment.

Generally, a normal or desirable TG range for a human patient is <150 mg/dL of blood, with <100 being considered ideal. In some embodiments, the patient selected for treatment or treated is identified or determined to have an amount or level of TG of ≥150 mg/dl. In some embodiments, the patient selected for treatment or treated is identified or determined to have an amount or level of TG in the range of 150 to 199 mg/dL, which is considered borderline high TG levels. In some embodiments, the patient selected for treatment or treated is identified or determined to have an amount or level of TG in the range of 200 to 499 mg/dL, which is considered high TG levels. In some embodiments, the patient selected for treatment or treated is identified or determined to have an amount or level of TG in the range of 500 mg/dL or higher (i.e., ≥500 mg/dL), which is considered very high TG levels. In some embodiments, the patient selected for treatment or treated is identified or determined to have an amount or level of TG which is ≥150 mg/dl, ≥200 mg/dl or >500 mg/dl. In some embodiments, the patient selected for treatment or treated is identified or determined to have an amount of level of TG of 200 to 499 mg/dL, or 500 mg/dL or higher. In some embodiments, the patient selected for treatment or treated is identified or determined to have an amount or level of TG which is ≥200 mg/dL. In some embodiments of the methods herein, an oligonucleotide (e.g., dsRNAi oligonucleotide) herein, or a pharmaceutical composition comprising the oligonucleotide, is administered to a subject having a disease, disorder or condition associated with KHK expression such that an amount or level of cholesterol (e.g., total cholesterol, LDL cholesterol, and/or HDL cholesterol) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of cholesterol prior to administration of the oligonucleotide or pharmaceutical composition. In some embodiments, an amount or level of cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of cholesterol in a subject (e.g., a reference or control subject) not receiving the oligonucleotide or pharmaceutical composition or receiving a control oligonucleotide, pharmaceutical composition or treatment.

Generally, a normal or desirable cholesterol range (total cholesterol) for an adult human patient is <200 mg/dl of blood. In some embodiments, the patient selected for treatment or treated is identified or determined to have an amount or level of cholesterol of ≥200 mg/dL. In some embodiments, the patient selected for treatment or treated is identified or determined to have an amount or level of cholesterol in the range of 200 to 239 mg/dl, which is considered borderline high cholesterol levels. In some embodiments, the patient selected for treatment or treated is identified or determined to have an amount or level of cholesterol in the range of 240 mg/dl and higher (i.e., ≥240 mg/dL), which is considered high cholesterol levels. In some embodiments, the patient selected from treatment or treated is identified or determined to have an amount or level of cholesterol of 200 to 239 mg/dl, or 240 mg/dL or higher. In some embodiments, the patient selected for treatment or treated is identified or determined to have an amount or level of cholesterol which is ≥200 mg/dl or ≥240 mg/dL or higher.

In some embodiments of the methods herein, an oligonucleotide herein, or a pharmaceutical composition comprising the oligonucleotide, is administered to a subject having a disease, disorder, or condition associated with KHK expression such that an amount or level of liver fibrosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of liver fibrosis prior to administration of the oligonucleotide or pharmaceutical composition. In some embodiments, an amount or level of liver fibrosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of liver fibrosis in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with KHK expression such that an amount or level of liver steatosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of liver steatosis prior to administration of the oligonucleotide or pharmaceutical composition. In some embodiments, an amount or level of liver steatosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of liver steatosis in a subject (e.g., a reference or control subject) not receiving the oligonucleotide or pharmaceutical composition or receiving a control oligonucleotide, pharmaceutical composition or treatment.

Suitable methods for determining KHK expression, the amount or level of KHK mRNA, KHK protein, KHK activity, TG, plasma glucose or cholesterol amount or activity in the subject, or in a sample from the subject, are known in the art. Further, the Examples set forth herein illustrate methods for determining KHK expression.

In some embodiments, KHK expression, the amount or level of KHK mRNA, KHK protein, KHK activity, TG, plasma glucose, or cholesterol, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other appropriate biological material obtained or isolated from the subject. In some embodiments, KHK expression, the amount or level of KHK mRNA, KHK protein, KHK activity, TG, plasma glucose or cholesterol or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), or more than one type of sample (e.g., a liver biopsy sample and one or more other type(s) of biopsy sample).

Generally, a normal or desirable blood sugar level for a human patient is <140 mg/dL. Blood sugar levels between 140 and 199 mg/dL two hours after eating indicates pre-diabetes, and >200 mg/dL indicates diabetes. In some embodiments, the patient selected for treatment or treated is identified or determined to have a level of blood sugar between about 140 mg/dl and about 199 mg/dL, which is considered pre-diabetes. In some embodiments, the patient selected for treatment or treated is identified or determined to have a level of blood sugar ≥200 mg/dL, which is considered diabetes. In some embodiments of the methods herein, an oligonucleotide (e.g., dsRNAi oligonucleotide) herein, or a pharmaceutical composition comprising the oligonucleotide, is administered to a subject having a disease, disorder or condition associated with KHK expression such that an amount or level of blood sugar is reduced to a normal or pre-diabetes range.

Examples of a disease, disorder or condition associated with KHK expression include, but are not limited to, glucose intolerance, pre-diabetes, type-1 diabetes, type-2 diabetes, metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn's disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn's disease induced fibrosis, ulcerative colitis-induced fibrosis, intestine (e.g. small intestine) fibrosis, colon fibrosis, stomach fibrosis, disease of elevated uric acid (e.g. hyperuricemia, gout), sugar craving, alcohol craving, aldolase B deficiency, hereditary fructose intolerance, chronic kidney disease, diabetic nephropathy, kidney fibrosis, liver failure, liver function loss, coagulopathy, steatohepatitis, disorders of glycemic control, and other KHK-associated metabolic-related disorders and diseases. Of particular interest herein are metabolic syndrome, hypertriglyceridemia, NAFLD, NASH, obesity, or a combination thereof.

Because of their high specificity, the oligonucleotides herein (e.g., dsRNAi oligonucleotides) specifically target mRNAs of target genes of cells and tissue(s), or organs(s) (e.g., liver). In preventing disease, the target gene may be one which is required for initiation or maintenance of the disease or which has been identified as being associated with a higher risk of contracting the disease. In treating disease, the oligonucleotide can be brought into contact with the cells, tissue(s), or organ(s) (e.g., liver) exhibiting or responsible for mediating the disease. For example, an oligonucleotide substantially identical to all or part of a wild-type (i.e., native) or mutated gene associated with a disorder or condition associated with KHK expression may be brought into contact with or introduced into a cell or tissue type of interest such as a hepatocyte or other liver cell.

In some embodiments, the target gene may be a target gene from any mammal, such as a human target. Any gene may be silenced according to the method described herein.

Methods described herein typically involve administering to a subject an effective amount of an oligonucleotide herein (e.g., a dsRNAi oligonucleotide), that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

As a non-limiting set of examples, the oligonucleotides herein (e.g., dsRNAi oligonucleotides) would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments the oligonucleotides herein are administered alone or in combination. In some embodiments the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

In some embodiments, a single dose of one or more oligonucleotides (e.g., dsRNAi oligonucleotides) herein, or a pharmaceutical composition comprising the oligonucleotide(s), is administered to a subject having a disease, disorder, or condition associated with KHK expression such that an amount or level of KHK mRNA and/or KHK protein, preferably of KHK protein, is reduced in the subject. Said reduction of an amount or level of KHK mRNA and/or KHK protein may be determined by comparison with the amount or level of KHK mRNA and/or KHK protein in a subject (e.g., a reference or control subject) not receiving the oligonucleotide(s) or pharmaceutical composition or receiving one or more control oligonucleotides or pharmaceutical compositions or treatments, or—preferably—by comparison with the amount or level of KHK mRNA and/or KHK protein prior to administration of the oligonucleotide(s) or pharmaceutical composition. Said amount or level of KHK mRNA and/or KHK protein may be determined from liver biopsy samples from the subject. Said single dose may be administered subcutaneously. Said dose of the oligonucleotide(s) may be below 10 mg/kg bodyweight of the subject, e.g. 6 mg/kg or below, in particular from 0.01 mg/kg to 5 mg/kg. Said reduction of an amount or level of KHK mRNA and/or KHK protein may be detectable more than 10 days after the single dose administration of the oligonucleotide(s), e.g. it may remain detectable at day 28, 56, and/or 84 after administration. Said reduction of an amount or level of KHK mRNA and/or KHK protein may be, e.g., at least about 30%, about 35%, about 40%, about 45%, about 50%, about 55%, about 60%, about 65%, about 70%, about 75%, about 80%, about 85%, about 90%, about 95%, about 99% or greater than 99%. In a preferred embodiment, the reduction of an amount or level of KHK mRNA and/or KHK protein remains detectable at day 28, optionally at day 56 and/or 84, after subcutaneous administration of a single dose of one or more oligonucleotides (e.g., dsRNAi oligonucleotides) herein, or a pharmaceutical composition comprising the oligonucleotide(s).

### Kits

In some embodiments, the disclosure provides a kit comprising an oligonucleotide herein, and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein, and a pharmaceutically acceptable carrier, or a pharmaceutical composition comprising the oligonucleotide and instructions for treating or delaying progression of a disease, disorder or condition associated with KHK expression in a subject in need thereof.

### Definitions

As used herein, the term “antisense oligonucleotide” encompasses a nucleic acid-based molecule which has a sequence complementary to all or part of the target mRNA, in particular seed sequence thereby capable of forming a duplex with a mRNA. Thus, the term “antisense oligonucleotide”, as used herein, may be referred to as “complementary nucleic acid-based inhibitor”.

As used herein, “approximately” or “about”, as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer”, “administering”, “administration” and the like refers to providing a substance (e.g., an oligonucleotide) to a subject in a manner that is pharmacologically useful (e.g., to treat a condition in the subject).

As used herein, “attenuate”, “attenuating”, “attenuation” and the like refers to reducing or effectively halting. As a non-limiting example, one or more of the treatments herein may reduce or effectively halt the onset or progression of dyslipidemia/hypertriglyceridemia/hyperlipidemia, NAFLD, NASH, or glucose intolerance in a subject. This attenuation may be exemplified by, for example, a decrease in one or more aspects (e.g., symptoms, tissue characteristics, and cellular, inflammatory or immunological activity, etc.) of dyslipidemia/hypertriglyceridemia/hyperlipidemia, NAFLD, NASH, or glucose intolerance, no detectable progression (worsening) of one or more aspects of dyslipidemia/hypertriglyceridemia/hyperlipidemia, NAFLD, NASH, or glucose intolerance, or no detectable aspects of dyslipidemia/hypertriglyceridemia/hyperlipidemia, NAFLD, NASH, or glucose intolerance in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, the phrase “glucose intolerance” refers to a metabolic condition resulting in higher-than-normal levels of blood glucose. Glucose intolerance can include type 1, type 1.5, and type 2 diabetes.

As used herein, “hepatocyte” or “hepatocytes” refers to cells of the parenchymal tissues of the liver. These cells make up about 70%-85% of the liver's mass and manufacture serum albumin, FBN and the prothrombin group of clotting factors (except for Factors 3 and 4). Markers for hepatocyte lineage cells include, but are not limited to, transthyretin (Ttr), glutamine synthetase (Glul), hepatocyte nuclear factor 1a (Hnf1a) and hepatocyte nuclear factor 4a (Hnf4a). Markers for mature hepatocytes may include, but are not limited to, cytochrome P450 (Cyp3a11), fumarylacetoacetate hydrolase (Fah), glucose 6-phosphate (G6p), albumin (Alb) and OC2-2F8. See, e.g., Huch et al. (2013) Nature 494:247-50.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCI4), acetaminophen (paracetamol), vinyl chloride, arsenic, chloroform, nonsteroidal anti-inflammatory drugs (such as aspirin and phenylbutazone).

As used herein, the term “ketohexokinase” or “KHK” refers to an enzyme, specifically a hepatic fructokinase, that catalyzes the phosphorylation of fructose. The KHK gene encodes two protein isoforms (KHK-A and KHK-C). The two products are generated from the same primary transcript by alternative splicing. The term “KHK” is intended to refer to both isoforms unless stated otherwise. ‘KHK’ may also refer to the gene which encodes the protein.

As used herein, “labile linker” refers to a linker that can be cleaved (e.g., by acidic pH). A “fairly stable linker” refers to a linker that cannot be cleaved.

As used herein, “liver inflammation” or “hepatitis” refers to a physical condition in which the liver becomes swollen, dysfunctional and/or painful, especially as a result of injury or infection, as may be caused by exposure to a hepatotoxic agent. Symptoms may include jaundice (yellowing of the skin or eyes), fatigue, weakness, nausea, vomiting, appetite reduction and weight loss. Liver inflammation, if left untreated, may progress to fibrosis, cirrhosis, liver failure or liver cancer.

As used herein, “liver fibrosis”, “Liver Fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”).

As used herein, “Metabolic syndrome′ or “metabolic liver disease” refers to a disorder characterized by a cluster of associated medical conditions and associated pathologies including, but not limited to the following medical conditions: abdominal obesity, elevated blood pressure, elevated fasting plasma glucose, high serum triglycerides, liver fibrosis, and low levels of high-density lipoprotein (HDL) levels. As used herein, the term metabolic syndrome or metabolic liver disease may encompass a wide array of direct and indirect manifestations, diseases and pathologies associated with metabolic syndrome and metabolic liver disease, with an expanded list of conditions used throughout the document.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified internucleotide linkage may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (DsiRNA), antisense oligonucleotide, short siRNA or ss siRNA. In some embodiments, a double-stranded (dsRNA) is an RNAi oligonucleotide.

As used herein, “overhang” (or “overhang sequence”) refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In certain embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Patent Publication No. 2019-0177729. Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “reduced expression” of a gene (e.g., KHK) refers to a decrease in the amount or level of RNA transcript (e.g., KHK mRNA) or protein encoded by the gene and/or a decrease in the amount or level of activity of the gene in a cell, a population of cells, a sample, or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising KHK mRNA) may result in a decrease in the amount or level of KHK mRNA, protein and/or activity (e.g., via degradation of KHK mRNA by the RNAi pathway) when compared to a cell that is not treated with the dsRNA.

Similarly, and as used herein, “reducing expression” refers to an act that results in reduced expression of a gene (e.g., KHK).

As used herein, “reduction of KHK expression” refers to a decrease in the amount or level of KHK mRNA, KHK protein and/or KHK activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementarity to a mRNA target sequence. In some embodiments, the region of complementarity is full complementary. In some embodiments, the region of complementarity is partially complementary (e.g., up to 3 nucleotide mismatches).

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., KHK mRNA) or (b) a single-stranded oligonucleotide having a single antisense strand, where that antisense strand (or part of that antisense strand) is used by the Ago2 endonuclease in the cleavage of a target mRNA (e.g., KHK mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide, or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM Na₂HPO₄ to a hairpin comprising a duplex of at least 2 base pairs (bp) in length. In some embodiments, a tetraloop can confer a Tm of at least about 50° C., at least about 55° C., at least about 56° C., at least about 58° C., at least about 60° C., at least about 65° C. or at least about 75° C. in 10 mM NaH₂PO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) NATURE 346:680-82; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden (1985) Nucleic Acids Res. 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-8471; Antao et al. (1991) NUCLEIC ACIDS RES. 19:5901-5905). Examples of DNA tetraloops include the d (GNNA) family of tetraloops (e.g., d (GTTA), the d (GNRA)) family of tetraloops, the d (GNAB) family of tetraloops, the d (CNNG) family of tetraloops, and the d (TNCG) family of tetraloops (e.g., d (TTCG)). See, e.g., Nakano et al. (2002) BIOCHEM. 41:14281-92; Okabe et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

While the disclosure has been described with reference to the specific embodiments set forth in the following Examples, it should be understood by those skilled in the art that various changes may be made, and equivalents may be substituted without departing from the true spirit and scope of the disclosure. Further, the following Examples are offered by way of illustration and are not intended to limit the scope of the disclosure in any manner. In addition, modifications may be made to adapt to a situation, material, composition of matter, process, process step or steps, to the objective, spirit, and scope of the disclosure. All such modifications are intended to be within the scope of the disclosure. Standard techniques well known in the art or the techniques specifically described below were utilized.

RNAi agents targeting KHK have been described and tested in vitro (e.g., WO 2015123264 and WO 2020060986). The following studies describe the identification of novel dsRNAi agents useful for reducing or inhibiting KHK expression based on in vitro and in vivo screening, including studies in non-human primates. The novel dsRNAi agents comprise 36mer sense strands and 22mer antisense strands with a stem loop having a nicked tetraloop conjugated to GalNAc moieties at the 3′end of the sense strand for reducing KHK mRNA. The presence of a nick within the stem loop provides a precut antisense strand to form a pre-processed binding substrate for the Dicer enzyme, allowing Dicer to efficiently bind and hand off the double stranded molecule to Ago2. The tetraloop provides a thermodynamically stabilizing element to prevent the loop from opening and exposing the 5′-end of the antisense strand and the 3′-end of the sense strand, thereby providing increased nuclease resistance. Accordingly, the present dsRNAi agents are particularly useful for inhibiting KHK expression in vitro and in vivo as described in the following examples.

In comparison to dsRNAi agents described in the prior art, the dsRNAi agents presented herein may, in particular, show improved in vitro and/or in vivo reduction or inhibition of KHK expression as determined on the KHK mRNA and/or KHK protein level. Such improvement may relate to the size and/or duration of the inhibitory action. Thus, for instance, for medical uses of the dsRNAi agents according to this invention, lower doses and/or lower dose frequencies may be applicable. Also, dsRNAi agents presented herein may benefit from advantageous safety and tolerability features like high specificity, low off-target effects or reduced immunogenicity.

### Example 1: Preparation of Double-Stranded RNAi Oligonucleotides

### Oligonucleotide Synthesis and Purification

The double-stranded RNAi (dsRNA) oligonucleotides described in the foregoing Examples are chemically synthesized using methods described herein. Generally, dsRNAi oligonucleotides are synthesized using solid phase oligonucleotide synthesis methods as described for 19-23mer siRNAs (see, e.g., Scaringe et al. (1990) NUCLEIC ACIDS RES. 18:5433-5441 and Usman et al. (1987) J. AM. CHEM. SOC. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) COLD SPRING HARB PERSPECT BIOL. 9 (1): a023812; Beaucage S.L., Caruthers M. H., Studies on Nucleotide Chemistry V: Deoxynucleoside Phosphoramidites—A New Class of Key Intermediates for Deoxypolynucleotide Synthesis, TETRAHEDRON LETT. 1981; 22:1859-62. doi: 10.1016/S0040-4039 (01) 90461-7). dsRNAi oligonucleotides having a 19mer core sequence were formatted into constructs having a 25mer sense strand and a 27mer antisense strand to allow for processing by the RNAi machinery. The 19mer core sequence is complementary to a region in the KHK mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A: B to 52:48 Buffers A: B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DET Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 uM concentration) in duplex buffer consisting of 100 mM potassium acetate, 30 mM HEPES, pH 7.5. Complementary sense and antisense strands were mixed in equal molar amounts to yield a final solution of, for example, 50 M duplex. Samples were heated to 100° C. for 5′ in RNA buffer (IDT) and were allowed to cool to room temperature before use. The dsRNA oligonucleotides were stored at −20° C. Single strand RNA oligomers were stored lyophilized or in nuclease-free water at −80° C.

### Example 2: Generation of KHK-Targeting Double-Stranded (DS) RNAi Oligonucleotides

Identification of KHK mRNA Target Sequences

Ketohexokinase (KHK) is an enzyme involved in fructose metabolism. KHK has two isoforms, differing by one alternative exon, with distinct substrates and mechanisms of action. The isoform KHK-A is encoded by Exon 3A whereas the KHK-C isoform is encoded by Exon 3C. To generate RNAi oligonucleotide inhibitors of KHK-A and KHK-C expression, a computer-based algorithm was used to computationally identify KHK mRNA target sequences suitable for assaying inhibition of KHK expression by the RNAi pathway. The algorithm provided RNAi oligonucleotide guide (antisense) strand sequences each having a region of complementarity to a suitable KHK target sequence of human KHK mRNA (e.g., SEQ ID NO: 1; Table 1). Some of the guide strand sequences identified by the algorithm were also complementary to the corresponding KHK target sequence of monkey and/or mouse KHK mRNA (SEQ ID NO: 2 and 3, respectively; Table 1). KHK RNAi oligonucleotides comprising a region of complementarity to homologous KHK mRNA target sequences with nucleotide sequence similarity are predicted to have the ability to target homologous KHK mRNAs.

TABLE 1
Sequences of Human, Monkey and Mouse KHK mRNA

| Species | Ref Seq # | SEQ ID NO |
|---|---|---|
| Human (Hs) | NM_006488.3 | 1 |
| Cynomolgus monkey (Mf) | XM_005576322.2 | 2 |
| Mouse (Mm) | NM_008439.4 | 3 |

RNAi oligonucleotides (formatted as DsiRNA oligonucleotides) were generated as described in Example 1 for evaluation in vitro. Each DsiRNA was generated with the same modification pattern, and each with a unique guide strand having a region of complementarity to a KHK target sequence identified by the algorithm (Table 2). Modifications for the sense and anti-sense DsiRNA included the following (X-any nucleotide; m-2′-O-methyl modified nucleotide; r-ribosyl modified nucleotide):

Sense Strand:
rXmXrXmXrXrXrXrXrXrXrXrXrXmXrXmXrXrXrXrXrXrXrXXX
Anti-sense Strand:
mXmXmXmXrXrXrXrXrXrXmXrXmXrXrXrXrXrXrXrXrXrXmXrX
mXmXmX

### In Vitro Cell-Based Assays

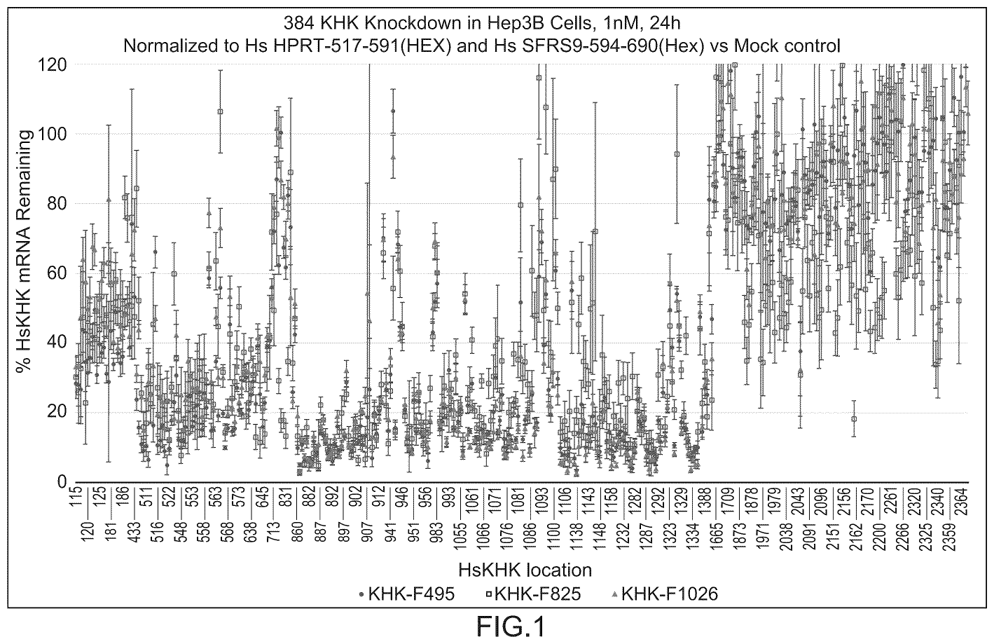

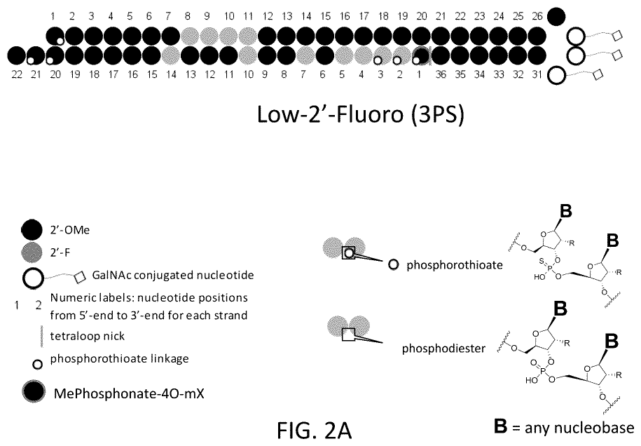

The ability of each of the modified DsiRNA in Table 2 to reduce KHK mRNA was measured using in vitro cell-based assays. Briefly, human hepatoma (Hep3B) cells expressing endogenous human KHK gene were transfected with each of the DsiRNAs listed in Table 2 (Sense Strand SEQ ID NOs: 4-387) at 1 nM in separate wells of a multi-well cell-culture plate. Cells were maintained for 24 hours following transfection with the modified DsiRNA, and then the amount of remaining KHK mRNA from the transfected cells was determined using TAQMAN®-based qPCR assays. Two qPCR assays, a 3′ assay (Forward-1026; TGGAGGTGGAGAAGCCA, Reverse-1157; GACCATACAAGCCCCTCAAG, Probe-1080; TGGTGTTTGTCAGCAAAGATGTGGC) and a 5′ assay (Forward-496; AGGAAGCTCTGGGAGTA, Reverse-596; CCTCCTTAGGGTACTTGTC, Probe-518; ATGGAAGAGAAGCAGATCCTGTGCG) were used to determine KHK mRNA levels as measured using PCR probes conjugated to 6-carboxy-fluorescein (FAM). Each primer pair (KHK-825 for KHK-C isoform, NM_006488.3) and KHK-All (both isoforms) (KHK-F495, KHK-F1026 for KHK-All (both isoforms) was assayed for % remaining RNA as shown in Table 2 and FIG. 1. DsiRNAs resulting in less than or equal to 10% KHK mRNA remaining in DsiRNA-transfected cells when compared to mock-transfected cells were considered DsiRNA “hits”. The Hep3B cell-based assay evaluating the ability of the DsiRNAs listed in Table 2 to inhibit KHK expression identified several candidate DsiRNAs.

Taken together, these results show that DsiRNAs designed to target human KHK mRNA inhibit KHK expression in cells, as determined by a reduced amount of KHK mRNA in DsiRNA-transfected cells relative to control cells. These results demonstrate that the nucleotide sequences comprising the DsiRNA are useful for generating RNAi oligonucleotides to inhibit KHK expression. Further, these results demonstrate that multiple KHK mRNA target sequences are suitable for the RNAi-mediated inhibition of KHK expression.

TABLE 2
Analysis of KHK mRNA in HepB3 cells

| SEQ ID NO | SEQ ID NO | | KHK-F495 | | KHK-F1026 | | KHK-825 | | % average |
|---|---|---|---|---|---|---|---|---|---|
| (Sense | (Anti-sense | | % | | % | | % | | of all |
| Strand) | Strand) | DsiRNA name | remaining | SEM | remaining | SEM | remaining | SEM | assays |
| 4 | 388 | KHK-115-154 | 28.3 | 2.4 | 33.1 | 2.7 | 30.22 | 6.148 | 30.5 |
| 5 | 389 | KHK-116-155 | 27.8 | 5.9 | 33.3 | 6.5 | 25.691 | 8.728 | 28.9 |
| 6 | 390 | KHK-117-156 | 26.7 | 9.9 | 47.5 | 14.6 | 32.656 | 13.903 | 35.6 |
| 7 | 391 | KHK-118-157 | 43.7 | 6.3 | 63.9 | 6.4 | 47.299 | 13.144 | 51.6 |
| 8 | 392 | KHK-119-158 | 34.6 | 8.6 | 57.5 | 14.7 | 22.791 | 11.895 | 38.3 |
| 9 | 393 | KHK-120-159 | 31.4 | 3.8 | 49.7 | 3.5 | 40.388 | 5.289 | 40.5 |
| 10 | 394 | KHK-121-160 | 35.8 | 4.4 | 53.2 | 5.7 | 43.359 | 8.043 | 44.1 |
| 11 | 395 | KHK-122-161 | 41.8 | 3.7 | 67.6 | 6.5 | 39.75 | 5.587 | 49.7 |
| 12 | 396 | KHK-123-162 | 49.5 | 3.7 | 66.7 | 4.6 | 41.386 | 7.102 | 52.5 |
| 13 | 397 | KHK-124-163 | 30.9 | 2.5 | 49.6 | 4.4 | 40.966 | 7.589 | 40.5 |
| 14 | 398 | KHK-125-164 | 39.5 | 3.7 | 52.5 | 5.1 | 43.748 | 9.465 | 45.3 |
| 15 | 399 | KHK-126-165 | 37.1 | 2.8 | 57.9 | 5.2 | 53.25 | 7.484 | 49.4 |
| 16 | 400 | KHK-127-166 | 38.6 | 3.1 | 63.0 | 4.3 | 45.393 | 5.073 | 49.0 |
| 17 | 401 | KHK-128-167 | 31.1 | 2.6 | 44.6 | 3.3 | 47.943 | 5.962 | 41.2 |
| 18 | 402 | KHK-179 | 28.9 | 23.1 | 81.2 | 21.2 | 62.845 | 5.928 | 57.6 |
| 19 | 403 | KHK-181-220 | 43.4 | 5.3 | 57.6 | 5.6 | 56.679 | 10.519 | 52.5 |
| 20 | 404 | KHK-182-221 | 33.8 | 1.8 | 40.4 | 3.4 | 50.153 | 10.698 | 41.5 |
| 21 | 405 | KHK-183-222 | 48.9 | 3.5 | 57.7 | 5.4 | 58.074 | 7.904 | 54.9 |
| 22 | 406 | KHK-184-223 | 37.0 | 2.6 | 38.5 | 3.0 | 49.417 | 4.396 | 41.6 |
| 23 | 407 | KHK-185-224 | 34.3 | 4.0 | 44.8 | 4.3 | 53.186 | 7.765 | 44.1 |
| 24 | 408 | KHK-186-225 | 36.1 | 4.9 | 42.4 | 5.0 | 51.087 | 6.631 | 43.2 |
| 25 | 409 | KHK-187-226 | 48.5 | 3.9 | 48.2 | 4.5 | 81.75 | 6.116 | 59.5 |
| 26 | 410 | KHK-188-227 | 50.9 | 2.8 | 50.5 | 5.6 | 75.858 | 6.936 | 59.1 |
| 27 | 411 | KHK-431-470 | 38.7 | 2.5 | 42.1 | 3.8 | 52.142 | 5.549 | 44.3 |
| 28 | 412 | KHK-432-471 | 74.1 | 38.8 | 65.5 | 11.0 | 50.476 | 11.385 | 63.3 |
| 29 | 413 | KHK-433-472 | 53.1 | 8.6 | 72.4 | 9.0 | 47.428 | 12.036 | 57.7 |
| 30 | 414 | KHK-507-545-376-218 | 23.8 | 3.1 | 31.1 | 2.5 | 84.241 | 10.972 | 46.4 |
| 31 | 415 | KHK-508-546-377-219 | 15.7 | 1.8 | 43.7 | 2.6 | 52.076 | 6.767 | 37.2 |
| 32 | 416 | KHK-509-547-378-220 | 10.9 | 2.4 | 24.5 | 4.2 | 25.526 | 5.12 | 20.3 |
| 33 | 417 | KHK-510-548-379-221 | 9.1 | 3.2 | 15.0 | 5.0 | 15.853 | 4.385 | 13.3 |
| 34 | 418 | KHK-511-549-380-222 | 10.4 | 3.0 | 23.8 | 3.5 | 17.016 | 6.998 | 17.1 |
| 35 | 419 | KHK-512-550-381-223 | 6.5 | 2.2 | 28.8 | 8.3 | 33.059 | 5.266 | 22.8 |
| 36 | 420 | KHK-513-551-382-224 | 25.4 | 8.6 | 25.2 | 3.2 | 22.514 | 5.225 | 24.4 |
| 37 | 421 | KHK-514-552-383-225 | 16.4 | 4.8 | 33.5 | 11.4 | 45.353 | 14.944 | 31.7 |
| 38 | 422 | KHK-515-553-384-226 | 66.1 | 4.5 | 47.0 | 3.6 | 150.28 | 13.471 | 87.8 |
| 39 | 423 | KHK-516-554-385-227 | 10.6 | 2.6 | 19.4 | 5.6 | 27.145 | 7.112 | 19.0 |
| 40 | 424 | KHK-517-555-386-228 | 12.0 | 3.4 | 24.7 | 6.3 | 24.382 | 9.127 | 20.4 |
| 41 | 425 | KHK-518-556-387-229 | 15.9 | 5.6 | 23.1 | 6.5 | 16.128 | 6.295 | 18.4 |
| 42 | 426 | KHK-520-558-389-231 | 8.9 | 1.9 | 15.6 | 3.4 | 20.589 | 4.844 | 15.0 |
| 43 | 427 | KHK-521-559-390-232 | 5.0 | 2.9 | 23.6 | 6.4 | 23.218 | 8.661 | 17.3 |
| 44 | 428 | KHK-522-560-391-233 | 9.7 | 1.7 | 12.3 | 2.7 | 12.226 | 3.342 | 11.4 |
| 45 | 429 | KHK-541-579 | 17.8 | 1.5 | 26.0 | 2.6 | 27.272 | 5.808 | 23.7 |
| 46 | 430 | KHK-544-582 | 22.9 | 2.7 | 18.4 | 1.9 | 59.795 | 8.985 | 33.7 |
| 47 | 431 | KHK-546-584 | 30.6 | 5.8 | 42.2 | 7.1 | 35.433 | 9.834 | 36.1 |
| 48 | 432 | KHK-547-585 | 12.2 | 1.9 | 17.8 | 2.6 | 20.431 | 7.549 | 16.8 |
| 49 | 433 | KHK-548-586 | 11.8 | 2.3 | 25.1 | 7.8 | 7.622 | 2.199 | 14.8 |
| 50 | 434 | KHK-549-587 | 12.2 | 2.0 | 15.0 | 3.6 | 14.783 | 3.155 | 14.0 |
| 51 | 435 | KHK-550-588 | 15.9 | 6.2 | 23.7 | 8.1 | 18.365 | 7.143 | 19.3 |
| 52 | 436 | KHK-551-589 | 24.7 | 4.2 | 34.8 | 6.1 | 14.11 | 5.994 | 24.5 |
| 53 | 437 | KHK-552-590 | 24.6 | 4.3 | 30.5 | 5.3 | 22.483 | 8.443 | 25.9 |
| 54 | 438 | KHK-553-591 | 16.1 | 1.6 | 27.3 | 4.3 | 21.753 | 4.168 | 21.7 |
| 55 | 439 | KHK-554-592 | 18.9 | 6.7 | 26.3 | 9.0 | 20.976 | 8.622 | 22.1 |
| 56 | 440 | KHK-555-593 | 22.2 | 7.1 | 27.2 | 10.4 | 24.612 | 15.246 | 24.7 |
| 57 | 441 | KHK-556-594 | 25.9 | 7.5 | 32.3 | 9.9 | 24.205 | 11.232 | 27.5 |
| 58 | 442 | KHK-557-595 | 15.1 | 1.9 | 18.9 | 2.2 | 29.27 | 5.8 | 21.1 |
| 59 | 443 | KHK-558-596 | 25.6 | 8.0 | 24.2 | 11.2 | 29.227 | 13.255 | 26.4 |
| 60 | 444 | KHK-559-597 | 21.3 | 1.9 | 29.7 | 1.8 | 23.268 | 4.973 | 24.8 |
| 61 | 445 | KHK-560-598 | 58.6 | 2.8 | 77.3 | 4.2 | 61.287 | 4.768 | 65.7 |
| 62 | 446 | KHK-561-599 | 14.7 | 2.2 | 28.9 | 3.3 | 23.131 | 4.888 | 22.3 |
| 63 | 447 | KHK-562-600 | 15.1 | 5.1 | 21.2 | 6.5 | 22.347 | 6.503 | 19.6 |
| 64 | 448 | KHK-563-601 | 34.7 | 1.7 | 47.5 | 2.5 | 63.417 | 8.195 | 48.5 |
| 65 | 449 | KHK-564-602 | 19.2 | 1.8 | 30.1 | 2.4 | 44.737 | 9.177 | 31.3 |
| 66 | 450 | KHK-565-603 | 55.7 | 3.1 | 73.0 | 5.6 | 106.349 | 11.887 | 78.3 |
| 67 | 451 | KHK-566-604 | 19.7 | 1.0 | 30.1 | 2.8 | 31.606 | 2.125 | 27.1 |
| 68 | 452 | KHK-567-605 | 9.9 | 0.5 | 15.4 | 1.3 | 14.15 | 2.218 | 13.2 |
| 69 | 453 | KHK-568-606 | 15.6 | 1.0 | 19.6 | 1.1 | 21.246 | 2.662 | 18.8 |
| 70 | 454 | KHK-569-607 | 45.3 | 5.4 | 53.3 | 5.9 | 42.462 | 12.958 | 47.0 |
| 71 | 455 | KHK-570-608 | 14.6 | 1.4 | 21.6 | 2.3 | 24.089 | 3.615 | 20.1 |
| 72 | 456 | KHK-571-609 | 15.4 | 1.8 | 17.4 | 1.9 | 28.812 | 4.966 | 20.6 |
| 73 | 457 | KHK-572-610 | 27.2 | 1.2 | 33.3 | 2.2 | 38.47 | 4.931 | 33.0 |
| 74 | 458 | KHK-573-611 | 20.6 | 1.7 | 23.1 | 2.1 | 50.396 | 5.757 | 31.4 |
| 75 | 459 | KHK-574-612 | 19.8 | 1.6 | 27.3 | 2.2 | 29.683 | 5.49 | 25.6 |
| 76 | 460 | KHK-575-613 | 31.1 | 1.9 | 31.2 | 2.4 | 37.359 | 5.282 | 33.2 |
| 77 | 461 | KHK-576-614 | 30.0 | 4.0 | 32.2 | 1.9 | 19.014 | 3.763 | 27.1 |
| 78 | 462 | KHK-577-615 | 20.8 | 1.9 | 31.6 | 2.8 | 22.807 | 5.999 | 25.1 |
| 79 | 463 | KHK-638-676 | 25.1 | 2.0 | 27.0 | 2.0 | 38.226 | 3.255 | 30.1 |
| 80 | 464 | KHK-641-679 | 29.0 | 1.7 | 33.5 | 2.0 | 30.662 | 5.567 | 31.0 |
| 81 | 465 | KHK-642-680 | 22.6 | 2.6 | 31.3 | 2.7 | 12.983 | 3.632 | 22.3 |
| 82 | 466 | KHK-643-681 | 39.1 | 2.1 | 41.5 | 2.4 | 38.644 | 6.762 | 39.7 |
| 83 | 467 | KHK-644-682 | 25.3 | 3.2 | 34.7 | 7.5 | 11.343 | 4.407 | 23.8 |
| 84 | 468 | KHK-645-683 | 15.9 | 5.0 | 21.0 | 3.1 | 21.433 | 1.586 | 19.5 |
| 85 | 469 | KHK-646-684 | 22.8 | 1.8 | 30.8 | 2.2 | 13.876 | 3.967 | 22.5 |
| 86 | 470 | KHK-647-685 | 38.8 | 1.5 | 41.9 | 2.4 | 32.316 | 6.062 | 37.7 |
| 87 | 471 | KHK-650-688 | 40.6 | 2.1 | 42.3 | 3.2 | 41.675 | 12.029 | 41.5 |
| 88 | 472 | KHK-676-714 | 56.0 | 2.4 | 52.8 | 2.8 | 71.792 | 10.911 | 60.2 |
| 89 | 473 | KHK-713-722 | 71.9 | 3.5 | 78.9 | 3.4 | 49.345 | 10.259 | 66.7 |
| 90 | 474 | KHK-826-835 | 87.0 | 6.4 | 101.5 | 5.1 | 76.866 | 14.977 | 88.5 |
| 91 | 475 | KHK-827-836 | 62.3 | 2.7 | 98.9 | 8.8 | 29.087 | 3.652 | 63.4 |
| 92 | 476 | KHK-829-838 | 100.3 | 4.5 | 86.8 | 5.3 | 17.67 | 3.397 | 68.3 |
| 93 | 477 | KHK-830-839 | 67.4 | 3.2 | 81.8 | 3.6 | 17.686 | 2.276 | 55.6 |
| 94 | 478 | KHK-831-840 | 61.7 | 3.4 | 69.8 | 5.2 | 13.313 | 3.649 | 48.3 |
| 95 | 479 | KHK-832-841 | 82.3 | 4.4 | 80.0 | 4.4 | 34.692 | 4.011 | 65.7 |
| 96 | 480 | KHK-857-895 | 73.2 | 10.7 | 53.0 | 13.9 | 88.908 | 21.326 | 71.7 |
| 97 | 481 | KHK-858-896 | 25.9 | 1.6 | 23.5 | 2.4 | 34.15 | 5.07 | 27.8 |
| 98 | 482 | KHK-859-897 | 42.4 | 2.4 | 51.3 | 4.2 | 47.048 | 2.926 | 46.9 |
| 99 | 483 | KHK-860-898-729-571 | 9.9 | 1.8 | 19.1 | 2.7 | 13.298 | 2.656 | 14.1 |
| 100 | 484 | KHK-861-899-730-572 | 5.0 | 0.6 | 3.3 | 0.4 | 2.869 | 0.815 | 3.7 |
| 101 | 485 | KHK-862-900 | 10.8 | 1.5 | 12.2 | 0.9 | 12.047 | 3.121 | 11.7 |
| 102 | 486 | KHK-865 | 6.3 | 0.7 | 7.6 | 0.5 | 5.2 | 0.828 | 6.4 |
| 103 | 487 | KHK-880 | 11.8 | 0.8 | 11.5 | 1.6 | 15.705 | 2.293 | 13.0 |
| 104 | 488 | KHK-882-920 | 6.5 | 0.8 | 6.5 | 0.9 | 4.934 | 1.748 | 6.0 |
| 105 | 489 | KHK-883-921 | 6.0 | 1.0 | 5.3 | 1.1 | 5.884 | 1.209 | 5.7 |
| 106 | 490 | KHK-884-922 | 11.6 | 1.4 | 13.2 | 1.4 | 8.24 | 2.55 | 11.0 |
| 107 | 491 | KHK-885-923 | 7.9 | 0.7 | 9.9 | 1.0 | 7.119 | 2.941 | 8.3 |
| 108 | 492 | KHK-886-924 | 10.7 | 7.4 | 10.9 | 7.0 | 4.814 | 1.257 | 8.8 |
| 109 | 493 | KHK-887-925 | 14.2 | 0.7 | 16.2 | 1.1 | 22.141 | 2.292 | 17.5 |
| 110 | 494 | KHK-888-926 | 13.3 | 1.1 | 17.9 | 1.4 | 17.895 | 1.465 | 16.4 |
| 111 | 495 | KHK-889-927 | 11.5 | 0.8 | 12.3 | 0.8 | 13.253 | 2.142 | 12.3 |
| 112 | 496 | KHK-890-928 | 8.5 | 0.7 | 7.1 | 0.9 | 9.332 | 1.26 | 8.3 |
| 113 | 497 | KHK-891-929 | 9.0 | 1.6 | 8.1 | 1.3 | 10.279 | 2.136 | 9.1 |
| 114 | 498 | KHK-892-930 | 6.0 | 1.0 | 9.4 | 1.2 | 7.1 | 2.124 | 7.5 |
| 115 | 499 | KHK-893-931 | 11.1 | 1.0 | 12.6 | 1.2 | 8.773 | 1.823 | 10.8 |
| 116 | 500 | KHK-894-932 | 16.1 | 2.3 | 19.0 | 1.7 | 8.757 | 1.518 | 14.6 |
| 117 | 501 | KHK-895-933 | 9.2 | 0.5 | 7.4 | 0.4 | 14.47 | 1.915 | 10.3 |
| 118 | 502 | KHK-896-934 | 10.4 | 1.1 | 10.5 | 1.5 | 19.901 | 4.905 | 13.6 |
| 119 | 503 | KHK-897-935 | 13.1 | 0.8 | 14.2 | 0.9 | 24.106 | 2.363 | 17.1 |
| 120 | 504 | KHK-898-936 | 28.7 | 1.4 | 32.0 | 2.9 | 25.261 | 2.143 | 28.7 |
| 121 | 505 | KHK-899-937 | 10.3 | 0.8 | 10.6 | 1.1 | 13.265 | 2.804 | 11.4 |
| 122 | 506 | KHK-900-938 | 9.2 | 0.9 | 10.4 | 0.8 | 8.036 | 1.468 | 9.2 |
| 123 | 507 | KHK-901-939 | 10.7 | 2.1 | 15.3 | 3.3 | 9.39 | 2.386 | 11.8 |
| 124 | 508 | KHK-902-940 | 17.6 | 1.2 | 17.4 | 2.2 | 13.271 | 2.387 | 16.1 |
| 125 | 509 | KHK-903-941 | 11.5 | 0.8 | 15.2 | 1.2 | 6.246 | 1.494 | 11.0 |
| 126 | 510 | KHK-904-942 | 16.1 | 3.0 | 20.6 | 2.5 | 12.783 | 3.392 | 16.5 |
| 127 | 511 | KHK-905-943 | 10.7 | 1.7 | 20.3 | 0.9 | 12.273 | 1.446 | 14.4 |
| 128 | 512 | KHK-906-944 | 13.2 | 2.9 | 24.9 | 2.4 | 6.582 | 2.052 | 14.9 |
| 129 | 513 | KHK-907-945 | 18.6 | 11.2 | 54.3 | 31.6 | 14.417 | 4.546 | 29.1 |
| 130 | 514 | KHK-908-946-777-619 | 26.7 | 14.6 | 46.4 | 21.7 | 125.267 | 92.567 | 66.1 |
| 131 | 515 | KHK-909-947-778-620 | 7.0 | 2.5 | 12.9 | 4.2 | 10.062 | 3.153 | 10.0 |
| 132 | 516 | KHK-910-948-779-621 | 19.9 | 3.0 | 19.2 | 2.6 | 19.331 | 4.315 | 19.5 |
| 133 | 517 | KHK-911-949-780-622 | 14.0 | 1.3 | 12.6 | 1.0 | 13.083 | 3.238 | 13.2 |
| 134 | 518 | KHK-912-950-781-623 | 20.4 | 1.1 | 18.3 | 1.3 | 21.258 | 3.237 | 20.0 |
| 135 | 519 | KHK-913-951-782-624 | 23.3 | 0.9 | 23.6 | 1.2 | 28.036 | 3.803 | 25.0 |
| 136 | 520 | KHK-914-952-783-625 | 70.2 | 6.9 | 69.6 | 6.1 | 65.824 | 7.388 | 68.5 |
| 137 | 521 | KHK-939-977 | 26.7 | 4.5 | 28.7 | 3.9 | 22.079 | 3.952 | 25.8 |
| 138 | 522 | KHK-940-978 | 14.8 | 2.5 | 18.1 | 3.2 | 11.124 | 3.4 | 14.7 |
| 139 | 523 | KHK-941-979 | 31.0 | 2.1 | 35.9 | 2.4 | 26.216 | 2.217 | 31.1 |
| 140 | 524 | KHK-942-980 | 106.5 | 6.3 | 93.3 | 6.2 | 55.665 | 9.171 | 85.2 |
| 141 | 525 | KHK-943-981 | 15.6 | 1.6 | 14.3 | 1.3 | 14.943 | 2.853 | 14.9 |
| 142 | 526 | KHK-944-982 | 68.2 | 2.2 | 64.0 | 3.2 | 71.816 | 5.94 | 68.0 |
| 143 | 527 | KHK-945-983 | 44.6 | 1.4 | 42.6 | 2.1 | 60.527 | 7.442 | 49.2 |
| 144 | 528 | KHK-946-984 | 42.2 | 2.1 | 43.0 | 2.0 | 44.669 | 6.993 | 43.3 |
| 145 | 529 | KHK-947-985 | 21.0 | 1.1 | 22.3 | 1.9 | 18.992 | 1.862 | 20.8 |
| 146 | 530 | KHK-948-986-817 | 12.7 | 1.7 | 13.3 | 2.9 | 11.454 | 2.967 | 12.5 |
| 147 | 531 | KHK-949-987-818 | 9.1 | 0.5 | 9.8 | 0.8 | 16.011 | 13.037 | 11.7 |
| 148 | 532 | KHK-950-988-819 | 9.6 | 1.2 | 8.5 | 0.8 | 12.475 | 3.586 | 10.2 |
| 149 | 533 | KHK-951-989-820 | 23.5 | 2.2 | 25.3 | 1.9 | 23.247 | 6.755 | 24.0 |
| 150 | 534 | KHK-952-990-821 | 14.5 | 0.8 | 18.3 | 1.6 | 15.126 | 4.553 | 16.0 |
| 151 | 535 | KHK-953-991-822 | 23.0 | 1.5 | 25.9 | 2.2 | 25.674 | 5.773 | 24.9 |
| 152 | 536 | KHK-954-992-823 | 14.1 | 1.5 | 16.9 | 2.7 | 17.22 | 2.41 | 16.1 |
| 153 | 537 | KHK-955-993-824 | 13.0 | 2.4 | 14.4 | 2.5 | 10.614 | 3.054 | 12.7 |
| 154 | 538 | KHK-956-994 | 11.8 | 2.7 | 11.9 | 3.0 | 17.221 | 5.593 | 13.6 |
| 155 | 539 | KHK-957-995 | 6.1 | 2.1 | 10.2 | 1.7 | 14.207 | 1.553 | 10.2 |
| 156 | 540 | KHK-958-996 | 17.4 | 3.2 | 17.9 | 1.0 | 26.913 | 3.733 | 20.7 |
| 157 | 541 | KHK-978-1016 | 43.0 | 3.9 | 50.1 | 4.1 | 41.716 | 3.912 | 45.0 |
| 158 | 542 | KHK-982-1020 | 51.8 | 2.6 | 69.1 | 5.3 | 67.745 | 3.674 | 62.9 |
| 159 | 543 | KHK-983-1021 | 57.0 | 6.9 | 60.3 | 7.5 | 60.141 | 8.62 | 59.2 |
| 160 | 544 | KHK-984-1022 | 15.4 | 2.1 | 16.8 | 2.1 | 17.339 | 3.931 | 16.5 |
| 161 | 545 | KHK-985-1023 | 17.4 | 2.3 | 22.7 | 7.7 | 16.976 | 8.222 | 19.0 |
| 162 | 546 | KHK-991-1029 | 21.4 | 2.9 | 22.7 | 3.9 | 27.54 | 9.397 | 23.9 |
| 163 | 547 | KHK-992-1030 | 11.2 | 1.3 | 11.7 | 1.4 | 18.836 | 2.724 | 13.9 |
| 164 | 548 | KHK-993-1031 | 32.3 | 5.5 | 26.5 | 4.5 | 19.635 | 5.352 | 26.1 |
| 165 | 549 | KHK-999-1037 | 20.8 | 1.8 | 24.4 | 1.8 | 26.652 | 2.248 | 24.0 |
| 166 | 550 | KHK-1000-1038 | 15.3 | 0.8 | 20.9 | 2.4 | 15.57 | 2.369 | 17.3 |
| 167 | 551 | KHK-1019-1057 | 29.8 | 4.4 | 29.5 | 4.4 | 36.461 | 4.748 | 31.9 |
| 168 | 552 | KHK-1054-1092 | 17.3 | 2.7 | 14.9 | 1.9 | 19.554 | 3.963 | 17.2 |
| 169 | 553 | KHK-1055-1093 | 21.0 | 1.7 | 22.2 | 2.4 | 20.314 | 4.35 | 21.2 |
| 170 | 554 | KHK-1057-1095 | 8.8 | 1.5 | 8.0 | 1.4 | 12.267 | 1.962 | 9.7 |
| 171 | 555 | KHK-1058-1096 | 51.6 | 3.0 | 52.5 | 4.1 | 54.022 | 5.889 | 52.7 |
| 172 | 556 | KHK-1059-1097 | 22.0 | 2.3 | 13.9 | 1.9 | 23.814 | 3.884 | 19.9 |
| 173 | 557 | KHK-1060-1098 | 14.7 | 0.7 | 10.4 | 0.8 | 26.531 | 6.527 | 17.2 |
| 174 | 558 | KHK-1061-1099 | 28.3 | 1.7 | 22.1 | 1.5 | 40.784 | 3.797 | 30.4 |
| 175 | 559 | KHK-1062-1100 | 11.6 | 0.7 | 12.0 | 1.1 | 20.499 | 2.193 | 14.7 |
| 176 | 560 | KHK-1063-1101 | 12.6 | 0.8 | 9.8 | 1.2 | 15.613 | 1.439 | 12.7 |
| 177 | 561 | KHK-1064-1102 | 12.7 | 2.2 | 9.7 | 2.5 | 18.761 | 3.098 | 13.7 |
| 178 | 562 | KHK-1065-1103 | 15.2 | 2.3 | 13.0 | 2.0 | 29.056 | 2.117 | 19.1 |
| 179 | 563 | KHK-1066-1104 | 14.0 | 1.4 | 13.8 | 2.9 | 16.23 | 2.914 | 14.7 |
| 180 | 564 | KHK-1067-1105 | 13.7 | 1.2 | 10.0 | 1.2 | 8.155 | 3.486 | 10.6 |
| 181 | 565 | KHK-1068-1106 | 14.0 | 0.8 | 11.4 | 1.1 | 28.373 | 4.283 | 17.9 |
| 182 | 566 | KHK-1069-1107 | 11.8 | 0.6 | 9.8 | 0.7 | 17.654 | 3.053 | 13.1 |
| 183 | 567 | KHK-1070-1108 | 20.2 | 2.0 | 14.4 | 2.7 | 30.393 | 6.07 | 21.7 |
| 184 | 568 | KHK-1071-1109 | 30.0 | 3.2 | 22.0 | 1.5 | 39.098 | 8.12 | 30.4 |
| 185 | 569 | KHK-1072-1110 | 14.5 | 0.7 | 12.9 | 0.8 | 41.196 | 15.367 | 22.8 |
| 186 | 570 | KHK-1073-1111 | 17.7 | 1.3 | 14.6 | 1.7 | 23.629 | 3.262 | 18.6 |
| 187 | 571 | KHK-1074-1112-943-785 | 25.1 | 2.1 | 23.0 | 2.4 | 34.784 | 6.555 | 27.6 |
| 188 | 572 | KHK-1075-1113-944-786 | 12.4 | 2.5 | 8.4 | 1.9 | 9.594 | 2.863 | 10.1 |
| 189 | 573 | KHK-1076-1114-945-787 | 9.8 | 1.0 | 7.5 | 1.0 | 11.44 | 3.242 | 9.6 |
| 190 | 574 | KHK-1077-1115-946-788 | 13.8 | 1.6 | 11.9 | 1.4 | 19.983 | 4.226 | 15.2 |
| 191 | 575 | KHK-1078-1116-947-789 | 12.2 | 1.4 | 10.2 | 1.5 | 19.236 | 3.613 | 13.9 |
| 192 | 576 | KHK-1079-1117-948-790 | 23.0 | 1.6 | 20.2 | 1.4 | 36.758 | 3.387 | 26.6 |
| 193 | 577 | KHK-1080-1118-949-791 | 12.4 | 0.7 | 10.3 | 0.9 | 23.489 | 2.532 | 15.4 |
| 194 | 578 | KHK-1081-1119-950-792 | 19.0 | 2.8 | 15.4 | 1.4 | 35.094 | 5.069 | 23.2 |
| 195 | 579 | KHK-1082-1120-951-793 | 51.6 | 12.7 | 36.2 | 7.1 | 79.445 | 13.29 | 55.8 |
| 196 | 580 | KHK-1083-1121-952-794 | 12.4 | 0.9 | 7.6 | 1.6 | 29.815 | 4.341 | 16.6 |
| 197 | 581 | KHK-1084-1122-953-795 | 17.8 | 1.5 | 15.4 | 1.3 | 34.538 | 5.134 | 22.6 |
| 198 | 582 | KHK-1085-1123-954-796 | 20.4 | 2.5 | 19.1 | 2.4 | 28.082 | 3.898 | 22.5 |
| 199 | 583 | KHK-1086-1124-955-797 | 9.8 | 1.4 | 9.1 | 1.9 | 22.862 | 5.973 | 13.9 |
| 200 | 584 | KHK-1087-1125-956-798 | 25.1 | 2.4 | 26.2 | 5.9 | 60.678 | 13 | 37.3 |
| 201 | 585 | KHK-1090-1128 | 15.7 | 2.5 | 14.2 | 4.3 | 47.765 | 6.748 | 25.9 |
| 202 | 586 | KHK-1091-1129 | 17.1 | 1.5 | 16.0 | 1.2 | 47.935 | 10.554 | 27.0 |
| 203 | 587 | KHK-1092-1130 | 59.1 | 13.1 | 81.9 | 15.1 | 116.084 | 17.529 | 85.7 |
| 204 | 588 | KHK-1093-1131 | 68.9 | 6.5 | 72.1 | 6.2 | 135.298 | 14.786 | 92.1 |
| 205 | 589 | KHK-1095-1133 | 39.3 | 2.2 | 34.2 | 2.9 | 49.369 | 9.398 | 41.0 |
| 206 | 590 | KHK-1096-1134 | 54.0 | 3.8 | 58.2 | 5.6 | 107.545 | 13.331 | 73.2 |
| 207 | 591 | KHK-1097-1135 | 26.5 | 3.4 | 22.1 | 3.0 | 39.738 | 6.746 | 29.5 |
| 208 | 592 | KHK-1099-1137 | 19.4 | 2.2 | 21.9 | 2.9 | 37.312 | 8.866 | 26.2 |
| 209 | 593 | KHK-1100-1138 | 31.5 | 3.9 | 31.8 | 4.6 | 86.882 | 29.059 | 50.1 |
| 210 | 594 | KHK-1101-1139 | 60.8 | 5.1 | 65.8 | 3.9 | 89.898 | 14.227 | 72.2 |
| 211 | 595 | KHK-1102-1140 | 29.8 | 1.9 | 25.1 | 2.9 | 49.962 | 4.639 | 34.9 |
| 212 | 596 | KHK-1103-1141 | 9.9 | 1.3 | 5.7 | 0.9 | 14.357 | 4.377 | 10.0 |
| 213 | 597 | KHK-1104-1142 | 7.9 | 0.5 | 5.3 | 0.7 | 14.498 | 2.508 | 9.2 |
| 214 | 598 | KHK-1106-1144 | 12.0 | 2.1 | 6.8 | 2.4 | 17.615 | 5.575 | 12.1 |
| 215 | 599 | KHK-1107-1145 | 7.6 | 1.5 | 3.5 | 1.2 | 12.097 | 3.884 | 7.7 |
| 216 | 600 | KHK-1135-1173 | 9.8 | 2.7 | 6.3 | 2.1 | 20.368 | 5.935 | 12.2 |
| 217 | 601 | KHK-1136-1174 | 55.0 | 8.2 | 57.6 | 8.7 | 51.67 | 14.619 | 54.8 |
| 218 | 602 | KHK-1137-1175 | 6.9 | 1.5 | 5.5 | 0.9 | 14.082 | 4.459 | 8.8 |
| 219 | 603 | KHK-1138-1176 | 3.4 | 1.2 | 2.7 | 1.0 | 20.618 | 4.034 | 8.9 |
| 220 | 604 | KHK-1139-1177 | 14.2 | 4.2 | 10.3 | 2.6 | 45.243 | 8.325 | 23.2 |
| 221 | 605 | KHK-1140-1178 | 22.3 | 7.0 | 14.9 | 4.7 | 58.511 | 10.307 | 31.9 |
| 222 | 606 | KHK-1141-1179 | 16.8 | 4.6 | 18.2 | 4.1 | 34.609 | 7.425 | 23.2 |
| 223 | 607 | KHK-1142-1180 | 10.0 | 4.1 | 8.0 | 2.3 | 33.362 | 7.336 | 17.1 |
| 224 | 608 | KHK-1143-1181 | 13.7 | 2.2 | 12.5 | 4.2 | 28.204 | 4.236 | 18.1 |
| 225 | 609 | KHK-1144-1182 | 11.7 | 2.1 | 14.6 | 4.6 | 49.769 | 18.354 | 25.3 |
| 226 | 610 | KHK-1145-1183 | 19.0 | 6.3 | 22.7 | 6.7 | 51.464 | 20.568 | 31.1 |
| 227 | 611 | KHK-1146-1184 | 16.8 | 4.7 | 14.7 | 5.5 | 71.937 | 37.067 | 34.5 |
| 228 | 612 | KHK-1147-1185 | 9.0 | 1.6 | 9.1 | 1.1 | 11.22 | 4.674 | 9.7 |
| 229 | 613 | KHK-1148-1186 | 8.8 | 1.7 | 6.0 | 1.8 | 18.481 | 8.166 | 11.1 |
| 230 | 614 | KHK-1149-1187 | 20.1 | 3.1 | 18.3 | 2.8 | 36.481 | 11.397 | 24.9 |
| 231 | 615 | KHK-1153-1191 | 24.4 | 3.7 | 20.5 | 3.0 | 29.434 | 6.394 | 24.8 |
| 232 | 616 | KHK-1154-1192 | 11.9 | 2.5 | 8.9 | 2.2 | 22.093 | 6.996 | 14.3 |
| 233 | 617 | KHK-1157-1195 | 14.5 | 2.2 | 11.9 | 2.2 | 26.024 | 4.864 | 17.5 |
| 234 | 618 | KHK-1158-1196 | 9.3 | 1.4 | 5.0 | 1.5 | 8.547 | 5.09 | 7.6 |
| 235 | 619 | KHK-1159-1197 | 7.3 | 1.3 | 11.6 | 1.3 | 18.971 | 4.052 | 12.6 |
| 236 | 620 | KHK-1161-1199 | 9.9 | 1.0 | 4.8 | 0.7 | 16.083 | 3.57 | 10.3 |
| 237 | 621 | KHK-1163-1201 | 13.7 | 1.3 | 11.9 | 1.4 | 28.564 | 6.393 | 18.0 |
| 238 | 622 | KHK-1164-1202 | 14.6 | 2.9 | 10.0 | 2.5 | 17.654 | 4.993 | 14.1 |
| 239 | 623 | KHK-1232-1270 | 12.0 | 2.4 | 13.2 | 2.6 | 29.824 | 4.644 | 18.4 |
| 240 | 624 | KHK-1278-1316-1147-989 | 14.0 | 2.6 | 8.5 | 3.2 | 16.096 | 5.812 | 12.9 |
| 241 | 625 | KHK-1279-1317-1148-990 | 9.4 | 2.2 | 8.2 | 2.0 | 23.969 | 10.352 | 13.9 |
| 242 | 626 | KHK-1280-1318-1149-991 | 4.5 | 1.4 | 5.3 | 1.2 | 11.007 | 1.786 | 6.9 |
| 243 | 627 | KHK-1281-1319-1150-992 | 4.0 | 1.3 | 7.4 | 2.3 | 30.368 | 6.698 | 13.9 |
| 244 | 628 | KHK-1282-1320-1151-993 | 10.0 | 1.5 | 9.4 | 2.9 | 10.4 | 2.173 | 10.0 |
| 245 | 629 | KHK-1283-1321 | 23.7 | 3.6 | 25.9 | 5.1 | 20.361 | 6.111 | 23.3 |
| 246 | 630 | KHK-1284-1322 | 17.5 | 1.2 | 15.1 | 1.9 | 23.591 | 3.59 | 18.8 |
| 247 | 631 | KHK-1285-1323 | 15.8 | 1.4 | 19.5 | 1.2 | 13.69 | 2.902 | 16.3 |
| 248 | 632 | KHK-1286-1324 | 17.7 | 2.8 | 18.7 | 2.8 | 18.507 | 3.994 | 18.3 |
| 249 | 633 | KHK-1287-1325 | 9.2 | 3.3 | 7.7 | 3.0 | 10.381 | 3.571 | 9.1 |
| 250 | 634 | KHK-1288-1326 | 8.4 | 2.9 | 3.8 | 1.7 | 11.461 | 3.398 | 7.9 |
| 251 | 635 | KHK-1289-1327 | 8.4 | 2.9 | 6.4 | 4.6 | 11.992 | 3.025 | 8.9 |
| 252 | 636 | KHK-1290-1328 | 8.7 | 1.5 | 6.7 | 1.2 | 8.926 | 3.258 | 8.1 |
| 253 | 637 | KHK-1291-1329 | 6.9 | 0.7 | 3.8 | 1.0 | 7.55 | 2.469 | 6.1 |
| 254 | 638 | KHK-1292-1330 | 17.7 | 1.1 | 12.8 | 1.2 | 30.785 | 7.501 | 20.5 |
| 255 | 639 | KHK-1293-1331 | 11.6 | 1.3 | 9.7 | 1.6 | 15.795 | 2.45 | 12.4 |
| 256 | 640 | KHK-1294-1332 | 25.7 | 1.4 | 34.2 | 2.2 | 33.058 | 7.279 | 31.0 |
| 257 | 641 | KHK-1295-1333 | 13.1 | 1.5 | 12.2 | 2.1 | 15.793 | 3.001 | 13.7 |
| 258 | 642 | KHK-1297-1335 | 21.1 | 1.3 | 21.8 | 1.5 | 24.699 | 4.755 | 22.5 |
| 259 | 643 | KHK-1323-1361 | 49.4 | 8.1 | 57.3 | 8.3 | 44.887 | 11.548 | 50.5 |
| 260 | 644 | KHK-1325-1363 | 38.7 | 2.6 | 32.1 | 3.3 | 28.739 | 4.127 | 33.2 |
| 261 | 645 | KHK-1326-1364 | 10.7 | 0.8 | 8.2 | 0.9 | 20.863 | 2.809 | 13.3 |
| 262 | 646 | KHK-1327-1365 | 54.1 | 2.2 | 52.2 | 3.4 | 94.086 | 19.883 | 66.8 |
| 263 | 647 | KHK-1328-1366 | 40.5 | 1.7 | 45.3 | 3.8 | 44.867 | 7.194 | 43.5 |
| 264 | 648 | KHK-1329-1367 | 17.2 | 1.4 | 14.4 | 1.0 | 32.257 | 2.561 | 21.3 |
| 265 | 649 | KHK-1330-1368 | 15.6 | 1.2 | 18.9 | 1.9 | 19.171 | 3.863 | 17.9 |
| 266 | 650 | KHK-1331-1369 | 15.4 | 1.6 | 12.9 | 2.0 | 42.066 | 5.461 | 23.4 |
| 267 | 651 | KHK-1332-1370 | 12.0 | 0.8 | 8.5 | 0.9 | 10.488 | 3.467 | 10.3 |
| 268 | 652 | KHK-1333-1371 | 7.2 | 0.9 | 3.7 | 0.9 | 8.179 | 2.058 | 6.3 |
| 269 | 653 | KHK-1334-1372 | 8.7 | 1.5 | 6.5 | 1.0 | 7.49 | 0.985 | 7.6 |
| 270 | 654 | KHK-1335-1373 | 9.7 | 0.6 | 5.2 | 1.1 | 9.403 | 1.153 | 8.1 |
| 271 | 655 | KHK-1336-1374 | 10.0 | 1.8 | 5.2 | 1.4 | 14.512 | 2.647 | 9.9 |
| 272 | 656 | KHK-1385-1423 | 47.5 | 3.5 | 49.0 | 4.8 | 44.122 | 7.678 | 46.9 |
| 273 | 657 | KHK-1387-1425 | 14.4 | 2.1 | 15.9 | 3.0 | 22.666 | 4.122 | 17.7 |
| 274 | 658 | KHK-1388-1426 | 27.0 | 2.9 | 28.2 | 3.3 | 34.575 | 4.985 | 29.9 |
| 275 | 659 | KHK-1389-1427 | 25.1 | 3.0 | 29.5 | 2.8 | 18.759 | 3.577 | 24.5 |
| 276 | 660 | KHK-1538-1588 | 81.1 | 15.3 | 73.7 | 17.3 | 71.289 | 16.403 | 75.3 |
| 277 | 661 | KHK-1540-1590 | 46.8 | 4.2 | 35.5 | 4.6 | 23.533 | 8.593 | 35.3 |
| 278 | 662 | KHK-1542-1592 | 80.6 | 4.7 | 89.1 | 3.3 | 85.445 | 7.942 | 85.0 |
| 279 | 663 | KHK-1665-1708 | 84.8 | 4.3 | 86.6 | 8.9 | 116.186 | 21.343 | 95.8 |
| 280 | 664 | KHK-1666-1709 | 97.0 | 2.5 | 99.6 | 3.9 | 115.899 | 10.593 | 104.2 |
| 281 | 665 | KHK-1667-1710 | 99.5 | 4.4 | 109.4 | 5.7 | 123.463 | 8.991 | 110.8 |
| 282 | 666 | KHK-1707-1750 | 91.1 | 3.5 | 107.3 | 6.3 | 123.75 | 26.01 | 107.4 |
| 283 | 667 | KHK-1708-1751 | 72.5 | 5.7 | 85.8 | 8.2 | 76.118 | 10.985 | 78.1 |
| 284 | 668 | KHK-1709-1752 | 136.2 | 5.8 | 114.5 | 5.9 | 75.202 | 14.025 | 108.7 |
| 285 | 669 | KHK-1869-1918 | 118.1 | 21.1 | 111.2 | 20.6 | 97.088 | 18.195 | 108.8 |
| 286 | 670 | KHK-1870-1919 | 90.2 | 8.7 | 83.2 | 11.2 | 87.62 | 22.519 | 87.0 |
| 287 | 671 | KHK-1871-1920 | 81.7 | 4.6 | 80.6 | 4.6 | 119.805 | 13.019 | 94.1 |
| 288 | 672 | KHK-1872-1921 | 94.5 | 4.4 | 87.4 | 5.7 | 74.492 | 13.782 | 85.4 |
| 289 | 673 | KHK-1873-1922 | 93.2 | 5.4 | 90.9 | 4.6 | 88.62 | 13.98 | 90.9 |
| 290 | 674 | KHK-1874-1923 | 93.2 | 6.3 | 90.7 | 4.9 | 74.793 | 8.913 | 86.2 |
| 291 | 675 | KHK-1875-1924 | 86.5 | 8.5 | 76.3 | 8.2 | 45.965 | 7.621 | 69.6 |
| 292 | 676 | KHK-1876-1925 | 73.7 | 9.7 | 61.1 | 8.7 | 34.813 | 6.807 | 56.6 |
| 293 | 677 | KHK-1877-1926 | 72.7 | 3.9 | 51.0 | 5.3 | 45.122 | 9.468 | 56.3 |
| 294 | 678 | KHK-1878-1927 | 75.8 | 4.8 | 79.7 | 8.0 | 54.716 | 8.031 | 70.1 |
| 295 | 679 | KHK-1879-1928 | 90.6 | 8.4 | 77.0 | 9.0 | 86.62 | 13.219 | 84.7 |
| 296 | 680 | KHK-1880-1929 | 100.4 | 8.5 | 84.5 | 9.8 | 85.501 | 13.022 | 90.1 |
| 297 | 681 | KHK-1900-1949 | 104.9 | 12.1 | 96.5 | 15.3 | 70.712 | 8.039 | 90.7 |
| 298 | 682 | KHK-1905-1954 | 71.4 | 17.0 | 71.0 | 22.1 | 35.273 | 14.001 | 59.2 |
| 299 | 683 | KHK-1971-2025 | 77.5 | 25.4 | 128.3 | 28.3 | 34.399 | 9.559 | 80.1 |
| 300 | 684 | KHK-1974-2028 | 74.2 | 6.0 | 64.4 | 7.7 | 50.763 | 6.122 | 63.1 |
| 301 | 685 | KHK-1975-2029 | 80.8 | 7.8 | 80.5 | 7.1 | 55.779 | 10.606 | 72.3 |
| 302 | 686 | KHK-1976-2030 | 69.2 | 6.8 | 73.5 | 8.5 | 53.873 | 16.663 | 65.5 |
| 303 | 687 | KHK-1978-2032 | 73.7 | 10.3 | 73.6 | 10.0 | 57.334 | 7.876 | 68.2 |
| 304 | 688 | KHK-1979-2033 | 81.2 | 9.5 | 95.1 | 13.6 | 43.016 | 8.767 | 73.1 |
| 305 | 689 | KHK-2032-2086 | 94.2 | 6.3 | 124.6 | 9.4 | 99.842 | 10.374 | 106.2 |
| 306 | 690 | KHK-2035-2089 | 66.5 | 6.2 | 72.2 | 14.5 | 47.134 | 6.316 | 61.9 |
| 307 | 691 | KHK-2036-2090 | 82.4 | 9.1 | 110.4 | 15.5 | 56.532 | 12.458 | 83.1 |
| 308 | 692 | KHK-2037-2091 | 88.9 | 8.1 | 72.5 | 7.9 | 48.451 | 10.859 | 70.0 |
| 309 | 693 | KHK-2038-2092 | 74.2 | 3.6 | 58.7 | 4.7 | 44.437 | 4.32 | 59.1 |
| 310 | 694 | KHK-2039-2093 | 75.1 | 5.3 | 76.3 | 8.1 | 51.597 | 6.117 | 67.7 |
| 311 | 695 | KHK-2040-2094 | 75.9 | 8.5 | 70.0 | 11.2 | 57.499 | 6.477 | 67.8 |
| 312 | 696 | KHK-2041-2095 | 80.5 | 4.1 | 78.1 | 3.5 | 78.593 | 10.192 | 79.1 |
| 313 | 697 | KHK-2042-2096 | 79.1 | 5.2 | 84.7 | 4.0 | 88.699 | 8.352 | 84.2 |
| 314 | 698 | KHK-2043-2097 | 72.0 | 3.8 | 70.0 | 2.7 | 83.791 | 8.891 | 75.3 |
| 315 | 699 | KHK-2044-2098 | 37.6 | 12.9 | 32.3 | 13.4 | 30.83 | 15.258 | 33.6 |
| 316 | 700 | KHK-2045-2099 | 101.3 | 8.7 | 87.4 | 11.3 | 54.839 | 12.261 | 81.2 |
| 317 | 701 | KHK-2067-2121 | 88.2 | 5.1 | 78.4 | 2.6 | 75.916 | 8.438 | 80.8 |
| 318 | 702 | KHK-2069-2123 | 83.1 | 3.7 | 84.9 | 5.2 | 63.679 | 10.343 | 77.2 |
| 319 | 703 | KHK-2091-2145 | 83.8 | 5.4 | 87.1 | 8.9 | 53.463 | 10.34 | 74.8 |
| 320 | 704 | KHK-2092-2146 | 85.4 | 6.2 | 89.7 | 7.8 | 68.656 | 5.01 | 81.3 |
| 321 | 705 | KHK-2093-2147 | 102.7 | 21.4 | 65.4 | 19.9 | 71.693 | 14.857 | 79.9 |
| 322 | 706 | KHK-2094-2148 | 88.8 | 6.4 | 94.1 | 6.7 | 53.85 | 10.392 | 78.9 |
| 323 | 707 | KHK-2095-2149 | 76.2 | 21.4 | 97.2 | 25.9 | 47.372 | 14.735 | 73.6 |
| 324 | 708 | KHK-2096-2150 | 87.9 | 16.4 | 78.6 | 13.2 | 49.454 | 14.398 | 72.0 |
| 325 | 709 | KHK-2105 | 92.1 | 7.7 | 90.8 | 11.5 | 97.683 | 10.156 | 93.5 |
| 326 | 710 | KHK-2148-2197 | 86.5 | 5.8 | 79.3 | 16.0 | 76.198 | 8.142 | 80.7 |
| 327 | 711 | KHK-2149-2198 | 71.3 | 3.7 | 73.8 | 4.2 | 55.558 | 6.731 | 66.9 |
| 328 | 712 | KHK-2150-2199 | 92.1 | 7.1 | 97.5 | 4.9 | 75.703 | 6.126 | 88.4 |
| 329 | 713 | KHK-2151-2200 | 96.2 | 3.7 | 108.0 | 10.3 | 91.908 | 5.852 | 98.7 |
| 330 | 714 | KHK-2152-2201 | 78.7 | 6.5 | 74.7 | 9.6 | 42.766 | 6.332 | 65.4 |
| 331 | 715 | KHK-2153-2202 | 95.2 | 14.8 | 73.9 | 13.1 | 47.169 | 10.902 | 72.1 |
| 332 | 716 | KHK-2154-2203 | 114.1 | 11.9 | 92.3 | 7.2 | 61.728 | 10.943 | 89.4 |
| 333 | 717 | KHK-2155-2204 | 92.1 | 8.4 | 83.1 | 3.8 | 119.537 | 11.18 | 98.2 |
| 334 | 718 | KHK-2156-2205 | 104.7 | 5.4 | 91.6 | 4.7 | 148.445 | 15.208 | 114.9 |
| 335 | 719 | KHK-2157-2206 | 94.2 | 8.4 | 92.0 | 10.4 | 68.735 | 7.132 | 85.0 |
| 336 | 720 | KHK-2159-2208 | 85.4 | 4.5 | 78.4 | 6.2 | 62.397 | 10.642 | 75.4 |
| 337 | 721 | KHK-2160-2209 | 72.7 | 1.9 | 81.8 | 5.3 | 56.483 | 11.255 | 70.3 |
| 338 | 722 | KHK-2161-2210 | 93.7 | 14.6 | 74.3 | 10.9 | 18.252 | 5.102 | 62.1 |
| 339 | 723 | KHK-2162-2211 | 106.7 | 11.3 | 127.3 | 17.9 | 53.455 | 15.254 | 95.8 |
| 340 | 724 | KHK-2163-2212 | 79.5 | 8.2 | 91.6 | 6.4 | 49.199 | 6.236 | 73.5 |
| 341 | 725 | KHK-2164-2213 | 101.1 | 13.4 | 115.2 | 20.4 | 84.893 | 29.662 | 100.4 |
| 342 | 726 | KHK-2165-2214 | 97.0 | 10.8 | 102.1 | 9.9 | 76.079 | 10.525 | 91.7 |
| 343 | 727 | KHK-2166-2215 | 91.6 | 20.4 | 89.3 | 22.7 | 55.353 | 11.894 | 78.8 |
| 344 | 728 | KHK-2170-2219 | 75.9 | 4.5 | 89.7 | 4.6 | 68.461 | 8.991 | 78.0 |
| 345 | 729 | KHK-2196-2245 | 60.3 | 2.9 | 65.1 | 4.4 | 43.35 | 5.951 | 56.3 |
| 346 | 730 | KHK-2197-2246 | 85.4 | 8.4 | 98.9 | 9.4 | 65.81 | 6.865 | 83.4 |
| 347 | 731 | KHK-2198-2247 | 89.4 | 15.0 | 108.1 | 10.3 | 44.371 | 7.323 | 80.6 |
| 348 | 732 | KHK-2199-2248 | 97.2 | 14.8 | 91.3 | 16.1 | 49.493 | 8.874 | 79.3 |
| 349 | 733 | KHK-2200-2249 | 104.7 | 10.7 | 111.8 | 13.5 | 47.327 | 5.488 | 87.9 |
| 350 | 734 | KHK-2201-2250 | 100.3 | 11.7 | 102.7 | 13.8 | 52.984 | 11.652 | 85.3 |
| 351 | 735 | KHK-2205 | 96.9 | 17.0 | 88.0 | 10.9 | 55.021 | 8.208 | 80.0 |
| 352 | 736 | KHK-2238 | 89.1 | 8.0 | 99.6 | 8.2 | 113.917 | 11.636 | 100.9 |
| 353 | 737 | KHK-2260-2309 | 111.5 | 13.2 | 110.2 | 11.9 | 95.452 | 20.407 | 105.7 |
| 354 | 738 | KHK-2261-2310 | 103.6 | 9.1 | 106.9 | 12.4 | 97.581 | 15.894 | 102.7 |
| 355 | 739 | KHK-2262-2311 | 141.3 | 14.6 | 132.9 | 11.8 | 112.052 | 19.366 | 128.7 |
| 356 | 740 | KHK-2263-2312 | 104.0 | 10.5 | 80.5 | 11.4 | 59.852 | 7.342 | 81.4 |
| 357 | 741 | KHK-2264-2313 | 100.7 | 17.1 | 88.6 | 15.5 | 53.023 | 12.056 | 80.8 |
| 358 | 742 | KHK-2265-2314 | 103.2 | 11.2 | 103.8 | 11.9 | 60.929 | 8.309 | 89.3 |
| 359 | 743 | KHK-2266-2315 | 119.8 | 8.9 | 110.3 | 8.5 | 66.846 | 9.19 | 99.0 |
| 360 | 744 | KHK-2299 | 77.7 | 3.2 | 72.0 | 5.9 | 69.804 | 11.442 | 73.2 |
| 361 | 745 | KHK-2317-2366 | 81.2 | 3.0 | 84.4 | 7.7 | 66.04 | 7.299 | 77.2 |
| 362 | 746 | KHK-2318-2367 | 86.6 | 3.7 | 97.0 | 5.2 | 66.519 | 4.573 | 83.4 |
| 363 | 747 | KHK-2319-2368 | 127.5 | 12.5 | 102.9 | 8.9 | 82.338 | 11.524 | 104.2 |
| 364 | 748 | KHK-2320-2369 | 98.9 | 11.1 | 94.2 | 15.6 | 59.154 | 17.115 | 84.1 |
| 365 | 749 | KHK-2321-2370 | 127.6 | 13.8 | 127.6 | 16.5 | 81.979 | 19.313 | 112.4 |
| 366 | 750 | KHK-2322-2371 | 83.3 | 9.1 | 68.5 | 8.6 | 78.252 | 14.344 | 76.7 |
| 367 | 751 | KHK-2323-2372 | 83.2 | 7.3 | 79.8 | 7.7 | 57.202 | 9.062 | 73.4 |
| 368 | 752 | KHK-2324-2373 | 95.0 | 3.0 | 101.4 | 5.2 | 118.194 | 16.285 | 104.9 |
| 369 | 753 | KHK-2325-2374 | 123.0 | 15.6 | 153.7 | 23.1 | 188.498 | 24.768 | 155.1 |
| 370 | 754 | KHK-2326-2375 | 94.5 | 12.0 | 101.2 | 8.2 | 110.056 | 29.627 | 101.9 |
| 371 | 755 | KHK-2332 | 96.2 | 11.9 | 122.2 | 10.2 | 120.096 | 15.829 | 112.8 |
| 372 | 756 | KHK-2333 | 97.9 | 12.7 | 73.0 | 9.9 | 50.041 | 16.617 | 73.7 |
| 373 | 757 | KHK-2335 | 104.4 | 9.0 | 81.2 | 7.5 | 33.77 | 6.814 | 73.1 |
| 374 | 758 | KHK-2340 | 64.4 | 14.3 | 45.7 | 12.3 | 34.661 | 10.468 | 48.2 |
| 375 | 759 | KHK-2341 | 61.9 | 10.6 | 53.7 | 6.9 | 43.579 | 8.14 | 53.1 |
| 376 | 760 | KHK-2346 | 104.5 | 9.2 | 92.5 | 12.6 | 104.386 | 32.315 | 100.4 |
| 377 | 761 | KHK-2352 | 78.5 | 6.7 | 83.6 | 8.0 | 97.544 | 18.168 | 86.6 |
| 378 | 762 | KHK-2358 | 78.8 | 7.0 | 75.7 | 9.9 | 64.974 | 13.327 | 73.1 |
| 379 | 763 | KHK-2359 | 89.5 | 9.0 | 91.8 | 8.2 | 71.314 | 7.358 | 84.2 |
| 380 | 764 | KHK-2360 | 132.4 | 26.2 | 82.8 | 8.7 | 159.973 | 15.729 | 125.0 |
| 381 | 765 | KHK-2361 | 110.4 | 8.5 | 87.7 | 7.8 | 87.634 | 7.799 | 95.3 |
| 382 | 766 | KHK-2362 | 92.5 | 12.8 | 72.7 | 7.2 | 84.427 | 12.833 | 83.2 |
| 383 | 767 | KHK-2363 | 100.4 | 9.2 | 76.1 | 14.4 | 52.066 | 18.068 | 76.2 |
| 384 | 768 | KHK-2364 | 116.4 | 9.8 | 88.5 | 8.3 | 120.931 | 26.855 | 108.6 |
| 385 | 769 | KHK-2365 | 100.5 | 5.7 | 92.9 | 5.2 | 132.668 | 7.14 | 108.7 |
| 386 | 770 | KHK-2366 | 129.3 | 7.2 | 113.5 | 5.7 | 135.163 | 16.2 | 126.0 |
| 387 | 771 | KHK-2367 | 123.3 | 9.0 | 105.9 | 9.2 | 136.356 | 9.025 | 121.9 |

### Example 3: RNAi Oligonucleotide Inhibition of Both KHK Isoforms In Vivo

The in vitro screening assay in Example 2 validated the ability of KHK DsiRNA to knock-down both isoforms of KHK (KHK-All). To confirm the ability of the RNAi oligonucleotides to knockdown both KHK-A and KHK—C isoforms, a side-by-side HDI mouse model was used. First, the nucleotide sequences comprising a subset of the 384 DsiRNAs identified in Example 2, and that recognize human/NHP-conserved KHK, were used to generate corresponding double-stranded RNAi oligonucleotides comprising a nicked tetraloop GalNAc-conjugated structure (referred to herein as “GalNAc-conjugated KHK oligonucleotides” or “GalNAc-KHK constructs”) having a 36-mer passenger strand and a 22-mer guide strand (Table 3). Specifically, to generate the 22-mer guide strand, the 19-mer core antisense strand sequences used in Example 2 (e.g., SEQ ID NOs: 948-953) were modified to have a phosphorylated uracil at the 5′ end and two guanines at the 3′ end. To generate the 36-mer passenger strand, an adenine corresponding to the phosphorylated uracil in the antisense strand and a 16-mer stem loop (SEQ ID NO: 871) were added to the 3′ end of the 19-mer core sense strand sequences used in Example 2 (e.g., SEQ ID NOs: 942-947). Further, the nucleotide sequences comprising the passenger strand and guide strand of the GalNAc-conjugated KHK oligonucleotides have a distinct pattern of modified nucleotides and phosphorothioate linkages (e.g., see FIG. 2A, FIG. 2B and Table 3 for schematics of the generic structure and key of chemical modifications; referred to herein as Low-2′-Fluoro (3PS) and Low-2′-Fluoro (2PS), respectively, together as the Low-2′-Fluoro pattern for GalNAc-conjugated KHK oligonucleotides). The three adenosine nucleotides comprising the tetraloop are each conjugated to a GalNAc moiety (CAS #: 14131-60-3). The modification pattern is represented below in two interchangeable modification keys.

Low-2′-Fluoro (3PS) Modification Pattern for
GalNAc-KHK Constructs (5′ Antisense 3PS)
Sense Strand:
5′-mX-S-mX-mX-mX-mX-mX-mX-fX-fX-fX-fX-mX-mX-
mX-mX-mX-mX-mX-mX-mX-mX-mX-mX-mX-mX-mX-mX-
[ademA-GalNAc]- [ademA-GalNAc]- [ademA-
GalNAc]-mX-mX-mX-mX-mX-mX-3′.
Hybridized to:
Antisense Strand:
5′-[MePhosphonate-4O-mX]-S-fX-S-fX-S-fX-fX-
mX-fX-mX-mX-fX-mX-mX-mX-fX-mX-mX-mX-mX-mX-
mX-S-mX-S-mX-3′
(Modification key: Table 3).
Or, represented as:
Sense Strand:
[mXs][mX][mX][mX][mX][mX][mX][fX][fX][fX]
[fX][mX][mX][mX][mX][mX][mX][mX][mX][mX]
[mX][mX][mX][mX][mX][mX][mX][ademA-GalNAc]
[ademA-GalNAc][ademA-GalNAc][mX][mX][mX]
[mX][mX][mX]
Hybridized to:
Antisense Strand:
[MePhosphonate-4O-mXs][fXs][fXs][fX][fX]
[mX][fX][mX][mX][fX][mX][mX][mX][fX][mX]
[mX][mX][mX][mX][mXs][mXs][mX]
(Modification key: Table 3).
Low-2′Fluoro (2PS) Modification Pattern
for GalNAc-KHK Constructs (5′ Antisense
2PS)
Sense Strand:
5′-mX-S-mX-mX-mX-mX-mX-mX-fX-fX-fX-fX-mX-
mX-mX-mX-mX-mX-mX-mX-mX-mX-mX-mX-mX-mX-
mX-mX-[ademA-GalNAc]- [ademA-GalNAc]-
[ademA-GalNAc]-mX-mX-mX-mX-mX-mX-3′.
Hybridized to:
Antisense Strand:
5′-[MePhosphonate-4O-mX]-S-fX-S-fX-fX-fX-
mX-fX-mX-mX-fX-mX-mX-mX-fX-mX-mX-mX-mX-
mX-mX-S-mX-S-mX-3′
(Modification key: Table 3).
Or, represented as:
Sense Strand:
[mXs][mX][mX][mX][mX][mX][mX][fX][fX][fX]
[fX][mX][mX][mX][mX][mX][mX][mX][mX][mX]
[mX][mX][mX][mX][mX][mX][mX][ademA-GalNAc]
[ademA-GalNAc][ademA-GalNAc][mX][mX][mX]
[mX][mX][mX]
Hybridized to:
Antisense Strand:
[MePhosphonate-4O-mXs][fXs][fX][fX][fX]
[mX][fX][mX][mX][fX][mX][mX][mX][fX][mX]
[mX][mX][mX][mX][mXs][mXs][mX]

TABLE 3
Modification Key

| | |
|---|---|
| Symbol | Modification/linkage |

Key 1

| | |
|---|---|
| mX | 2′-O-methyl modified nucleotide |
| fX | 2′-fluoro modified nucleotide |
| -S- | phosphorothioate linkage |
| — | phosphodiester linkage |
| [MePhosphonate- | 5′-methoxyphosphonate-4′-oxy modified nucleotide |
| 4O-mX] | |
| ademA-GalNAc | GalNAc attached to an adenine nucleotide |

Key 2

| | |
|---|---|
| [mXs] | 2′-O-methyl modified nucleotide with a |
| | phosphorothioate linkage to the neighboring nucleotide |
| [fXs] | 2′-fluoro modified nucleotide with a |
| | phosphorothioate linkage to the neighboring nucleotide |
| [mX] | 2′-O-methyl modified nucleotide with phosphodiester |
| | linkages to neighboring nucleotides |
| [fX] | 2′-fluoro modified nucleotide with phosphodiester |
| | linkages to neighboring nucleotides |

The GalNAc-KHK constructs were then used to evaluate inhibition efficacy in mice. Specifically, 6-8-week-old female CD-1 mice (n=5) were subcutaneously administered the indicated GalNAc-conjugated KHK oligonucleotides (Table 4) at a dose of 2 mg/kg formulated in PBS. A control group of mice (n=5) were administered only PBS. Three days later (72 hours), the mice were hydrodynamically injected (HDI) either with a DNA plasmid (pCMV6-KHK-C, Cat #: RC223488, OriGene) encoding the full human KHK gene (NM_006488.3) (25 μg) or plasmid (pCMV6-KHK-A, Cat #;RC202424, OriGene) encoding the full human KHK-A gene (NM_000221) under control of a ubiquitous cytomegalovirus (CMV) promoter sequence. One day after introduction of the DNA plasmid, liver samples from HDI mice were collected. The values were normalized for transfection efficiency using the NeoR gene included on the DNA plasmid.

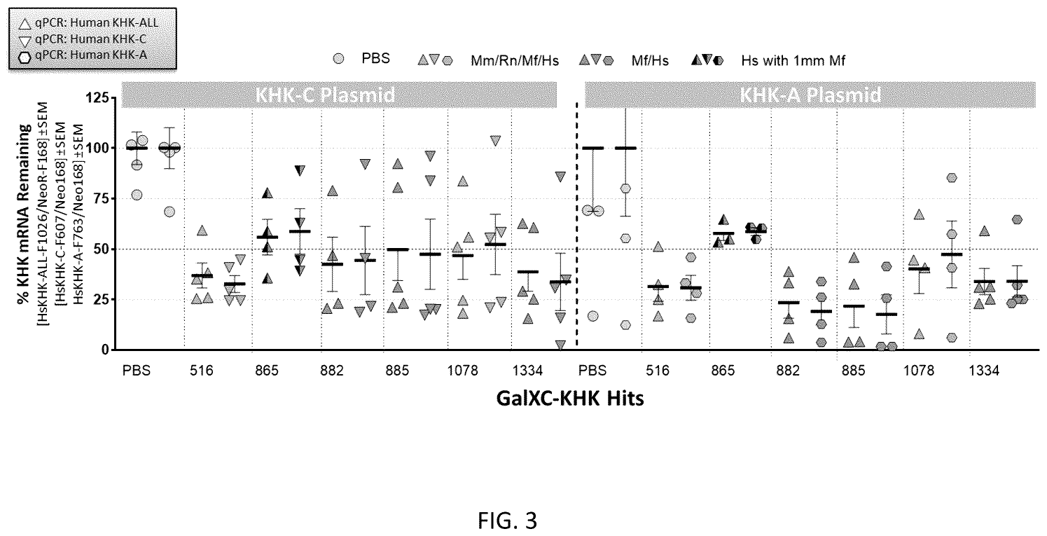

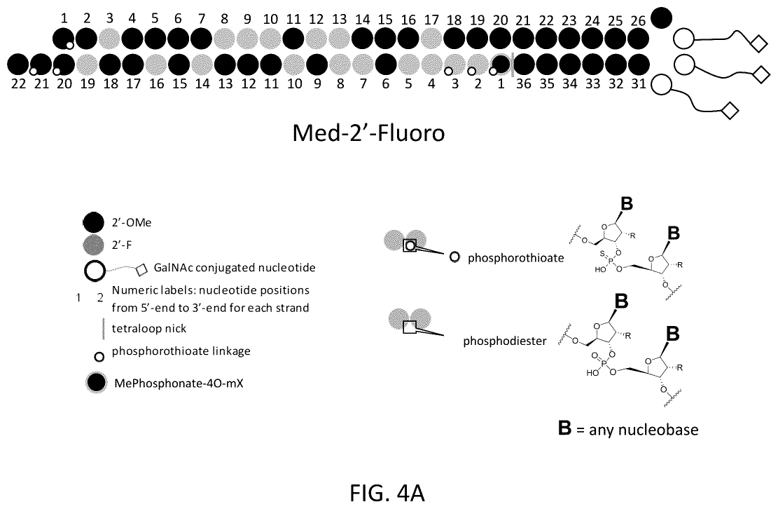

Total RNA isolated from mouse livers were used to assess relative KHK mRNA expressions by qRT-PCR. The TaqMan RT-qPCR probes from Life Technologies were used to evaluate [3′ assay (Forward-1026; TGGAGGTGGAGAAGCCA (SEQ ID NO: 865), Reverse-1157; GACCATACAAGCCCCTCAAG (SEQ ID NO:866), Probe-1080; TGGTGTTTGTCAGCAAAGATGTGGC (SEQ ID NO:867)) and a 5′ assay (Forward-496; AGGAAGCTCTGGGAGTA (SEQ ID NO: 868), Reverse-596; CCTCCTTAGGGTACTTGTC (SEQ ID NO: 869), Probe-518; ATGGAAGAGAAGCAGATCCTGTGCG (SEQ ID NO: 870))]. The values were normalized for transfection efficiency using the NeoR gene included on the DNA plasmid. HDI mice were generated as described above but using a human KHK-A plasmid or a human KHK-C plasmid. The mice were treated in groups of 5 with the GaINAc-KHK constructs in Table 4 (with the Low-2′-Fluoro modification pattern). Livers were collected and mRNA measured using primer pairs recognizing KHK-All, KHK-C, or KHK-A. The results confirmed that GalNAc-KHK constructs designed to target all KHK transcripts demonstrate successful knockdown in both the human KHK-A and KHK-C HDI mouse models (FIG. 3).

TABLE 4
GalNAc-KHK Constructs Evaluated In
KHK-C and KHK-A HDI Mouse Models

| | Sense | Anti-sense | Sense | Anti-sense |
|---|---|---|---|---|
| | Strand | Strand | Strand | Strand |
| | Unmodified | Unmodified | Modified | Modified |
| Name | SEQ ID NO | SEQ ID NO | SEQ ID NO | SEQ ID NO |
| KHK-516 | 39 | 423 | 775 | 820 |
| KHK-865 | 102 | 486 | 779 | 824 |
| KHK-882 | 104 | 488 | 780 | 825 |
| KHK-885 | 107 | 491 | 782 | 827 |
| KHK-1078 | 191 | 575 | 785 | 830 |
| KHK-1334 | 269 | 653 | 804 | 849 |

### Example 4: Changes in Modification Pattern of KHK-Targeting RNAi Oligonucleotides Maintains mRNA Inhibition Efficacy

To assess whether modification patterns may impact the targeting efficiency and stability of GalNAc-KHK constructs, two unique patterns were analyzed in HDI mice. Specifically, the modification patterns used were the Low-2′-fluoro pattern described in Example 3 (see FIG. 2A and FIG. 2B) and a Med-2′-fluoro pattern (see FIG. 4A).

Med-2′-Fluoro Modification Pattern for GalNAc-
KHK Constructs
Sense Strand:
5′-mX-S-mX-fX-mX-mX-mX-mX-fX-fX-fX-mX-fX-fX-
mX-mX-mX-fX-mX-mX-mX-mX-mX-mX-mX-mX-mX-mX-
[ademA-GalNAc]- [ademA-GalNAc]- [ademA-
GalNAc]-mX-mX-mX-mX-mX-mX-3′.
Hybridized to:
Antisense Strand:
5′-[MePhosphonate-4O-mX]-S-fX-S-fX-S-fX-fX-
mX-fX-fX-mX-fX-mX-mX-mX-fX-mX-fX-mX-mX-fX-
mX-S-mX-S-mX-3′
(Modification key: Table 3).
Or, represented as:
Sense Strand:
[mXs][mX][fX][mX][mX][mX][mX][fX][fX][fX]
[mX][fX][fX][mX][mX][mX][fX][mX][mX][mX]
[mX][mX][mX][mX][mX][mX][mX][ademA-GalNAc]
[ademA-GalNAc][ademA-GalNAc][mX][mX][mX]
[mX][mX][mX]
Hybridized to:
Antisense Strand:
[MePhosphonate-4O-mXs][fXs][fXs][fX][fX]
[mX][fX][fX][mX][fX][mX][mX][mX][fX][mX]
[fX][mX][mX][fX][mXs][mXs][mX]
(Modification key: Table 3).

HDI mice were generated as described in Example 3. Mice were treated with Low-2′-Fluoro or Med-2′-Fluoro modified KHK constructs (Table 5). 72 hours after treatment, mice were hydrodynamically injected with [pcDNA3.1-KHK-C, encoding the full human KHK gene (NM_006488)]. Livers were collected and processed as described in Example 3. A group of GalNAc-KHK constructs (KHK-0861,-0865,-0882,-0883,-0885) were mixed together and used as a positive control for inhibition. Both modification patterns resulted in inhibition of KHK mRNA in mice (FIG. 4B-4E). These results demonstrate that both modification patterns provided knockdown of the target mRNA.

TABLE 5
GalNAc-KHK Constructs for Modification Pattern Assay

| | | Sense | Anti-sense | Sense | Anti-sense |
|---|---|---|---|---|---|
| | | Strand | Strand | Strand | Strand |
| | Modifi- | Unmodi- | Unmodi- | Modified | Modified |
| | cation | fied SEQ | fied SEQ | SEQ | SEQ |
| Name | pattern | ID NO | ID NO | ID NO | ID NO |
| KHK-861 | Low-2′F | 100 | 484 | 778 | 823 |
| KHK-861 | Med-2′F | 100 | 484 | 808 | 853 |
| KHK-865 | Low-2′F | 102 | 486 | 779 | 824 |
| KHK-865 | Med-2′F | 102 | 486 | 809 | 854 |
| KHK-882 | Low-2′F | 104 | 488 | 780 | 820 |
| KHK-882 | Med-2′F | 104 | 488 | 810 | 855 |
| KHK-883 | Low-2′F | 105 | 489 | 781 | 826 |
| KHK-883 | Med-2′F | 105 | 489 | 811 | 856 |
| KHK-885 | Low-2′F | 107 | 491 | 782 | 827 |
| KHK-885 | Med-2′F | 107 | 491 | 812 | 857 |
| KHK-1288 | Low-2′F | 250 | 634 | 787 | 832 |
| KHK-1288 | Med-2′F | 250 | 634 | 816 | 861 |
| KHK-1290 | Low-2′F | 252 | 636 | 788 | 833 |
| KHK-1290 | Med-2′F | 252 | 636 | 817 | 862 |
| KHK-1334 | Low-2′F | 269 | 653 | 804 | 849 |
| KHK-1334 | Med-2′F | 269 | 653 | 818 | 863 |
| KHK-516 | Med-2′F | 39 | 423 | 805 | 850 |
| KHK-804 | Med-2′F | N/A | N/A | 806 | 851 |
| KHK-829 | Med-2′F | 92 | 476 | 807 | 852 |
| KHK-1076 | Med-2′F | 189 | 573 | 814 | 859 |
| KHK-1078 | Med-2′F | 191 | 575 | 815 | 860 |

### Example 5: RNAi Oligonucleotide Inhibition of KHK Expression In Vivo

### Mouse HDI KHK Knockdown Screening Studies

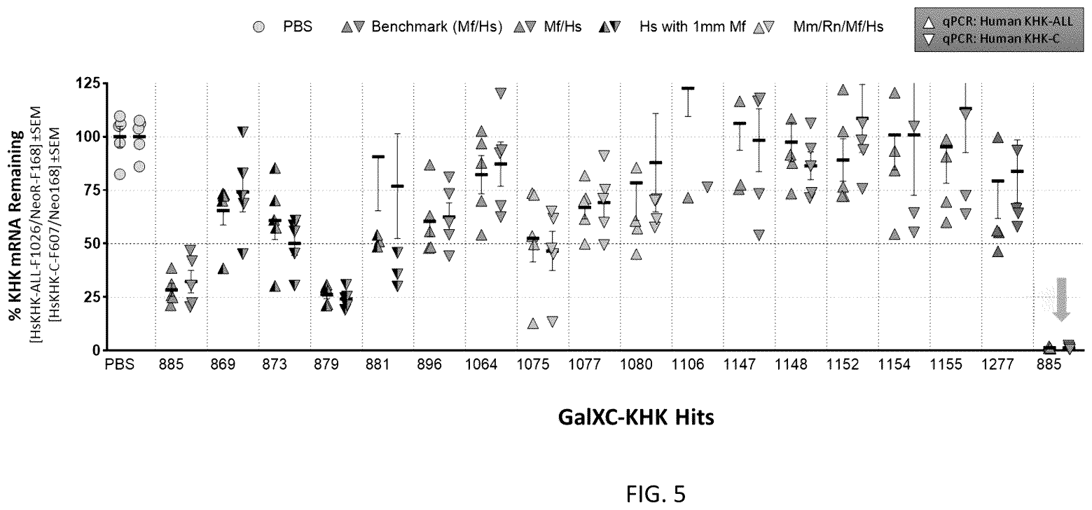

The GalNAc-conjugated KHK oligonucleotides listed in Table 6 were evaluated in 5 HDI mice as described in Example 3. GalNAc-KHK construct treatment effectively reduced KHK-All mRNA (FIG. 5). When using primers specific for the KHK-C isoform, the GalNAc-KHK constructs were still effective at reducing mRNA (FIG. 5).

TABLE 6
GalNAc-KHK Constructs Assayed in HDI Model

| | Sense Strand | Antisense Strand |
|---|---|---|
| Name | Modified SEQ ID NO | Modified SEQ ID NO |
| KHK-885 | 782 | 827 |
| KHK-869 | 795 | 840 |
| KHK-873 | 796 | 841 |
| KHK-879 | 797 | 842 |
| KHK-881 | 798 | 843 |
| KHK-896 | 799 | 844 |
| KHK-1064 | 800 | 845 |
| KHK-1075 | 784 | 829 |
| KHK-1077 | 801 | 846 |
| KHK-1080 | 802 | 847 |
| KHK-1106 | 803 | 848 |
| KHK-1147 | 794 | 839 |
| KHK-1148 | 789 | 834 |
| KHK-1152 | 790 | 835 |
| KHK-1154 | 791 | 836 |
| KHK-1155 | 792 | 837 |
| KHK-1277 | 793 | 838 |

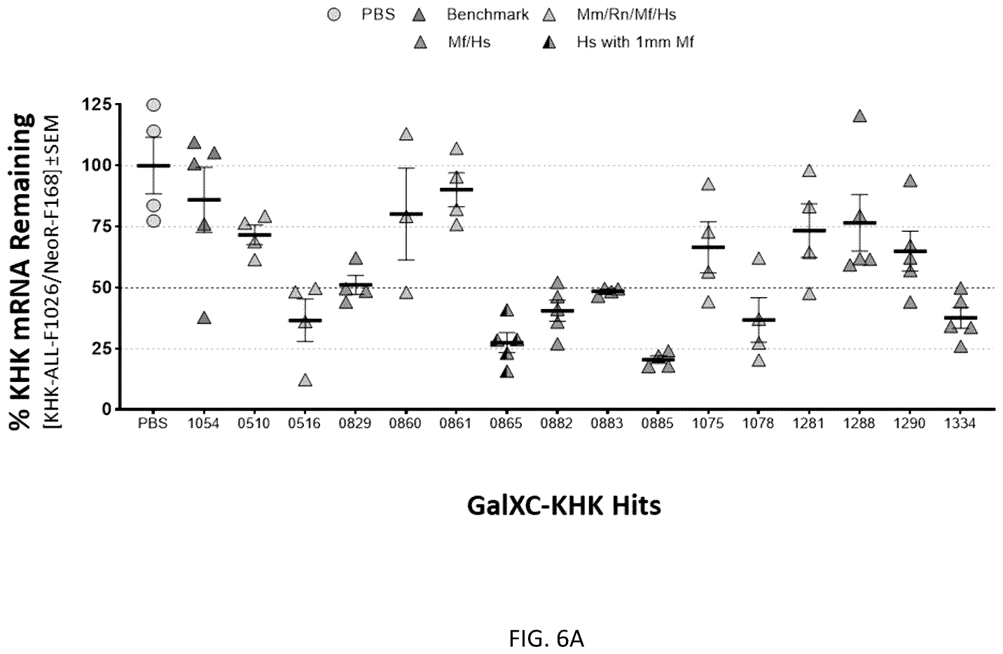

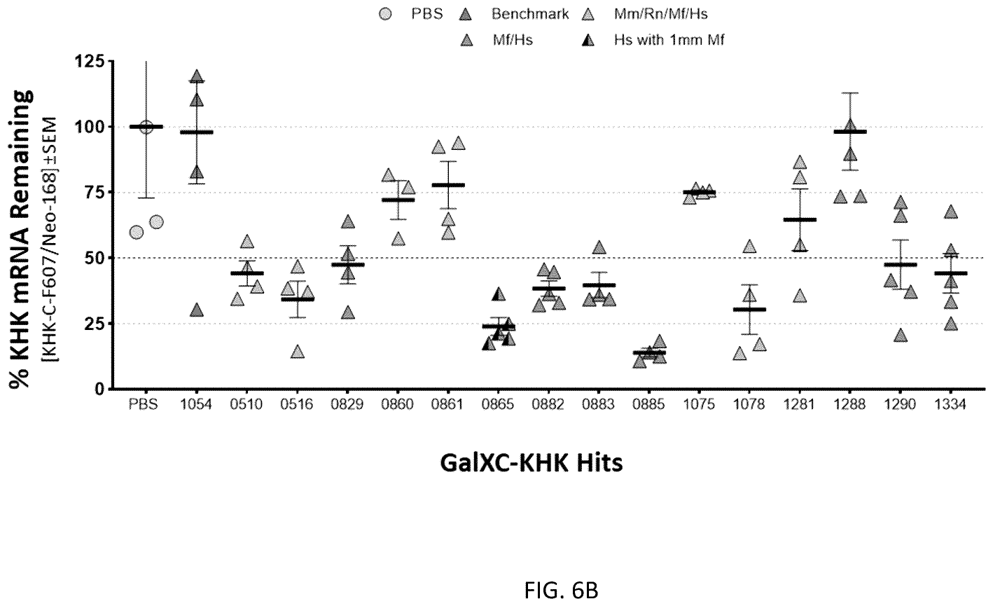

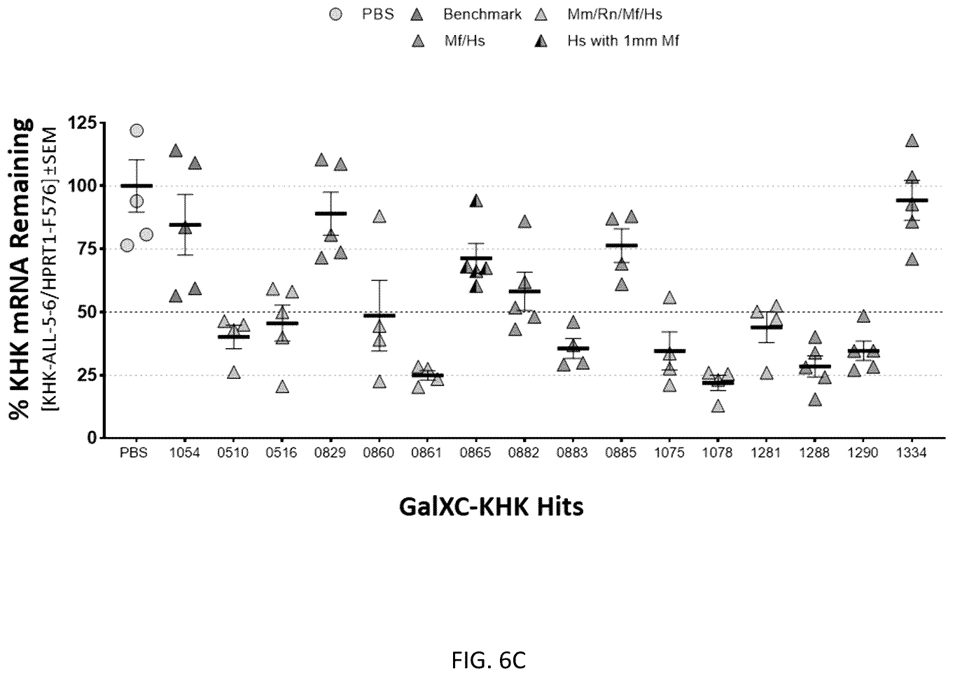

Additional constructs (Table 7) were assayed using the same methods and found effective knock-down for KHK-All and KHK-C(FIGS. 6A and 6B). Similarly, endogenous mouse KHK was reduced by GalNAc-KHK constructs which align with mouse KHK mRNA (FIG. 6C). Overall, both HDI studies identified GalNAc-KHK constructs effective at reducing KHK mRNA in vivo.

TABLE 7
GalNAc-KHK Constructs Assayed in HDI Model

| | Sense Strand | Antisense Strand |
|---|---|---|
| Name | Modified SEQ ID NO | Modified SEQ ID NO |
| KHK-1054 | 783 | 828 |
| KHK-510 | 774 | 819 |
| KHK-516 | 775 | 820 |
| KHK-829 | 776 | 821 |
| KHK-860 | 777 | 822 |
| KHK-861 | 778 | 823 |
| KHK-865 | 779 | 824 |
| KHK-882 | 780 | 825 |
| KHK-883 | 781 | 826 |
| KHK-885 | 782 | 827 |
| KHK-1075 | 784 | 829 |
| KHK-1078 | 785 | 830 |
| KHK-1281 | 786 | 831 |
| KHK-1288 | 787 | 832 |
| KHK-1290 | 788 | 833 |
| KHK-1334 | 804 | 849 |

### Example 6: RNAi Oligonucleotide Inhibition of KHK Expression and Studies in Non-human Primates

### Single-dose Non-Human Primate (NHP) Studies

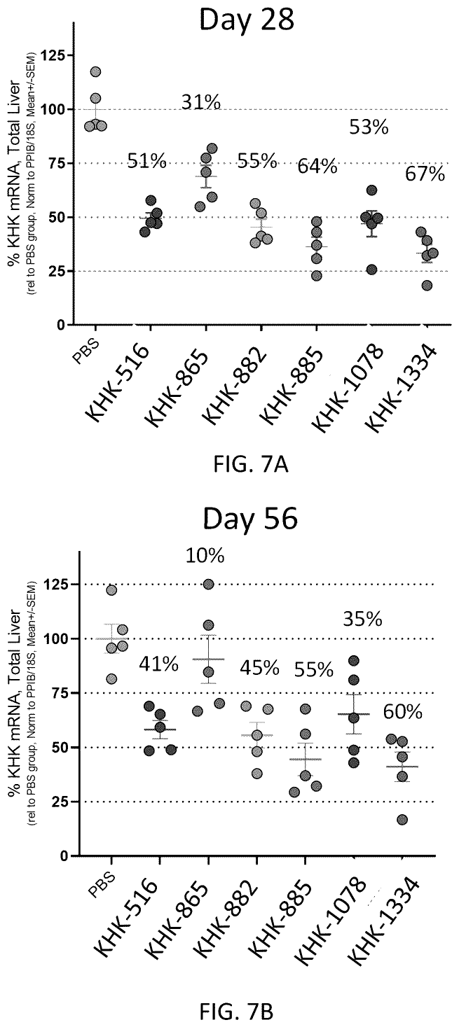

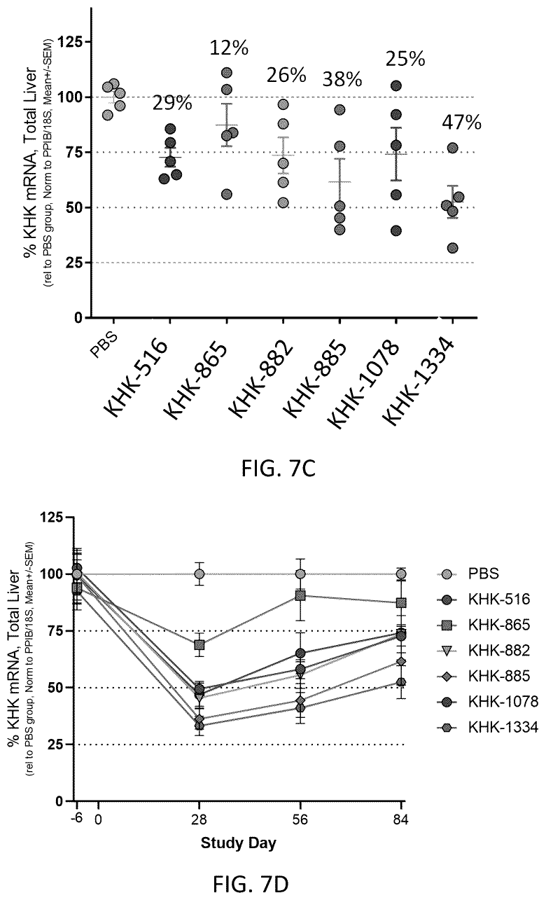

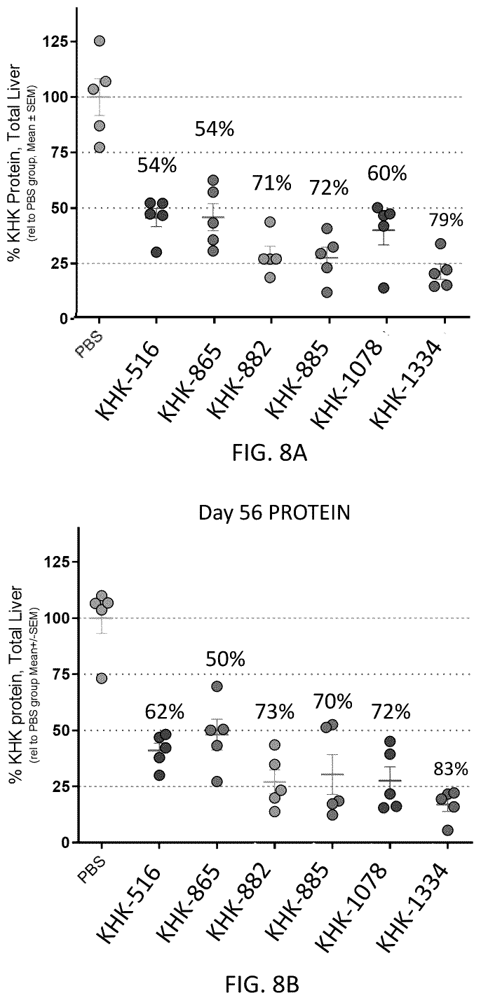

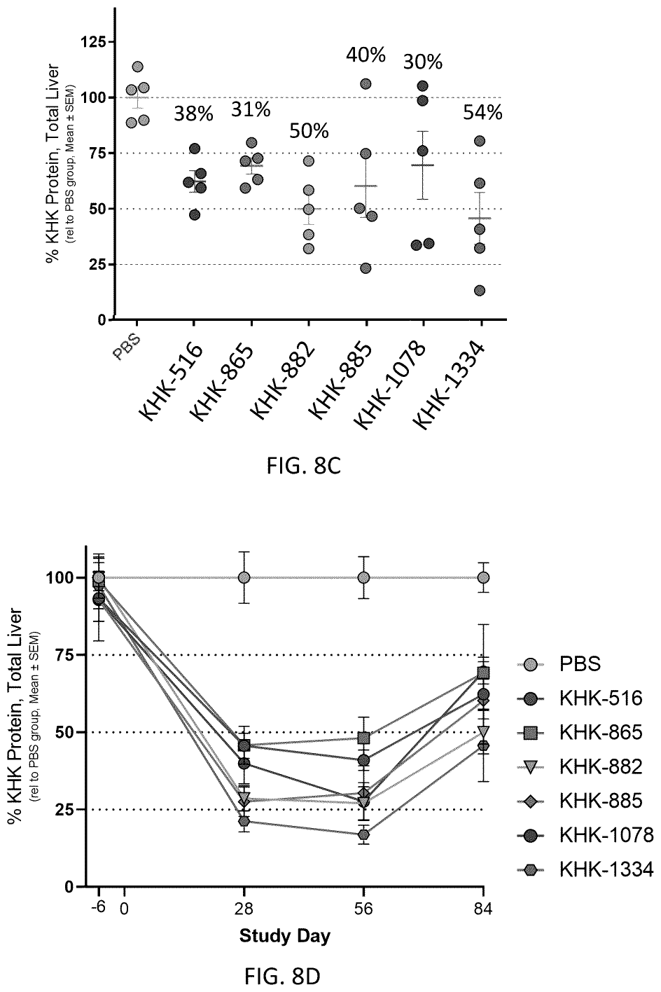

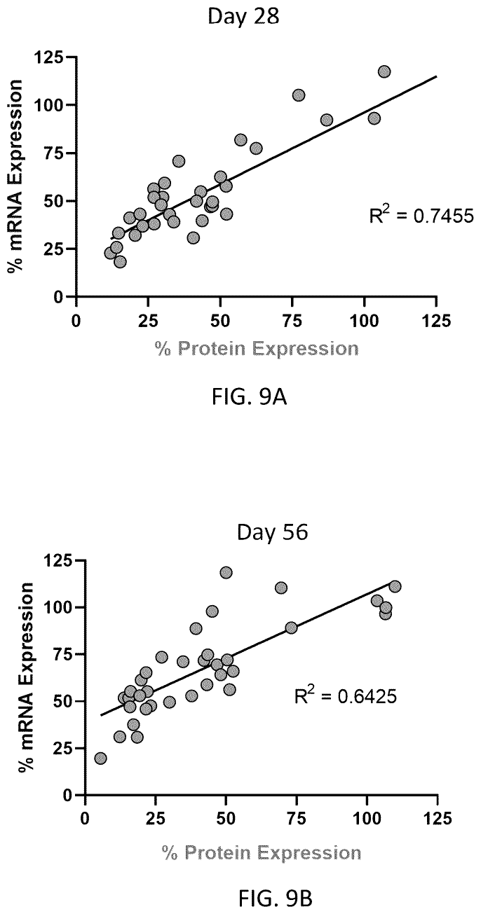

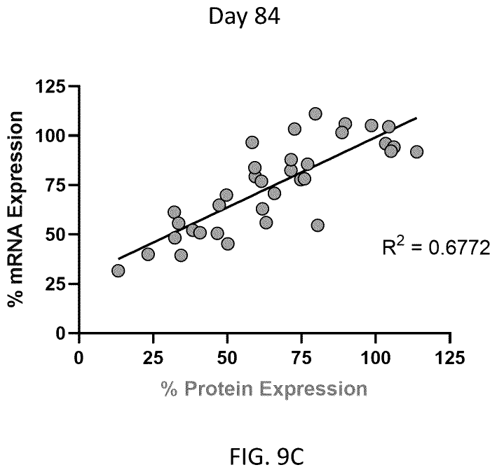

Effective GalNAc-KHK constructs identified in the HDI mouse studies were assayed for targeting efficiency in non-human primates. Specifically, GalNAc-conjugated KHK oligonucleotides listed in Table 8 were evaluated in non-naïve cynomolgus monkeys (Macaca fascicularis). In this study, the monkeys were grouped so that their mean body weights (about 5.4 kg) were comparable between the control and experimental groups. Each cohort contained at least two female and at least two male subjects. The GalNAc-conjugated KHK oligonucleotides were administered subcutaneously at a dose of 6 mg/kg on Study Day 0. Blood samples were collected one week prior to dosing (Day −7), on the dosing date (Day 0) and days 28, 56 and 84 after dosing. Ultrasound-guided core needle liver biopsies were collected on Study Days-7, 28, 56 and 84. At each time point, total RNA derived from the liver biopsy samples was subjected to qRT-PCR analysis to measure KHK mRNA in oligonucleotide-treated monkeys relative to those treated with a comparable volume of PBS. To normalize the data, the measurements were made relative to the geometric mean of two reference genes, PPIB and 18S rRNA. The following TaqMan qPCR probes purchased from Life Technologies, Inc, were used to evaluate gene expressions: Forward-TGCCTTCATGGGCTCAATG (SEQ ID NO: 772); Reverse-TCGGCCACCAGGAAGTCA (SEQ ID NO: 773); Fam probe-CCCTGGCCATGTTG (SEQ ID NO:864)). As shown in FIG. 7A (Day 28), treating NHPs with the GalNAc-conjugated KHK oligonucleotides listed in Table 8 inhibited KHK expression in the liver, as determined by a reduced amount of KHK mRNA in liver samples from oligonucleotide-treated NHPs relative to NHPs treated with PBS. The mean percent reduction of KHK mRNA in the liver samples of treated NHPs is indicated above the set of data points for each treatment group. Days 56 and 84 were also measured (FIGS. 7B and 7C) and a plot of the mean values over each time point is shown in FIG. 7D. For all time points evaluated, almost all the tested GalNAc-conjugated KHK oligonucleotides significantly inhibited KHK mRNA expression. In the same samples, KHK protein levels were detected using rabbit anti-Ketohexokinase (Abcam, AB197593) and anti-rabbit Detection Module for Sally Sue (Protein Simple, cat #DM-001). As shown in FIGS. 8A-8C, at the 28-day timepoint, GalNAc-KHK constructs inhibit KHK protein expression, as normalized to the vinculin control and slowly increases by Day 86. These results demonstrate that treating NHPs with the GalNAc-conjugated KHK oligonucleotides reduces the amount of KHK mRNA in the liver and concomitantly reduces the amount of KHK protein in the liver. However, this correlation is reduced over time after the initial dose (FIGS. 9A-9C).

Taken together, these results show that GalNAc-conjugated KHK oligonucleotides designed to target human total KHK mRNA inhibit total KHK expression in vivo (as determined by the reduction of the amount of KHK mRNA and protein).

TABLE 8
Single-dose GalNAc-KHK Constructs for NHP Study

| Name | Sense strand SEQ ID NO | Anti-sense strand SEQ ID NO |
|---|---|---|
| KHK-516 | 775 | 820 |
| KHK-865 | 779 | 824 |
| KHK-882 | 780 | 825 |
| KHK-885 | 782 | 827 |
| KHK-1078 | 785 | 830 |
| KHK-1334 | 804 | 849 |

SEQUENCE LISTING

| | Descrip- | | | |
|---|---|---|---|---|
| | tion | | | |
| | Species | | | |
| | (Hs-Mf- | | | SEQ |
| | Ms-Rn | | | ID |
| Name | (rat)) | Strand | Sequence | NO |
| Human | Human | N/A | AGGCAGGGCTGCAGATGCGAGGCCCAGC | 1 |
| (Hs) KHK | (Hs) | | TGTACCTCGCGTGTCCCGGGTCGGGAGTC | |
| nucleotide | | | GGAGACGCAGGTGCAGGAGAGTGCGGGG | |
| sequence | | | CAAGTAGCGCATTTTCTCTTTGCATTCTCG | |
| NM_00648 | | | AGATCGCTTAGCCGCGCTTTAAAAAGGTTT | |
| 8.3 | | | GCATCAGCTGTGAGTCCATCTGACAAGCG | |
| | | | AGGAAACTAAGGCTGAGAAGTGGGAGGC | |
| | | | GTTGCCATCTGCAGGCCCAGGCAACCTGC | |
| | | | TACGGGAAGACCGGGGACCAAGACCTCT | |
| | | | GGGTTGGCTTTCCTAGACCCGCTCGGGTC | |
| | | | TTCGGGTGTCGCGAGGAAGGGCCCTGCT | |
| | | | CCTTTCGTTCCCTGCACCCCTGGCCGCTG | |
| | | | CAGGTGGCTCCCTGGAGGAGGAGCTCCC | |
| | | | ACGCGGAGGAGGAGCCAGGGCAGCTGGG | |
| | | | AGCGGGGACACCATCCTCCTGGATAAGAG | |
| | | | GCAGAGGCCGGGAGGAACCCCGTCAGCC | |
| | | | GGGCGGGCAGGAAGCTCTGGGAGTAGCC | |
| | | | TCATGGAAGAGAAGCAGATCCTGTGCGTG | |
| | | | GGGCTAGTGGTGCTGGACGTCATCAGCCT | |
| | | | GGTGGACAAGTACCCTAAGGAGGACTCGG | |
| | | | AGATAAGGTGTTTGTCCCAGAGATGGCAG | |
| | | | CGCGGAGGCAACGCGTCCAACTCCTGCA | |
| | | | CCGTTCTCTCCCTGCTCGGAGCCCCCTGT | |
| | | | GCCTTCATGGGCTCAATGGCTCCTGGCCA | |
| | | | TGTTGCTGACTTCCTGGTGGCCGACTTCA | |
| | | | GGCGGCGGGGCGTGGACGTGTCTCAGGT | |
| | | | GGCCTGGCAGAGCAAGGGGGACACCCCC | |
| | | | AGCTCCTGCTGCATCATCAACAACTCCAAT | |
| | | | GGCAACCGTACCATTGTGCTCCATGACAC | |
| | | | GAGCCTGCCAGATGTGTCTGCTACAGACT | |
| | | | TTGAGAAGGTTGATCTGACCCAGTTCAAGT | |
| | | | GGATCCACATTGAGGGCCGGAACGCATCG | |
| | | | GAGCAGGTGAAGATGCTGCAGCGGATAGA | |
| | | | CGCACACAACACCAGGCAGCCTCCAGAGC | |
| | | | AGAAGATCCGGGTGTCCGTGGAGGTGGA | |
| | | | GAAGCCACGAGAGGAGCTCTTCCAGCTGT | |
| | | | TTGGCTACGGAGACGTGGTGTTTGTCAGC | |
| | | | AAAGATGTGGCCAAGCACTTGGGGTTCCA | |
| | | | GTCAGCAGAGGAAGCCTTGAGGGGCTTGT | |
| | | | ATGGTCGTGTGAGGAAAGGGGCTGTGCTT | |
| | | | GTCTGTGCCTGGGCTGAGGAGGGCGCCG | |
| | | | ACGCCCTGGGCCCTGATGGCAAATTGCTC | |
| | | | CACTCGGATGCTTTCCCGCCACCCCGCGT | |
| | | | GGTGGATACACTGGGAGCTGGAGACACCT | |
| | | | TCAATGCCTCCGTCATCTTCAGCCTCTCCC | |
| | | | AGGGGAGGAGCGTGCAGGAAGCACTGAG | |
| | | | ATTCGGGTGCCAGGTGGCCGGCAAGAAG | |
| | | | TGTGGCCTGCAGGGCTTTGATGGCATCGT | |
| | | | GTGAGAGCAGGTGCCGGCTCCTCACACAC | |
| | | | CATGGAGACTACCATTGCGGCTGCATCGC | |
| | | | CTTCTCCCCTCCATCCAGCCTGGCGTCCA | |
| | | | GGTTGCCCTGTTCAGGGGACAGATGCAAG | |
| | | | CTGTGGGGAGGACTCTGCCTGTGTCCTGT | |
| | | | GTTCCCCACAGGGAGAGGCTCTGGGGGG | |
| | | | ATGGCTGGGGGATGCAGAGCCTCAGAGC | |
| | | | AAATAAATCTTCCTCAGAGCCAGCTTCTCC | |
| | | | TCTCAATGTCTGAACTGCTCTGGCTGGGC | |
| | | | ATTCCTGAGGCTCTGACTCTTCGATCCTCC | |
| | | | CTCTTTGTGTCCATTCCCCAAATTAACCTC | |
| | | | TCCGCCCAGGCCCAGAGGAGGGGCTGCC | |
| | | | TGGGCTAGAGCAGCGAGAAGTGCCCTGG | |
| | | | GCTTGCCACCAGCTCTGCCCTGGCTGGG | |
| | | | GAGGACACTCGGTGCCCCACACCCAGTGA | |
| | | | ACCTGCCAAAGAAACCGTGAGAGCTCTTC | |
| | | | GGGGCCCTGCGTTGTGCAGACTCTATTCC | |
| | | | CACAGCTCAGAAGCTGGGAGTCCACACCG | |
| | | | CTGAGCTGAACTGACAGGCCAGTGGGGG | |
| | | | GCAGGGGTGCGCCTCCTCTGCCCTGCCC | |
| | | | ACCAGCCTGTGATTTGATGGGGTCTTCATT | |
| | | | GTCCAGAAATACCTCCTCCCGCTGACTGC | |
| | | | CCCAGAGCCTGAAAGTCTCACCCTTGGAG | |
| | | | CCCACCTTGGAATTAAGGGCGTGCCTCAG | |
| | | | CCACAAATGTGACCCAGGATACAGAGTGT | |
| | | | TGCTGTCCTCAGGGAGGTCCGATCTGGAA | |
| | | | CACATATTGGAATTGGGGCCAACTCCAATA | |
| | | | TAGGGTGGGTAAGGCCTTATAATGTAAAG | |
| | | | AGCATATAATGTAAAGGGCTTTAGAGTGAG | |
| | | | ACAGACCTGGATTAAAATCTGCCATTTAAT | |
| | | | TAGCTGCATATCACCTTAGGGTACAGCACT | |
| | | | TAACGCAATCTGCCTCAATTTCTTCATCTG | |
| | | | TCAAATGGAACCAATTCTGCTTGGCTACAG | |
| | | | AATTATTGTGAGGATAAAATCATATATAAAA | |
| | | | TGCCCAGCATGATGCCTGATGTGTA | |
| Cyno- | Cyno- | N/A | GGGGCCGGGCAGCCGCGACCACGGTCTT | 2 |
| molgus | molgus | | CAGGCAGGGCTGCAGATGCAGGCCCAGC | |
| monkey | monkey | | TCTACCTCGCGGGTCCAGGGTCGGGAGT | |
| (Mf) | (Mf) | | CCGAGACGCAGGTGCAGCAGAGGGCGGG | |
| KHK | | | GCACGTAGCGCATTTCCAGCGCATTTTCT | |
| nucleotide | | | CTTTGCATTCTCGAGATCGCTTAGCCGCG | |
| sequence | | | CTTTAGAAGGGTTTGCATCAGCTCCGAGT | |
| XM_00557 | | | CCATCTGACAAGCGAGGAAACTGAGGCTG | |
| 6322.2 | | | AGAAGTGGGAGGCGTTGCCATCTGCAGG | |
| | | | CCCAGGCAACCTGCTACGGGAAGACCGG | |
| | | | GGGCCAAGACCTCCGGGTTGGCTTTCCCA | |
| | | | GGCCAGCTTGGGTCTTCGGGTGTCGGGA | |
| | | | GCAAAGGCCCAGCTCCTTTCGTTTCCTGC | |
| | | | ACCCCTCGCCGCTGCAGGTGGCTCCCCG | |
| | | | GAGGAGGAGCTCCCACGCGGAGGAGGAG | |
| | | | CCAGGGCAGCTGGGAGCGAGGACACCAT | |
| | | | CCTCCTGGATAACAGGCAGAGGCCGGGA | |
| | | | GGAACCCGTCAGTCGGGCGGGCAGGAAG | |
| | | | CTCTGGGATCAGCCTCATGGAAGAGAAGC | |
| | | | AGATCCTGTGCGTGGGGCTAGTGGTGCTG | |
| | | | GACGTCATCAGCCTGGTGGACAAGTACCC | |
| | | | TAAGGAGGACTCAGAGATAAGGTGCTTGT | |
| | | | CCCAGAGATGGCAACGCGGAGGCAACGC | |
| | | | GTCCAACTCCTGCACCGTTCTCTCCCTGC | |
| | | | TCGGAGCCCCCTGTGCCTTCATGGGCTCA | |
| | | | ATGGCCCCTGGCCATGTTGCTGACTTCCT | |
| | | | GGTGGCCGACTTCAGGCGGCGGGGTGTG | |
| | | | GACGTGTCTCAGGTGGCCTGGCAGAGCAA | |
| | | | GGGGGACACCCCCAGCTCCTGCTGCATCA | |
| | | | TCAACAACTCCAATGGCAACCGTACCATTG | |
| | | | TGCTCCATGACACGAGCCTGCCAGATGTG | |
| | | | TCTGCTACGGACTTTGAGAAGGTTGATCT | |
| | | | GACCCAGTTCAAGTGGATCCACATTGAGG | |
| | | | GCCGGAATGCATCGGAGCAGGTGAAGAT | |
| | | | GCTGCAGCGGATAGACGCGCACAACACCA | |
| | | | GGCAGCCTCCAGAGCAGAAGATCCGGGT | |
| | | | GTCCGTGGAGGTGGAGAAGCCACAAGAG | |
| | | | GAGCTCTTTCAGCTGTTTGGCTACGGAGA | |
| | | | CGTGGTGTTTGTCAGCAAAGATGTGGCCA | |
| | | | AGCACTTGGGGTTCCAGTCAGCAGGGGAA | |
| | | | GCCCTGAGGGGCTTGTATGGTCGTGTGAG | |
| | | | GAAAGGGGCTGTGCTTGTCTGTGCCTGGG | |
| | | | CTGAGGAGGGCGCCGACGCCCTGGGCCC | |
| | | | TGATGGCAAACTGATCCACTCGGATGCTTT | |
| | | | CCCGCCACCCCGCGTGGTGGATACCCTG | |
| | | | GGGGCTGGAGACACCTTCAATGCCTCCGT | |
| | | | CATCTTCAGCCTCTCCCAGGGGAGGAGCG | |
| | | | TGCAGGAAGCACTGAGATTCGGATGCCAG | |
| | | | GTGGCCGGCAAGAAGTGTGGCCAGCAGG | |
| | | | GCTTTGATGGCATCGTGTCAGAGCCGGTG | |
| | | | CGGTAGGAGGTGCCGGCTCCCCGCACAC | |
| | | | TATGGAGGCTGACATTGCGGCTGCATCGC | |
| | | | CTTCTCCCCTCCATCCAGCCTGGCATCCA | |
| | | | GGTTGCCCTGCTCAGGGGACAGATGCAG | |
| | | | GCTGTGGGGAGGACTCCGCCTGTGTCCT | |
| | | | GTGTTCCCCACACGTCTCTCCCTGCAGAG | |
| | | | CCTCAGAGCGAATAAATCTTCCTCGGAGC | |
| | | | CAGCTTCCCCTGGCAGCTTCTGTCCTCGA | |
| | | | TGTCTGAACTGCTCTGGCTGGGCATTCCT | |
| | | | GAGGCTCTGACTCTCCAGTCCTCCCTCCT | |
| | | | CGTGTGCATTCCCCAAATTAACCTCTCCAC | |
| | | | CCAGGCCCAGAGGAGGGGCTGCCTGGGC | |
| | | | TATAGCAGCAAGAAGTGCCCCAGGCTTGC | |
| | | | CGCCAGCTCTGCCCTGGCTGGGGAGGAC | |
| | | | ACTCAGTGCCCCATACCCAGCGAACCTGC | |
| | | | CAAAGAACCAGAAGCCATGAGAGCTCTTT | |
| | | | GGGGCCCTGCGTTGTGCAGACTCTATTCC | |
| | | | CATAGCTCAGAAGCTGGGAGTCCACACGG | |
| | | | CTGAGCCAAACTGACAGGCCAGTGGGGG | |
| | | | GCGAGGGGGTGGGGCGCCTCCTCTGCCC | |
| | | | TGCCCACCAGCCTGTGATTTGGTGGCGTC | |
| | | | TTTGTTGTCCAAAAATATCTCCTCCCGCTG | |
| | | | ACTGCCCCAGAGCCTGAAAGTCTCACCCG | |
| | | | TGGAGCCCACCTTGGAATTAAGGGGATGC | |
| | | | CTCAGCCACAAATGTGACCCAAGATAGAG | |
| | | | TGTTGTCCTCAGGGAGGTCGGATCTGGAA | |
| | | | CACATATTGGAATTGGGGCCAACTCCAATA | |
| | | | TAGAGTGGATAAGGCCTTATAATGTAAAGA | |
| | | | GCACATAAGGTAAAGGGCTTTAGAGTCAG | |
| | | | ACAGACCTAGATTCAAATCTGCCATTTAAT | |
| | | | TAGCTGCATGTCACCTGAGGGTACAGCGC | |
| | | | TTAACACAATCCGCCTCAATTTCTTCATCT | |
| | | | GTCAAATGGAGCCAATTCTGCCTGGCTAC | |
| | | | AGAATTATTGCGAGGATAAAATCATGTA | |
| Mouse | Mouse | N/A | GAGGGAGAGAACGCTTGCTTCTGTGCTCC | 3 |
| (Mm) KHK | (Mm) | | GCCTGCGAAGGCGAAGTTTCTGTTGCCAG | |
| nucleotide | | | ACTGTGCTAGTCCGGGTGGTCCAGGGTCT | |
| sequence | | | GCAGCAGGCGCAGAGGGATCGGAAAGGC | |
| NM_00843 | | | GATGCATTACTAGTGCGCTTTCGCTTTGAC | |
| 9.4 | | | AGCTGAGGCGGAAAAGTGAGAGGGCCTG | |
| | | | CCATTGGCCGGGCTAGGTAACCCACCCTT | |
| | | | GCAAAGCAGAAAGCTCCCTGCGGGAGGA | |
| | | | GTTCTGCACGCAGAGGAGGAGCCAAGGTA | |
| | | | GCCAGTGAGAAGTTGGGACACGGTCCTCC | |
| | | | AGTAGATAAGAGGCAGAGCCCAGCAGGAA | |
| | | | CCCCCTCTGCTTGCGGGTAGGAAGCTTGG | |
| | | | GGAGCAGCCTCATGGAAGAGAAGCAGATC | |
| | | | CTGTGCGTGGGGCTGGTGGTGCTGGACA | |
| | | | TCATCAATGTGGTGGACAAATACCCAGAG | |
| | | | GAAGACACGGATCGCAGGTGCCTGTCCCA | |
| | | | GAGATGGCAGCGTGGAGGCAACGCATCC | |
| | | | AACTCCTGCACTGTCCTTTCCTTGCTTGGA | |
| | | | GCCCGCTGTGCCTTCATGGGCTCTTTGGC | |
| | | | CCCTGGCCACGTTGCCGACTTCCTGGTGG | |
| | | | CTGACTTCAGGCAGAGGGGCGTGGATGT | |
| | | | GTCTCAAGTGACTTGGCAGAGCCAGGGAG | |
| | | | ATACCCCTTGCTCTTGCTGCATCGTCAACA | |
| | | | ACTCCAATGGCTCCCGTACCATTATACTCT | |
| | | | ACGACACGAACCTGCCAGATGTGTCTGCT | |
| | | | AAGGACTTTGAGAAGGTCGATCTGACCCG | |
| | | | GTTCAAGTGGATCCACATTGAGGGCCGGA | |
| | | | ATGCATCGGAACAGGTGAAGATGCTGCAG | |
| | | | CGGATAGAGGAGCACAATGCCAAGCAGCC | |
| | | | TCTGCCACAGAAGGTCCGGGTGTCGGTG | |
| | | | GAGATAGAGAAGCCCCGTGAGGAGCTCTT | |
| | | | CCAGTTGTTTAGCTATGGTGAGGTGGTGT | |
| | | | TTGTCAGCAAAGATGTGGCCAAGCACCTG | |
| | | | GGGTTCCAGTCAGCAGTGGAGGCCCTGA | |
| | | | GGGGCTTGTACAGTCGAGTGAAGAAAGGG | |
| | | | GCTACGCTTGTCTGTGCCTGGGCTGAGGA | |
| | | | GGGTGCCGATGCCCTGGGCCCCGATGGT | |
| | | | CAGCTGCTCCACTCAGATGCCTTCCCACC | |
| | | | GCCCCGAGTAGTAGACACTCTTGGGGCTG | |
| | | | GAGACACCTTCAATGCCTCTGTCATCTTCA | |
| | | | GCCTCTCGAAGGGAAACAGCATGCAAGAG | |
| | | | GCCCTGAGATTCGGGTGCCAGGTGGCTG | |
| | | | GCAAGAAGTGTGGCTTGCAGGGGTTTGAT | |
| | | | GGCATTGTGTGAGAGGCAAGCGGCACCA | |
| | | | GCTCGATACCTCAGAGGCTGGCACCATGC | |
| | | | CTGCCACTGCCTTCTCTACTTCCTCCAGCT | |
| | | | TAGCATCCAGCTGCCATTCCCCGGCAGGT | |
| | | | GTGGGATGTGGGACAGCCTCTGTCTGTGT | |
| | | | CTGCGTCTCTGTATACCTATCTCCTCTCTG | |
| | | | CAGATACCTGGAGCAAATAAATCTTCCCCT | |
| | | | GAGCCAGC | |
| KHK-115- | Hs-Mf | 25 mer | AGCGCAUUUUCUCUUUGCAUUCUCG | 4 |
| 154 | commons | Sense | | |
| | | Strand | | |
| KHK-116- | Hs-Mf | 25 mer | GCGCAUUUUCUCUUUGCAUUCUCGA | 5 |
| 155 | commons | Sense | | |
| | | Strand | | |
| KHK-117- | Hs-Mf | 25 mer | CGCAUUUUCUCUUUGCAUUCUCGAG | 6 |
| 156 | commons | Sense | | |
| | | Strand | | |
| KHK-118- | Hs-Mf | 25 mer | GCAUUUUCUCUUUGCAUUCUCGAGA | 7 |
| 157 | commons | Sense | | |
| | | Strand | | |
| KHK-119- | Hs-Mf | 25 mer | CAUUUUCUCUUUGCAUUCUCGAGAT | 8 |
| 158 | commons | Sense | | |
| | | Strand | | |
| KHK-120- | Hs-Mf | 25 mer | AUUUUCUCUUUGCAUUCUCGAGATC | 9 |
| 159 | commons | Sense | | |
| | | Strand | | |
| KHK-121- | Hs-Mf | 25 mer | UUUUCUCUUUGCAUUCUCGAGAUCG | 10 |
| 160 | commons | Sense | | |
| | | Strand | | |
| KHK-122- | Hs-Mf | 25 mer | UUUCUCUUUGCAUUCUCGAGAUCGC | 11 |
| 161 | commons | Sense | | |
| | | Strand | | |
| KHK-123- | Hs-Mf | 25 mer | UUCUCUUUGCAUUCUCGAGAUCGCT | 12 |
| 162 | commons | Sense | | |
| | | Strand | | |
| KHK-124- | Hs-Mf | 25 mer | UCUCUUUGCAUUCUCGAGAUCGCTT | 13 |
| 163 | commons | Sense | | |
| | | Strand | | |
| KHK-125- | Hs-Mf | 25 mer | CUCUUUGCAUUCUCGAGAUCGCUTA | 14 |
| 164 | commons | Sense | | |
| | | Strand | | |
| KHK-126- | Hs-Mf | 25 mer | UCUUUGCAUUCUCGAGAUCGCUUAG | 15 |
| 165 | commons | Sense | | |
| | | Strand | | |
| KHK-127- | Hs-Mf | 25 mer | CUUUGCAUUCUCGAGAUCGCUUAGC | 16 |
| 166 | commons | Sense | | |
| | | Strand | | |
| KHK-128- | Hs-Mf | 25 mer | UUUGCAUUCUCGAGAUCGCUUAGCC | 17 |
| 167 | commons | Sense | | |
| | | Strand | | |
| KHK-179 | Hs | 25 mer | GUGAGUCCAUCUGACAAGCGAGGAA | 18 |
| | unique | Sense | | |
| | | Strand | | |
| KHK-181- | Hs-Mf | 25 mer | GAGUCCAUCUGACAAGCGAGGAAAC | 19 |
| 220 | commons | Sense | | |
| | | Strand | | |
| KHK-182- | Hs-Mf | 25 mer | AGUCCAUCUGACAAGCGAGGAAACT | 20 |
| 221 | commons | Sense | | |
| | | Strand | | |
| KHK-183- | Hs-Mf | 25 mer | GUCCAUCUGACAAGCGAGGAAACTA | 21 |
| 222 | commons | Sense | | |
| | | Strand | | |
| KHK-184- | Hs-Mf | 25 mer | UCCAUCUGACAAGCGAGGAAACUAA | 22 |
| 223 | commons | Sense | | |
| | | Strand | | |
| KHK-185- | Hs-Mf | 25 mer | CCAUCUGACAAGCGAGGAAACUAAG | 23 |
| 224 | commons | Sense | | |
| | | Strand | | |
| KHK-186- | Hs-Mf | 25 mer | CAUCUGACAAGCGAGGAAACUAAGG | 24 |
| 225 | commons | Sense | | |
| | | Strand | | |
| KHK-187- | Hs-Mf | 25 mer | AUCUGACAAGCGAGGAAACUAAGGC | 25 |
| 226 | commons | Sense | | |
| | | Strand | | |
| KHK-188- | Hs-Mf | 25 mer | UCUGACAAGCGAGGAAACUAAGGCT | 26 |
| 227 | commons | Sense | | |
| | | Strand | | |
| KHK-431- | Hs-Mf | 25 mer | GGACACCAUCCUCCUGGAUAAGAGG | 27 |
| 470 | commons | Sense | | |
| | | Strand | | |
| KHK-432- | Hs-Mf | 25 mer | GACACCAUCCUCCUGGAUAAGAGGC | 28 |
| 471 | commons | Sense | | |
| | | Strand | | |
| KHK-433- | Hs-Mf | 25 mer | ACACCAUCCUCCUGGAUAAGAGGCA | 29 |
| 472 | commons | Sense | | |
| | | Strand | | |
| KHK-507- | Hs-Mf- | 25 mer | AGCCUCAUGGAAGAGAAGCAGAUCC | 30 |
| 545-376- | Mm-Rn | Sense | | |
| 218 | commons | Strand | | |
| KHK-508- | Hs-Mf- | 25 mer | GCCUCAUGGAAGAGAAGCAGAUCCT | 31 |
| 546-377- | Mm-Rn | Sense | | |
| 219 | commons | Strand | | |
| KHK-509- | Hs-Mf- | 25 mer | CCUCAUGGAAGAGAAGCAGAUCCTG | 32 |
| 547-378- | Mm-Rn | Sense | | |
| 220 | commons | Strand | | |
| KHK-510- | Hs-Mf- | 25 mer | CUCAUGGAAGAGAAGCAGAUCCUGT | 33 |
| 548-379- | Mm-Rn | Sense | | |
| 221 | commons | Strand | | |
| KHK-511- | Hs-Mf- | 25 mer | UCAUGGAAGAGAAGCAGAUCCUGTG | 34 |
| 549-380- | Mm-Rn | Sense | | |
| 222 | commons | Strand | | |
| KHK-512- | Hs-Mf- | 25 mer | CAUGGAAGAGAAGCAGAUCCUGUGC | 35 |
| 550-381- | Mm-Rn | Sense | | |
| 223 | commons | Strand | | |
| KHK-513- | Hs-Mf- | 25 mer | AUGGAAGAGAAGCAGAUCCUGUGCG | 36 |
| 551-382- | Mm-Rn | Sense | | |
| 224 | commons | Strand | | |
| KHK-514- | Hs-Mf- | 25 mer | UGGAAGAGAAGCAGAUCCUGUGCGT | 37 |
| 552-383- | Mm-Rn | Sense | | |
| 225 | commons | Strand | | |
| KHK-515- | Hs-Mf- | 25 mer | GGAAGAGAAGCAGAUCCUGUGCGTG | 38 |
| 553-384- | Mm-Rn | Sense | | |
| 226 | commons | Strand | | |
| KHK-516- | Hs-Mf- | 25 mer | GAAGAGAAGCAGAUCCUGUGCGUGG | 39 |
| 554-385- | Mm-Rn | Sense | | |
| 227 | commons | Strand | | |
| KHK-517- | Hs-Mf- | 25 mer | AAGAGAAGCAGAUCCUGUGCGUGGG | 40 |
| 555-386- | Mm-Rn | Sense | | |
| 228 | commons | Strand | | |
| KHK-518- | Hs-Mf- | 25 mer | AGAGAAGCAGAUCCUGUGCGUGGGG | 41 |
| 556-387- | Mm-Rn | Sense | | |
| 229 | commons | Strand | | |
| KHK-520- | Hs-Mf- | 25 mer | AGAAGCAGAUCCUGUGCGUGGGGCT | 42 |
| 558-389- | Mm-Rn | Sense | | |
| 231 | commons | Strand | | |
| KHK-521- | Hs-Mf- | 25 mer | GAAGCAGAUCCUGUGCGUGGGGCTA | 43 |
| 559-390- | Mm-Rn | Sense | | |
| 232 | commons | Strand | | |
| KHK-522- | Hs-Mf- | 25 mer | AAGCAGAUCCUGUGCGUGGGGCUAG | 44 |
| 560-391- | Mm-Rn | Sense | | |
| 233 | commons | Strand | | |
| KHK-541- | Hs-Mf | 25 mer | GGCUAGUGGUGCUGGACGUCAUCAG | 45 |
| 579 | commons | Sense | | |
| | | Strand | | |
| KHK-544- | Hs-Mf | 25 mer | UAGUGGUGCUGGACGUCAUCAGCCT | 46 |
| 582 | commons | Sense | | |
| | | Strand | | |
| KHK-546- | Hs-Mf | 25 mer | GUGGUGCUGGACGUCAUCAGCCUGG | 47 |
| 584 | commons | Sense | | |
| | | Strand | | |
| KHK-547- | Hs-Mf | 25 mer | UGGUGCUGGACGUCAUCAGCCUGGT | 48 |
| 585 | commons | Sense | | |
| | | Strand | | |
| KHK-548- | Hs-Mf | 25 mer | GGUGCUGGACGUCAUCAGCCUGGTG | 49 |
| 586 | commons | Sense | | |
| | | Strand | | |
| KHK-549- | Hs-Mf | 25 mer | GUGCUGGACGUCAUCAGCCUGGUGG | 50 |
| 587 | commons | Sense | | |
| | | Strand | | |
| KHK-550- | Hs-Mf | 25 mer | UGCUGGACGUCAUCAGCCUGGUGGA | 51 |
| 588 | commons | Sense | | |
| | | Strand | | |
| KHK-551- | Hs-Mf | 25 mer | GCUGGACGUCAUCAGCCUGGUGGAC | 52 |
| 589 | commons | Sense | | |
| | | Strand | | |
| KHK-552- | Hs-Mf | 25 mer | CUGGACGUCAUCAGCCUGGUGGACA | 53 |
| 590 | commons | Sense | | |
| | | Strand | | |
| KHK-553- | Hs-Mf | 25 mer | UGGACGUCAUCAGCCUGGUGGACAA | 54 |
| 591 | commons | Sense | | |
| | | Strand | | |
| KHK-554- | Hs-Mf | 25 mer | GGACGUCAUCAGCCUGGUGGACAAG | 55 |
| 592 | commons | Sense | | |
| | | Strand | | |
| KHK-555- | Hs-Mf | 25 mer | GACGUCAUCAGCCUGGUGGACAAGT | 56 |
| 593 | commons | Sense | | |
| | | Strand | | |
| KHK-556- | Hs-Mf | 25 mer | ACGUCAUCAGCCUGGUGGACAAGTA | 57 |
| 594 | commons | Sense | | |
| | | Strand | | |
| KHK-557- | Hs-Mf | 25 mer | CGUCAUCAGCCUGGUGGACAAGUAC | 58 |
| 595 | commons | Sense | | |
| | | Strand | | |
| KHK-558- | Hs-Mf | 25 mer | GUCAUCAGCCUGGUGGACAAGUACC | 59 |
| 596 | commons | Sense | | |
| | | Strand | | |
| KHK-559- | Hs-Mf | 25 mer | UCAUCAGCCUGGUGGACAAGUACCC | 60 |
| 597 | commons | Sense | | |
| | | Strand | | |
| KHK-560- | Hs-Mf | 25 mer | CAUCAGCCUGGUGGACAAGUACCCT | 61 |
| 598 | commons | Sense | | |
| | | Strand | | |
| KHK-561- | Hs-Mf | 25 mer | AUCAGCCUGGUGGACAAGUACCCTA | 62 |
| 599 | commons | Sense | | |
| | | Strand | | |
| KHK-562- | Hs-Mf | 25 mer | UCAGCCUGGUGGACAAGUACCCUAA | 63 |
| 600 | commons | Sense | | |
| | | Strand | | |
| KHK-563- | Hs-Mf | 25 mer | CAGCCUGGUGGACAAGUACCCUAAG | 64 |
| 601 | commons | Sense | | |
| | | Strand | | |
| KHK-564- | Hs-Mf | 25 mer | AGCCUGGUGGACAAGUACCCUAAGG | 65 |
| 602 | commons | Sense | | |
| | | Strand | | |
| KHK-565- | Hs-Mf | 25 mer | GCCUGGUGGACAAGUACCCUAAGGA | 66 |
| 603 | commons | Sense | | |
| | | Strand | | |
| KHK-566- | Hs-Mf | 25 mer | CCUGGUGGACAAGUACCCUAAGGAG | 67 |
| 604 | commons | Sense | | |
| | | Strand | | |
| KHK-567- | Hs-Mf | 25 mer | CUGGUGGACAAGUACCCUAAGGAGG | 68 |
| 605 | commons | Sense | | |
| | | Strand | | |
| KHK-568- | Hs-Mf | 25 mer | UGGUGGACAAGUACCCUAAGGAGGA | 69 |
| 606 | commons | Sense | | |
| | | Strand | | |
| KHK-569- | Hs-Mf | 25 mer | GGUGGACAAGUACCCUAAGGAGGAC | 70 |
| 607 | commons | Sense | | |
| | | Strand | | |
| KHK-570- | Hs-Mf | 25 mer | GUGGACAAGUACCCUAAGGAGGACT | 71 |
| 608 | commons | Sense | | |
| | | Strand | | |
| KHK-571- | Hs-Mf | 25 mer | UGGACAAGUACCCUAAGGAGGACTC | 72 |
| 609 | commons | Sense | | |
| | | Strand | | |
| KHK-572- | Hs-Mf | 25 mer | GGACAAGUACCCUAAGGAGGACUCG | 73 |
| 610 | commons | Sense | | |
| | | Strand | | |
| KHK-573- | Hs-Mf | 25 mer | GACAAGUACCCUAAGGAGGACUCGG | 74 |
| 611 | commons | Sense | | |
| | | Strand | | |
| KHK-574- | Hs-Mf | 25 mer | ACAAGUACCCUAAGGAGGACUCGGA | 75 |
| 612 | commons | Sense | | |
| | | Strand | | |
| KHK-575- | Hs-Mf | 25 mer | CAAGUACCCUAAGGAGGACUCGGAG | 76 |
| 613 | commons | Sense | | |
| | | Strand | | |
| KHK-576- | Hs-Mf | 25 mer | AAGUACCCUAAGGAGGACUCGGAGA | 77 |
| 614 | commons | Sense | | |
| | | Strand | | |
| KHK-577- | Hs-Mf | 25 mer | AGUACCCUAAGGAGGACUCGGAGAT | 78 |
| 615 | commons | Sense | | |
| | | Strand | | |
| KHK-638- | Hs-Mf | 25 mer | CGCGUCCAACUCCUGCACCGUUCTC | 79 |
| 676 | commons | Sense | | |
| | | Strand | | |
| KHK-641- | Hs-Mf | 25 mer | GUCCAACUCCUGCACCGUUCUCUCC | 80 |
| 679 | commons | Sense | | |
| | | Strand | | |
| KHK-642- | Hs-Mf | 25 mer | UCCAACUCCUGCACCGUUCUCUCCC | 81 |
| 680 | commons | Sense | | |
| | | Strand | | |
| KHK-643- | Hs-Mf | 25 mer | CCAACUCCUGCACCGUUCUCUCCCT | 82 |
| 681 | commons | Sense | | |
| | | Strand | | |
| KHK-644- | Hs-Mf | 25 mer | CAACUCCUGCACCGUUCUCUCCCTG | 83 |
| 682 | commons | Sense | | |
| | | Strand | | |
| KHK-645- | Hs-Mf | 25 mer | AACUCCUGCACCGUUCUCUCCCUGC | 84 |
| 683 | commons | Sense | | |
| | | Strand | | |
| KHK-646- | Hs-Mf | 25 mer | ACUCCUGCACCGUUCUCUCCCUGCT | 85 |
| 684 | commons | Sense | | |
| | | Strand | | |
| KHK-647- | Hs-Mf | 25 mer | CUCCUGCACCGUUCUCUCCCUGCTC | 86 |
| 685 | commons | Sense | | |
| | | Strand | | |
| KHK-650- | Hs-Mf | 25 mer | CUGCACCGUUCUCUCCCUGCUCGGA | 87 |
| 688 | commons | Sense | | |
| | | Strand | | |
| KHK-676- | Hs-Mf | 25 mer | CCCCCUGUGCCUUCAUGGGCUCAAT | 88 |
| 714 | commons | Sense | | |
| | | Strand | | |
| KHK-713- | Hs-Mf | 25 mer | UGUUGCUGACUUCCUGGUGGCCGAC | 89 |
| 722 | commons | Sense | | |
| | | Strand | | |
| KHK-826- | Hs-Mf | 25 mer | AUGGCAACCGUACCAUUGUGCUCCA | 90 |
| 835 | commons | Sense | | |
| | | Strand | | |
| KHK-827- | Hs-Mf | 25 mer | UGGCAACCGUACCAUUGUGCUCCAT | 91 |
| 836 | commons | Sense | | |
| | | Strand | | |
| KHK-829- | Hs-Mf | 25 mer | GCAACCGUACCAUUGUGCUCCAUGA | 92 |
| 838 | commons | Sense | | |
| | | Strand | | |
| KHK-830- | Hs-Mf | 25 mer | CAACCGUACCAUUGUGCUCCAUGAC | 93 |
| 839 | commons | Sense | | |
| | | Strand | | |
| KHK-831- | Hs-Mf | 25 mer | AACCGUACCAUUGUGCUCCAUGACA | 94 |
| 840 | commons | Sense | | |
| | | Strand | | |
| KHK-832- | Hs-Mf | 25 mer | ACCGUACCAUUGUGCUCCAUGACAC | 95 |
| 841 | commons | Sense | | |
| | | Strand | | |
| KHK-857- | Hs-Mf | 25 mer | GAGCCUGCCAGAUGUGUCUGCUACA | 96 |
| 895 | commons | Sense | | |
| | | Strand | | |
| KHK-858- | Hs-Mf | 25 mer | AGCCUGCCAGAUGUGUCUGCUACAG | 97 |
| 896 | commons | Sense | | |
| | | Strand | | |
| KHK-859- | Hs-Mf | 25 mer | GCCUGCCAGAUGUGUCUGCUACAGA | 98 |
| 897 | commons | Sense | | |
| | | Strand | | |
| KHK-860- | Hs-Mf- | 25 mer | CCUGCCAGAUGUGUCUGCUACAGAC | 99 |
| 898-729- | Mm-Rn | Sense | | |
| 571 | commons | Strand | | |
| KHK-861- | Hs-Mf- | 25 mer | CUGCCAGAUGUGUCUGCUACAGACT | 100 |
| 899-730- | Mm-Rn | Sense | | |
| 572 | commons | Strand | | |
| KHK-862- | Hs-Mf | 25 mer | UGCCAGAUGUGUCUGCUACAGACTT | 101 |
| 900 | commons | Sense | | |
| | | Strand | | |
| KHK-865 | Hs | 25 mer | CAGAUGUGUCUGCUACAGACUUUGA | 102 |
| | unique | Sense | | |
| | | Strand | | |
| KHK-880 | Hs | 25 mer | CAGACUUUGAGAAGGUUGAUCUGAC | 103 |
| | unique | Sense | | |
| | | Strand | | |
| KHK-882- | Hs-Mf | 25 mer | GACUUUGAGAAGGUUGAUCUGACCC | 104 |
| 920 | commons | Sense | | |
| | | Strand | | |
| KHK-883- | Hs-Mf | 25 mer | ACUUUGAGAAGGUUGAUCUGACCCA | 105 |
| 921 | commons | Sense | | |
| | | Strand | | |
| KHK-884- | Hs-Mf | 25 mer | CUUUGAGAAGGUUGAUCUGACCCAG | 106 |
| 922 | commons | Sense | | |
| | | Strand | | |
| KHK-885- | Hs-Mf | 25 mer | UUUGAGAAGGUUGAUCUGACCCAGT | 107 |
| 923 | commons | Sense | | |
| | | Strand | | |
| KHK-886- | Hs-Mf | 25 mer | UUGAGAAGGUUGAUCUGACCCAGTT | 108 |
| 924 | commons | Sense | | |
| | | Strand | | |
| KHK-887- | Hs-Mf | 25 mer | UGAGAAGGUUGAUCUGACCCAGUTC | 109 |
| 925 | commons | Sense | | |
| | | Strand | | |
| KHK-888- | Hs-Mf | 25 mer | GAGAAGGUUGAUCUGACCCAGUUCA | 110 |
| 926 | commons | Sense | | |
| | | Strand | | |
| KHK-889- | Hs-Mf | 25 mer | AGAAGGUUGAUCUGACCCAGUUCAA | 111 |
| 927 | commons | Sense | | |
| | | Strand | | |
| KHK-890- | Hs-Mf | 25 mer | GAAGGUUGAUCUGACCCAGUUCAAG | 112 |
| 928 | commons | Sense | | |
| | | Strand | | |
| KHK-891- | Hs-Mf | 25 mer | AAGGUUGAUCUGACCCAGUUCAAGT | 113 |
| 929 | commons | Sense | | |
| | | Strand | | |
| KHK-892- | Hs-Mf | 25 mer | AGGUUGAUCUGACCCAGUUCAAGTG | 114 |
| 930 | commons | Sense | | |
| | | Strand | | |
| KHK-893- | Hs-Mf | 25 mer | GGUUGAUCUGACCCAGUUCAAGUGG | 115 |
| 931 | commons | Sense | | |
| | | Strand | | |
| KHK-894- | Hs-Mf | 25 mer | GUUGAUCUGACCCAGUUCAAGUGGA | 116 |
| 932 | commons | Sense | | |
| | | Strand | | |
| KHK-895- | Hs-Mf | 25 mer | UUGAUCUGACCCAGUUCAAGUGGAT | 117 |
| 933 | commons | Sense | | |
| | | Strand | | |
| KHK-896- | Hs-Mf | 25 mer | UGAUCUGACCCAGUUCAAGUGGATC | 118 |
| 934 | commons | Sense | | |
| | | Strand | | |
| KHK-897- | Hs-Mf | 25 mer | GAUCUGACCCAGUUCAAGUGGAUCC | 119 |
| 935 | commons | Sense | | |
| | | Strand | | |
| KHK-898- | Hs-Mf | 25 mer | AUCUGACCCAGUUCAAGUGGAUCCA | 120 |
| 936 | commons | Sense | | |
| | | Strand | | |
| KHK-899- | Hs-Mf | 25 mer | UCUGACCCAGUUCAAGUGGAUCCAC | 121 |
| 937 | commons | Sense | | |
| | | Strand | | |
| KHK-900- | Hs-Mf | 25 mer | CUGACCCAGUUCAAGUGGAUCCACA | 122 |
| 938 | commons | Sense | | |
| | | Strand | | |
| KHK-901- | Hs-Mf | 25 mer | UGACCCAGUUCAAGUGGAUCCACAT | 123 |
| 939 | commons | Sense | | |
| | | Strand | | |
| KHK-902- | Hs-Mf | 25 mer | GACCCAGUUCAAGUGGAUCCACATT | 124 |
| 940 | commons | Sense | | |
| | | Strand | | |
| KHK-903- | Hs-Mf | 25 mer | ACCCAGUUCAAGUGGAUCCACAUTG | 125 |
| 941 | commons | Sense | | |
| | | Strand | | |
| KHK-904- | Hs-Mf | 25 mer | CCCAGUUCAAGUGGAUCCACAUUGA | 126 |
| 942 | commons | Sense | | |
| | | Strand | | |
| KHK-905- | Hs-Mf | 25 mer | CCAGUUCAAGUGGAUCCACAUUGAG | 127 |
| 943 | commons | Sense | | |
| | | Strand | | |
| KHK-906- | Hs-Mf | 25 mer | CAGUUCAAGUGGAUCCACAUUGAGG | 128 |
| 944 | commons | Sense | | |
| | | Strand | | |
| KHK-907- | Hs-Mf | 25 mer | AGUUCAAGUGGAUCCACAUUGAGGG | 129 |
| 945 | commons | Sense | | |
| | | Strand | | |
| KHK-908- | Hs-Mf- | 25 mer | GUUCAAGUGGAUCCACAUUGAGGGC | 130 |
| 946-777- | Mm-Rn | Sense | | |
| 619 | commons | Strand | | |
| KHK-909- | Hs-Mf- | 25 mer | UUCAAGUGGAUCCACAUUGAGGGCC | 131 |
| 947-778- | Mm-Rn | Sense | | |
| 620 | commons | Strand | | |
| KHK-910- | Hs-Mf- | 25 mer | UCAAGUGGAUCCACAUUGAGGGCCG | 132 |
| 948-779- | Mm-Rn | Sense | | |
| 621 | commons | Strand | | |
| KHK-911- | Hs-Mf- | 25 mer | CAAGUGGAUCCACAUUGAGGGCCGG | 133 |
| 949-780- | Mm-Rn | Sense | | |
| 622 | commons | Strand | | |
| KHK-912- | Hs-Mf- | 25 mer | AAGUGGAUCCAGAUUGAGGGCCGGA | 134 |
| 950-781- | Mm-Rn | Sense | | |
| 623 | commons | Strand | | |
| KHK-913- | Hs-Mf- | 25 mer | AGUGGAUCCACAUUGAGGGCCGGAA | 135 |
| 951-782- | Mm-Rn | Sense | | |
| 624 | commons | Strand | | |
| KHK-914- | Hs-Mf- | 25 mer | GUGGAUCCACAUUGAGGGCCGGAAC | 136 |
| 952-783- | Mm-Rn | Sense | | |
| 625 | commons | Strand | | |
| KHK-939- | Hs-Mf | 25 mer | GCAUCGGAGCAGGUGAAGAUGCUGC | 137 |
| 977 | commons | Sense | | |
| | | Strand | | |
| KHK-940- | Hs-Mf | 25 mer | CAUCGGAGCAGGUGAAGAUGCUGCA | 138 |
| 978 | commons | Sense | | |
| | | Strand | | |
| KHK-941- | Hs-Mf | 25 mer | AUCGGAGCAGGUGAAGAUGCUGCAG | 139 |
| 979 | commons | Sense | | |
| | | Strand | | |
| KHK-942- | Hs-Mf | 25 mer | UCGGAGCAGGUGAAGAUGCUGCAGC | 140 |
| 980 | commons | Sense | | |
| | | Strand | | |
| KHK-943- | Hs-Mf | 25 mer | CGGAGCAGGUGAAGAUGCUGCAGCG | 141 |
| 981 | commons | Sense | | |
| | | Strand | | |
| KHK-944- | Hs-Mf | 25 mer | GGAGCAGGUGAAGAUGCUGCAGCGG | 142 |
| 982 | commons | Sense | | |
| | | Strand | | |
| KHK-945- | Hs-Mf | 25 mer | GAGCAGGUGAAGAUGCUGCAGCGGA | 143 |
| 983 | commons | Sense | | |
| | | Strand | | |
| KHK-946- | Hs-Mf | 25 mer | AGCAGGUGAAGAUGCUGCAGCGGAT | 144 |
| 984 | commons | Sense | | |
| | | Strand | | |
| KHK-947- | Hs-Mf | 25 mer | GCAGGUGAAGAUGCUGCAGCGGATA | 145 |
| 985 | commons | Sense | | |
| | | Strand | | |
| KHK-948- | Hs-Mf- | 25 mer | CAGGUGAAGAUGCUGCAGCGGAUAG | 146 |
| 986-817 | Mm | Sense | | |
| | commons | Strand | | |
| KHK-949- | Hs-Mf- | 25 mer | AGGUGAAGAUGCUGCAGCGGAUAGA | 147 |
| 987-818 | Mm | Sense | | |
| | commons | Strand | | |
| KHK-950- | Hs-Mf- | 25 mer | GGUGAAGAUGCUGCAGCGGAUAGAC | 148 |
| 988-819 | Mm | Sense | | |
| | commons | Strand | | |
| KHK-951- | Hs-Mf- | 25 mer | GUGAAGAUGCUGCAGCGGAUAGACG | 149 |
| 989-820 | Mm | Sense | | |
| | commons | Strand | | |
| KHK-952- | Hs-Mf- | 25 mer | UGAAGAUGCUGCAGCGGAUAGACGC | 150 |
| 990-821 | Mm | Sense | | |
| | commons | Strand | | |
| KHK-953- | Hs-Mf- | 25 mer | GAAGAUGCUGCAGCGGAUAGACGCA | 151 |
| 991-822 | Mm | Sense | | |
| | commons | Strand | | |
| KHK-954- | Hs-Mf- | 25 mer | AAGAUGCUGCAGCGGAUAGACGCAC | 152 |
| 992-823 | Mm | Sense | | |
| | commons | Strand | | |
| KHK-955- | Hs-Mf- | 25 mer | AGAUGCUGCAGCGGAUAGACGCACA | 153 |
| 993-824 | Mm | Sense | | |
| | commons | Strand | | |
| KHK-956- | Hs-Mf | 25 mer | GAUGCUGCAGCGGAUAGACGCACAC | 154 |
| 994 | commons | Sense | | |
| | | Strand | | |
| KHK-957- | Hs-Mf | 25 mer | AUGCUGCAGCGGAUAGACGCACACA | 155 |
| 995 | commons | Sense | | |
| | | Strand | | |
| KHK-958- | Hs-Mf | 25 mer | UGCUGCAGCGGAUAGACGCACACAA | 156 |
| 996 | commons | Sense | | |
| | | Strand | | |
| KHK-978- | Hs-Mf | 25 mer | CACAACACCAGGCAGCCUCCAGAGC | 157 |
| 1016 | commons | Sense | | |
| | | Strand | | |
| KHK-982- | Hs-Mf | 25 mer | ACACCAGGCAGCCUCCAGAGCAGAA | 158 |
| 1020 | commons | Sense | | |
| | | Strand | | |
| KHK-983- | Hs-Mf | 25 mer | CACCAGGCAGCCUCCAGAGCAGAAG | 159 |
| 1021 | commons | Sense | | |
| | | Strand | | |
| KHK-984- | Hs-Mf | 25 mer | ACCAGGCAGCCUCCAGAGCAGAAGA | 160 |
| 1022 | commons | Sense | | |
| | | Strand | | |
| KHK-985- | Hs-Mf | 25 mer | CCAGGCAGCCUCCAGAGCAGAAGAT | 161 |
| 1023 | commons | Sense | | |
| | | Strand | | |
| KHK-991- | Hs-Mf | 25 mer | AGCCUCCAGAGCAGAAGAUCCGGGT | 162 |
| 1029 | commons | Sense | | |
| | | Strand | | |
| KHK-992- | Hs-Mf | 25 mer | GCCUCCAGAGCAGAAGAUCCGGGTG | 163 |
| 1030 | commons | Sense | | |
| | | Strand | | |
| KHK-993- | Hs-Mf | 25 mer | CCUCCAGAGCAGAAGAUCCGGGUGT | 164 |
| 1031 | commons | Sense | | |
| | | Strand | | |
| KHK-999- | Hs-Mf | 25 mer | GAGCAGAAGAUCCGGGUGUCCGUGG | 165 |
| 1037 | commons | Sense | | |
| | | Strand | | |
| KHK-1000- | Hs-Mf | 25 mer | AGCAGAAGAUCCGGGUGUCCGUGGA | 166 |
| 1038 | commons | Sense | | |
| | | Strand | | |
| KHK-1019- | Hs-Mf | 25 mer | CGUGGAGGUGGAGAAGCCACGAGAG | 167 |
| 1057 | commons | Sense | | |
| | | Strand | | |
| KHK-1054- | Hs-Mf | 25 mer | AGCUGUUUGGCUACGGAGACGUGGT | 168 |
| 1092 | commons | Sense | | |
| | | Strand | | |
| KHK-1055- | Hs-Mf | 25 mer | GCUGUUUGGCUACGGAGACGUGGTG | 169 |
| 1093 | commons | Sense | | |
| | | Strand | | |
| KHK-1057- | Hs-Mf | 25 mer | UGUUUGGCUACGGAGACGUGGUGTT | 170 |
| 1095 | commons | Sense | | |
| | | Strand | | |
| KHK-1058- | Hs-Mf | 25 mer | GUUUGGCUACGGAGACGUGGUGUTT | 171 |
| 1096 | commons | Sense | | |
| | | Strand | | |
| KHK-1059- | Hs-Mf | 25 mer | UUUGGCUACGGAGACGUGGUGUUTG | 172 |
| 1097 | commons | Sense | | |
| | | Strand | | |
| KHK-1060- | Hs-Mf | 25 mer | UUGGCUACGGAGACGUGGUGUUUGT | 173 |
| 1098 | commons | Sense | | |
| | | Strand | | |
| KHK-1061- | Hs-Mf | 25 mer | UGGCUACGGAGACGUGGUGUUUGTC | 174 |
| 1099 | commons | Sense | | |
| | | Strand | | |
| KHK-1062- | Hs-Mf | 25 mer | GGCUACGGAGACGUGGUGUUUGUCA | 175 |
| 1100 | commons | Sense | | |
| | | Strand | | |
| KHK-1063- | Hs-Mf | 25 mer | GCUACGGAGACGUGGUGUUUGUCAG | 176 |
| 1101 | commons | Sense | | |
| | | Strand | | |
| KHK-1064- | Hs-Mf | 25 mer | CUACGGAGACGUGGUGUUUGUCAGC | 177 |
| 1102 | commons | Sense | | |
| | | Strand | | |
| KHK-1065- | Hs-Mf | 25 mer | UACGGAGACGUGGUGUUUGUCAGCA | 178 |
| 1103 | commons | Sense | | |
| | | Strand | | |
| KHK-1066- | Hs-Mf | 25 mer | ACGGAGACGUGGUGUUUGUCAGCAA | 179 |
| 1104 | commons | Sense | | |
| | | Strand | | |
| KHK-1067- | Hs-Mf | 25 mer | CGGAGACGUGGUGUUUGUCAGCAAA | 180 |
| 1105 | commons | Sense | | |
| | | Strand | | |
| KHK-1068- | Hs-Mf | 25 mer | GGAGACGUGGUGUUUGUCAGCAAAG | 181 |
| 1106 | commons | Sense | | |
| | | Strand | | |
| KHK-1069- | Hs-Mf | 25 mer | GAGACGUGGUGUUUGUCAGCAAAGA | 182 |
| 1107 | commons | Sense | | |
| | | Strand | | |
| KHK-1070- | Hs-Mf | 25 mer | AGACGUGGUGUUUGUCAGCAAAGAT | 183 |
| 1108 | commons | Sense | | |
| | | Strand | | |
| KHK-1071- | Hs-Mf | 25 mer | GACGUGGUGUUUGUCAGCAAAGATG | 184 |
| 1109 | commons | Sense | | |
| | | Strand | | |
| KHK-1072- | Hs-Mf | 25 mer | ACGUGGUGUUUGUCAGCAAAGAUGT | 185 |
| 1110 | commons | Sense | | |
| | | Strand | | |
| KHK-1073- | Hs-Mf | 25 mer | CGUGGUGUUUGUCAGCAAAGAUGTG | 186 |
| 1111 | commons | Sense | | |
| | | Strand | | |
| KHK-1074- | Hs-Mf- | 25 mer | GUGGUGUUUGUCAGCAAAGAUGUGG | 187 |
| 1112-943- | Mm-Rn | Sense | | |
| 785 | commons | Strand | | |
| KHK-1075- | Hs-Mf- | 25 mer | UGGUGUUUGUCAGCAAAGAUGUGGC | 188 |
| 1113-944- | Mm-Rn | Sense | | |
| 786 | commons | Strand | | |
| KHK-1076- | Hs-Mf- | 25 mer | GGUGUUUGUCAGCAAAGAUGUGGCC | 189 |
| 1114-945- | Mm-Rn | Sense | | |
| 787 | commons | Strand | | |
| KHK-1077- | Hs-Mf- | 25 mer | GUGUUUGUCAGCAAAGAUGUGGCCA | 190 |
| 1115-946- | Mm-Rn | Sense | | |
| 788 | commons | Strand | | |
| KHK-1078- | Hs-Mf- | 25 mer | UGUUUGUCAGCAAAGAUGUGGCCAA | 191 |
| 1116-947- | Mm-Rn | Sense | | |
| 789 | commons | Strand | | |
| KHK-1079- | Hs-Mf- | 25 mer | GUUUGUCAGCAAAGAUGUGGCCAAG | 192 |
| 1117-948- | Mm-Rn | Sense | | |
| 790 | commons | Strand | | |
| KHK-1080- | Hs-Mf- | 25 mer | UUUGUCAGCAAAGAUGUGGCCAAGC | 193 |
| 11 18-949- | Mm-Rn | Sense | | |
| 791 | commons | Strand | | |
| KHK-1081- | Hs-Mf- | 25 mer | UUGUCAGCAAAGAUGUGGCCAAGCA | 194 |
| 1119-950- | Mm-Rn | Sense | | |
| 792 | commons | Strand | | |
| KHK-1082- | Hs-Mf- | 25 mer | UGUCAGCAAAGAUGUGGCCAAGCAC | 195 |
| 1120-951- | Mm-Rn | Sense | | |
| 793 | commons | Strand | | |
| KHK-1083- | Hs-Mf- | 25 mer | GUCAGCAAAGAUGUGGCCAAGCACT | 196 |
| 1121-952- | Mm-Rn | Sense | | |
| 794 | commons | Strand | | |
| KHK-1084- | Hs-Mf- | 25 mer | UCAGCAAAGAUGUGGCCAAGCACTT | 197 |
| 1122-953- | Mm-Rn | Sense | | |
| 795 | commons | Strand | | |
| KHK-1085- | Hs-Mf- | 25 mer | CAGCAAAGAUGUGGCCAAGCACUTG | 198 |
| 1123-954- | Mm-Rn | Sense | | |
| 796 | commons | Strand | | |
| KHK-1086- | Hs-Mf- | 25 mer | AGCAAAGAUGUGGCCAAGCACUUGG | 199 |
| 1124-955- | Mm-Rn | Sense | | |
| 797 | commons | Strand | | |
| KHK-1087- | Hs-Mf- | 25 mer | GCAAAGAUGUGGCCAAGCACUUGGG | 200 |
| 1125-956- | Mm-Rn | Sense | | |
| 798 | commons | Strand | | |
| KHK-1090- | Hs-Mf | 25 mer | AAGAUGUGGCCAAGCACUUGGGGTT | 201 |
| 1128 | commons | Sense | | |
| | | Strand | | |
| KHK-1091- | Hs-Mf | 25 mer | AGAUGUGGCCAAGCACUUGGGGUTC | 202 |
| 1129 | commons | Sense | | |
| | | Strand | | |
| KHK-1092- | Hs-Mf | 25 mer | GAUGUGGCCAAGCACUUGGGGUUCC | 203 |
| 1130 | commons | Sense | | |
| | | Strand | | |
| KHK-1093- | Hs-Mf | 25 mer | AUGUGGCCAAGCACUUGGGGUUCCA | 204 |
| 1131 | commons | Sense | | |
| | | Strand | | |
| KHK-1095- | Hs-Mf | 25 mer | GUGGCCAAGCACUUGGGGUUCCAGT | 205 |
| 1133 | commons | Sense | | |
| | | Strand | | |
| KHK-1096- | Hs-Mf | 25 mer | UGGCCAAGCACUUGGGGUUCCAGTC | 206 |
| 1134 | commons | Sense | | |
| | | Strand | | |
| KHK-1097- | Hs-Mf | 25 mer | GGCCAAGCACUUGGGGUUCCAGUCA | 207 |
| 1135 | commons | Sense | | |
| | | Strand | | |
| KHK-1099- | Hs-Mf | 25 mer | CCAAGCACUUGGGGUUCCAGUCAGC | 208 |
| 1137 | commons | Sense | | |
| | | Strand | | |
| KHK-1100- | Hs-Mf | 25 mer | CAAGCACUUGGGGUUCCAGUCAGCA | 209 |
| 1138 | commons | Sense | | |
| | | Strand | | |
| KHK-1101- | Hs-Mf | 25 mer | AAGCACUUGGGGUUCCAGUCAGCAG | 210 |
| 1139 | commons | Sense | | |
| | | Strand | | |
| KHK-1102- | Hs-Mf | 25 mer | AGCACUUGGGGUUCCAGUCAGCAGA | 211 |
| 1140 | commons | Sense | | |
| | | Strand | | |
| KHK-1103- | Hs-Mf | 25 mer | GCACUUGGGGUUCCAGUCAGCAGAG | 212 |
| 1141 | commons | Sense | | |
| | | Strand | | |
| KHK-1104- | Hs-Mf | 25 mer | CACUUGGGGUUCCAGUCAGCAGAGG | 213 |
| 1142 | commons | Sense | | |
| | | Strand | | |
| KHK-1106- | Hs-Mf | 25 mer | CUUGGGGUUCCAGUCAGCAGAGGAA | 214 |
| 1144 | commons | Sense | | |
| | | Strand | | |
| KHK-1107- | Hs-Mf | 25 mer | UUGGGGUUCCAGUCAGCAGAGGAAG | 215 |
| 1145 | commons | Sense | | |
| | | Strand | | |
| KHK-1135- | Hs-Mf | 25 mer | UGAGGGGCUUGUAUGGUCGUGUGAG | 216 |
| 1173 | commons | Sense | | |
| | | Strand | | |
| KHK-1136- | Hs-Mf | 25 mer | GAGGGGCUUGUAUGGUCGUGUGAGG | 217 |
| ll74 | commons | Sense | | |
| | | Strand | | |
| KHK-1137- | Hs-Mf | 25 mer | AGGGGCUUGUAUGGUCGUGUGAGGA | 218 |
| 1175 | commons | Sense | | |
| | | Strand | | |
| KHK-1138- | Hs-Mf | 25 mer | GGGGCUUGUAUGGUCGUGUGAGGAA | 219 |
| 1176 | commons | Sense | | |
| | | Strand | | |
| KHK-1139- | Hs-Mf | 25 mer | GGGCUUGUAUGGUCGUGUGAGGAAA | 220 |
| 1177 | commons | Sense | | |
| | | Strand | | |
| KHK-1140- | Hs-Mf | 25 mer | GGCUUGUAUGGUCGUGUGAGGAAAG | 221 |
| 1178 | commons | Sense | | |
| | | Strand | | |
| KHK-1141- | Hs-Mf | 25 mer | GCUUGUAUGGUCGUGUGAGGAAAGG | 222 |
| 1179 | commons | Sense | | |
| | | Strand | | |
| KHK-1142- | Hs-Mf | 25 mer | CUUGUAUGGUCGUGUGAGGAAAGGG | 223 |
| 1180 | commons | Sense | | |
| | | Strand | | |
| KHK-1143- | Hs-Mf | 25 mer | UUGUAUGGUCGUGUGAGGAAAGGGG | 224 |
| 1181 | commons | Sense | | |
| | | Strand | | |
| KHK-1144- | Hs-Mf | 25 mer | UGUAUGGUCGUGUGAGGAAAGGGGC | 225 |
| 1182 | commons | Sense | | |
| | | Strand | | |
| KHK-1145- | Hs-Mf | 25 mer | GUAUGGUCGUGUGAGGAAAGGGGCT | 226 |
| 1183 | commons | Sense | | |
| | | Strand | | |
| KHK-1146- | Hs-Mf | 25 mer | UAUGGUCGUGUGAGGAAAGGGGCTG | 227 |
| 1184 | commons | Sense | | |
| | | Strand | | |
| KHK-1147- | Hs-Mf | 25 mer | AUGGUCGUGUGAGGAAAGGGGCUGT | 228 |
| 1185 | commons | Sense | | |
| | | Strand | | |
| KHK-1148- | Hs-Mf | 25 mer | UGGUCGUGUGAGGAAAGGGGCUGTG | 229 |
| 1186 | commons | Sense | | |
| | | Strand | | |
| KHK-1149- | Hs-Mf | 25 mer | GGUCGUGUGAGGAAAGGGGCUGUGC | 230 |
| 1187 | commons | Sense | | |
| | | Strand | | |
| KHK-1153- | Hs-Mf | 25 mer | GUGUGAGGAAAGGGGCUGUGCUUGT | 231 |
| 1191 | commons | Sense | | |
| | | Strand | | |
| KHK-1154- | Hs-Mf | 25 mer | UGUGAGGAAAGGGGCUGUGCUUGTC | 232 |
| 1192 | commons | Sense | | |
| | | Strand | | |
| KHK-1157- | Hs-Mf | 25 mer | GAGGAAAGGGGCUGUGCUUGUCUGT | 233 |
| 1195 | commons | Sense | | |
| | | Strand | | |
| KHK-1158- | Hs-Mf | 25 mer | AGGAAAGGGGCUGUGCUUGUCUGTG | 234 |
| 1196 | commons | Sense | | |
| | | Strand | | |
| KHK-1159- | Hs-Mf | 25 mer | GGAAAGGGGCUGUGCUUGUCUGUGC | 235 |
| 1197 | commons | Sense | | |
| | | Strand | | |
| KHK-1161- | Hs-Mf | 25 mer | AAAGGGGCUGUGCUUGUCUGUGCCT | 236 |
| 1199 | commons | Sense | | |
| | | Strand | | |
| KHK-1163- | Hs-Mf | 25 mer | AGGGGCUGUGCUUGUCUGUGCCUGG | 237 |
| 1201 | commons | Sense | | |
| | | Strand | | |
| KHK-1164- | Hs-Mf | 25 mer | GGGGCUGUGCUUGUCUGUGCCUGGG | 238 |
| 1202 | commons | Sense | | |
| | | Strand | | |
| KHK-1232- | Hs-Mf | 25 mer | CCACUCGGAUGCUUUCCCGCCACCC | 239 |
| 1270 | commons | Sense | | |
| | | Strand | | |
| KHK-1278- | Hs-Mf- | 25 mer | GCUGGAGACACCUUCAAUGCCUCCG | 240 |
| 1316-1147- | Mm-Rn | Sense | | |
| 989 | commons | Strand | | |
| KHK-1279- | Hs-Mf- | 25 mer | CUGGAGACACCUUCAAUGCCUCCGT | 241 |
| 1317-1148- | Mm-Rn | Sense | | |
| 990 | commons | Strand | | |
| KHK-1280- | Hs-Mf- | 25 mer | UGGAGACACCUUCAAUGCCUCCGTC | 242 |
| 1318-1149- | Mm-Rn | Sense | | |
| 991 | commons | Strand | | |
| KHK-1281- | Hs-Mf- | 25 mer | GGAGACACCUUCAAUGCCUCCGUCA | 243 |
| 1319-1150- | Mm-Rn | Sense | | |
| 992 | commons | Strand | | |
| KHK-1282- | Hs-Mf- | 25 mer | GAGACACCUUCAAUGCCUCCGUCAT | 244 |
| 1320-1151- | Mm-Rn | Sense | | |
| 993 | commons | Strand | | |
| KHK-1283- | Hs-Mf | 25 mer | AGACACCUUCAAUGCCUCCGUCATC | 245 |
| 1321 | commons | Sense | | |
| | | Strand | | |
| KHK-1284- | Hs-Mf | 25 mer | GACACCUUCAAUGCCUCCGUCAUCT | 246 |
| 1322 | commons | Sense | | |
| | | Strand | | |
| KHK-1285- | Hs-Mf | 25 mer | ACACCUUCAAUGCCUCCGUCAUCTT | 247 |
| 1323 | commons | Sense | | |
| | | Strand | | |
| KHK-1286- | Hs-Mf | 25 mer | CACCUUCAAUGCCUCCGUCAUCUTC | 248 |
| 1324 | commons | Sense | | |
| | | Strand | | |
| KHK-1287- | Hs-Mf | 25 mer | ACCUUCAAUGCCUCCGUCAUCUUCA | 249 |
| 1325 | commons | Sense | | |
| | | Strand | | |
| KHK-1288- | Hs-Mf | 25 mer | CCUUCAAUGCCUCCGUCAUCUUCAG | 250 |
| 1326 | commons | Sense | | |
| | | Strand | | |
| KHK-1289- | Hs-Mf | 25 mer | CUUCAAUGCCUCCGUCAUCUUCAGC | 251 |
| 1327 | commons | Sense | | |
| | | Strand | | |
| KHK-1290- | Hs-Mf | 25 mer | UUCAAUGCCUCCGUCAUCUUCAGCC | 252 |
| 1328 | commons | Sense | | |
| | | Strand | | |
| KHK-1291- | Hs-Mf | 25 mer | UCAAUGCCUCCGUCAUCUUCAGCCT | 253 |
| 1329 | commons | Sense | | |
| | | Strand | | |
| KHK-1292- | Hs-Mf | 25 mer | CAAUGCCUCCGUCAUCUUCAGCCTC | 254 |
| 1330 | commons | Sense | | |
| | | Strand | | |
| KHK-1293- | Hs-Mf | 25 mer | AAUGCCUCCGUCAUCUUCAGCCUCT | 255 |
| 1331 | commons | Sense | | |
| | | Strand | | |
| KHK-1294- | Hs-Mf | 25 mer | AUGCCUCCGUCAUCUUCAGCCUCTC | 256 |
| 1332 | commons | Sense | | |
| | | Strand | | |
| KHK-1295- | Hs-Mf | 25 mer | UGCCUCCGUCAUCUUCAGCCUCUCC | 257 |
| 1333 | commons | Sense | | |
| | | Strand | | |
| KHK-1297- | Hs-Mf | 25 mer | CCUCCGUCAUCUUCAGCCUCUCCCA | 258 |
| 1335 | commons | Sense | | |
| | | Strand | | |
| KHK-1323- | Hs-Mf | 25 mer | GGGAGGAGCGUGCAGGAAGCACUGA | 259 |
| 1361 | commons | Sense | | |
| | | Strand | | |
| KHK-1325- | Hs-Mf | 25 mer | GAGGAGCGUGCAGGAAGCACUGAGA | 260 |
| 1363 | commons | Sense | | |
| | | Strand | | |
| KHK-1326- | Hs-Mf | 25 mer | AGGAGCGUGCAGGAAGCACUGAGAT | 261 |
| 1364 | commons | Sense | | |
| | | Strand | | |
| KHK-1327- | Hs-Mf | 25 mer | GGAGCGUGCAGGAAGCACUGAGATT | 262 |
| 1365 | commons | Sense | | |
| | | Strand | | |
| KHK-1328- | Hs-Mf | 25 mer | GAGCGUGCAGGAAGCACUGAGAUTC | 263 |
| 1366 | commons | Sense | | |
| | | Strand | | |
| KHK-1329- | Hs-Mf | 25 mer | AGCGUGCAGGAAGCACUGAGAUUCG | 264 |
| 1367 | commons | Sense | | |
| | | Strand | | |
| KHK-1330- | Hs-Mf | 25 mer | GCGUGCAGGAAGCACUGAGAUUCGG | 265 |
| 1368 | commons | Sense | | |
| | | Strand | | |
| KHK-1331- | Hs-Mf | 25 mer | CGUGCAGGAAGCACUGAGAUUCGGG | 266 |
| 1369 | commons | Sense | | |
| | | Strand | | |
| KHK-1332- | Hs-Mf | 25 mer | GUGCAGGAAGCACUGAGAUUCGGGT | 267 |
| 1370 | commons | Sense | | |
| | | Strand | | |
| KHK-1333- | Hs-Mf | 25 mer | UGCAGGAAGCACUGAGAUUCGGGTG | 268 |
| 1371 | commons | Sense | | |
| | | Strand | | |
| KHK-1334- | Hs-Mf | 25 mer | GCAGGAAGCACUGAGAUUCGGGUGC | 269 |
| 1372 | commons | Sense | | |
| | | Strand | | |
| KHK-1335- | Hs-Mf | 25 mer | CAGGAAGCACUGAGAUUCGGGUGCC | 270 |
| 1373 | commons | Sense | | |
| | | Strand | | |
| KHK-1336- | Hs-Mf | 25 mer | AGGAAGCACUGAGAUUCGGGUGCCA | 271 |
| 1374 | commons | Sense | | |
| | | Strand | | |
| KHK-1385- | Hs-Mf | 25 mer | GCAGGGCUUUGAUGGCAUCGUGUGA | 272 |
| 1423 | commons | Sense | | |
| | | Strand | | |
| KHK-1387- | Hs-Mf | 25 mer | AGGGCUUUGAUGGCAUCGUGUGAGA | 273 |
| 1425 | commons | Sense | | |
| | | Strand | | |
| KHK-1388- | Hs-Mf | 25 mer | GGGCUUUGAUGGCAUCGUGUGAGAG | 274 |
| 1426 | commons | Sense | | |
| | | Strand | | |
| KHK-1389- | Hs-Mf | 25 mer | GGCUUUGAUGGCAUCGUGUGAGAGC | 275 |
| 1427 | commons | Sense | | |
| | | Strand | | |
| KHK-1538- | Hs-Mf | 25 mer | GCCUGUGUCCUGUGUUCCCCACAGG | 276 |
| 1588 | commons | Sense | | |
| | | Strand | | |
| KHK-1540- | Hs-Mf | 25 mer | CUGUGUCCUGUGUUCCCCACAGGGA | 277 |
| 1590 | commons | Sense | | |
| | | Strand | | |
| KHK-1542- | Hs-Mf | 25 mer | GUGUCCUGUGUUCCCCACAGGGAGA | 278 |
| 1592 | commons | Sense | | |
| | | Strand | | |
| KHK-1665- | Hs-Mf | 25 mer | CAUUCCUGAGGCUCUGACUCUUCGA | 279 |
| 1708 | commons | Sense | | |
| | | Strand | | |
| KHK-1666- | Hs-Mf | 25 mer | AUUCCUGAGGCUCUGACUCUUCGAT | 280 |
| 1709 | commons | Sense | | |
| | | Strand | | |
| KHK-1667- | Hs-Mf | 25 mer | UUCCUGAGGCUCUGACUCUUCGATC | 281 |
| 1710 | commons | Sense | | |
| | | Strand | | |
| KHK-1707- | Hs-Mf | 25 mer | CAUUCCCCAAAUUAACCUCUCCGCC | 282 |
| 1750 | commons | Sense | | |
| | | Strand | | |
| KHK-1708- | Hs-Mf | 25 mer | AUUCCCCAAAUUAACCUCUCCGCCC | 283 |
| 1751 | commons | Sense | | |
| | | Strand | | |
| KHK-1709- | Hs-Mf | 25 mer | UUCCCCAAAUUAACCUCUCCGCCCA | 284 |
| 1752 | commons | Sense | | |
| | | Strand | | |
| KHK-1869- | Hs-Mf | 25 mer | GGGCCCUGCGUUGUGCAGACUCUAT | 285 |
| 1918 | commons | Sense | | |
| | | Strand | | |
| KHK-1870- | Hs-Mf | 25 mer | GGCCCUGCGUUGUGCAGACUCUATT | 286 |
| 1919 | commons | Sense | | |
| | | Strand | | |
| KHK-1871- | Hs-Mf | 25 mer | GCCCUGCGUUGUGCAGACUCUAUTC | 287 |
| 1920 | commons | Sense | | |
| | | Strand | | |
| KHK-1872- | Hs-Mf | 25 mer | CCCUGCGUUGUGCAGACUCUAUUCC | 288 |
| 1921 | commons | Sense | | |
| | | Strand | | |
| KHK-1873- | Hs-Mf | 25 mer | CCUGCGUUGUGCAGACUCUAUUCCC | 289 |
| 1922 | commons | Sense | | |
| | | Strand | | |
| KHK-1874- | Hs-Mf | 25 mer | CUGCGUUGUGCAGACUCUAUUCCCA | 290 |
| 1923 | commons | Sense | | |
| | | Strand | | |
| KHK-1875- | Hs-Mf | 25 mer | UGCGUUGUGCAGACUCUAUUCCCAC | 291 |
| 1924 | commons | Sense | | |
| | | Strand | | |
| KHK-1876- | Hs-Mf | 25 mer | GCGUUGUGCAGACUCUAUUCCCACA | 292 |
| 1925 | commons | Sense | | |
| | | Strand | | |
| KHK-1877- | Hs-Mf | 25 mer | CGUUGUGCAGACUCUAUUCCCACAG | 293 |
| 1926 | commons | Sense | | |
| | | Strand | | |
| KHK-1878- | Hs-Mf | 25 mer | GUUGUGCAGACUCUAUUCCCACAGC | 294 |
| 1927 | commons | Sense | | |
| | | Strand | | |
| KHK-1879- | Hs-Mf | 25 mer | UUGUGCAGACUCUAUUCCCACAGCT | 295 |
| 1928 | commons | Sense | | |
| | | Strand | | |
| KHK-1880- | Hs-Mf | 25 mer | UGUGCAGACUCUAUUCCCACAGCTC | 296 |
| 1929 | commons | Sense | | |
| | | Strand | | |
| KHK-1900- | Hs-Mf | 25 mer | AGCUCAGAAGCUGGGAGUCCACACC | 297 |
| 1949 | commons | Sense | | |
| | | Strand | | |
| KHK-1905- | Hs-Mf | 25 mer | AGAAGCUGGGAGUCCACACCGCUGA | 298 |
| 1954 | commons | Sense | | |
| | | Strand | | |
| KHK-1971- | Hs-Mf | 25 mer | CUGCCCUGCCCACCAGCCUGUGATT | 299 |
| 2025 | commons | Sense | | |
| | | Strand | | |
| KHK-1974- | Hs-Mf | 25 mer | CCCUGCCCACCAGCCUGUGAUUUGA | 300 |
| 2028 | commons | Sense | | |
| | | Strand | | |
| KHK-1975- | Hs-Mf | 25 mer | CCUGCCCACCAGCCUGUGAUUUGAT | 301 |
| 2029 | commons | Sense | | |
| | | Strand | | |
| KHK-1976- | Hs-Mf | 25 mer | CUGCCCACCAGCCUGUGAUUUGATG | 302 |
| 2030 | commons | Sense | | |
| | | Strand | | |
| KHK-1978- | Hs-Mf | 25 mer | GCCCACCAGCCUGUGAUUUGAUGGG | 303 |
| 2032 | commons | Sense | | |
| | | Strand | | |
| KHK-1979- | Hs-Mf | 25 mer | CCCACCAGCCUGUGAUUUGAUGGGG | 304 |
| 2033 | commons | Sense | | |
| | | Strand | | |
| KHK-2032- | Hs-Mf | 25 mer | GCUGACUGCCCCAGAGCCUGAAAGT | 305 |
| 2086 | commons | Sense | | |
| | | Strand | | |
| KHK-2035- | Hs-Mf | 25 mer | GACUGCCCCAGAGCCUGAAAGUCTC | 306 |
| 2089 | commons | Sense | | |
| | | Strand | | |
| KHK-2036- | Hs-Mf | 25 mer | ACUGCCCCAGAGCCUGAAAGUCUCA | 307 |
| 2090 | commons | Sense | | |
| | | Strand | | |
| KHK-2037- | Hs-Mf | 25 mer | CUGCCCCAGAGCCUGAAAGUCUCAC | 308 |
| 2091 | commons | Sense | | |
| | | Strand | | |
| KHK-2038- | Hs-Mf | 25 mer | UGCCCCAGAGCCUGAAAGUCUCACC | 309 |
| 2092 | commons | Sense | | |
| | | Strand | | |
| KHK-2039- | Hs-Mf | 25 mer | GCCCCAGAGCCUGAAAGUCUCACCC | 310 |
| 2093 | commons | Sense | | |
| | | Strand | | |
| KHK-2040- | Hs-Mf | 25 mer | CCCCAGAGCCUGAAAGUCUCACCCT | 311 |
| 2094 | commons | Sense | | |
| | | Strand | | |
| KHK-2041- | Hs-Mf | 25 mer | CCCAGAGCCUGAAAGUCUCACCCTT | 312 |
| 2095 | commons | Sense | | |
| | | Strand | | |
| KHK-2042- | Hs-Mf | 25 mer | CCAGAGCCUGAAAGUCUCACCCUTG | 313 |
| 2096 | commons | Sense | | |
| | | Strand | | |
| KHK-2043- | Hs-Mf | 25 mer | CAGAGGCUGAAAGUCUCACCCUUGG | 314 |
| 2097 | commons | Sense | | |
| | | Strand | | |
| KHK-2044- | Hs-Mf | 25 mer | AGAGCCUGAAAGUCUCACCCUUGGA | 315 |
| 2098 | commons | Sense | | |
| | | Strand | | |
| KHK-2045- | Hs-Mf | 25 mer | GAGCCUGAAAGUCUCACCCUUGGAG | 316 |
| 2099 | commons | Sense | | |
| | | Strand | | |
| KHK-2067- | Hs-Mf | 25 mer | GAGCCCACCUUGGAAUUAAGGGCGT | 317 |
| 2121 | commons | Sense | | |
| | | Strand | | |
| KHK-2069- | Hs-Mf | 25 mer | GCCCACCUUGGAAUUAAGGGCGUGC | 318 |
| 2123 | commons | Sense | | |
| | | Strand | | |
| KHK-2091- | Hs-Mf | 25 mer | UGCCUCAGCCACAAAUGUGACCCAG | 319 |
| 2145 | commons | Sense | | |
| | | Strand | | |
| KHK-2092- | Hs-Mf | 25 mer | GCCUCAGCCACAAAUGUGACCCAGG | 320 |
| 2146 | commons | Sense | | |
| | | Strand | | |
| KHK-2093- | Hs-Mf | 25 mer | CCUCAGCCACAAAUGUGACCCAGGA | 321 |
| 2147 | commons | Sense | | |
| | | Strand | | |
| KHK-2094- | Hs-Mf | 25 mer | CUCAGCCACAAAUGUGACCCAGGAT | 322 |
| 2148 | commons | Sense | | |
| | | Strand | | |
| KHK-2095- | Hs-Mf | 25 mer | UCAGCCACAAAUGUGACCCAGGATA | 323 |
| 2149 | commons | Sense | | |
| | | Strand | | |
| KHK-2096- | Hs-Mf | 25 mer | CAGCCACAAAUGUGACCCAGGAUAC | 324 |
| 2150 | commons | Sense | | |
| | | Strand | | |
| KHK-2105 | Hs | 25 mer | AUGUGACCCAGGAUACAGAGUGUTG | 325 |
| | unique | Sense | | |
| | | Strand | | |
| KHK-2148- | Hs-Mf | 25 mer | GAUCUGGAACACAUAUUGGAAUUGG | 326 |
| 2197 | commons | Sense | | |
| | | Strand | | |
| KHK-2149- | Hs-Mf | 25 mer | AUCUGGAACACAUAUUGGAAUUGGG | 327 |
| 2198 | commons | Sense | | |
| | | Strand | | |
| KHK-2150- | Hs-Mf | 25 mer | UCUGGAACACAUAUUGGAAUUGGGG | 328 |
| 2199 | commons | Sense | | |
| | | Strand | | |
| KHK-2151- | Hs-Mf | 25 mer | CUGGAACACAUAUUGGAAUUGGGGC | 329 |
| 2200 | commons | Sense | | |
| | | Strand | | |
| KHK-2152- | Hs-Mf | 25 mer | UGGAACACAUAUUGGAAUUGGGGCC | 330 |
| 2201 | commons | Sense | | |
| | | Strand | | |
| KHK-2153- | Hs-Mf | 25 mer | GGAACACAUAUUGGAAUUGGGGCCA | 331 |
| 2202 | commons | Sense | | |
| | | Strand | | |
| KHK-2154- | Hs-Mf | 25 mer | GAACACAUAUUGGAAUUGGGGCCAA | 332 |
| 2203 | commons | Sense | | |
| | | Strand | | |
| KHK-2155- | Hs-Mf | 25 mer | AACACAUAUUGGAAUUGGGGCCAAC | 333 |
| 2204 | commons | Sense | | |
| | | Strand | | |
| KHK-2156- | Hs-Mf | 25 mer | ACACAUAUUGGAAUUGGGGCCAACT | 334 |
| 2205 | commons | Sense | | |
| | | Strand | | |
| KHK-2157- | Hs-Mf | 25 mer | CACAUAUUGGAAUUGGGGCCAACTC | 335 |
| 2206 | commons | Sense | | |
| | | Strand | | |
| KHK-2159- | Hs-Mf | 25 mer | CAUAUUGGAAUUGGGGCCAACUCCA | 336 |
| 2208 | commons | Sense | | |
| | | Strand | | |
| KHK-2160- | Hs-Mf | 25 mer | AUAUUGGAAUUGGGGCCAACUCCAA | 337 |
| 2209 | commons | Sense | | |
| | | Strand | | |
| KHK-2161- | Hs-Mf | 25 mer | UAUUGGAAUUGGGGCCAACUCCAAT | 338 |
| 2210 | commons | Sense | | |
| | | Strand | | |
| KHK-2162- | Hs-Mf | 25 mer | AUUGGAAUUGGGGCCAACUCCAATA | 339 |
| 2211 | commons | Sense | | |
| | | Strand | | |
| KHK-2163- | Hs-Mf | 25 mer | UUGGAAUUGGGGCCAACUCCAAUAT | 340 |
| 2212 | commons | Sense | | |
| | | Strand | | |
| KHK-2164- | Hs-Mf | 25 mer | UGGAAUUGGGGCCAACUCCAAUATA | 341 |
| 2213 | commons | Sense | | |
| | | Strand | | |
| KHK-2165- | Hs-Mf | 25 mer | GGAAUUGGGGCCAACUCCAAUAUAG | 342 |
| 2214 | commons | Sense | | |
| | | Strand | | |
| KHK-2166- | Hs-Mf | 25 mer | GAAUUGGGGCCAACUCCAAUAUAGG | 343 |
| 2215 | commons | Sense | | |
| | | Strand | | |
| KHK-2170- | Hs-Mf | 25 mer | UGGGGCCAACUCCAAUAUAGGGUGG | 344 |
| 2219 | commons | Sense | | |
| | | Strand | | |
| KHK-2196- | Hs-Mf | 25 mer | UAAGGCCUUAUAAUGUAAAGAGCAT | 345 |
| 2245 | commons | Sense | | |
| | | Strand | | |
| KHK-2197- | Hs-Mf | 25 mer | AAGGCCUUAUAAUGUAAAGAGCATA | 346 |
| 2246 | commons | Sense | | |
| | | Strand | | |
| KHK-2198- | Hs-Mf | 25 mer | AGGCCUUAUAAUGUAAAGAGCAUAT | 347 |
| 2247 | commons | Sense | | |
| | | Strand | | |
| KHK-2199- | Hs-Mf | 25 mer | GGCCUUAUAAUGUAAAGAGCAUATA | 348 |
| 2248 | commons | Sense | | |
| | | Strand | | |
| KHK-2200- | Hs-Mf | 25 mer | GCCUUAUAAUGUAAAGAGCAUAUAA | 349 |
| 2249 | commons | Sense | | |
| | | Strand | | |
| KHK-2201- | Hs-Mf | 25 mer | CCUUAUAAUGUAAAGAGCAUAUAAT | 350 |
| 2250 | commons | Sense | | |
| | | Strand | | |
| KHK-2205 | Hs | 25 mer | AUAAUGUAAAGAGCAUAUAAUGUAA | 351 |
| | unique | Sense | | |
| | | Strand | | |
| KHK-2238 | Hs | 25 mer | AGAGUGAGACAGACCUGGAUUAAAA | 352 |
| | unique | Sense | | |
| | | Strand | | |
| KHK-2260- | Hs-Mf | 25 mer | AAAUCUGCCAUUUAAUUAGCUGCAT | 353 |
| 2309 | commons | Sense | | |
| | | Strand | | |
| KHK-2261- | Hs-Mf | 25 mer | AAUCUGCCAUUUAAUUAGCUGCATA | 354 |
| 2310 | commons | Sense | | |
| | | Strand | | |
| KHK-2262- | Hs-Mf | 25 mer | AUCUGCCAUUUAAUUAGCUGCAUAT | 355 |
| 2311 | commons | Sense | | |
| | | Strand | | |
| KHK-2263- | Hs-Mf | 25 mer | UCUGCCAUUUAAUUAGCUGCAUATC | 356 |
| 2312 | commons | Sense | | |
| | | Strand | | |
| KHK-2264- | Hs-Mf | 25 mer | CUGCCAUUUAAUUAGCUGCAUAUCA | 357 |
| 2313 | commons | Sense | | |
| | | Strand | | |
| KHK-2265- | Hs-Mf | 25 mer | UGCCAUUUAAUUAGCUGCAUAUCAC | 358 |
| 2314 | commons | Sense | | |
| | | Strand | | |
| KHK-2266- | Hs-Mf | 25 mer | GCCAUUUAAUUAGCUGCAUAUCACC | 359 |
| 2315 | commons | Sense | | |
| | | Strand | | |
| KHK-2299 | Hs | 25 mer | CAGCACUUAACGCAAUCUGCCUCAA | 360 |
| | unique | Sense | | |
| | | Strand | | |
| KHK-2317- | Hs-Mf | 25 mer | GCCUCAAUUUCUUCAUCUGUCAAAT | 361 |
| 2366 | commons | Sense | | |
| | | Strand | | |
| KHK-2318- | Hs-Mf | 25 mer | CCUCAAUUUCUUCAUCUGUCAAATG | 362 |
| 2367 | commons | Sense | | |
| | | Strand | | |
| KHK-2319- | Hs-Mf | 25 mer | CUCAAUUUCUUCAUCUGUCAAAUGG | 363 |
| 2368 | commons | Sense | | |
| | | Strand | | |
| KHK-2320- | Hs-Mf | 25 mer | UCAAUUUCUUCAUCUGUCAAAUGGA | 364 |
| 2369 | commons | Sense | | |
| | | Strand | | |
| KHK-2321- | Hs-Mf | 25 mer | CAAUUUCUUCAUCUGUCAAAUGGAA | 365 |
| 2370 | commons | Sense | | |
| | | Strand | | |
| KHK-2322- | Hs-Mf | 25 mer | AAUUUCUUCAUCUGUCAAAUGGAAC | 366 |
| 2371 | commons | Sense | | |
| | | Strand | | |
| KHK-2323- | Hs-Mf | 25 mer | AUUUCUUCAUCUGUCAAAUGGAACC | 367 |
| 2372 | commons | Sense | | |
| | | Strand | | |
| KHK-2324- | Hs-Mf | 25 mer | UUUCUUCAUCUGUCAAAUGGAACCA | 368 |
| 2373 | commons | Sense | | |
| | | Strand | | |
| KHK-2325- | Hs-Mf | 25 mer | UUCUUCAUCUGUCAAAUGGAACCAA | 369 |
| 2374 | commons | Sense | | |
| | | Strand | | |
| KHK-2326- | Hs-Mf | 25 mer | UGUUCAUCUGUCAAAUGGAACCAAT | 370 |
| 2375 | commons | Sense | | |
| | | Strand | | |
| KHK-2332 | Hs | 25 mer | UCUGUCAAAUGGAACCAAUUCUGCT | 371 |
| | unique | Sense | | |
| | | Strand | | |
| KHK-2333 | Hs | 25 mer | CUGUCAAAUGGAACCAAUUCUGCTT | 372 |
| | unique | Sense | | |
| | | Strand | | |
| KHK-2335 | Hs | 25 mer | GUCAAAUGGAACCAAUUCUGCUUGG | 373 |
| | unique | Sense | | |
| | | Strand | | |
| KHK-2340 | Hs | 25 mer | AUGGAACCAAUUCUGCUUGGCUACA | 374 |
| | unique | Sense | | |
| | | Strand | | |
| KHK-2341 | Hs | 25 mer | UGGAACCAAUUCUGCUUGGCUACAG | 375 |
| | unique | Sense | | |
| | | Strand | | |
| KHK-2346 | Hs | 25 mer | CCAAUUCUGCUUGGCUACAGAAUTA | 376 |
| | unique | Sense | | |
| | | Strand | | |
| KHK-2352 | Hs | 25 mer | CUGCUUGGCUACAGAAUUAUUGUGA | 377 |
| | unique | Sense | | |
| | | Strand | | |
| KHK-2358 | Hs | 25 mer | GGCUACAGAAUUAUUGUGAGGAUAA | 378 |
| | unique | Sense | | |
| | | Strand | | |
| KHK-2359 | Hs | 25 mer | GCUACAGAAUUAUUGUGAGGAUAAA | 379 |
| | unique | Sense | | |
| | | Strand | | |
| KHK-2360 | Hs | 25 mer | CUACAGAAUUAUUGUGAGGAUAAAA | 380 |
| | unique | Sense | | |
| | | Strand | | |
| KHK-2361 | Hs | 25 mer | UACAGAAUUAUUGUGAGGAUAAAAT | 381 |
| | unique | Sense | | |
| | | Strand | | |
| KHK-2362 | Hs | 25 mer | ACAGAAUUAUUGUGAGGAUAAAATC | 382 |
| | unique | Sense | | |
| | | Strand | | |
| KHK-2363 | Hs | 25 mer | CAGAAUUAUUGUGAGGAUAAAAUCA | 383 |
| | unique | Sense | | |
| | | Strand | | |
| KHK-2364 | Hs | 25 mer | AGAAUUAUUGUGAGGAUAAAAUCAT | 384 |
| | unique | Sense | | |
| | | Strand | | |
| KHK-2365 | Hs | 25 mer | GAAUUAUUGUGAGGAUAAAAUCATA | 385 |
| | unique | Sense | | |
| | | Strand | | |
| KHK-2366 | Hs | 25 mer | AAUUAUUGUGAGGAUAAAAUCAUAT | 386 |
| | unique | Sense | | |
| | | Strand | | |
| KHK-2367 | Hs | 25 mer | AUUAUUGUGAGGAUAAAAUCAUATA | 387 |
| | unique | Sense | | |
| | | Strand | | |
| KHK-115- | Hs-Mf | 27mer | CGAGAAUGCAAAGAGAAAAUGCGCUAC | 388 |
| 154 | commons | Anti-sense | | |
| | | Strand | | |
| KHK-116- | Hs-Mf | 27mer | UCGAGAAUGCAAAGAGAAAAUGCGCUA | 389 |
| 155 | commons | Anti-sense | | |
| | | Strand | | |
| KHK-117- | Hs-Mf | 27mer | CUCGAGAAUGCAAAGAGAAAAUGCGCU | 390 |
| 156 | commons | Anti-sense | | |
| | | Strand | | |
| KHK-118- | Hs-Mf | 27mer | UCUCGAGAAUGCAAAGAGAAAAUGCGC | 391 |
| 157 | commons | Anti-sense | | |
| | | Strand | | |
| KHK-119- | Hs-Mf | 27mer | AUCUCGAGAAUGCAAAGAGAAAAUGCG | 392 |
| 158 | commons | Anti-sense | | |
| | | Strand | | |
| KHK-120- | Hs-Mf | 27mer | GAUCUGGAGAAUGCAAAGAGAAAAUGC | 393 |
| 159 | commons | Anti-sense | | |
| | | Strand | | |
| KHK-121- | Hs-Mf | 27mer | CGAUCUGGAGAAUGCAAAGAGAAAAUG | 394 |
| 160 | commons | Anti-sense | | |
| | | Strand | | |
| KHK-122- | Hs-Mf | 27mer | GCGAUCUCGAGAAUGCAAAGAGAAAAU | 395 |
| 161 | commons | Anti-sense | | |
| | | Strand | | |
| KHK-123- | Hs-Mf | 27mer | AGCGAUCUCGAGAAUGCAAAGAGAAAA | 396 |
| 162 | commons | Anti-sense | | |
| | | Strand | | |
| KHK-124- | Hs-Mf | 27mer | AAGCGAUCUCGAGAAUGCAAAGAGAAA | 397 |
| 163 | commons | Anti-sense | | |
| | | Strand | | |
| KHK-125- | Hs-Mf | 27mer | UAAGCGAUCUCGAGAAUGCAAAGAGAA | 398 |
| 164 | commons | Anti-sense | | |
| | | Strand | | |
| KHK-126- | Hs-Mf | 27mer | CUAAGCGAUCUCGAGAAUGCAAAGAGA | 399 |
| 165 | commons | Anti-sense | | |
| | | Strand | | |
| KHK-127- | Hs-Mf | 27mer | GCUAAGCGAUCUCGAGAAUGCAAAGAG | 400 |
| 166 | commons | Anti-sense | | |
| | | Strand | | |
| KHK-128- | Hs-Mf | 27mer | GGCUAAGCGAUCUCGAGAAUGCAAAGA | 401 |
| 167 | commons | Anti-sense | | |
| | | Strand | | |
| KHK-179 | Hs | 27mer | UUCCUCGCUUGUCAGAUGGACUCACAG | 402 |
| | unique | Anti-sense | | |
| | | Strand | | |
| KHK-181- | Hs-Mf | 27mer | GUUUCCUCGCUUGUCAGAUGGACUCAC | 403 |
| 220 | commons | Anti-sense | | |
| | | Strand | | |
| KHK-182- | Hs-Mf | 27mer | AGUUUCCUCGCUUGUCAGAUGGACUCA | 404 |
| 221 | commons | Anti-sense | | |
| | | Strand | | |
| KHK-183- | Hs-Mf | 27mer | UAGUUUCCUCGCUUGUCAGAUGGACUC | 405 |
| 222 | commons | Anti-sense | | |
| | | Strand | | |
| KHK-184- | Hs-Mf | 27mer | UUAGUUUCCUCGCUUGUCAGAUGGACU | 406 |
| 223 | commons | Anti-sense | | |
| | | Strand | | |
| KHK-185- | Hs-Mf | 27mer | CUUAGUUUCCUCGCUUGUCAGAUGGAC | 407 |
| 224 | commons | Anti-sense | | |
| | | Strand | | |
| KHK-186- | Hs-Mf | 27mer | CCUUAGUUUCCUCGCUUGUCAGAUGGA | 408 |
| 225 | commons | Anti-sense | | |
| | | Strand | | |
| KHK-187- | Hs-Mf | 27mer | GCCUUAGUUUCCUCGCUUGUCAGAUGG | 409 |
| 226 | commons | Anti-sense | | |
| | | Strand | | |
| KHK-188- | Hs-Mf | 27mer | AGCCUUAGUUUCCUCGCUUGUCAGAUG | 410 |
| 227 | commons | Anti-sense | | |
| | | Strand | | |
| KHK-431- | Hs-Mf | 27mer | CCUCUUAUCCAGGAGGAUGGUGUCCCC | 411 |
| 470 | commons | Anti-sense | | |
| | | Strand | | |
| KHK-432- | Hs-Mf | 27mer | GCCUCUUAUCCAGGAGGAUGGUGUCCC | 412 |
| 471 | commons | Anti-sense | | |
| | | Strand | | |
| KHK-433- | Hs-Mf | 27mer | UGCCUCUUAUCCAGGAGGAUGGUGUCC | 413 |
| 472 | commons | Anti-sense | | |
| | | Strand | | |
| KHK-507- | Hs-Mf- | 27mer | GGAUCUGCUUCUCUUCCAUGAGGCUAC | 414 |
| 545-376- | Mm-Rn | Anti-sense | | |
| 218 | commons | Strand | | |
| KHK-508- | Hs-Mf- | 27mer | AGGAUCUGCUUCUCUUCCAUGAGGCUA | 415 |
| 546-377- | Mm-Rn | Anti-sense | | |
| 219 | commons | Strand | | |
| KHK-509- | Hs-Mf- | 27mer | CAGGAUCUGCUUCUCUUCCAUGAGGCU | 416 |
| 547-378- | Mm-Rn | Anti-sense | | |
| 220 | commons | Strand | | |
| KHK-510- | Hs-Mf- | 27mer | ACAGGAUCUGCUUCUCUUCCAUGAGGC | 417 |
| 548-379- | Mm-Rn | Anti-sense | | |
| 221 | commons | Strand | | |
| KHK-511- | Hs-Mf- | 27mer | CACAGGAUCUGCUUCUCUUCCAUGAGG | 418 |
| 549-380- | Mm-Rn | Anti-sense | | |
| 222 | commons | Strand | | |
| KHK-512- | Hs-Mf- | 27mer | GCACAGGAUCUGCUUCUCUUCCAUGAG | 419 |
| 550-381- | Mm-Rn | Anti-sense | | |
| 223 | commons | Strand | | |
| KHK-513- | Hs-Mf- | 27mer | CGCACAGGAUCUGCUUCUCUUCCAUGA | 420 |
| 551-382- | Mm-Rn | Anti-sense | | |
| 224 | commons | Strand | | |
| KHK-514- | Hs-Mf- | 27mer | AGGCACAGGAUCUGCUUCUCUUCCAUG | 421 |
| 552-383- | Mm-Rn | Anti-sense | | |
| 225 | commons | Strand | | |
| KHK-515- | Hs-Mf- | 27mer | CAGGCACAGGAUCUGCUUCUCUUCCAU | 422 |
| 553-384- | Mm-Rn | Anti-sense | | |
| 226 | commons | Strand | | |
| KHK-516- | Hs-Mf- | 27mer | CCAGGCACAGGAUCUGCUUCUCUUCCA | 423 |
| 554-385- | Mm-Rn | Anti-sense | | |
| 227 | commons | Strand | | |
| KHK-517- | Hs-Mf- | 27mer | CCCACGCACAGGAUCUGCUUCUCUUCC | 424 |
| 555-386- | Mm-Rn | Anti-sense | | |
| 228 | commons | Strand | | |
| KHK-518- | Hs-Mf- | 27mer | CCCCACGCACAGGAUCUGCUUCUCUUC | 425 |
| 556-387- | Mm-Rn | Anti-sense | | |
| 229 | commons | Strand | | |
| KHK-520- | Hs-Mf- | 27mer | AGCCCCACGCACAGGAUCUGCUUCUCU | 426 |
| 558-389- | Mm-Rn | Anti-sense | | |
| 231 | commons | Strand | | |
| KHK-521- | Hs-Mf- | 27mer | UAGCCCCACGCACAGGAUCUGCUUCUC | 427 |
| 559-390- | Mm-Rn | Anti-sense | | |
| 232 | commons | Strand | | |
| KHK-522- | Hs-Mf- | 27mer | CUAGCCCCACGCACAGGAUCUGCUUCU | 428 |
| 560-391- | Mm-Rn | Anti-sense | | |
| 233 | commons | Strand | | |
| KHK-541- | Hs-Mf | 27mer | CUGAUGACGUCCAGCACCACUAGCCCC | 429 |
| 579 | commons | Anti-sense | | |
| | | Strand | | |
| KHK-544- | Hs-Mf | 27mer | AGGCUGAUGACGUCCAGCACCACUAGC | 430 |
| 582 | commons | Anti-sense | | |
| | | Strand | | |
| KHK-546- | Hs-Mf | 27mer | CCAGGCUGAUGACGUCCAGCACCACUA | 431 |
| 584 | commons | Anti-sense | | |
| | | Strand | | |
| KHK-547- | Hs-Mf | 27mer | ACCAGGCUGAUGACGUCCAGCACCACU | 432 |
| 585 | commons | Anti-sense | | |
| | | Strand | | |
| KHK-548- | Hs-Mf | 27mer | CACCAGGCUGAUGACGUCCAGCACCAC | 433 |
| 586 | commons | Anti-sense | | |
| | | Strand | | |
| KHK-549- | Hs-Mf | 27mer | CCACCAGGCUGAUGACGUCCAGCACCA | 434 |
| 587 | commons | Anti-sense | | |
| | | Strand | | |
| KHK-550- | Hs-Mf | 27mer | UCCACCAGGCUGAUGACGUCCAGCACC | 435 |
| 588 | commons | Anti-sense | | |
| | | Strand | | |
| KHK-551- | Hs-Mf | 27mer | GUCCACCAGGCUGAUGACGUCCAGCAC | 436 |
| 589 | commons | Anti-sense | | |
| | | Strand | | |
| KHK-552- | Hs-Mf | 27mer | UGUCCACCAGGCUGAUGACGUCCAGCA | 437 |
| 590 | commons | Anti-sense | | |
| | | Strand | | |
| KHK-553- | Hs-Mf | 27mer | UUGUCCACCAGGCUGAUGACGUCCAGC | 438 |
| 591 | commons | Anti-sense | | |
| | | Strand | | |
| KHK-554- | Hs-Mf | 27mer | CUUGUCCACCAGGCUGAUGACGUCCAG | 439 |
| 592 | commons | Anti-sense | | |
| | | Strand | | |
| KHK-555- | Hs-Mf | 27mer | ACUUGUCCACCAGGCUGAUGACGUCCA | 440 |
| 593 | commons | Anti-sense | | |
| | | Strand | | |
| KHK-556- | Hs-Mf | 27mer | UACUUGUCCACCAGGCUGAUGACGUCC | 441 |
| 594 | commons | Anti-sense | | |
| | | Strand | | |
| KHK-557- | Hs-Mf | 27mer | GUACUUGUCCACCAGGCUGAUGACGUC | 442 |
| 595 | commons | Anti-sense | | |
| | | Strand | | |
| KHK-558- | Hs-Mf | 27mer | GGUACUUGUCCACCAGGCUGAUGACGU | 443 |
| 596 | commons | Anti-sense | | |
| | | Strand | | |
| KHK-559- | Hs-Mf | 27mer | GGGUACUUGUCCACCAGGCUGAUGACG | 444 |
| 597 | commons | Anti-sense | | |
| | | Strand | | |
| KHK-560- | Hs-Mf | 27mer | AGGGUACUUGUCCACCAGGCUGAUGAC | 445 |
| 598 | commons | Anti-sense | | |
| | | Strand | | |
| KHK-561- | Hs-Mf | 27mer | UAGGGUACUUGUCCACCAGGCUGAUGA | 446 |
| 599 | commons | Anti-sense | | |
| | | Strand | | |
| KHK-562- | Hs-Mf | 27mer | UUAGGGUACUUGUCCACCAGGCUGAUG | 447 |
| 600 | commons | Anti-sense | | |
| | | Strand | | |
| KHK-563- | Hs-Mf | 27mer | CUUAGGGUACUUGUCCACCAGGCUGAU | 448 |
| 601 | commons | Anti-sense | | |
| | | Strand | | |
| KHK-564- | Hs-Mf | 27mer | CCUUAGGGUACUUGUCCACCAGGCUGA | 449 |
| 602 | commons | Anti-sense | | |
| | | Strand | | |
| KHK-565- | Hs-Mf | 27mer | UCCUUAGGGUACUUGUCCACCAGGCUG | 450 |
| 603 | commons | Anti-sense | | |
| | | Strand | | |
| KHK-566- | Hs-Mf | 27mer | CUCCUUAGGGUACUUGUCCACCAGGCU | 451 |
| 604 | commons | Anti-sense | | |
| | | Strand | | |
| KHK-567- | Hs-Mf | 27mer | CCUCCUUAGGGUACUUGUCCACCAGGC | 452 |
| 605 | commons | Anti-sense | | |
| | | Strand | | |
| KHK-568- | Hs-Mf | 27mer | UCCUCCUUAGGGUACUUGUCCACCAGG | 453 |
| 606 | commons | Anti-sense | | |
| | | Strand | | |
| KHK-569- | Hs-Mf | 27mer | GUCCUCCUUAGGGUACUUGUCCACCAG | 454 |
| 607 | commons | Anti-sense | | |
| | | Strand | | |
| KHK-570- | Hs-Mf | 27mer | AGUCCUCCUUAGGGUACUUGUCCACCA | 455 |
| 608 | commons | Anti-sense | | |
| | | Strand | | |
| KHK-571- | Hs-Mf | 27mer | GAGUCCUCCUUAGGGUACUUGUCCACC | 456 |
| 609 | commons | Anti-sense | | |
| | | Strand | | |
| KHK-572- | Hs-Mf | 27mer | CGAGUCCUCCUUAGGGUACUUGUCCAC | 457 |
| 610 | commons | Anti-sense | | |
| | | Strand | | |
| KHK-573- | Hs-Mf | 27mer | CCGAGUCCUCCUUAGGGUACUUGUCCA | 458 |
| 611 | commons | Anti-sense | | |
| | | Strand | | |
| KHK-574- | Hs-Mf | 27mer | UCCGAGUCCUCCUUAGGGUACUUGUCC | 459 |
| 612 | commons | Anti-sense | | |
| | | Strand | | |
| KHK-575- | Hs-Mf | 27mer | CUCCGAGUCCUCCUUAGGGUACUUGUC | 460 |
| 613 | commons | Anti-sense | | |
| | | Strand | | |
| KHK-576- | Hs-Mf | 27mer | UCUCCGAGUCCUCCUUAGGGUACUUGU | 461 |
| 614 | commons | Anti-sense | | |
| | | Strand | | |
| KHK-577- | Hs-Mf | 27mer | AUCUCCGAGUCCUCCUUAGGGUACUUG | 462 |
| 615 | commons | Anti-sense | | |
| | | Strand | | |
| KHK-638- | Hs-Mf | 27mer | GAGAACGGUGCAGGAGUUGGACGCGUU | 463 |
| 676 | commons | Anti-sense | | |
| | | Strand | | |
| KHK-641- | Hs-Mf | 27mer | GGAGAGAACGGUGCAGGAGUUGGACGC | 464 |
| 679 | commons | Anti-sense | | |
| | | Strand | | |
| KHK-642- | Hs-Mf | 27mer | GGGAGAGAACGGUGCAGGAGUUGGACG | 465 |
| 680 | commons | Anti-sense | | |
| | | Strand | | |
| KHK-643- | Hs-Mf | 27mer | AGGGAGAGAACGGUGCAGGAGUUGGAC | 466 |
| 681 | commons | Anti-sense | | |
| | | Strand | | |
| KHK-644- | Hs-Mf | 27mer | CAGGGAGAGAACGGUGCAGGAGUUGGA | 467 |
| 682 | commons | Anti-sense | | |
| | | Strand | | |
| KHK-645- | Hs-Mf | 27mer | GCAGGGAGAGAACGGUGCAGGAGUUGG | 468 |
| 683 | commons | Anti-sense | | |
| | | Strand | | |
| KHK-646- | Hs-Mf | 27mer | AGCAGGGAGAGAACGGUGCAGGAGUUG | 469 |
| 684 | commons | Anti-sense | | |
| | | Strand | | |
| KHK-647- | Hs-Mf | 27mer | GAGCAGGGAGAGAACGGUGCAGGAGUU | 470 |
| 685 | commons | Anti-sense | | |
| | | Strand | | |
| KHK-650- | Hs-Mf | 27mer | UCCGAGCAGGGAGAGAACGGUGCAGGA | 471 |
| 688 | commons | Anti-sense | | |
| | | Strand | | |
| KHK-676- | Hs-Mf | 27mer | AUUGAGCCCAUGAAGGCACAGGGGGCU | 472 |
| 714 | commons | Anti-sense | | |
| | | Strand | | |
| KHK-713- | Hs-Mf | 27mer | GUCGGCCACCAGGAAGUCAGCAACAUG | 473 |
| 722 | commons | Anti-sense | | |
| | | Strand | | |
| KHK-826- | Hs-Mf | 27mer | UGGAGCACAAUGGUACGGUUGCCAUUG | 474 |
| 835 | commons | Anti-sense | | |
| | | Strand | | |
| KHK-827- | Hs-Mf | 27mer | AUGGAGCACAAUGGUACGGUUGCCAUU | 475 |
| 836 | commons | Anti-sense | | |
| | | Strand | | |
| KHK-829- | Hs-Mf | 27mer | UCAUGGAGCACAAUGGUACGGUUGCCA | 476 |
| 838 | commons | Anti-sense | | |
| | | Strand | | |
| KHK-830- | Hs-Mf | 27mer | GUCAUGGAGCACAAUGGUACGGUUGCC | 477 |
| 839 | commons | Anti-sense | | |
| | | Strand | | |
| KHK-831- | Hs-Mf | 27mer | UGUCAUGGAGCACAAUGGUACGGUUGC | 478 |
| 840 | commons | Anti-sense | | |
| | | Strand | | |
| KHK-832- | Hs-Mf | 27mer | GUGUCAUGGAGCACAAUGGUACGGUUG | 479 |
| 841 | commons | Anti-sense | | |
| | | Strand | | |
| KHK-857- | Hs-Mf | 27mer | UGUAGCAGACACAUCUGGCAGGCUCGU | 480 |
| 895 | commons | Anti-sense | | |
| | | Strand | | |
| KHK-858- | Hs-Mf | 27mer | CUGUAGCAGACACAUCUGGCAGGCUCG | 481 |
| 896 | commons | Anti-sense | | |
| | | Strand | | |
| KHK-859- | Hs-Mf | 27mer | UCUGUAGCAGACACAUCUGGCAGGCUC | 482 |
| 897 | commons | Anti-sense | | |
| | | Strand | | |
| KHK-860- | Hs-Mf- | 27mer | GUCUGUAGCAGACACAUCUGGCAGGCU | 483 |
| 898-729- | Mm-Rn | Anti-sense | | |
| 571 | commons | Strand | | |
| KHK-861- | Hs-Mf- | 27mer | AGUCUGUAGCAGACACAUCUGGCAGGC | 484 |
| 899-730- | Mm-Rn | Anti-sense | | |
| 572 | commons | Strand | | |
| KHK-862- | Hs-Mf | 27mer | AAGUCUGUAGCAGACACAUCUGGCAGG | 485 |
| 900 | commons | Anti-sense | | |
| | | Strand | | |
| KHK-865 | Hs | 27mer | UCAAAGUCUGUAGCAGACACAUCUGGC | 486 |
| | unique | Anti-sense | | |
| | | Strand | | |
| KHK-880 | Hs | 27mer | GUCAGAUCAACCUUCUCAAAGUCUGUA | 487 |
| | unique | Anti-sense | | |
| | | Strand | | |
| KHK-882- | Hs-Mf | 27mer | GGGUCAGAUCAACCUUCUCAAAGUCUG | 488 |
| 920 | commons | Anti-sense | | |
| | | Strand | | |
| KHK-883- | Hs-Mf | 27mer | UGGGUCAGAUCAACCUUCUCAAAGUCU | 489 |
| 921 | commons | Anti-sense | | |
| | | Strand | | |
| KHK-884- | Hs-Mf | 27mer | CUGGGUCAGAUCAACCUUCUCAAAGUC | 490 |
| 922 | commons | Anti-sense | | |
| | | Strand | | |
| KHK-885- | Hs-Mf | 27mer | ACUGGGUCAGAUCAACCUUCUCAAAGU | 491 |
| 923 | commons | Anti-sense | | |
| | | Strand | | |
| KHK-886- | Hs-Mf | 27mer | AACUGGGUCAGAUCAACCUUCUCAAAG | 492 |
| 924 | commons | Anti-sense | | |
| | | Strand | | |
| KHK-887- | Hs-Mf | 27mer | GAACUGGGUCAGAUCAACCUUCUCAAA | 493 |
| 925 | commons | Anti-sense | | |
| | | Strand | | |
| KHK-888- | Hs-Mf | 27mer | UGAACUGGGUCAGAUCAACCUUCUCAA | 494 |
| 926 | commons | Anti-sense | | |
| | | Strand | | |
| KHK-889- | Hs-Mf | 27mer | UUGAACUGGGUCAGAUCAACCUUCUCA | 495 |
| 927 | commons | Anti-sense | | |
| | | Strand | | |
| KHK-890- | Hs-Mf | 27mer | CUUGAACUGGGUCAGAUCAACCUUCUC | 496 |
| 928 | commons | Anti-sense | | |
| | | Strand | | |
| KHK-891- | Hs-Mf | 27mer | ACUUGAACUGGGUCAGAUCAACCUUCU | 497 |
| 929 | commons | Anti-sense | | |
| | | Strand | | |
| KHK-892- | Hs-Mf | 27mer | CACUUGAACUGGGUCAGAUCAACCUUC | 498 |
| 930 | commons | Anti-sense | | |
| | | Strand | | |
| KHK-893- | Hs-Mf | 27mer | CCACUUGAACUGGGUCAGAUCAACCUU | 499 |
| 931 | commons | Anti-sense | | |
| | | Strand | | |
| KHK-894- | Hs-Mf | 27mer | UCCACUUGAACUGGGUCAGAUCAACCU | 500 |
| 932 | commons | Anti-sense | | |
| | | Strand | | |
| KHK-895- | Hs-Mf | 27mer | AUCCACUUGAACUGGGUCAGAUCAACC | 501 |
| 933 | commons | Anti-sense | | |
| | | Strand | | |
| KHK-896- | Hs-Mf | 27mer | GAUCCACUUGAACUGGGUCAGAUCAAC | 502 |
| 934 | commons | Anti-sense | | |
| | | Strand | | |
| KHK-897- | Hs-Mf | 27mer | GGAUCCACUUGAACUGGGUCAGAUCAA | 503 |
| 935 | commons | Anti-sense | | |
| | | Strand | | |
| KHK-898- | Hs-Mf | 27mer | UGGAUCCACUUGAACUGGGUCAGAUCA | 504 |
| 936 | commons | Anti-sense | | |
| | | Strand | | |
| KHK-899- | Hs-Mf | 27mer | GUGGAUCCACUUGAACUGGGUCAGAUC | 505 |
| 937 | commons | Anti-sense | | |
| | | Strand | | |
| KHK-900- | Hs-Mf | 27mer | UGUGGAUCCACUUGAACUGGGUCAGAU | 506 |
| 938 | commons | Anti-sense | | |
| | | Strand | | |
| KHK-901- | Hs-Mf | 27mer | AUGUGGAUCCACUUGAACUGGGUCAGA | 507 |
| 939 | commons | Anti-sense | | |
| | | Strand | | |
| KHK-902- | Hs-Mf | 27mer | AAUGUGGAUCCACUUGAACUGGGUCAG | 508 |
| 940 | commons | Anti-sense | | |
| | | Strand | | |
| KHK-903- | Hs-Mf | 27mer | CAAUGUGGAUCCACUUGAACUGGGUCA | 509 |
| 941 | commons | Anti-sense | | |
| | | Strand | | |
| KHK-904- | Hs-Mf | 27mer | UCAAUGUGGAUCCACUUGAACUGGGUC | 510 |
| 942 | commons | Anti-sense | | |
| | | Strand | | |
| KHK-905- | Hs-Mf | 27mer | CUCAAUGUGGAUCCACUUGAACUGGGU | 511 |
| 943 | commons | Anti-sense | | |
| | | Strand | | |
| KHK-906- | Hs-Mf | 27mer | CCUCAAUGUGGAUCCACUUGAACUGGG | 512 |
| 944 | commons | Anti-sense | | |
| | | Strand | | |
| KHK-907- | Hs-Mf | 27mer | CCCUCAAUGUGGAUCCACUUGAACUGG | 513 |
| 945 | commons | Anti-sense | | |
| | | Strand | | |
| KHK-908- | Hs-Mf- | 27mer | GCCCUCAAUGUGGAUCCACUUGAACUG | 514 |
| 946-777- | Mm-Rn | Anti-sense | | |
| 619 | commons | Strand | | |
| KHK-909- | Hs-Mf- | 27mer | GGCCCUCAAUGUGGAUCCACUUGAACU | 515 |
| 947-778- | Mm-Rn | Anti-sense | | |
| 620 | commons | Strand | | |
| KHK-910- | Hs-Mf- | 27mer | CGGCCCUCAAUGUGGAUCCACUUGAAC | 516 |
| 948-779- | Mm-Rn | Anti-sense | | |
| 621 | commons | Strand | | |
| KHK-911- | Hs-Mf- | 27mer | CCGGCCCUCAAUGUGGAUCCACUUGAA | 517 |
| 949-780- | Mm-Rn | Anti-sense | | |
| 622 | commons | Strand | | |
| KHK-912- | Hs-Mf- | 27mer | UCCGGCCCUCAAUGUGGAUCCACUUGA | 518 |
| 950-781- | Mm-Rn | Anti-sense | | |
| 623 | commons | Strand | | |
| KHK-913- | Hs-Mf- | 27mer | UUCCGGCCCUCAAUGUGGAUCCACUUG | 519 |
| 951-782- | Mm-Rn | Anti-sense | | |
| 624 | commons | Strand | | |
| KHK-914- | Hs-Mf- | 27mer | GUUCCGGCCCUCAAUGUGGAUCCACUU | 520 |
| 952-783- | Mm-Rn | Anti-sense | | |
| 625 | commons | Strand | | |
| KHK-939- | Hs-Mf | 27mer | GCAGCAUCUUCACCUGCUCCGAUGCGU | 521 |
| 977 | commons | Anti-sense | | |
| | | Strand | | |
| KHK-940- | Hs-Mf | 27mer | UGCAGCAUCUUCACCUGCUCCGAUGCG | 522 |
| 978 | commons | Anti-sense | | |
| | | Strand | | |
| KHK-941- | Hs-Mf | 27mer | CUGCAGCAUCUUCACCUGCUCCGAUGC | 523 |
| 979 | commons | Anti-sense | | |
| | | Strand | | |
| KHK-942- | Hs-Mf | 27mer | GCUGCAGCAUCUUCACCUGCUCCGAUG | 524 |
| 980 | commons | Anti-sense | | |
| | | Strand | | |
| KHK-943- | Hs-Mf | 27mer | CGCUGCAGCAUCUUCACCUGCUCCGAU | 525 |
| 981 | commons | Anti-sense | | |
| | | Strand | | |
| KHK-944- | Hs-Mf | 27mer | CCGCUGCAGCAUCUUCACCUGCUCCGA | 526 |
| 982 | commons | Anti-sense | | |
| | | Strand | | |
| KHK-945- | Hs-Mf | 27mer | UCCGCUGCAGCAUCUUCACCUGCUCCG | 527 |
| 983 | commons | Anti-sense | | |
| | | Strand | | |
| KHK-946- | Hs-Mf | 27mer | AUCCGCUGCAGCAUCUUCACCUGCUCC | 528 |
| 984 | commons | Anti-sense | | |
| | | Strand | | |
| KHK-947- | Hs-Mf | 27mer | UAUCCGCUGCAGCAUCUUCACCUGCUC | 529 |
| 985 | commons | Anti-sense | | |
| | | Strand | | |
| KHK-948- | Hs-Mf- | 27mer | CUAUCCGCUGCAGCAUCUUCACCUGCU | 530 |
| 986-817 | Mm | Anti-sense | | |
| | commons | Strand | | |
| KHK-949- | Hs-Mf- | 27mer | UCUAUCCGCUGCAGCAUCUUCACCUGC | 531 |
| 987-818 | Mm | Anti-sense | | |
| | commons | Strand | | |
| KHK-950- | Hs-Mf- | 27mer | GUCUAUCCGCUGCAGCAUCUUCACCUG | 532 |
| 988-819 | Mm | Anti-sense | | |
| | commons | Strand | | |
| KHK-951- | Hs-Mf- | 27mer | CGUCUAUCCGCUGCAGCAUCUUCACCU | 533 |
| 989-820 | Mm | Anti-sense | | |
| | commons | Strand | | |
| KHK-952- | Hs-Mf- | 27mer | GCGUCUAUCCGCUGCAGCAUCUUCACC | 534 |
| 990-821 | Mm | Anti-sense | | |
| | commons | Strand | | |
| KHK-953- | Hs-Mf- | 27mer | UGCGUCUAUCCGCUGCAGCAUCUUCAC | 535 |
| 991-822 | Mm | Anti-sense | | |
| | commons | Strand | | |
| KHK-954- | Hs-Mf- | 27mer | GUGCGUCUAUCCGCUGCAGCAUCUUCA | 536 |
| 992-823 | Mm | Anti-sense | | |
| | commons | Strand | | |
| KHK-955- | Hs-Mf- | 27mer | UGUGCGUCUAUCCGCUGCAGCAUCUUC | 537 |
| 993-824 | Mm | Anti-sense | | |
| | commons | Strand | | |
| KHK-956- | Hs-Mf | 27mer | GUGUGCGUCUAUCCGCUGCAGCAUCUU | 538 |
| 994 | commons | Anti-sense | | |
| | | Strand | | |
| KHK-957- | Hs-Mf | 27mer | UGUGUGCGUCUAUCCGCUGCAGCAUCU | 539 |
| 995 | commons | Anti-sense | | |
| | | Strand | | |
| KHK-958- | Hs-Mf | 27mer | UUGUGUGCGUCUAUCCGCUGCAGCAUC | 540 |
| 996 | commons | Anti-sense | | |
| | | Strand | | |
| KHK-978- | Hs-Mf | 27mer | GCUCUGGAGGCUGCCUGGUGUUGUGUG | 541 |
| 1016 | commons | Anti-sense | | |
| | | Strand | | |
| KHK-982- | Hs-Mf | 27mer | UUCUGCUCUGGAGGCUGCCUGGUGUUG | 542 |
| 1020 | commons | Anti-sense | | |
| | | Strand | | |
| KHK-983- | Hs-Mf | 27mer | CUUCUGCUCUGGAGGCUGCCUGGUGUU | 543 |
| 1021 | commons | Anti-sense | | |
| | | Strand | | |
| KHK-984- | Hs-Mf | 27mer | UCUUCUGCUCUGGAGGCUGCCUGGUGU | 544 |
| 1022 | commons | Anti-sense | | |
| | | Strand | | |
| KHK-985- | Hs-Mf | 27mer | AUCUUCUGCUCUGGAGGCUGCCUGGUG | 545 |
| 1023 | commons | Anti-sense | | |
| | | Strand | | |
| KHK-991- | Hs-Mf | 27mer | ACCCGGAUCUUCUGCUCUGGAGGCUGC | 546 |
| 1029 | commons | Anti-sense | | |
| | | Strand | | |
| KHK-992- | Hs-Mf | 27mer | CACCCGGAUCUUCUGCUCUGGAGGCUG | 547 |
| 1030 | commons | Anti-sense | | |
| | | Strand | | |
| KHK-993- | Hs-Mf | 27mer | ACACCCGGAUCUUCUGCUCUGGAGGCU | 548 |
| 1031 | commons | Anti-sense | | |
| | | Strand | | |
| KHK-999- | Hs-Mf | 27mer | CCACGGACACCCGGAUCUUCUGCUCUG | 549 |
| 1037 | commons | Anti-sense | | |
| | | Strand | | |
| KHK-1000- | Hs-Mf | 27mer | UCCACGGACACCCGGAUCUUCUGCUCU | 550 |
| 1038 | commons | Anti-sense | | |
| | | Strand | | |
| KHK-1019- | Hs-Mf | 27mer | CUCUCGUGGCUUCUCCACCUCCACGGA | 551 |
| 1057 | commons | Anti-sense | | |
| | | Strand | | |
| KHK-1054- | Hs-Mf | 27mer | ACCACGUCUCCGUAGCCAAACAGCUGG | 552 |
| 1092 | commons | Anti-sense | | |
| | | Strand | | |
| KHK-1055- | Hs-Mf | 27mer | CACCACGUCUCCGUAGCCAAACAGCUG | 553 |
| 1093 | commons | Anti-sense | | |
| | | Strand | | |
| KHK-1057- | Hs-Mf | 27mer | AACACCACGUCUCCGUAGCCAAACAGC | 554 |
| 1095 | commons | Anti-sense | | |
| | | Strand | | |
| KHK-1058- | Hs-Mf | 27mer | AAACACCACGUCUCCGUAGCCAAACAG | 555 |
| 1096 | commons | Anti-sense | | |
| | | Strand | | |
| KHK-1059- | Hs-Mf | 27mer | CAAACACCACGUCUCCGUAGCCAAACA | 556 |
| 1097 | commons | Anti-sense | | |
| | | Strand | | |
| KHK-1060- | Hs-Mf | 27mer | ACAAACACCACGUCUCCGUAGCCAAAC | 557 |
| 1098 | commons | Anti-sense | | |
| | | Strand | | |
| KHK-1061- | Hs-Mf | 27mer | GACAAACACCACGUCUCCGUAGCCAAA | 558 |
| 1099 | commons | Anti-sense | | |
| | | Strand | | |
| KHK-1062- | Hs-Mf | 27mer | UGACAAACACCACGUCUCCGUAGCCAA | 559 |
| 1100 | commons | Anti-sense | | |
| | | Strand | | |
| KHK-1063- | Hs-Mf | 27mer | CUGACAAACACCACGUCUCCGUAGCCA | 560 |
| 1101 | commons | Anti-sense | | |
| | | Strand | | |
| KHK-1064- | Hs-Mf | 27mer | GCUGACAAACACCACGUCUCCGUAGCC | 561 |
| 1102 | commons | Anti-sense | | |
| | | Strand | | |
| KHK-1065- | Hs-Mf | 27mer | UGCUGACAAACACCACGUCUCCGUAGC | 562 |
| 1103 | commons | Anti-sense | | |
| | | Strand | | |
| KHK-1066- | Hs-Mf | 27mer | UUGCUGACAAACACCACGUCUCCGUAG | 563 |
| 1104 | commons | Anti-sense | | |
| | | Strand | | |
| KHK-1067- | Hs-Mf | 27mer | UUUGCUGACAAACACCACGUCUCCGUA | 564 |
| 1105 | commons | Anti-sense | | |
| | | Strand | | |
| KHK-1068- | Hs-Mf | 27mer | CUUUGCUGACAAACACCACGUCUCCGU | 565 |
| 1106 | commons | Anti-sense | | |
| | | Strand | | |
| KHK-1069- | Hs-Mf | 27mer | UCUUUGCUGACAAACACCACGUCUCCG | 566 |
| 1107 | commons | Anti-sense | | |
| | | Strand | | |
| KHK-1070- | Hs-Mf | 27mer | AUCUUUGCUGACAAACACCACGUCUCC | 567 |
| 1108 | commons | Anti-sense | | |
| | | Strand | | |
| KHK-1071- | Hs-Mf | 27mer | CAUCUUUGCUGACAAACACCACGUCUC | 568 |
| 1109 | commons | Anti-sense | | |
| | | Strand | | |
| KHK-1072- | Hs-Mf | 27mer | ACAUCUUUGCUGACAAACACCACGUCU | 569 |
| 1110 | commons | Anti-sense | | |
| | | Strand | | |
| KHK-1073- | Hs-Mf | 27mer | CACAUCUUUGCUGACAAACACCACGUC | 570 |
| 1111 | commons | Anti-sense | | |
| | | Strand | | |
| KHK-1074- | Hs-Mf- | 27mer | CCACAUCUUUGCUGACAAACACCACGU | 571 |
| 1112-943- | Mm-Rn | Anti-sense | | |
| 785 | commons | Strand | | |
| KHK-1075- | Hs-Mf- | 27mer | GCCACAUCUUUGCUGACAAACACCACG | 572 |
| 1113-944- | Mm-Rn | Anti-sense | | |
| 786 | commons | Strand | | |
| KHK-1076- | Hs-Mf- | 27mer | GGCCACAUCUUUGCUGACAAACACCAC | 573 |
| 1114-945- | Mm-Rn | Anti-sense | | |
| 787 | commons | Strand | | |
| KHK-1077- | Hs-Mf- | 27mer | UGGCCACAUCUUUGCUGACAAACACCA | 574 |
| 1115-946- | Mm-Rn | Anti-sense | | |
| 788 | commons | Strand | | |
| KHK-1078- | Hs-Mf- | 27mer | UUGGCCACAUCUUUGCUGACAAACACC | 575 |
| 1116-947- | Mm-Rn | Anti-sense | | |
| 789 | commons | Strand | | |
| KHK-1079- | Hs-Mf- | 27mer | CUUGGCCACAUCUUUGCUGACAAACAC | 576 |
| 1117-948- | Mm-Rn | Anti-sense | | |
| 790 | commons | Strand | | |
| KHK-1080- | Hs-Mf- | 27mer | GCUUGGCCACAUCUUUGCUGACAAACA | 577 |
| 1118-949- | Mm-Rn | Anti-sense | | |
| 791 | commons | Strand | | |
| KHK-1081- | Hs-Mf- | 27mer | UGCUUGGCCACAUCUUUGCUGACAAAC | 578 |
| 1119-950- | Mm-Rn | Anti-sense | | |
| 792 | commons | Strand | | |
| KHK-1082- | Hs-Mf- | 27mer | GUGCUUGGCCACAUCUUUGCUGACAAA | 579 |
| 1120-951- | Mm-Rn | Anti-sense | | |
| 793 | commons | Strand | | |
| KHK-1083- | Hs-Mf- | 27mer | AGUGCUUGGCCACAUCUUUGCUGACAA | 580 |
| 1121-952- | Mm-Rn | Anti-sense | | |
| 794 | commons | Strand | | |
| KHK-1084- | Hs-Mf- | 27mer | AAGUGCUUGGCCACAUCUUUGCUGACA | 581 |
| 1122-953- | Mm-Rn | Anti-sense | | |
| 795 | commons | Strand | | |
| KHK-1085- | Hs-Mf- | 27mer | CAAGUGCUUGGCCACAUCUUUGCUGAC | 582 |
| 1123-954- | Mm-Rn | Anti-sense | | |
| 796 | commons | Strand | | |
| KHK-1086- | Hs-Mf- | 27mer | CCAAGUGCUUGGCCACAUCUUUGCUGA | 583 |
| 1124-955- | Mm-Rn | Anti-sense | | |
| 797 | commons | Strand | | |
| KHK-1087- | Hs-Mf- | 27mer | CCCAAGUGCUUGGCCACAUCUUUGCUG | 584 |
| 1125-956- | Mm-Rn | Anti-sense | | |
| 798 | commons | Strand | | |
| KHK-1090- | Hs-Mf | 27mer | AACCCCAAGUGCUUGGCCACAUCUUUG | 585 |
| 1128 | commons | Anti-sense | | |
| | | Strand | | |
| KHK-1091- | Hs-Mf | 27mer | GAACCCCAAGUGCUUGGCCACAUCUUU | 586 |
| 1129 | commons | Anti-sense | | |
| | | Strand | | |
| KHK-1092- | Hs-Mf | 27mer | GGAACCCCAAGUGCUUGGCCACAUCUU | 587 |
| 1130 | commons | Anti-sense | | |
| | | Strand | | |
| KHK-1093- | Hs-Mf | 27mer | UGGAACCCCAAGUGCUUGGCCACAUCU | 588 |
| 1131 | commons | Anti-sense | | |
| | | Strand | | |
| KHK-1095- | Hs-Mf | 27mer | ACUGGAACCCCAAGUGCUUGGCCACAU | 589 |
| 1133 | commons | Anti-sense | | |
| | | Strand | | |
| KHK-1096- | Hs-Mf | 27mer | GACUGGAACCCCAAGUGCUUGGCCACA | 590 |
| 1134 | commons | Anti-sense | | |
| | | Strand | | |
| KHK-1097- | Hs-Mf | 27mer | UGACUGGAACCCCAAGUGCUUGGCCAC | 591 |
| 1135 | commons | Anti-sense | | |
| | | Strand | | |
| KHK-1099- | Hs-Mf | 27mer | GCUGACUGGAACCCCAAGUGCUUGGCC | 592 |
| 1137 | commons | Anti-sense | | |
| | | Strand | | |
| KHK-1100- | Hs-Mf | 27mer | UGCUGACUGGAACCCCAAGUGCUUGGC | 593 |
| 1138 | commons | Anti-sense | | |
| | | Strand | | |
| KHK-1101- | Hs-Mf | 27mer | CUGCUGACUGGAACCCCAAGUGCUUGG | 594 |
| 1139 | commons | Anti-sense | | |
| | | Strand | | |
| KHK-1102- | Hs-Mf | 27mer | UCUGCUGACUGGAACCCCAAGUGCUUG | 595 |
| 1140 | commons | Anti-sense | | |
| | | Strand | | |
| KHK-1103- | Hs-Mf | 27mer | CUCUGCUGACUGGAACCCCAAGUGCUU | 596 |
| 1141 | commons | Anti-sense | | |
| | | Strand | | |
| KHK-1104- | Hs-Mf | 27mer | CCUCUGCUGACUGGAACCCCAAGUGCU | 597 |
| 1142 | commons | Anti-sense | | |
| | | Strand | | |
| KHK-1106- | Hs-Mf | 27mer | UUCCUCUGCUGACUGGAACCCCAAGUG | 598 |
| 1144 | commons | Anti-sense | | |
| | | Strand | | |
| KHK-1107- | Hs-Mf | 27mer | CUUCCUCUGCUGACUGGAACCCCAAGU | 599 |
| 1145 | commons | Anti-sense | | |
| | | Strand | | |
| KHK-1135- | Hs-Mf | 27mer | CUCACACGACCAUACAAGCCCCUCAAG | 600 |
| 1173 | commons | Anti-sense | | |
| | | Strand | | |
| KHK-1136- | Hs-Mf | 27mer | CCUCACACGACCAUACAAGCCCCUCAA | 601 |
| ll74 | commons | Anti-sense | | |
| | | Strand | | |
| KHK-1137- | Hs-Mf | 27mer | UCCUCACACGACCAUACAAGCCCCUCA | 602 |
| 1175 | commons | Anti-sense | | |
| | | Strand | | |
| KHK-1138- | Hs-Mf | 27mer | UUCCUCACACGACCAUACAAGCCCCUC | 603 |
| 1176 | commons | Anti-sense | | |
| | | Strand | | |
| KHK-1139- | Hs-Mf | 27mer | UUUCCUCACACGACCAUACAAGCCCCU | 604 |
| 1177 | commons | Anti-sense | | |
| | | Strand | | |
| KHK-1140- | Hs-Mf | 27mer | CUUUCCUCACACGACCAUACAAGCCCC | 605 |
| 1178 | commons | Anti-sense | | |
| | | Strand | | |
| KHK-1141- | Hs-Mf | 27mer | CCUUUCCUCACACGACCAUACAAGCCC | 606 |
| 1179 | commons | Anti-sense | | |
| | | Strand | | |
| KHK-1142- | Hs-Mf | 27mer | CCCUUUCCUCACACGACCAUACAAGCC | 607 |
| 1180 | commons | Anti-sense | | |
| | | Strand | | |
| KHK-1143- | Hs-Mf | 27mer | CCCCUUUCCUCACACGACCAUACAAGC | 608 |
| 1181 | commons | Anti-sense | | |
| | | Strand | | |
| KHK-1144- | Hs-Mf | 27mer | GCCCCUUUCCUCACACGACCAUACAAG | 609 |
| 1182 | commons | Anti-sense | | |
| | | Strand | | |
| KHK-1145- | Hs-Mf | 27mer | AGCCCCUUUCCUCACACGACCAUACAA | 610 |
| 1183 | commons | Anti-sense | | |
| | | Strand | | |
| KHK-1146- | Hs-Mf | 27mer | CAGCCCCUUUCCUCACACGACCAUACA | 611 |
| 1184 | commons | Anti-sense | | |
| | | Strand | | |
| KHK-1147- | Hs-Mf | 27mer | ACAGCCCCUUUCCUCACACGACCAUAC | 612 |
| 1185 | commons | Anti-sense | | |
| | | Strand | | |
| KHK-1148- | Hs-Mf | 27mer | CACAGCCCCUUUCCUCACACGACCAUA | 613 |
| 1186 | commons | Anti-sense | | |
| | | Strand | | |
| KHK-1149- | Hs-Mf | 27mer | GCACAGCCCCUUUCCUCACACGACCAU | 614 |
| 1187 | commons | Anti-sense | | |
| | | Strand | | |
| KHK-1153- | Hs-Mf | 27mer | ACAAGCACAGCCCCUUUCCUCACACGA | 615 |
| 1191 | commons | Anti-sense | | |
| | | Strand | | |
| KHK-1154- | Hs-Mf | 27mer | GACAAGCACAGCCCCUUUCCUCACACG | 616 |
| 1192 | commons | Anti-sense | | |
| | | Strand | | |
| KHK-1157- | Hs-Mf | 27mer | ACAGACAAGCACAGCCCCUUUCCUCAC | 617 |
| 1195 | commons | Anti-sense | | |
| | | Strand | | |
| KHK-1158- | Hs-Mf | 27mer | CACAGACAAGCACAGCCCCUUUCCUCA | 618 |
| 1196 | commons | Anti-sense | | |
| | | Strand | | |
| KHK-1159- | Hs-Mf | 27mer | GCACAGACAAGCACAGCCCCUUUCCUC | 619 |
| 1197 | commons | Anti-sense | | |
| | | Strand | | |
| KHK-1161- | Hs-Mf | 27mer | AGGCACAGACAAGCACAGCCCCUUUCC | 620 |
| 1199 | commons | Anti-sense | | |
| | | Strand | | |
| KHK-1163- | Hs-Mf | 27mer | CCAGGCACAGACAAGCACAGCCCCUUU | 621 |
| 1201 | commons | Anti-sense | | |
| | | Strand | | |
| KHK-1164- | Hs-Mf | 27mer | CCCAGGCACAGACAAGCACAGCCCCUU | 622 |
| 1202 | commons | Anti-sense | | |
| | | Strand | | |
| KHK-1232- | Hs-Mf | 27mer | GGGUGGCGGGAAAGCAUCCGAGUGGAG | 623 |
| 1270 | commons | Anti-sense | | |
| | | Strand | | |
| KHK-1278- | Hs-Mf- | 27mer | CGGAGGCAUUGAAGGUGUCUCCAGCUC | 624 |
| 1316-1147- | Mm-Rn | Anti-sense | | |
| 989 | commons | Strand | | |
| KHK-1279- | Hs-Mf- | 27mer | ACGGAGGCAUUGAAGGUGUCUCCAGCU | 625 |
| 1317-1148- | Mm-Rn | Anti-sense | | |
| 990 | commons | Strand | | |
| KHK-1280- | Hs-Mf- | 27mer | GACGGAGGCAUUGAAGGUGUCUCCAGC | 626 |
| 1318-1149- | Mm-Rn | Anti-sense | | |
| 991 | commons | Strand | | |
| KHK-1281- | Hs-Mf- | 27mer | UGACGGAGGCAUUGAAGGUGUCUCCAG | 627 |
| 1319-1150- | Mm-Rn | Anti-sense | | |
| 992 | commons | Strand | | |
| KHK-1282- | Hs-Mf- | 27mer | AUGACGGAGGCAUUGAAGGUGUCUCCA | 628 |
| 1320-1151- | Mm-Rn | Anti-sense | | |
| 993 | commons | Strand | | |
| KHK-1283- | Hs-Mf | 27mer | GAUGACGGAGGCAUUGAAGGUGUCUCC | 629 |
| 1321 | commons | Anti-sense | | |
| | | Strand | | |
| KHK-1284- | Hs-Mf | 27mer | AGAUGACGGAGGCAUUGAAGGUGUCUC | 630 |
| 1322 | commons | Anti-sense | | |
| | | Strand | | |
| KHK-1285- | Hs-Mf | 27mer | AAGAUGACGGAGGCAUUGAAGGUGUCU | 631 |
| 1323 | commons | Anti-sense | | |
| | | Strand | | |
| KHK-1286- | Hs-Mf | 27mer | GAAGAUGACGGAGGCAUUGAAGGUGUC | 632 |
| 1324 | commons | Anti-sense | | |
| | | Strand | | |
| KHK-1287- | Hs-Mf | 27mer | UGAAGAUGACGGAGGCAUUGAAGGUGU | 633 |
| 1325 | commons | Anti-sense | | |
| | | Strand | | |
| KHK-1288- | Hs-Mf | 27mer | CUGAAGAUGACGGAGGCAUUGAAGGUG | 634 |
| 1326 | commons | Anti-sense | | |
| | | Strand | | |
| KHK-1289- | Hs-Mf | 27mer | GCUGAAGAUGACGGAGGCAUUGAAGGU | 635 |
| 1327 | commons | Anti-sense | | |
| | | Strand | | |
| KHK-1290- | Hs-Mf | 27mer | GGCUGAAGAUGACGGAGGCAUUGAAGG | 636 |
| 1328 | commons | Anti-sense | | |
| | | Strand | | |
| KHK-1291- | Hs-Mf | 27mer | AGGCUGAAGAUGACGGAGGCAUUGAAG | 637 |
| 1329 | commons | Anti-sense | | |
| | | Strand | | |
| KHK-1292- | Hs-Mf | 27mer | GAGGCUGAAGAUGACGGAGGCAUUGAA | 638 |
| 1330 | commons | Anti-sense | | |
| | | Strand | | |
| KHK-1293- | Hs-Mf | 27mer | AGAGGCUGAAGAUGACGGAGGCAUUGA | 639 |
| 1331 | commons | Anti-sense | | |
| | | Strand | | |
| KHK-1294- | Hs-Mf | 27mer | GAGAGGCUGAAGAUGACGGAGGCAUUG | 640 |
| 1332 | commons | Anti-sense | | |
| | | Strand | | |
| KHK-1295- | Hs-Mf | 27mer | GGAGAGGCUGAAGAUGACGGAGGCAUU | 641 |
| 1333 | commons | Anti-sense | | |
| | | Strand | | |
| KHK-1297- | Hs-Mf | 27mer | UGGGAGAGGCUGAAGAUGACGGAGGCA | 642 |
| 1335 | commons | Anti-sense | | |
| | | Strand | | |
| KHK-1323- | Hs-Mf | 27mer | UCAGUGCUUCCUGCACGCUCCUCCCCU | 643 |
| 1361 | commons | Anti-sense | | |
| | | Strand | | |
| KHK-1325- | Hs-Mf | 27mer | UCUCAGUGCUUCCUGCACGCUCCUCCC | 644 |
| 1363 | commons | Anti-sense | | |
| | | Strand | | |
| KHK-1326- | Hs-Mf | 27mer | AUCUCAGUGCUUCCUGCACGCUCCUCC | 645 |
| 1364 | commons | Anti-sense | | |
| | | Strand | | |
| KHK-1327- | Hs-Mf | 27mer | AAUCUCAGUGCUUCCUGCACGCUCCUC | 646 |
| 1365 | commons | Anti-sense | | |
| | | Strand | | |
| KHK-1328- | Hs-Mf | 27mer | GAAUCUCAGUGCUUCCUGCACGCUCCU | 647 |
| 1366 | commons | Anti-sense | | |
| | | Strand | | |
| KHK-1329- | Hs-Mf | 27mer | CGAAUCUCAGUGCUUCCUGCACGCUCC | 648 |
| 1367 | commons | Anti-sense | | |
| | | Strand | | |
| KHK-1330- | Hs-Mf | 27mer | CCGAAUCUCAGUGCUUCCUGCACGCUC | 649 |
| 1368 | commons | Anti-sense | | |
| | | Strand | | |
| KHK-1331- | Hs-Mf | 27mer | CCCGAAUCUCAGUGCUUCCUGCACGCU | 650 |
| 1369 | commons | Anti-sense | | |
| | | Strand | | |
| KHK-1332- | Hs-Mf | 27mer | ACCCGAAUCUCAGUGCUUCCUGCACGC | 651 |
| 1370 | commons | Anti-sense | | |
| | | Strand | | |
| KHK-1333- | Hs-Mf | 27mer | CACCCGAAUCUCAGUGCUUCCUGCACG | 652 |
| 1371 | commons | Anti-sense | | |
| | | Strand | | |
| KHK-1334- | Hs-Mf | 27mer | GCACCCGAAUCUCAGUGCUUCCUGCAC | 653 |
| 1372 | commons | Anti-sense | | |
| | | Strand | | |
| KHK-1335- | Hs-Mf | 27mer | GGCACCCGAAUCUCAGUGCUUCCUGCA | 654 |
| 1373 | commons | Anti-sense | | |
| | | Strand | | |
| KHK-1336- | Hs-Mf | 27mer | UGGCACCCGAAUCUCAGUGCUUCCUGC | 655 |
| 1374 | commons | Anti-sense | | |
| | | Strand | | |
| KHK-1385- | Hs-Mf | 27mer | UCACACGAUGCCAUCAAAGCCCUGCAG | 656 |
| 1423 | commons | Anti-sense | | |
| | | Strand | | |
| KHK-1387- | Hs-Mf | 27mer | UCUCACACGAUGCCAUCAAAGCCCUGC | 657 |
| 1425 | commons | Anti-sense | | |
| | | Strand | | |
| KHK-1388- | Hs-Mf | 27mer | CUCUCACACGAUGCCAUCAAAGCCCUG | 658 |
| 1426 | commons | Anti-sense | | |
| | | Strand | | |
| KHK-1389- | Hs-Mf | 27mer | GCUCUCACACGAUGCCAUCAAAGCCCU | 659 |
| 1427 | commons | Anti-sense | | |
| | | Strand | | |
| KHK-1538- | Hs-Mf | 27mer | CCUGUGGGGAACACAGGACACAGGCAG | 660 |
| 1588 | commons | Anti-sense | | |
| | | Strand | | |
| KHK-1540- | Hs-Mf | 27mer | UCCCUGUGGGGAACACAGGACACAGGC | 661 |
| 1590 | commons | Anti-sense | | |
| | | Strand | | |
| KHK-1542- | Hs-Mf | 27mer | UCUCCCUGUGGGGAACACAGGACACAG | 662 |
| 1592 | commons | Anti-sense | | |
| | | Strand | | |
| KHK-1665- | Hs-Mf | 27mer | UCGAAGAGUCAGAGCCUCAGGAAUGCC | 663 |
| 1708 | commons | Anti-sense | | |
| | | Strand | | |
| KHK-1666- | Hs-Mf | 27mer | AUCGAAGAGUCAGAGCCUCAGGAAUGC | 664 |
| 1709 | commons | Anti-sense | | |
| | | Strand | | |
| KHK-1667- | Hs-Mf | 27mer | GAUCGAAGAGUCAGAGCCUCAGGAAUG | 665 |
| 1710 | commons | Anti-sense | | |
| | | Strand | | |
| KHK-1707- | Hs-Mf | 27mer | GGCGGAGAGGUUAAUUUGGGGAAUGGA | 666 |
| 1750 | commons | Anti-sense | | |
| | | Strand | | |
| KHK-1708- | Hs-Mf | 27mer | GGGCGGAGAGGUUAAUUUGGGGAAUGG | 667 |
| 1751 | commons | Anti-sense | | |
| | | Strand | | |
| KHK-1709- | Hs-Mf | 27mer | UGGGCGGAGAGGUUAAUUUGGGGAAUG | 668 |
| 1752 | commons | Anti-sense | | |
| | | Strand | | |
| KHK-1869- | Hs-Mf | 27mer | AUAGAGUCUGCACAACGCAGGGCCCCG | 669 |
| 1918 | commons | Anti-sense | | |
| | | Strand | | |
| KHK-1870- | Hs-Mf | 27mer | AAUAGAGUCUGCACAACGCAGGGCCCC | 670 |
| 1919 | commons | Anti-sense | | |
| | | Strand | | |
| KHK-1871- | Hs-Mf | 27mer | GAAUAGAGUCUGCACAACGCAGGGCCC | 671 |
| 1920 | commons | Anti-sense | | |
| | | Strand | | |
| KHK-1872- | Hs-Mf | 27mer | GGAAUAGAGUCUGCACAACGCAGGGCC | 672 |
| 1921 | commons | Anti-sense | | |
| | | Strand | | |
| KHK-1873- | Hs-Mf | 27mer | GGGAAUAGAGUCUGCACAACGCAGGGC | 673 |
| 1922 | commons | Anti-sense | | |
| | | Strand | | |
| KHK-1874- | Hs-Mf | 27mer | UGGGAAUAGAGUCUGCACAACGCAGGG | 674 |
| 1923 | commons | Anti-sense | | |
| | | Strand | | |
| KHK-1875- | Hs-Mf | 27mer | GUGGGAAUAGAGUCUGCACAACGCAGG | 675 |
| 1924 | commons | Anti-sense | | |
| | | Strand | | |
| KHK-1876- | Hs-Mf | 27mer | UGUGGGAAUAGAGUCUGCACAACGCAG | 676 |
| 1925 | commons | Anti-sense | | |
| | | Strand | | |
| KHK-1877- | Hs-Mf | 27mer | CUGUGGGAAUAGAGUCUGCACAACGCA | 677 |
| 1926 | commons | Anti-sense | | |
| | | Strand | | |
| KHK-1878- | Hs-Mf | 27mer | GCUGUGGGAAUAGAGUCUGCACAACGC | 678 |
| 1927 | commons | Anti-sense | | |
| | | Strand | | |
| KHK-1879- | Hs-Mf | 27mer | AGCUGUGGGAAUAGAGUCUGCACAACG | 679 |
| 1928 | commons | Anti-sense | | |
| | | Strand | | |
| KHK-1880- | Hs-Mf | 27mer | GAGCUGUGGGAAUAGAGUCUGCACAAC | 680 |
| 1929 | commons | Anti-sense | | |
| | | Strand | | |
| KHK-1900- | Hs-Mf | 27mer | GGUGUGGACUCCCAGCUUCUGAGCUGU | 681 |
| 1949 | commons | Anti-sense | | |
| | | Strand | | |
| KHK-1905- | Hs-Mf | 27mer | UCAGCGGUGUGGACUCCCAGCUUCUGA | 682 |
| 1954 | commons | Anti-sense | | |
| | | Strand | | |
| KHK-1971- | Hs-Mf | 27mer | AAUCACAGGCUGGUGGGCAGGGCAGAG | 683 |
| 2025 | commons | Anti-sense | | |
| | | Strand | | |
| KHK-1974- | Hs-Mf | 27mer | UCAAAUCACAGGCUGGUGGGCAGGGCA | 684 |
| 2028 | commons | Anti-sense | | |
| | | Strand | | |
| KHK-1975- | Hs-Mf | 27mer | AUCAAAUCACAGGCUGGUGGGCAGGGC | 685 |
| 2029 | commons | Anti-sense | | |
| | | Strand | | |
| KHK-1976- | Hs-Mf | 27mer | CAUCAAAUCACAGGCUGGUGGGCAGGG | 686 |
| 2030 | commons | Anti-sense | | |
| | | Strand | | |
| KHK-1978- | Hs-Mf | 27mer | CCCAUCAAAUCACAGGCUGGUGGGCAG | 687 |
| 2032 | commons | Anti-sense | | |
| | | Strand | | |
| KHK-1979- | Hs-Mf | 27mer | CCCCAUCAAAUCACAGGCUGGUGGGCA | 688 |
| 2033 | commons | Anti-sense | | |
| | | Strand | | |
| KHK-2032- | Hs-Mf | 27mer | ACUUUCAGGCUCUGGGGCAGUCAGCGG | 689 |
| 2086 | commons | Anti-sense | | |
| | | Strand | | |
| KHK-2035- | Hs-Mf | 27mer | GAGACUUUCAGGCUCUGGGGCAGUCAG | 690 |
| 2089 | commons | Anti-sense | | |
| | | Strand | | |
| KHK-2036- | Hs-Mf | 27mer | UGAGACUUUCAGGCUCUGGGGCAGUCA | 691 |
| 2090 | commons | Anti-sense | | |
| | | Strand | | |
| KHK-2037- | Hs-Mf | 27mer | GUGAGACUUUCAGGCUCUGGGGCAGUC | 692 |
| 2091 | commons | Anti-sense | | |
| | | Strand | | |
| KHK-2038- | Hs-Mf | 27mer | GGUGAGACUUUCAGGCUCUGGGGCAGU | 693 |
| 2092 | commons | Anti-sense | | |
| | | Strand | | |
| KHK-2039- | Hs-Mf | 27mer | GGGUGAGACUUUCAGGCUCUGGGGCAG | 694 |
| 2093 | commons | Anti-sense | | |
| | | Strand | | |
| KHK-2040- | Hs-Mf | 27mer | AGGGUGAGACUUUCAGGCUCUGGGGCA | 695 |
| 2094 | commons | Anti-sense | | |
| | | Strand | | |
| KHK-2041- | Hs-Mf | 27mer | AAGGGUGAGACUUUCAGGCUCUGGGGC | 696 |
| 2095 | commons | Anti-sense | | |
| | | Strand | | |
| KHK-2042- | Hs-Mf | 27mer | CAAGGGUGAGACUUUCAGGCUCUGGGG | 697 |
| 2096 | commons | Anti-sense | | |
| | | Strand | | |
| KHK-2043- | Hs-Mf | 27mer | CCAAGGGUGAGACUUUCAGGCUCUGGG | 698 |
| 2097 | commons | Anti-sense | | |
| | | Strand | | |
| KHK-2044- | Hs-Mf | 27mer | UCCAAGGGUGAGACUUUCAGGCUCUGG | 699 |
| 2098 | commons | Anti-sense | | |
| | | Strand | | |
| KHK-2045- | Hs-Mf | 27mer | CUCCAAGGGUGAGACUUUCAGGCUCUG | 700 |
| 2099 | commons | Anti-sense | | |
| | | Strand | | |
| KHK-2067- | Hs-Mf | 27mer | ACGCCCUUAAUUCCAAGGUGGGCUCCA | 701 |
| 2121 | commons | Anti-sense | | |
| | | Strand | | |
| KHK-2069- | Hs-Mf | 27mer | GCACGCCCUUAAUUCCAAGGUGGGCUC | 702 |
| 2123 | commons | Anti-sense | | |
| | | Strand | | |
| KHK-2091- | Hs-Mf | 27mer | CUGGGUCACAUUUGUGGCUGAGGCACG | 703 |
| 2145 | commons | Anti-sense | | |
| | | Strand | | |
| KHK-2092- | Hs-Mf | 27mer | CCUGGGUCACAUUUGUGGCUGAGGCAC | 704 |
| 2146 | commons | Anti-sense | | |
| | | Strand | | |
| KHK-2093- | Hs-Mf | 27mer | UCCUGGGUCACAUUUGUGGCUGAGGCA | 705 |
| 2147 | commons | Anti-sense | | |
| | | Strand | | |
| KHK-2094- | Hs-Mf | 27mer | AUCCUGGGUCACAUUUGUGGCUGAGGC | 706 |
| 2148 | commons | Anti-sense | | |
| | | Strand | | |
| KHK-2095- | Hs-Mf | 27mer | UAUCCUGGGUCACAUUUGUGGCUGAGG | 707 |
| 2149 | commons | Anti-sense | | |
| | | Strand | | |
| KHK-2096- | Hs-Mf | 27mer | GUAUCCUGGGUCACAUUUGUGGCUGAG | 708 |
| 2150 | commons | Anti-sense | | |
| | | Strand | | |
| KHK-2105 | Hs | 27mer | CAACACUCUGUAUCCUGGGUCACAUUU | 709 |
| | unique | Anti-sense | | |
| | | Strand | | |
| KHK-2148- | Hs-Mf | 27mer | CCAAUUCCAAUAUGUGUUCCAGAUCGG | 710 |
| 2197 | commons | Anti-sense | | |
| | | Strand | | |
| KHK-2149- | Hs-Mf | 27mer | CCCAAUUCCAAUAUGUGUUCCAGAUCG | 711 |
| 2198 | commons | Anti-sense | | |
| | | Strand | | |
| KHK-2150- | Hs-Mf | 27mer | CCCCAAUUCCAAUAUGUGUUCCAGAUC | 712 |
| 2199 | commons | Anti-sense | | |
| | | Strand | | |
| KHK-2151- | Hs-Mf | 27mer | GCCCCAAUUCCAAUAUGUGUUCCAGAU | 713 |
| 2200 | commons | Anti-sense | | |
| | | Strand | | |
| KHK-2152- | Hs-Mf | 27mer | GGCCCCAAUUCCAAUAUGUGUUCCAGA | 714 |
| 2201 | commons | Anti-sense | | |
| | | Strand | | |
| KHK-2153- | Hs-Mf | 27mer | UGGCCCCAAUUCCAAUAUGUGUUCCAG | 715 |
| 2202 | commons | Anti-sense | | |
| | | Strand | | |
| KHK-2154- | Hs-Mf | 27mer | UUGGCCCCAAUUCCAAUAUGUGUUCCA | 716 |
| 2203 | commons | Anti-sense | | |
| | | Strand | | |
| KHK-2155- | Hs-Mf | 27mer | GUUGGCCCCAAUUCCAAUAUGUGUUCC | 717 |
| 2204 | commons | Anti-sense | | |
| | | Strand | | |
| KHK-2156- | Hs-Mf | 27mer | AGUUGGCCCCAAUUCCAAUAUGUGUUC | 718 |
| 2205 | commons | Anti-sense | | |
| | | Strand | | |
| KHK-2157- | Hs-Mf | 27mer | GAGUUGGCCCCAAUUCCAAUAUGUGUU | 719 |
| 2206 | commons | Anti-sense | | |
| | | Strand | | |
| KHK-2159- | Hs-Mf | 27mer | UGGAGUUGGCCCCAAUUCCAAUAUGUG | 720 |
| 2208 | commons | Anti-sense | | |
| | | Strand | | |
| KHK-2160- | Hs-Mf | 27mer | UUGGAGUUGGCCCCAAUUCCAAUAUGU | 721 |
| 2209 | commons | Anti-sense | | |
| | | Strand | | |
| KHK-2161- | Hs-Mf | 27mer | AUUGGAGUUGGCCCCAAUUCCAAUAUG | 722 |
| 2210 | commons | Anti-sense | | |
| | | Strand | | |
| KHK-2162- | Hs-Mf | 27mer | UAUUGGAGUUGGCCCCAAUUCCAAUAU | 723 |
| 2211 | commons | Anti-sense | | |
| | | Strand | | |
| KHK-2163- | Hs-Mf | 27mer | AUAUUGGAGUUGGCCCCAAUUCCAAUA | 724 |
| 2212 | commons | Anti-sense | | |
| | | Strand | | |
| KHK-2164- | Hs-Mf | 27mer | UAUAUUGGAGUUGGCCCCAAUUCCAAU | 725 |
| 2213 | commons | Anti-sense | | |
| | | Strand | | |
| KHK-2165- | Hs-Mf | 27mer | CUAUAUUGGAGUUGGCCCCAAUUCCAA | 726 |
| 2214 | commons | Anti-sense | | |
| | | Strand | | |
| KHK-2166- | Hs-Mf | 27mer | CCUAUAUUGGAGUUGGCCCCAAUUCCA | 727 |
| 2215 | commons | Anti-sense | | |
| | | Strand | | |
| KHK-2170- | Hs-Mf | 27mer | CCACCCUAUAUUGGAGUUGGCCCCAAU | 728 |
| 2219 | commons | Anti-sense | | |
| | | Strand | | |
| KHK-2196- | Hs-Mf | 27mer | AUGCUCUUUACAUUAUAAGGCCUUACC | 729 |
| 2245 | commons | Anti-sense | | |
| | | Strand | | |
| KHK-2197- | Hs-Mf | 27mer | UAUGCUCUUUACAUUAUAAGGCCUUAC | 730 |
| 2246 | commons | Anti-sense | | |
| | | Strand | | |
| KHK-2198- | Hs-Mf | 27mer | AUAUGCUCUUUACAUUAUAAGGCCUUA | 731 |
| 2247 | commons | Anti-sense | | |
| | | Strand | | |
| KHK-2199- | Hs-Mf | 27mer | UAUAUGCUCUUUACAUUAUAAGGCCUU | 732 |
| 2248 | commons | Anti-sense | | |
| | | Strand | | |
| KHK-2200- | Hs-Mf | 27mer | UUAUAUGCUCUUUACAUUAUAAGGCCU | 733 |
| 2249 | commons | Anti-sense | | |
| | | Strand | | |
| KHK-2201- | Hs-Mf | 27mer | AUUAUAUGCUCUUUACAUUAUAAGGCC | 734 |
| 2250 | commons | Anti-sense | | |
| | | Strand | | |
| KHK-2205 | Hs | 27mer | UUACAUUAUAUGCUCUUUACAUUAUAA | 735 |
| | unique | Anti-sense | | |
| | | Strand | | |
| KHK-2238 | Hs | 27mer | UUUUAAUCCAGGUCUGUCUCACUCUAA | 736 |
| | unique | Anti-sense | | |
| | | Strand | | |
| KHK-2260- | Hs-Mf | 27mer | AUGCAGCUAAUUAAAUGGCAGAUUUUA | 737 |
| 2309 | commons | Anti-sense | | |
| | | Strand | | |
| KHK-2261- | Hs-Mf | 27mer | UAUGCAGCUAAUUAAAUGGCAGAUUUU | 738 |
| 2310 | commons | Anti-sense | | |
| | | Strand | | |
| KHK-2262- | Hs-Mf | 27mer | AUAUGCAGCUAAUUAAAUGGCAGAUUU | 739 |
| 2311 | commons | Anti-sense | | |
| | | Strand | | |
| KHK-2263- | Hs-Mf | 27mer | GAUAUGCAGCUAAUUAAAUGGCAGAUU | 740 |
| 2312 | commons | Anti-sense | | |
| | | Strand | | |
| KHK-2264- | Hs-Mf | 27mer | UGAUAUGCAGCUAAUUAAAUGGCAGAU | 741 |
| 2313 | commons | Anti-sense | | |
| | | Strand | | |
| KHK-2265- | Hs-Mf | 27mer | GUGAUAUGCAGCUAAUUAAAUGGCAGA | 742 |
| 2314 | commons | Anti-sense | | |
| | | Strand | | |
| KHK-2266- | Hs-Mf | 27mer | GGUGAUAUGCAGCUAAUUAAAUGGCAG | 743 |
| 2315 | commons | Anti-sense | | |
| | | Strand | | |
| KHK-2299 | Hs | 27mer | UUGAGGCAGAUUGCGUUAAGUGCUGUA | 744 |
| | unique | Anti-sense | | |
| | | Strand | | |
| KHK-2317- | Hs-Mf | 27mer | AUUUGACAGAUGAAGAAAUUGAGGCAG | 745 |
| 2366 | commons | Anti-sense | | |
| | | Strand | | |
| KHK-2318- | Hs-Mf | 27mer | CAUUUGACAGAUGAAGAAAUUGAGGCA | 746 |
| 2367 | commons | Anti-sense | | |
| | | Strand | | |
| KHK-2319- | Hs-Mf | 27mer | CCAUUUGACAGAUGAAGAAAUUGAGGC | 747 |
| 2368 | commons | Anti-sense | | |
| | | Strand | | |
| KHK-2320- | Hs-Mf | 27mer | UCCAUUUGACAGAUGAAGAAAUUGAGG | 748 |
| 2369 | commons | Anti-sense | | |
| | | Strand | | |
| KHK-2321- | Hs-Mf | 27mer | UUCCAUUUGACAGAUGAAGAAAUUGAG | 749 |
| 2370 | commons | Anti-sense | | |
| | | Strand | | |
| KHK-2322- | Hs-Mf | 27mer | GUUCCAUUUGACAGAUGAAGAAAUUGA | 750 |
| 2371 | commons | Anti-sense | | |
| | | Strand | | |
| KHK-2323- | Hs-Mf | 27mer | GGUUCCAUUUGACAGAUGAAGAAAUUG | 751 |
| 2372 | commons | Anti-sense | | |
| | | Strand | | |
| KHK-2324- | Hs-Mf | 27mer | UGGUUCCAUUUGACAGAUGAAGAAAUU | 752 |
| 2373 | commons | Anti-sense | | |
| | | Strand | | |
| KHK-2325- | Hs-Mf | 27mer | UUGGUUCCAUUUGACAGAUGAAGAAAU | 753 |
| 2374 | commons | Anti-sense | | |
| | | Strand | | |
| KHK-2326- | Hs-Mf | 27mer | AUUGGUUCCAUUUGACAGAUGAAGAAA | 754 |
| 2375 | commons | Anti-sense | | |
| | | Strand | | |
| KHK-2332 | Hs | 27mer | AGCAGAAUUGGUUCCAUUUGACAGAUG | 755 |
| | unique | Anti-sense | | |
| | | Strand | | |
| KHK-2333 | Hs | 27mer | AAGCAGAAUUGGUUCCAUUUGACAGAU | 756 |
| | unique | Anti-sense | | |
| | | Strand | | |
| KHK-2335 | Hs | 27mer | CCAAGCAGAAUUGGUUCCAUUUGACAG | 757 |
| | unique | Anti-sense | | |
| | | Strand | | |
| KHK-2340 | Hs | 27mer | UGUAGCCAAGCAGAAUUGGUUCCAUUU | 758 |
| | unique | Anti-sense | | |
| | | Strand | | |
| KHK-2341 | Hs | 27mer | CUGUAGCCAAGCAGAAUUGGUUCCAUU | 759 |
| | unique | Anti-sense | | |
| | | Strand | | |
| KHK-2346 | Hs | 27mer | UAAUUCUGUAGCCAAGCAGAAUUGGUU | 760 |
| | unique | Anti-sense | | |
| | | Strand | | |
| KHK-2352 | Hs | 27mer | UCACAAUAAUUCUGUAGCCAAGCAGAA | 761 |
| | unique | Anti-sense | | |
| | | Strand | | |
| KHK-2358 | Hs | 27mer | UUAUCCUCACAAUAAUUCUGUAGCCAA | 762 |
| | unique | Anti-sense | | |
| | | Strand | | |
| KHK-2359 | Hs | 27mer | UUUAUCCUCACAAUAAUUCUGUAGCCA | 763 |
| | unique | Anti-sense | | |
| | | Strand | | |
| KHK-2360 | Hs | 27mer | UUUUAUCCUCACAAUAAUUCUGUAGCC | 764 |
| | unique | Anti-sense | | |
| | | Strand | | |
| KHK-2361 | Hs | 27mer | AUUUUAUCCUCACAAUAAUUCUGUAGC | 765 |
| | unique | Anti-sense | | |
| | | Strand | | |
| KHK-2362 | Hs | 27mer | GAUUUUAUCCUCACAAUAAUUCUGUAG | 766 |
| | unique | Anti-sense | | |
| | | Strand | | |
| KHK-2363 | Hs | 27mer | UGAUUUUAUCCUCACAAUAAUUCUGUA | 767 |
| | unique | Anti-sense | | |
| | | Strand | | |
| KHK-2364 | Hs | 27mer | AUGAUUUUAUCCUCACAAUAAUUCUGU | 768 |
| | unique | Anti-sense | | |
| | | Strand | | |
| KHK-2365 | Hs | 27mer | UAUGAUUUUAUCCUCACAAUAAUUCUG | 769 |
| | unique | Anti-sense | | |
| | | Strand | | |
| KHK-2366 | Hs | 27mer | AUAUGAUUUUAUCCUCACAAUAAUUCU | 770 |
| | unique | Anti-sense | | |
| | | Strand | | |
| KHK-2367 | Hs | 27mer | UAUAUGAUUUUAUCCUCACAAUAAUUC | 771 |
| | unique | Anti-sense | | |
| | | Strand | | |
| Forward | NHP | N/A | TGCCTTCATGGGCTCAATG | 772 |
| Primer | KHK | | | |
| Reverse | NHP | N/A | TCGGCCACCAGGAAGTCA | 773 |
| Primer | KHK | | | |
| KHK-510- | Hs-Mf- | Low-2′- | [mCs][mU][mC][mA][mU][mG][mG][fA] | 774 |
| 548-379- | Mm-Rn | Fluoro | [fA][fG][fA][mG][mA][mA][mG][mC] | |
| 221 | commons | Pattern | [mA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-516- | Hs-Mf- | Low-2′- | [mGs][mA][mA][mG][mA][mG][mA][fA] | 775 |
| 554-385- | Mm-Rn | Fluoro | [fG][fC][fA][mG][mA][mU][mC][mC] | |
| 227 | commons | Pattern | [mU][mG][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-829- | Hs-Mf | Low-2′- | [mGs][mC][mA][mA][mC][mC][mG][fU] | 776 |
| 838 | commons | Fluoro | [fA][fC][fC][mA][mU][mU][mG][mU] | |
| | | Pattern | [mG][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-860- | Hs-Mf- | Low-2′- | [mCs][mC][mU][mG][mC][mC][mA][fG] | 777 |
| 898-729- | Mm-Rn | Fluoro | [fA][fU][fG][mU][mG][mU][mC][mU] | |
| 571 | commons | Pattern | [mG][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-861- | Hs-Mf- | Low-2′- | [mCs][mU][mG][mC][mC][mA][mG][fA] | 778 |
| 899-730- | Mm-Rn | Fluoro | [fU][fG][fU][mG][mU][mC][mU][mG] | |
| 572 | commons | Pattern | [mC][mU][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-865 | Hs | Low-2′- | [mCs][mA][mG][mA][mU][mG][mU][fG] | 779 |
| | unique | Fluoro | [fU][fC][fU][mG][mC][mU][mA][mC] | |
| | | Pattern | [mA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-882- | Hs-Mf | Low-2′- | [mGs][mA][mC][mU][mU][mU][mG][fA] | 780 |
| 920 | commons | Fluoro | [fG][fA][fA][mG][mG][mU][mU][mG] | |
| | | Pattern | [mA][mU][mC][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-883- | Hs-Mf | Low-2′- | [mAs][mC][mU][mU][mU][mG][mA][fG] | 781 |
| 921 | commons | Fluoro | [fA][fA][fG][mG][mU][mU][mG][mA] | |
| | | Pattern | [mU][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-885- | Hs-Mf | Low-2′- | [mUs][mU][mU][mG][mA][mG][mA][fA] | 782 |
| 923 | commons | Fluoro | [fG][fG][fU][mU][mG][mA][mU][mC] | |
| | | Pattern | [mU][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1054- | Hs-Mf | Low-2′- | [mAs][mG][fC][mU][mG][mU][mU][fU] | 783 |
| 1092 | commons | Fluoro | [fG][fG][m][fU][fA][mC][mG][mG] | |
| | | Pattern | C[fA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1075- | Hs-Mf- | Low-2′- | [mUs][mG][mG][mU][mG][mU][mU][fU] | 784 |
| 1113-944- | Mm-Rn | Fluoro | [fG][fU][fC][mA][mG][mC][mA][mA] | |
| 786 | commons | Pattern | [mA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1078- | Hs-Mf- | Low-2′- | [mUs][mG][mU][mU][mU][mG][mU][fC] | 785 |
| 1116-947- | Mm-Rn | Fluoro | [fA][fG][fC][mA][mA][mA][mG][mA] | |
| 789 | commons | Pattern | [mU][mG][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1281- | Hs-Mf- | Low-2′- | [mGs][mG][mA][mG][mA][mC][mA][fC] | 786 |
| 1319-1150- | Mm-Rn | Fluoro | [fC][fU][fU][mC][mA][mA][mU][mG] | |
| 992 | commons | Pattern | [mC][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1288- | Hs-Mf | Low-2′- | [mCs][mC][mU][mU][mC][mA][mA][fU] | 787 |
| 1326 | commons | Fluoro | [fG][fC][fC][mU][mC][mC][mG][mU] | |
| | | Pattern | [mC][mA][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1290- | Hs-Mf | Low-2′- | [mUs][mU][mC][mA][mA][mU][mG][fC] | 788 |
| 1328 | commons | Fluoro | [fC][fU][fC][mC][mG][mU][mC][mA] | |
| | | Pattern | [mU][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1148- | Hs-Mf | Low-2′- | [mUs][mG][mG][mU][mC][mG][mU][fG] | 789 |
| 1186 | commons | Fluoro | [fU][fG][fA][mG][mG][mA][mA][mA] | |
| | | Pattern | [mG][mG][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1152- | Hs-Mf | Low-2′- | [mCs][mG][mU][mG][mU][mG][mA][fG] | 790 |
| 1190 | commons | Fluoro | [fG][fA][fA][mA][mG][mG][mG][mG] | |
| | | Pattern | [mC][mU][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1154- | Hs-Mf | Low-2′- | [mUs][mG][mU][mG][mA][mG][mG][fA] | 791 |
| 1192 | commons | Fluoro | [fA][fA][fG][mG][mG][mG][mC][mU] | |
| | | Pattern | [mG][mU][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1155- | Hs-Mf | Low-2′- | [mGs][mU][mG][mA][mG][mG][mA][fA] | 792 |
| 1193 | commons | Fluoro | [fA][fG][fG][mG][mG][mC][mU][mG] | |
| | | Pattern | [mU][mG][mC][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1277 | Hs | Low-2′- | [mAs][mG][mC][mU][mG][mG][mA][fG] | 793 |
| | unique | Fluoro | [fA][fC][fA][mC][mC][mU][mU][mC] | |
| | | Pattern | [mA][mA][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1147- | Hs-Mf | Low-2′- | [mAs][mU][mG][mG][mU][mC][mG][fU] | 794 |
| 1185 | commons | Fluoro | [fG][fU][fG][mA][mG][mG][mA][mA] | |
| | | Pattern | [mA][mG][mG][mA][G][mC][mA][mG] | |
| | | Modified | m[mC][mC][mG][ademA-GalNAc][ademA- | |
| | | Sense | GalNAc][ademA-GalNAc][mG][mG][mC] | |
| | | Strand | [mU][mG][mC] | |
| KHK-869- | Hs-Mf | Low-2′- | [mUs][mG][mU][mG][mU][mC][mU][fG] | 795 |
| 934 | commons | Fluoro | [fC][fU][fA][mC][mA][mG][mA][mC] | |
| | | Pattern | [mU][mU][mU][mA][mG][mC][mA][mG] | |
| | | Modified | [mC][mC][mG][ademA-GalNAc][ademA- | |
| | | Sense | GalNAc][ademA-alNAc][mG][mG][mC] | |
| | | Strand | G[mU][mG][mC] | |
| KHK-873 | Hs | Low-2′- | [mUs][mC][mU][mG][mC][mU][mA][fC] | 796 |
| | unique | Fluoro | [fA][fG][fA][mC][mU][mU][mU][mG] | |
| | | Pattern | [mA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-879 | Hs | Low-2′- | [mAs][mC][mA][mG][mA][mC][mU][fU] | 797 |
| | unique | Fluoro | [fU][fG][fA][mG][mA][mA][mG][mG] | |
| | | Pattern | [mU][mU][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-881 | Hs | Low-2′- | [mAs][mG][mA][mC][mU][mU][mU][fG] | 798 |
| | unique | Fluoro | [fA][fG][fA][mA][mG][mG][mU][mU] | |
| | | Pattern | [mG][mA][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-896- | Hs-Mf | Low-2′- | [mUs][mG][mA][mU][mC][mU][mG][fA] | 799 |
| 934 | commons | Fluoro | [fC][fC][fC][mA][mG][mU][mU][mC] | |
| | | Pattern | [mA][mA][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1064- | Hs-Mf | Low-2′- | [mCs][mU][mA][mC][mG][mG][mA][fG] | 800 |
| 1102 | commons | Fluoro | [fA][fC][fG][mU][mG][mG][mU][mG] | |
| | | Pattern | [mU][mU][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1077- | Hs-Mf- | Low-2′- | [mGs][mU][mG][mU][mU][mU][mG][fU] | 801 |
| 1115-946- | Mm-Rn | Fluoro | [fC][fA][fG][mC][mA][mA][mA][mG] | |
| 788 | commons | Pattern | [mA][mU][mG][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1080- | Hs-Mf- | Low-2′- | [mUs][mU][mU][mG][mU][mC][mA][fG] | 802 |
| 1118-949- | Mm-Rn | Fluoro | [fC][fA][fA][mA][mG][mA][mU][mG] | |
| 791 | commons | Pattern | [mU][mG][mG][mA][mG][mC][mA][mG] | |
| | | Modified | [mC][mC][mG][ademA-GalNAc][ademA- | |
| | | Sense | GalNAc][ademA-alNAc][mG][mG][mC] | |
| | | Strand | G[mU][mG][mC] | |
| KHK-1106- | Hs-Mf | Low-2′- | [mCs][mU][mU][mG][mG][mG][mG][fU] | 803 |
| 1144 | commons | Fluoro | [fU][fC][fC][mA][mG][mU][mC][mA] | |
| | | Pattern | [mG][mC][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1334- | Hs-Mf | Low-2′- | [mGs][mC][mA][mG][mG][mA][mA][fG] | 804 |
| 1372 | commons | Fluoro | [fC][fA][fC][mU][mG][mA][mG][mA] | |
| | | Pattern | [mU][mU][mC][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-516- | Hs-Mf- | Med-2′- | [mGs][mA][fA][mG][mA][mG][mA][fA] | 805 |
| 554-385- | Mm-Rn | Fluoro | [fG][fC][mA][fG][fA][mU][mC][mC] | |
| 227 | commons | Pattern | [fU][mG][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-804 | Hs | Med-2′- | [mUs][mG][fC][mU][mG][mC][mA][fU] | 806 |
| | unique | Fluoro | [fC][fA][mU][fC][fA][mA][mC][mA] | |
| | | Pattern | [fA][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-829- | Hs-Mf | Med-2′- | [mGs][mC][fA][mA][mC][mC][mG][fU] | 807 |
| 838 | commons | Fluoro | [fA][fC][mC][fA][fU][mU][mG][mU] | |
| | | Pattern | [fG][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-861- | Hs-Mf- | Med-2′- | [mCs][mU][fG][mC][mC][mA][mG][fA] | 808 |
| 899-730- | Mm-Rn | Fluoro | [fU][fG][mU][fG][fU][mC][mU][mG] | |
| 572 | commons | Pattern | [fC][mU][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-865 | Hs | Med-2′- | [mCs][mA][fG][mA][mU][mG][mU][fG] | 809 |
| | unique | Fluoro | [fU][fC][mU][fG][fC][mU][mA][mC] | |
| | | Pattern | [fA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-882- | Hs-Mf | Med-2′- | [mGs][mA][fC][mU][mU][mU][mG][fA] | 810 |
| 920 | commons | Fluoro | [fG][fA][mA][fG][fG][mU][mU][mG] | |
| | | Pattern | [fA][mU][mC][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-883- | Hs-Mf | Med-2′- | [mAs][mC][fU][mU][mU][mG][mA][fG] | 811 |
| 921 | commons | Fluoro | [fA][fA][mG][fG][fU][mU][mG][mA] | |
| | | Pattern | [fU][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-885- | Hs-Mf | Med-2′- | [mUs][mU][fU][mG][mA][mG][mA][fA] | 812 |
| 923 | commons | Fluoro | [fG][fG][mU][fU][fG][mA][mU][mC] | |
| | | Pattern | [fU][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1054- | Hs-Mf | Med-2′- | [mAs][mG][fC][mU][mG][mU][mU][fU] | 813 |
| 1092 | commons | Fluoro | [fG][fG][mC][fU][fA][mC][mG][mG] | |
| | | Pattern | [fA][mG][mA][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1076- | Hs-Mf- | Med-2′- | [mGs][mG][fU][mG][mU][mU][mU][fG] | 814 |
| 1114-945- | Mm-Rn | Fluoro | [fU][fC][mA][fG][fC][mA][mA][mA] | |
| 787 | commons | Pattern | [fG][mA][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1078- | Hs-Mf- | Med-2′- | [mUs][mG][fU][mU][mU][mG][mU][fC] | 815 |
| 1116-947- | Mm-Rn | Fluoro | [fA][fG][mC][fA][fA][mA][mG][mA] | |
| 789 | commons | Pattern | [fU][mG][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1288- | Hs-Mf | Med-2′- | [mCs][mC][fU][mU][mC][mA][mA][fU] | 816 |
| 1326 | commons | Fluoro | [fG][fC][mC][fU][fC][mC][mG][mU] | |
| | | Pattern | [fC][mA][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1290- | Hs-Mf | Med-2′- | [mUs][mU][fC][mA][mA][mU][mG][fC] | 817 |
| 1328 | commons | Fluoro | [fC][fU][mC][fC][fG][mU][mC][mA] | |
| | | Pattern | [fU][mC][mU][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-1334- | Hs-Mf | Med-2′- | [mGs][mC][fA][mG][mG][mA][mA][fG] | 818 |
| 1372 | commons | Fluoro | [fC][fA][mC][fU][fG][mA][mG][mA] | |
| | | Pattern | [fU][mU][mC][mA][mG][mC][mA][mG] | |
| | | Modified | [mC][mC][mG][ademA-GalNAc][ademA- | |
| | | Sense | GalNAc][ademA-GalNAc][mG][mG][mC] | |
| | | Strand | [mU][mG][mC] | |
| KHK-510- | Hs-Mf- | Low-2′- | [MePhosphonate-4O- | 819 |
| 548-379- | Mm-Rn | Fluoro | mUs][fUs][fCs][fU][fG][mC][fU][mU] | |
| 221 | commons | Pattern | [mC][fU][mC][mU][mU][fC][mC][mA] | |
| | | Modified | [mU][mG][mA][mGs][mGs][mG] | |
| | | Anti-sense | | |
| | | Strand | | |
| KHK-516- | Hs-Mf- | Low-2′- | [MePhosphonate-4O- | 820 |
| 554-385- | Mm-Rn | Fluoro | mUs][fAs][fC][fA][fG][mG][fA][mU] | |
| 227 | commons | Pattern | [mC][fU][mG][mC][mU][fU][mC][mU] | |
| | | Modified | [mC][mU][mU][mCs][mGs][mG] | |
| | | Anti-sense | | |
| | | Strand | | |
| KHK-829- | Hs-Mf | Low-2′- | [MePhosphonate-4O- | 821 |
| 838 | commons | Fluoro | mUs][fAs][fG][fC][fA][mC][fA][mA] | |
| | | Pattern | [mU][fG][mG][mU][mA][fC][mG][mG] | |
| | | Modified | [mU][mU][mG][mCs][mGs][mG] | |
| | | Anti-sense | | |
| | | Strand | | |
| KHK-860- | Hs-Mf- | Low-2′- | [MePhosphonate-4O- | 822 |
| 898-729- | Mm-Rn | Fluoro | mUs][fAs][fGs][fC][fA][mG][fA][mC] | |
| 571 | commons | Pattern | [mA][fC][mA][mU][mC][fU][mG][mG] | |
| | | Modified | [mC][mA][mG][mGs][mGs][mG] | |
| | | Anti-sense | | |
| | | Strand | | |
| KHK-861- | Hs-Mf- | Low-2′- | [MePhosphonate-4O- | 823 |
| 899-730- | Mm-Rn | Fluoro | mUs][fUs][fAs][fG][fC][mA][fG][mA] | |
| 572 | commons | Pattern | [mC][fA][mC][mA][mU][fC][mU][mG] | |
| | | Modified | [mG][mC][mA][mGs][mGs][mG] | |
| | | Anti-sense | | |
| | | Strand | | |
| KHK-865 | Hs | Low-2′- | [MePhosphonate-4O- | 824 |
| | unique | Fluoro | mUs][fUs][fCs][fU][fG][mU][fA][mG] | |
| | | Pattern | [mC][fA][mG][mA][mC][fA][mC][mA] | |
| | | Modified | [mU][mC][mU][mGs][mGs][mG] | |
| | | Anti-sense | | |
| | | Strand | | |
| KHK-882- | Hs-Mf | Low-2′- | [MePhosphonate-4O- | 825 |
| 920 | commons | Fluoro | mUs][fGs][fAs][fU][fC][mA][fA][mC] | |
| | | Pattern | [mC][fU][mU][mC][mU][fC][mA][mA] | |
| | | Modified | [mA][mG][mU][mCs][mGs][mG] | |
| | | Anti-sense | | |
| | | Strand | | |
| KHK-883- | Hs-Mf | Low-2′- | [MePhosphonate-4O- | 826 |
| 921 | commons | Fluoro | mUs][fAs][fGs][fA][fU][mC][fA][mA] | |
| | | Pattern | [mC][fC][mU][mU][mC][fU][mC][mA] | |
| | | Modified | [mA][mA][mG][mUs][mGs][mG] | |
| | | Anti-sense | | |
| | | Strand | | |
| KHK-885- | Hs-Mf | Low-2′- | [MePhosphonate-4O- | 827 |
| 923 | commons | Fluoro | mUs][fUs][fCs][fA][fG][mA][fU][mC] | |
| | | Pattern | [mA][fA][mC][mC][mU][fU][mC][mU] | |
| | | Modified | [mC][mA][mA][mAs][mGs][mG] | |
| | | Anti-sense | | |
| | | Strand | | |
| KHK-1054- | Hs-Mf | Low-2′- | [MePhosphonate-4O- | 828 |
| 1092 | commons | Fluoro | mUs][fUs][mCs][mU][fC][mC][fG][fU] | |
| | | Pattern | [mA][fG][mC][fC][mA][fA][mA][fC] | |
| | | Modified | [mA][mG][fC][mUs][mGs][mG] | |
| | | Anti-sense | | |
| | | Strand | | |
| KHK-1075- | Hs-Mf- | Low-2′- | [MePhosphonate-4O- | 829 |
| 1113-944- | Mm-Rn | Fluoro | mUs][fUs][fCs][fU][fU][mU][fG][mC] | |
| 786 | commons | Pattern | [mU][fG][mA][mC][mA][fA][mA][mC] | |
| | | Modified | [mA][mC][mC][mAs][mGs][mG] | |
| | | Anti-sense | | |
| | | Strand | | |
| KHK-1078- | Hs-Mf- | Low-2′- | [MePhosphonate-4O- | 830 |
| 1116-947- | Mm-Rn | Fluoro | mUs][fAs][fC][fA][fU][mC][fU][mU] | |
| 789 | commons | Pattern | [mU][fG][mC][mU][mG][fA][mC][mA] | |
| | | Modified | [mA][mA][mC][mAs][mGs][mG] | |
| | | Anti-sense | | |
| | | Strand | | |
| KHK-1281- | Hs-Mf- | Low-2′- | [MePhosphonate-4O- | 831 |
| 1319-1150- | Mm-Rn | Fluoro | mUs][fAs][fGs][fG][fC][mA][fU][mU] | |
| 992 | commons | Pattern | [mG][fA][mA][mG][mG][fU][mG][mU] | |
| | | Modified | [mC][mU][mC][mCs][mGs][mG] | |
| | | Anti-sense | | |
| | | Strand | | |
| KHK-1288- | Hs-Mf | Low-2′- | [MePhosphonate-4O- | 832 |
| 1326 | commons | Fluoro | mUs][fAs][fUs][fG][fA][mC][fG][mG] | |
| | | Pattern | [mA][fG][mG][mC][mA][fU][mU][mG] | |
| | | Modified | [mA][mA][mG][mGs][mGs][mG] | |
| | | Anti-sense | | |
| | | Strand | | |
| KHK-1290- | Hs-Mf | Low-2′- | [MePhosphonate-4O- | 833 |
| 1328 | commons | Fluoro | mUs][fAs][fGs][fA][fU][mG][fA][mC] | |
| | | Pattern | [mG][fG][mA][mG][mG][fC][mA][mU] | |
| | | Modified | [mU][mG][mA][mAs][mGs][mG] | |
| | | Anti-sense | | |
| | | Strand | | |
| KHK-1148- | Hs-Mf | Low-2′- | [MePhosphonate-4O- | 834 |
| 1186 | commons | Fluoro | mUs][fCs][fCs][fC][fU][mU][fU][mC] | |
| | | Pattern | [mC][fU][mC][mA][mC][fA][mC][mG] | |
| | | Modified | [mA][mC][mC][mAs][mGs][mG] | |
| | | Anti-sense | | |
| | | Strand | | |
| KHK-1152- | Hs-Mf | Low-2′- | [MePhosphonate-4O- | 835 |
| 1190 | commons | Fluoro | mUs][fCs][fAs][fG][fC][mC][fC][mC] | |
| | | Pattern | [mU][fU][mU][mC][mC][fU][mC][mA] | |
| | | Modified | [mC][mA][mC][mGs][mGs][mG] | |
| | | Anti-sense | | |
| | | Strand | | |
| KHK-1154- | Hs-Mf | Low-2′- | [MePhosphonate-4O- | 836 |
| 1192 | commons | Fluoro | mUs][fCs][fAs][fC][fA][mG][fC][mC] | |
| | | Pattern | [mC][fC][mU][mU][mU][fC][mC][mU] | |
| | | Modified | [mC][mA][mC][mAs][mGs][mG] | |
| | | Anti-sense | | |
| | | Strand | | |
| KHK-1155- | Hs-Mf | Low-2′- | [MePhosphonate-4O- | 837 |
| 1193 | commons | Fluoro | mUs][fGs][fCs][fA][fC][mA][fG][mC] | |
| | | Pattern | [mC][fC][mC][mU][mU][fU][mC][mC] | |
| | | Modified | [mU][mC][mA][mCs][mGs][mG] | |
| | | Anti-sense | | |
| | | Strand | | |
| KHK-1277 | Hs | Low-2′- | [MePhosphonate-4O- | 838 |
| | unique | Fluoro | mUs][fAs][fUs][fU][fG][mA][fA][mG] | |
| | | Pattern | [mG][fU][mG][mU][mC][fU][mC][mC] | |
| | | Modified | [mA][mG][mC][mUs][mGs][mG] | |
| | | Anti-sense | | |
| | | Strand | | |
| KHK-1147- | Hs-Mf | Low-2′- | [MePhosphonate-4O- | 839 |
| 1185 | commons | Fluoro | mUs][fCs][fCs][fU][fU][mU][fC][mC] | |
| | | Pattern | [mU][fC][mA][mC][mA][fC][mG][mA] | |
| | | Modified | [mC][mC][mA][mUs][mGs][mG] | |
| | | Anti-sense | | |
| | | Strand | | |
| KHK-869- | Hs-Mf | Low-2′- | [MePhosphonate-4O- | 840 |
| 934 | commons | Fluoro | mUs][fAs][fAs][fA][fG][mU][fC][mU] | |
| | | Pattern | [mG][fU][mA][mG][mC][fA][mG][mA] | |
| | | Modified | [mC][mA][mC][mAs][mGs][mG] | |
| | | Anti-sense | | |
| | | Strand | | |
| KHK-873 | Hs | Low-2′- | [MePhosphonate-4O- | 841 |
| | unique | Fluoro | mUs][fUs][fCs][fU][fC][mA][fA][mA] | |
| | | Pattern | [mG][fU][mC][mU][mG][fU][mA][mG] | |
| | | Modified | [mC][mA][mG][mAs][mGs][mG] | |
| | | Anti-sense | | |
| | | Strand | | |
| KHK-879 | Hs | Low-2′- | [MePhosphonate-4O- | 842 |
| | unique | Fluoro | mUs][fCs][fAs][fA][fC][mC][fU][mU] | |
| | | Pattern | [mC][fU][mC][mA][mA][fA][mG][mU] | |
| | | Modified | [mC][mU][mG][mUs][mGs][mG] | |
| | | Anti-sense | | |
| | | Strand | | |
| KHK-881 | Hs | Low-2′- | [MePhosphonate-4O- | 843 |
| | unique | Fluoro | mUs][fAs][fUs][fC][fA][mA][fC][mC] | |
| | | Pattern | [mU][fU][mC][mU][mC][fA][mA][mA] | |
| | | Modified | [mG][mU][mC][mUs][mGs][mG] | |
| | | Anti-sense | | |
| | | Strand | | |
| KHK-896- | Hs-Mf | Low-2′- | [MePhosphonate-4O- | 844 |
| 934 | commons | Fluoro | mUs][fCs][fUs][fU][fG][mA][fA][mC] | |
| | | Pattern | [mU][fG][mG][mG][mU][fC][mA][mG] | |
| | | Modified | [mA][mU][mC][mAs][mGs][mG] | |
| | | Anti-sense | | |
| | | Strand | | |
| KHK-1064- | Hs-Mf | Low-2′- | [MePhosphonate-4O- | 845 |
| 1102 | commons | Fluoro | mUs][fAs][fAs][fA][fC][mA][fC][mC] | |
| | | Pattern | [mA][fC][mG][mU][mC][fU][mC][mC] | |
| | | Modified | [mG][mU][mA][mGs][mGs][mG] | |
| | | Anti-sense | | |
| | | Strand | | |
| KHK-1077- | Hs-Mf- | Low-2′- | [MePhosphonate-4O- | 846 |
| 1115-946- | Mm-Rn | Fluoro | mUs][fCs][fAs][fU][fC][mU][fU][mU] | |
| 788 | commons | Pattern | [mG][fC][mU][mG][mA][fC][mA][mA] | |
| | | Modified | [mA][mC][mA][mCs][mGs][mG] | |
| | | Anti-sense | | |
| | | Strand | | |
| KHK-1080- | Hs-Mf- | Low-2′- | [MePhosphonate-4O- | 847 |
| 1118-949- | Mm-Rn | Fluoro | mUs][fCs][fCs][fA][fC][mA][fU][mC] | |
| 791 | commons | Pattern | [mU][fU][mU][mG][mC][fU][mG][mA] | |
| | | Modified | [mC][mA][mA][mAs][mGs][mG] | |
| | | Anti-sense | | |
| | | Strand | | |
| KHK-1106- | Hs-Mf | Low-2′- | [MePhosphonate-4O- | 848 |
| 1144 | commons | Fluoro | mUs][fUs][fGs][fC][fU][mG][fA][mC] | |
| | | Pattern | [mU][fG][mG][mA][mA][fC][mC][mC] | |
| | | Modified | [mC][mA][mA][mGs][mGs][mG] | |
| | | Anti-sense | | |
| | | Strand | | |
| KHK-1334- | Hs-Mf | Low-2′- | [MePhosphonate-4O- | 849 |
| 1372 | commons | Fluoro | mUs][fGs][fAs][fA][fU][mC][fU][mC] | |
| | | Pattern | [mA][fG][mU][mG][mC][fU][mU][mC] | |
| | | Modified | [mC][mU][mG][mCs][mGs][mG] | |
| | | Anti-sense | | |
| | | Strand | | |
| KHK-516- | Hs-Mf- | Med-2′- | [MePhosphonate-4O- | 850 |
| 554-385- | Mm-Rn | Fluoro | mUs][fAs][fCs][fA][fG][mG][fA][fU] | |
| 227 | commons | Pattern | [mC][fU][mG][mC][mU][fU][mC][fU] | |
| | | Modified | [mC][mU][fU][mCs][mGs][mG] | |
| | | Anti-sense | | |
| | | Strand | | |
| KHK-804 | Hs | Med-2′- | [MePhosphonate-4O- | 851 |
| | unique | Fluoro | mUs][fAs][fGs][fU][fU][mG][fU][fU] | |
| | | Pattern | [mG][fA][mU][mG][mA][fU][mG][fC] | |
| | | Modified | [mA][mG][fC][mAs][mGs][mG] | |
| | | Anti-sense | | |
| | | Strand | | |
| KHK-829- | Hs-Mf | Med-2′- | [MePhosphonate-4O- | 852 |
| 838 | commons | Fluoro | mUs][fAs][fGs][fC][fA][mC][fA][fA] | |
| | | Pattern | [mU][fG][mG][mU][mA][fC][mG][fG] | |
| | | Modified | [mU][mU][fG][mCs][mGs][mG] | |
| | | Anti-sense | | |
| | | Strand | | |
| KHK-861- | Hs-Mf- | Med-2′- | [MePhosphonate-4O- | 853 |
| 899-730- | Mm-Rn | Fluoro | mUs][fUs][fAs][fG][fC][mA][fG][fA] | |
| 572 | commons | Pattern | [mC][fA][mC][mA][mU][fC][mU][fG] | |
| | | Modified | [mG][mC][fA][mGs][mGs][mG] | |
| | | Anti-sense | | |
| | | Strand | | |
| KHK-865 | Hs | Med-2′- | [MePhosphonate-4O- | 854 |
| | unique | Fluoro | mUs][fUs][fCs][fU][fG][mU][fA][fG] | |
| | | Pattern | [mC][fA][mG][mA][mC][fA][mC][fA] | |
| | | Modified | [mU][mC][fU][mGs][mGs][mG] | |
| | | Anti-sense | | |
| | | Strand | | |
| KHK-882- | Hs-Mf | Med-2′- | [MePhosphonate-4O- | 855 |
| 920 | commons | Fluoro | mUs][fGs][fAs][fU][fC][mA][fA][fC] | |
| | | Pattern | [mC][fU][mU][mC][mU][fC][mA][fA] | |
| | | Modified | [mA][mG][fU][mCs][mGs][mG] | |
| | | Anti-sense | | |
| | | Strand | | |
| KHK-883- | Hs-Mf | Med-2′- | [MePhosphonate-4O- | 856 |
| 921 | commons | Fluoro | mUs][fAs][fGs][fA][fU][mC][fA][fA] | |
| | | Pattern | [mC][fC][mU][mU][mC][fU][mC][fA] | |
| | | Modified | [mA][mA][fG][mUs][mGs][mG] | |
| | | Anti-sense | | |
| | | Strand | | |
| KHK-885- | Hs-Mf | Med-2′- | [MePhosphonate-4O- | 857 |
| 923 | commons | Fluoro | mUs][fUs][fCs][fA][fG][mA][fU][fC] | |
| | | Pattern | [mA][fA][mC][mC][mU][fU][mC][fU] | |
| | | Modified | [mC][mA][fA][mAs][mGs][mG] | |
| | | Anti-sense | | |
| | | Strand | | |
| KHK-1054- | Hs-Mf | Med-2′- | [MePhosphonate-4O- | 858 |
| 1092 | commons | Fluoro | mUs][fUs][mCs][mU][fC][mC][fG][fU] | |
| | | Pattern | [mA][fG][mC][fC][mA][fA][mA][fC] | |
| | | Modified | [mA][mG][fC][mUs][mGs][mG] | |
| | | Anti-sense | | |
| | | Strand | | |
| KHK-1076- | Hs-Mf- | Med-2′- | [MePhosphonate-4O- | 859 |
| 1114-945- | Mm-Rn | Fluoro | mUs][fAs][fUs][fC][fU][mU][fU][fG] | |
| 787 | commons | Pattern | [mC][fU][mG][mA][mC][fA][mA][fA] | |
| | | Modified | [mC][mA][fC][mCs][mGs][mG] | |
| | | Anti-sense | | |
| | | Strand | | |
| KHK-1078- | Hs-Mf- | Med-2′- | [MePhosphonate-4O- | 860 |
| 1116-947- | Mm-Rn | Fluoro | mUs][fAs][fCs][fA][fU][mC][fU][fU] | |
| 789 | commons | Pattern | [mU][fG][mC][mU][mG][fA][mC][fA] | |
| | | Modified | [mA][mA][fC][mAs][mGs][mG] | |
| | | Anti-sense | | |
| | | Strand | | |
| KHK-1288- | Hs-Mf | Med-2′- | [MePhosphonate-4O- | 861 |
| 1326 | commons | Fluoro | mUs][fAs][fUs][fG][fA][mC][fG][fG] | |
| | | Pattern | [mA][fG][mG][mC][mA][fU][mU][fG] | |
| | | Modified | [mA][mA][fG][mGs][mGs][mG] | |
| | | Anti-sense | | |
| | | Strand | | |
| KHK-1290- | Hs-Mf | Med-2′- | [MePhosphonate-4O- | 862 |
| 1328 | commons | Fluoro | mUs][fAs][fGs][fA][fU][mG][fA][fC] | |
| | | Pattern | [mG][fG][mA][mG][mG][fC][mA][fU] | |
| | | Modified | [mU][mG][fA][mAs][mGs][mG] | |
| | | Anti-sense | | |
| | | Strand | | |
| KHK-1334- | Hs-Mf | Med-2′- | [MePhosphonate-4O- | 863 |
| 1372 | commons | Fluoro | mUs][fGs][fAs][fA][fU][mC][fU][fC] | |
| | | Pattern | [mA][fG][mU][mG][mC][fU][mU][fC] | |
| | | Modified | [mC][mU][fG][mCs][mGs][mG] | |
| | | Anti-sense | | |
| | | Strand | | |
| Fam Probe | Fam | N/A | CCCTGGCCATGTTG | 864 |
| | Probe | | | |
| | for NHP | | | |
| | KHK | | | |
| | primers | | | |
| Forward- | Mouse | N/A | TGGAGGTGGAGAAGCCA | 865 |
| 1026 | KHK | | | |
| Primer | | | | |
| Reverse- | Mouse | N/A | GACCATACAAGCCCCTCAAG | 866 |
| 1157 | KHK | | | |
| Primer | | | | |
| Probe- | Probe | N/A | TGGTGTTTGTCAGCAAAGATGTGGC | 867 |
| 1080 | for | | | |
| | Mouse | | | |
| | KHK | | | |
| | primers | | | |
| Forward- | 5′ | N/A | AGGAAGCTCTGGGAGTA | 868 |
| 496 | Assay | | | |
| Primer | | | | |
| Reverse- | 5′ | N/A | CCTCCTTAGGGTACTTGTC | 869 |
| 596 | Assay | | | |
| Primer | | | | |
| Probe-518 | 5′ | N/A | ATGGAAGAGAAGCAGATCCTGTGCG | 870 |
| | Assay | | | |
| | Probe | | | |
| Stem Loop | Un- | N/A | GCAGCCGAAAGGCUGC | 871 |
| | modified | | | |
| | stem | | | |
| | loop | | | |
| | sequence | | | |
| KHK-1277 | Hs | 36mer | AGCUGGAGACACCUUCAAUAGCAGCCGA | 872 |
| | unique | Sense | AAGGCUGC | |
| | | Strand | | |
| KHK-1155- | Hs-Mf | 36mer | GUGAGGAAAGGGGCUGUGCAGCAGCCGA | 873 |
| 1193 | commons | Sense | AAGGCUGC | |
| | | Strand | | |
| KHK-1152- | Hs-Mf | 36mer | CGUGUGAGGAAAGGGGCUGAGCAGCCGA | 874 |
| 1190 | commons | Sense | AAGGCUGC | |
| | | Strand | | |
| KHK-881 | Hs | 36mer | AGACUUUGAGAAGGUUGAUAGCAGCCGA | 875 |
| | unique | Sense | AAGGCUGC | |
| | | Strand | | |
| KHK-879 | Hs | 36mer | ACAGACUUUGAGAAGGUUGAGCAGCCGA | 876 |
| | unique | Sense | AAGGCUGC | |
| | | Strand | | |
| KHK-869 | Hs-Mf | 36mer | UGUGUCUGCUACAGACUUUAGCAGCCGA | 877 |
| | commons | Sense | AAGGCUGC | |
| | | Strand | | |
| KHK-873 | Hs | 36mer | UCUGCUACAGACUUUGAGAAGCAGCCGA | 878 |
| | unique | Sense | AAGGCUGC | |
| | | strand | | |
| KHK-1277 | Hs | 22mer | UAUUGAAGGUGUCUCCAGCUGG | 879 |
| | unique | anti-sense | | |
| | | strand | | |
| KHK-1155- | Hs-Mf | 22mer | UGCACAGCCCCUUUCCUCACGG | 880 |
| 1193 | commons | anti-sense | | |
| | | strand | | |
| KHK-1152- | Hs-Mf | 22mer | UCAGCCCCUUUCCUCACACGGG | 881 |
| 1190 | commons | anti-sense | | |
| | | strand | | |
| KHK-881 | Hs | 22mer | UAUCAACCUUCUCAAAGUCUGG | 882 |
| | unique | anti-sense | | |
| | | strand | | |
| KHK-879 | Hs | 22mer | UCAACCUUCUCAAAGUCUGUGG | 883 |
| | unique | anti-sense | | |
| | | strand | | |
| KHK-869 | Hs-Mf | 22mer | UAAAGUCUGUAGCAGACACAGG | 884 |
| | commons | anti-sense | | |
| | | strand | | |
| KHK-873 | Hs | 22mer | UUCUCAAAGUCUGUAGCAGAGG | 885 |
| | unique | anti-sense | | |
| | | strand | | |
| KHK-510- | Hs-Mf- | 36mer | CUCAUGGAAGAGAAGCAGAAGCAGCCGA | 886 |
| 548-379- | Mm-Rn | Sense | AAGGCUGC | |
| 221 | commons | strand | | |
| KHK-516- | Hs-Mf- | 36mer | GAAGAGAAGCAGAUCCUGUAGCAGCCGA | 887 |
| 554-385- | Mm-Rn | Sense | AAGGCUGC | |
| 227 | commons | strand | | |
| KHK-829- | Hs-Mf | 36mer | GCAACCGUACCAUUGUGCUAGCAGCCGA | 888 |
| 838 | commons | Sense | AAGGCUGC | |
| | | strand | | |
| KHK-860- | Hs-Mf- | 36mer | CCUGCCAGAUGUGUCUGCUAGCAGCCGA | 889 |
| 898-729- | Mm-Rn | Sense | AAGGCUGC | |
| 571 | commons | strand | | |
| KHK-861- | Hs-Mf- | 36mer | CUGCCAGAUGUGUCUGCUAAGCAGCCGA | 890 |
| 899-730- | Mm-Rn | Sense | AAGGCUGC | |
| 572 | commons | strand | | |
| KHK-865 | Hs | 36mer | CAGAUGUGUCUGCUACAGAAGCAGCCGA | 891 |
| | unique | Sense | AAGGCUGC | |
| | | strand | | |
| KHK-882- | Hs-Mf | 36mer | GACUUUGAGAAGGUUGAUCAGCAGCCGA | 892 |
| 920 | commons | Sense | AAGGCUGC | |
| | | strand | | |
| KHK-883- | Hs-Mf | 36mer | ACUUUGAGAAGGUUGAUCUAGCAGCCGA | 893 |
| 921 | commons | Sense | AAGGCUGC | |
| | | strand | | |
| KHK-885- | Hs-Mf | 36mer | UUUGAGAAGGUUGAUCUGAAGCAGCCGA | 894 |
| 923 | commons | Sense | AAGGCUGC | |
| | | strand | | |
| KHK-1054- | Hs-Mf | 36mer | AGCUGUUUGGCUACGGAGAAGCAGCCGA | 895 |
| 1092 | commons | Sense | AAGGCUGC | |
| | | strand | | |
| KHK-1075- | Hs-Mf- | 36mer | UGGUGUUUGUCAGCAAAGAAGCAGCCGA | 896 |
| 1113-944- | Mm-Rn | Sense | AAGGCUGC | |
| 786 | commons | strand | | |
| KHK-1078- | Hs-Mf- | 36mer | UGUUUGUCAGCAAAGAUGUAGCAGCCGA | 897 |
| 1116-947- | Mm-Rn | Sense | AAGGCUGC | |
| 789 | commons | strand | | |
| KHK-1281- | Hs-Mf- | 36mer | GGAGACACCUUCAAUGCCUAGCAGCCGA | 898 |
| 1319-1150- | Mm-Rn | Sense | AAGGCUGC | |
| 992 | commons | strand | | |
| KHK-1288- | Hs-Mf | 36mer | CCUUCAAUGCCUCCGUCAUAGCAGCCGA | 899 |
| 1326 | commons | Sense | AAGGCUGC | |
| | | strand | | |
| KHK-1290- | Hs-Mf | 36mer | UUCAAUGCCUCCGUCAUCUAGCAGCCGA | 900 |
| 1328 | commons | Sense | AAGGCUGC | |
| | | strand | | |
| KHK-1148- | Hs-Mf | 36mer | UGGUCGUGUGAGGAAAGGGAGCAGCCGA | 901 |
| 1186 | commons | Sense | AAGGCUGC | |
| | | strand | | |
| KHK-1154- | Hs-Mf | 36mer | UGUGAGGAAAGGGGCUGUGAGCAGCCGA | 902 |
| 1192 | commons | Sense | AAGGCUGC | |
| | | strand | | |
| KHK-1147- | Hs-Mf | 36mer | AUGGUCGUGUGAGGAAAGGAGCAGCCGA | 903 |
| 1185 | commons | Sense | AAGGCUGC | |
| | | strand | | |
| KHK-896- | Hs-Mf | 36mer | UGAUCUGACCCAGUUCAAGAGCAGCCGA | 904 |
| 934 | commons | Sense | AAGGCUGC | |
| | | strand | | |
| KHK-1064- | Hs-Mf | 36mer | CUACGGAGACGUGGUGUUUAGCAGCCGA | 905 |
| 1102 | commons | Sense | AAGGCUGC | |
| | | strand | | |
| KHK-1077- | Hs-Mf- | 36mer | GUGUUUGUCAGCAAAGAUGAGCAGCCGA | 906 |
| 1115-946- | Mm-Rn | Sense | AAGGCUGC | |
| 788 | commons | strand | | |
| KHK-1080- | Hs-Mf- | 36mer | UUUGUCAGCAAAGAUGUGGAGCAGCCGA | 907 |
| 11 18-949- | Mm-Rn | Sense | AAGGCUGC | |
| 791 | commons | strand | | |
| KHK-1106- | Hs-Mf | 36mer | CUUGGGGUUCCAGUCAGCAAGCAGCCGA | 908 |
| 1144 | commons | Sense | AAGGCUGC | |
| | | strand | | |
| KHK-1334- | Hs-Mf | 36mer | GCAGGAAGCACUGAGAUUCAGCAGCCGA | 909 |
| 1372 | commons | Sense | AAGGCUGC | |
| | | strand | | |
| KHK-804 | Hs | 36mer | UGCUGCAUCAUCAACAACUAGCAGCCGAA | 910 |
| | unique | Sense | AGGCUGC | |
| | | strand | | |
| KHK-1076- | Hs-Mf- | 36mer | GGUGUUUGUCAGCAAAGAUAGCAGCCGA | 911 |
| 1114-945- | Mm-Rn | Sense | AAGGCUGC | |
| 787 | commons | strand | | |
| KHK-510- | Hs-Mf- | 22mer | UUCUGCUUCUCUUCCAUGAGGG | 912 |
| 548-379- | Mm-Rn | anti-sense | | |
| 221 | commons | strand | | |
| KHK-516- | Hs-Mf- | 22mer | UACAGGAUCUGCUUCUCUUCGG | 913 |
| 554-385- | Mm-Rn | anti-sense | | |
| 227 | commons | strand | | |
| KHK-829- | Hs-Mf | 22mer | UAGCACAAUGGUACGGUUGCGG | 914 |
| 838 | commons | anti-sense | | |
| | | strand | | |
| KHK-860- | Hs-Mf- | 22mer | UAGCAGACACAUCUGGCAGGGG | 915 |
| 898-729- | Mm-Rn | anti-sense | | |
| 571 | commons | strand | | |
| KHK-861- | Hs-Mf- | 22mer | UUAGCAGACACAUCUGGCAGGG | 916 |
| 899-730- | Mm-Rn | anti-sense | | |
| 572 | commons | strand | | |
| KHK-865 | Hs | 22mer | UUCUGUAGCAGACACAUCUGGG | 917 |
| | unique | anti-sense | | |
| | | strand | | |
| KHK-882- | Hs-Mf | 22mer | UGAUCAACCUUCUCAAAGUCGG | 918 |
| 920 | commons | anti-sense | | |
| | | strand | | |
| KHK-883- | Hs-Mf | 22mer | UAGAUCAACCUUCUCAAAGUGG | 919 |
| 921 | commons | anti-sense | | |
| | | strand | | |
| KHK-885- | Hs-Mf | 22mer | UUCAGAUCAACCUUCUCAAAGG | 920 |
| 923 | commons | anti-sense | | |
| | | strand | | |
| KHK-1054- | Hs-Mf | 22mer | UUCUCCGUAGCCAAACAGCUGG | 921 |
| 1092 | commons | anti-sense | | |
| | | strand | | |
| KHK-1075- | Hs-Mf- | 22mer | UUCUUUGCUGACAAACACCAGG | 922 |
| 1113-944- | Mm-Rn | anti-sense | | |
| 786 | commons | strand | | |
| KHK-1078- | Hs-Mf- | 22mer | UACAUCUUUGCUGACAAACAGG | 923 |
| 1116-947- | Mm-Rn | anti-sense | | |
| 789 | commons | strand | | |
| KHK-1281- | Hs-Mf- | 22mer | UAGGCAUUGAAGGUGUCUCCGG | 924 |
| 1319-1150- | Mm-Rn | anti-sense | | |
| 992 | commons | strand | | |
| KHK-1288- | Hs-Mf | 22mer | UAUGACGGAGGCAUUGAAGGGG | 925 |
| 1326 | commons | anti-sense | | |
| | | strand | | |
| KHK-1290- | Hs-Mf | 22mer | UAGAUGACGGAGGCAUUGAAGG | 926 |
| 1328 | commons | anti-sense | | |
| | | strand | | |
| KHK-1148- | Hs-Mf | 22mer | UCCCUUUCCUCACACGACCAGG | 927 |
| 1186 | commons | anti-sense | | |
| | | strand | | |
| KHK-1154- | Hs-Mf | 22mer | UCACAGCCCCUUUCCUCACAGG | 928 |
| 1192 | commons | anti-sense | | |
| | | strand | | |
| KHK-1147- | Hs-Mf | 22mer | UCCUUUCCUCACACGACCAUGG | 929 |
| 1185 | commons | anti-sense | | |
| | | strand | | |
| KHK-873 | Hs | 22mer | UUCUCAAAGUCUGUAGCAGAGG | 930 |
| | unique | anti-sense | | |
| | | strand | | |
| KHK-896- | Hs-Mf | 22mer | UGUUGAACUGGGUCAGAUCAGG | 931 |
| 934 | commons | anti-sense | | |
| | | strand | | |
| KHK-1064- | Hs-Mf | 22mer | UAAACACCACGUCUCCGUAGGG | 932 |
| 1102 | commons | anti-sense | | |
| | | strand | | |
| KHK-1077- | Hs-Mf- | 22mer | UCAUCUUUGCUGACAAACACGG | 933 |
| 1115-946- | Mm-Rn | anti-sense | | |
| 788 | commons | strand | | |
| KHK-1080- | Hs-Mf- | 22mer | UCCACAUCUUUGCUGACAAAGG | 934 |
| 1118-949- | Mm-Rn | anti-sense | | |
| 791 | commons | strand | | |
| KHK-1106- | Hs-Mf | 22mer | UUGCUGACUGGAACCCCAAGGG | 935 |
| 1144 | commons | anti-sense | | |
| | | strand | | |
| KHK-1334- | Hs-Mf | 22mer | UGAAUCUCAGUGCUUCCUGCGG | 936 |
| 1372 | commons | anti-sense | | |
| | | strand | | |
| KHK-804 | Hs | 22mer | UAGUUGUUGAUGAUGCAGCAGG | 937 |
| | unique | anti-sense | | |
| | | strand | | |
| KHK-1076- | Hs-Mf- | 22mer | UAUCUUUGCUGACAAACACCGG | 938 |
| 1114-945- | Mm-Rn | anti-sense | | |
| 787 | commons | strand | | |
| MmKHK- | Forward | N/A | GCTCTTCCAGTTGTTTAGCTATGGT | 939 |
| ALL-5-6 | | | | |
| MmKHK- | Reverse | N/A | CAGGTGCTTGGCCACATCTT | 940 |
| ALL-5-6 | | | | |
| MmKHK- | Probe | N/A | AGGTGGTGTTTGTCAGC | 941 |
| ALL-5-6 | | | | |
| KHK-516- | Hs-Mf- | 19mer | GAAGAGAAGCAGAUCCUGU | 942 |
| 554-385- | Mm-Rn | Sense | | |
| 227 | commons | strand | | |
| KHK-865 | Hs | 19mer | CAGAUGUGUCUGCUACAGA | 943 |
| | unique | Sense | | |
| | | strand | | |
| KHK-882- | Hs-Mf | 19mer | GACUUUGAGAAGGUUGAUC | 944 |
| 920 | commons | Sense | | |
| | | strand | | |
| KHK-885- | Hs-Mf | 19mer | UUUGAGAAGGUUGAUCUGA | 945 |
| 923 | commons | Sense | | |
| | | strand | | |
| KHK-1078- | Hs-Mf- | 19mer | UGUUUGUCAGCAAAGAUGU | 946 |
| 1116-947- | Mm-Rn | Sense | | |
| 789 | commons | strand | | |
| KHK-1334- | Hs-Mf | 19mer | GCAGGAAGCACUGAGAUUC | 947 |
| 1372 | commons | Sense | | |
| | | strand | | |
| KHK-516- | Hs-Mf- | 19mer | ACAGGAUCUGCUUCUCUUC | 948 |
| 554-385- | Mm-Rn | anti-sense | | |
| 227 | commons | strand | | |
| KHK-865 | Hs | 19mer | UCUGUAGCAGACACAUCUG | 949 |
| | unique | anti-sense | | |
| | | strand | | |
| KHK-882- | Hs-Mf | 19mer | GAUCAACCUUCUCAAAGUC | 950 |
| 920 | commons | anti-sense | | |
| | | strand | | |
| KHK-885- | Hs-Mf | 19mer | UCAGAUCAACCUUCUCAAA | 951 |
| 923 | commons | anti-sense | | |
| | | strand | | |
| KHK-1078- | Hs-Mf- | 19mer | ACAUCUUUGCUGACAAACA | 952 |
| 1116-947- | Mm-Rn | anti-sense | | |
| 789 | commons | strand | | |
| KHK-1334- | Hs-Mf | 19mer | GAAUCUCAGUGCUUCCUGC | 953 |
| 1372 | commons | anti-sense | | |
| | | strand | | |

Particular aspects and embodiments of the present invention are described with reference to the following clauses:

1. A double stranded RNAi oligonucleotide for reducing ketohexokinase (KHK) expression, the oligonucleotide comprising an antisense strand and a sense strand, wherein the antisense strand and the sense strand form a duplex region, wherein the antisense strand comprises a region of complementarity to a KHK mRNA target sequence of any one of SEQ ID NOs: 4-387 and wherein the region of complementarity is at least 15 contiguous nucleotides in length, or a pharmaceutically acceptable salt thereof.

2. The RNAi oligonucleotide of clause 1, wherein the sense strand comprises a sequence set forth in any one of SEQ ID NOs: 4-387.

3. The RNAi oligonucleotide of clause 1 or 2, wherein the antisense strand comprises a sequence set forth in any one of SEQ ID NOs: 388-771.

4. A double stranded RNAi oligonucleotide for inhibiting expression of KHK, wherein said double stranded RNAi oligonucleotide comprises a sense strand and an antisense strand forming a duplex region, wherein said sense strand comprises at least 15 contiguous nucleotides differing by no more than 3 nucleotides from any one of the nucleotide sequences of SEQ ID NO:4-387 and said antisense strand comprises at least 15 contiguous nucleotides differing by no more than 3 nucleotides from any one of the nucleotide sequences of SEQ ID NO: 388-771, or a pharmaceutically acceptable salt thereof.

5. The RNAi oligonucleotide of any one of clauses 1-4, wherein the sense strand is 15 to 50 nucleotides in length.

6. The RNAi oligonucleotide of any one of clauses 1-4, wherein the sense strand is 18 to 36 nucleotides in length.

7. The RNAi oligonucleotide of any one of clauses 1-4, wherein the sense strand is 15 to 30 nucleotides in length.

8. The RNAi oligonucleotide of any one of clauses 1-7, wherein the antisense strand is 15-30 nucleotides in length.

9. The RNAi oligonucleotide of any one of clauses 1-8, wherein the antisense strand and the sense strand form a duplex region of at least 19 nucleotides in length, optionally at least 20 nucleotides in length.

10. The RNAi oligonucleotide of any one of clauses 1-3 and 5-9, wherein the region of complementarity is at least 19 contiguous nucleotides in length, optionally at least 20 nucleotides in length.

11. A double stranded RNAi oligonucleotide for reducing KHK expression, the oligonucleotide comprising:
(i) an antisense strand of 19-30 nucleotides in length, wherein the antisense strand comprises a nucleotide sequence comprising a region of complementarity to a KHK mRNA target sequence, wherein the region of complementarity is selected from SEQ ID NOs: 948-953, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

12. The RNAi oligonucleotide of any one of clauses 1-11, wherein the sense strand comprises at its 3′ end a stem-loop set forth as: S1-L-S2, wherein S1 is complementary to S2, and wherein L forms a loop between S1 and S2 of 3 to 5 nucleotides in length.

13. The RNAi oligonucleotide of clause 12, wherein L is a triloop or a tetraloop.

14. The RNAi oligonucleotide of clause 13, wherein L is a tetraloop.

15. The RNAi oligonucleotide of clause 14, wherein the tetraloop comprises the sequence 5′-GAAA-3′.

16. The RNAi oligonucleotide of any one of clauses 12-15, wherein the S1 and S2 are 1-10 nucleotides in length and have the same length.

17. The RNAi oligonucleotide of clause 16, wherein S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length.

18. The RNAi oligonucleotide of clause 17, wherein S1 and S2 are 6 nucleotides in length.

19. The RNAi oligonucleotide of any one of clauses 12-18, wherein the stem-loop comprises the sequence 5′-GCAGCCGAAAGGCUGC-3′ (SEQ ID NO: 871).

20. The RNAi oligonucleotide of any one of clauses 1-19, comprising a nicked tetraloop structure.

21. The RNAi oligonucleotide of any one of clauses 1-19, comprising a nick between the 3′ terminus of the sense strand and the 5′ terminus of the antisense strand.

22. The RNAi oligonucleotide of any one of clauses 1-21, wherein the antisense and sense strands are not covalently linked.

23. The RNAi oligonucleotide of any one of clauses 1-10 and 12-22, wherein the antisense strand comprises an overhang of one or more nucleotides in length at the 3′ terminus.

24. The RNAi oligonucleotide of any one of clauses 11-23, wherein the overhang comprises purine nucleotides.

25. The RNAi oligonucleotide of any one of clauses 11-24, wherein the overhang is 2 nucleotides in length.

26. The RNAi oligonucleotide of clause 25, wherein the 3′ overhang is selected from AA, GG, AG, and GA.

27. The RNAi oligonucleotide of clause 26, wherein the overhang is GG or AA.

28. The RNAi oligonucleotide of clause 26, wherein the overhang is GG.

29. The RNAi oligonucleotide of any one of the preceding clauses, wherein the oligonucleotide comprises at least one modified nucleotide.

30. The RNAi oligonucleotide of clause 29, wherein the modified nucleotide comprises a 2′-modification.

31. The RNAi oligonucleotide of clause 30, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

32. The RNAi oligonucleotide of any one of clauses 29-31, wherein about 10-15%, 10%, 11%, 12%, 13%, 14% or 15% of the nucleotides of the sense strand comprise a 2′-fluoro modification.

33. The RNAi oligonucleotide of any one of clauses 29-32, wherein about 25-35%, 25%, 26%, 27%, 28%, 29%, 30%, 31%, 32%, 33%, 34% or 35% of the nucleotides of the antisense strand comprise a 2′-fluoro modification.

34. The RNAi oligonucleotide of any one of clauses 29-33, wherein about 25-35%, 25%, 26%, 27%, 28%, 29%, 30%, 31%, 32%, 33%, 34% or 35% of the nucleotides of the oligonucleotide comprise a 2′-fluoro modification.

35. The RNAi oligonucleotide of any one of clauses 29-34, wherein all the nucleotides of the oligonucleotide are modified.

36. The RNAi oligonucleotide of any one of clauses 29-34, wherein the sense strand comprises 36 nucleotides with positions 1-36 numbered from 5′ to 3′, wherein positions 8, 9, 10 and 11 of the sense strand are modified.

37. The RNAi oligonucleotide of any one of clauses 29-34, wherein the sense strand comprises 36 nucleotides with positions 1-36 numbered from 5′ to 3′, wherein positions 3, 8, 9, 10, 12, 13 and 17 of the sense strand are modified.

38. The RNAi oligonucleotide of any one of clauses 29-34, wherein the antisense strand comprises 22 nucleotides with positions 1-22 numbered from 5′ to 3′, and wherein positions 2, 3, 4, 5, 7, 10 and 14 of the antisense strand are modified.

39. The RNAi oligonucleotide of any one of clauses 29-34, wherein the antisense strand comprises 22 nucleotides with positions 1-22 numbered from 5′ to 3′, and wherein positions 2-5, 7, 8, 10, 14, 16 and 19 of the antisense strand are modified.

40. The RNAi oligonucleotide of any one of clauses 36-39, where the modification is 2′-fluoro.

41. The RNAi oligonucleotide of any one of clauses 32-34 and 36-40, wherein the remaining nucleotides comprise a 2′-O-methyl modification.

42. The RNAi oligonucleotide of any one of the preceding clauses, wherein the oligonucleotide comprises at least one modified internucleotide linkage.

43. The RNAi oligonucleotide of clause 42, wherein the at least one modified internucleotide linkage is a phosphorothioate linkage.

44. The RNAi oligonucleotide of clause 43, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.

45. The RNAi oligonucleotide of clause 43 or 44, wherein the antisense strand is 22 nucleotides in length, and wherein the antisense strand comprises a phosphorothioate linkage between positions 20 and 21 and between positions 21 and 22, wherein positions are numbered 1-22 from 5′ to 3′.

46. The RNAi oligonucleotide of any one of clauses 1-45, wherein the antisense strand comprises a phosphorylated nucleotide at the 5′ terminus, wherein the phosphorylated nucleotide is selected from uridine and adenosine.

47. The RNAi oligonucleotide of clause 46, wherein the phosphorylated nucleotide is uridine.

48. The RNAi oligonucleotide of any one of the preceding clauses, wherein the 4′-carbon of the sugar of the 5′-terminal nucleotide of the antisense strand comprises a phosphate analog.

49. The RNAi oligonucleotide of clause 48, wherein the phosphate analog is oxymethylphosphonate, vinylphosphonate or malonyl phosphonate, optionally wherein the phosphate analog is a 4′-phosphate analog comprising 5′-methoxyphosphonate-4′-oxy.

50. The RNAi oligonucleotide of any one of the preceding clauses, wherein at least one nucleotide of the oligonucleotide is conjugated to one or more targeting ligands.

51. The RNAi oligonucleotide of clause 50, wherein each targeting ligand comprises a carbohydrate, amino sugar, cholesterol, polypeptide, or lipid.

52. The RNAi oligonucleotide of any one of clauses 11-51, wherein the stem loop comprises one or more targeting ligands conjugated to one or more nucleotides of the stem loop.

53. The RNAi oligonucleotide of clause 52, wherein the one or more targeting ligands is conjugated to one or more nucleotides of the loop.

54. The RNAi oligonucleotide of clause 53, wherein the loop comprises 4 nucleotides numbered 1-4 from 5′ to 3′, wherein nucleotides at positions 2, 3, and 4 each comprise one or more targeting ligands, wherein the targeting ligands are the same or different.

55. The RNAi oligonucleotide of any one of clauses 50-54, wherein each targeting ligand comprises a N-acetylgalactosamine (GalNAc) moiety.

56. The RNAi oligonucleotide of clause 55, wherein the GalNAc moiety is a monovalent GalNAc moiety, a bivalent GalNAc moiety, a trivalent GalNAc moiety or a tetravalent GalNAc moiety.

57. The RNAi oligonucleotide of any one of clauses 11-56, wherein up to 4 nucleotides of L of the stem-loop are each conjugated to a monovalent GalNAc moiety.

58. The RNAi oligonucleotide of any one of clauses 1-57, wherein the region of complementarity comprised by the antisense strand is fully complementary to the KHK mRNA target sequence at nucleotide positions 2-8 of the antisense strand, wherein nucleotide positions are numbered 5′ to 3′.

59. The RNAi oligonucleotide of any one of clauses 1-57, wherein the region of complementarity comprised by the antisense strand is fully complementary to the KHK mRNA target sequence at nucleotide positions 2-11 of the antisense strand, wherein nucleotide positions are numbered 5′ to 3′.

60. The RNAi oligonucleotide of any one of clauses 1-59, wherein the sense strand comprises a nucleotide sequence of any one of SEQ ID NOs: 872-878 and 886-911.

61. The RNAi oligonucleotide of any one of clauses 1-60, wherein the antisense strand comprises a nucleotide sequence of any one of SEQ ID NOs: 879-884 and 912-938.

62. The RNAi oligonucleotide of any one of clauses 1-61, wherein the sense and antisense strands comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(o) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd SEQ ID NOs: 898 and 924, respectively;
(ee) SEQ ID NOs: 899 and 925, respectively;
(ff) SEQ ID NOs: 900 and 926, respectively; and
(gg) SEQ ID NOs: 909 and 936, respectively.

63. The RNAi oligonucleotide of any one of clauses 1-62, wherein the sense and antisense strands comprise the nucleotide sequences set forth in SEQ ID NOs: 909 and 936, respectively.

64. The RNAi oligonucleotide of any one of clauses 1-62, wherein the sense and antisense strands comprise the nucleotide sequences set forth in SEQ ID NOs: 894 and 920, respectively.

65. The RNAi oligonucleotide of any one of clauses 1-62, wherein the sense and antisense strands comprise the nucleotide sequences set forth in SEQ ID NOs: 897 and 923, respectively.

66. The RNAi oligonucleotide of any one of clauses 1-62, wherein the sense and antisense strands comprise the nucleotide sequences set forth in SEQ ID NOs: 892 and 918, respectively.

67. The RNAi oligonucleotide of any one of clauses 1-62, wherein the sense and antisense strands comprise the nucleotide sequences set forth in SEQ ID NOs: 891 and 917, respectively.

68. The RNAi oligonucleotide of any one of clauses 1-62, wherein the sense and antisense strands comprise the nucleotide sequences set forth in SEQ ID NOs: 887 and 913, respectively.

69. The RNAi oligonucleotide of any one of clauses 1-59, wherein the antisense strand is 22 nucleotides in length.

70. The RNAi oligonucleotide of clause 69, wherein the antisense strand comprises a nucleotide sequence selected from SEQ ID NOs: 913, 917, 918, 920, 923 and 936.

71. The RNAi oligonucleotide of any one of clauses 1-59 and 69-70, wherein the sense strand comprises a nucleotide sequence selected from SEQ ID NOs: 942-947.

72. The RNAi oligonucleotide of any one of clauses 1-59 and 69-71, wherein the sense strand is 36 nucleotides in length.

73. The RNAi oligonucleotide of clause 72, wherein the sense strand comprises a nucleotide sequence selected from SEQ ID NOs: 887, 891, 892, 894, 897 and 909.

74. The RNAi oligonucleotide of any one of clauses 60-73, wherein the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog.

75. The RNAi oligonucleotide of any one of clauses 1-59, wherein the sense strand comprises a nucleotide sequence of any one of SEQ ID NOs: 774-804.

76. The RNAi oligonucleotide of any one of clauses 1-59 and 75, wherein the antisense strand comprises a nucleotide sequence of any one of SEQ ID NOs: 819-849.

77. The RNAi oligonucleotide of any one of clauses 1-59 and 75-76, wherein the sense and antisense strands comprise nucleotide sequences selected from the group consisting of:
(a) SEQ ID NOs: 774 and 819, respectively;
(b) SEQ ID NOs: 775 and 820, respectively;
(c) SEQ ID NOs: 776 and 821, respectively;
(d) SEQ ID NOs: 777 and 822, respectively;
(e) SEQ ID NOs: 778 and 823, respectively;
(f) SEQ ID NOs: 779 and 824, respectively;
(g) SEQ ID NOs: 780 and 825, respectively;
(h) SEQ ID NOs: 781 and 826, respectively;
(i) SEQ ID NOs: 782 and 827, respectively;
(j) SEQ ID NOs: 783 and 828, respectively;
(k) SEQ ID NOs: 784 and 829, respectively;
(l) SEQ ID NOs: 785 and 830, respectively;
(m) SEQ ID NOs: 786 and 831, respectively;
(n) SEQ ID NOs: 787 and 832, respectively;
(o) SEQ ID NOs: 788 and 833, respectively;
(p) SEQ ID NOs: 789 and 834, respectively;
(q) SEQ ID NOs: 790 and 835, respectively;
(r) SEQ ID NOs: 791 and 836, respectively;
(s) SEQ ID NOs: 792 and 837, respectively;
(t) SEQ ID NOs: 793 and 838, respectively;
(u) SEQ ID NOs: 794 and 839, respectively;
(v) SEQ ID NOs: 795 and 840, respectively;
(w) SEQ ID NOs: 796 and 841, respectively;
(x) SEQ ID NOs: 797 and 842, respectively;
(y) SEQ ID NOs: 798 and 843, respectively;
(z) SEQ ID NOs: 799 and 844, respectively;
(aa) SEQ ID NOs: 800 and 845, respectively;
(bb) SEQ ID NOs: 801 and 846, respectively;
(cc) SEQ ID NOs: 802 and 847, respectively;
(dd) SEQ ID NOs: 803 and 848, respectively; and
(ee) SEQ ID NOs: 804 and 849, respectively.

78. The RNAi oligonucleotide of any one of clauses 1-59 and 75-76, wherein the sense and antisense strands comprise the nucleotide sequences set forth in SEQ ID NOs: 804 and 849, respectively.

79. The RNAi oligonucleotide of any one of clauses 1-59 and 75-76, wherein the sense and antisense strands comprise the nucleotide sequences set forth in SEQ ID NOs: 782 and 827, respectively.

80. The RNAi oligonucleotide of any one of clauses 1-59 and 75-76, wherein the sense and antisense strands comprise the nucleotide sequences set forth in SEQ ID NOs: 775 and 820, respectively.

81. The RNAi oligonucleotide of any one of clauses 1-59 and 75-76, wherein the sense and antisense strands comprise the nucleotide sequences set forth in SEQ ID NOs: 779 and 824, respectively.

82. The RNAi oligonucleotide of any one of clauses 1-59 and 75-76, wherein the sense and antisense strands comprise the nucleotide sequences set forth in SEQ ID NOs: 780 and 825, respectively.

83. The RNAi oligonucleotide of any one of clauses 1-59 and 75-76, wherein the sense and antisense strands comprise the nucleotide sequences set forth in SEQ ID NOs: 785 and 830, respectively.

84. The RNAi oligonucleotide of any one of clauses 1-59, wherein the sense strand comprises a nucleotide sequence of any one of SEQ ID NOs: 805-818.

85. The RNAi oligonucleotide of any one of clauses 1-59 and 84, wherein the antisense strand comprises a nucleotide sequence of any one of SEQ ID NOs: 850-863

86. The RNAi oligonucleotide of any one of clauses 1-59 and 84-85, wherein the sense and antisense strands comprise nucleotide sequences selected from the group consisting of:
(a) SEQ ID NOs: 805 and 850, respectively;
(b) SEQ ID NOs: 806 and 851, respectively;
(c) SEQ ID NOs: 807 and 852, respectively;
(d) SEQ ID NOs: 808 and 853, respectively;
(e) SEQ ID NOs: 809 and 854, respectively;
(f) SEQ ID NOs: 810 and 855, respectively;
(g) SEQ ID NOs: 811 and 856, respectively;
(h) SEQ ID NOs: 812 and 857, respectively;
(i) SEQ ID NOs: 813 and 858, respectively;
(j) SEQ ID NOs: 814 and 859, respectively;
(k) SEQ ID NOs: 815 and 860, respectively;
(l) SEQ ID NOs: 816 and 861, respectively;
(m) SEQ ID NOs: 817 and 862, respectively and;
(n) SEQ ID NOs: 818 and 863, respectively.

87. The RNAi oligonucleotide of any one of clauses 1-59 and 84-86, wherein the sense and antisense strands comprise the nucleotide sequences set forth in SEQ ID NOs: 805 and 850, respectively.

88. The RNAi oligonucleotide of any one of clauses 1-59 and 84-86, wherein the sense and antisense strands comprise the nucleotide sequences set forth in SEQ ID NOs: 809 and 854, respectively.

89. The RNAi oligonucleotide of any one of clauses 1-59 and 84-86, wherein the sense and antisense strands comprise the nucleotide sequences set forth in SEQ ID NOs: 810 and 855, respectively.

90. The RNAi oligonucleotide of any one of clauses 1-59 and 84-86, wherein the sense and antisense strands comprise the nucleotide sequences set forth in SEQ ID NOs: 812 and 857, respectively.

91. The RNAi oligonucleotide of any one of clauses 1-59 and 84-86, wherein the sense and antisense strands comprise the nucleotide sequences set forth in SEQ ID NOs: 815 and 860, respectively.

92. The RNAi oligonucleotide of any one of clauses 1-59 and 84-86, wherein the sense and antisense strands comprise the nucleotide sequences set forth in SEQ ID NOs: 818 and 863, respectively.

93. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mG-S-mC-mA-mG-mG-mA-mA-fG-fC-fA-fC-mU-mG-mA-mG-mA-mU-mU-mC-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 804), and wherein the antisense strand comprises the sequence and all of the modifications of 5′-[MePhosphonate-40-mU]-S-fG-S-fA-S-fA-fU-mC-fU-mC-mA-fG-mU-mG-mC-fU-mU-mC-mC-mU-mG-mC-S-mG-S-mG-3′ (SEQ ID NO: 849), wherein mC, mA, mG, mU=2′-OMe ribonucleosides; fA, fC, fG, fU-2′F ribonucleosides; “-”=phosphodiester linkage, “—S-”=phosphorothioate linkage, and wherein ademA-GalNAc=

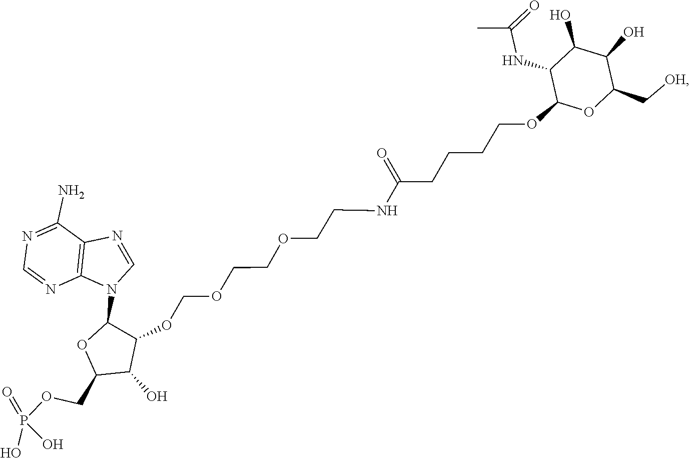 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

94. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mU-S-mU-mU-mG-mA-mG-mA-fA-fG-fG-fU-mU-mG-mA-mU-mC-mU-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 782), and wherein the antisense strand comprises the sequence and all of the modifications of 5′ [MePhosphonate-40-mU]-S-fU-S-fC-S-fA-fG-mA-fU-mC-mA-fA-mC-mC-mU-fU-mC-mU-mC-mA-mA-mA-S-mG-S-mG-3′ (SEQ ID NO: 827), wherein mC, mA, mG, mU=2′-OMe ribonucleosides; fA, fC, fG, fU=2′F ribonucleosides; “-”=phosphodiester linkage, “—S-”=phosphorothioate linkage, and wherein ademA-GalNAc=

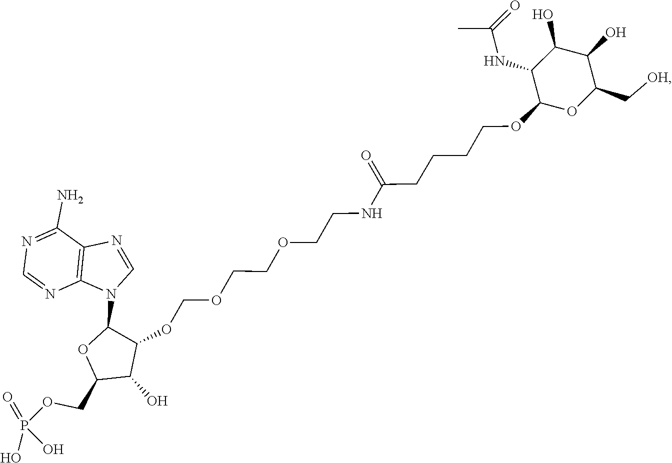 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

95. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mG-S-mA-mA-mG-mA-mG-mA-fA-fG-fC-fA-mG-mA-mU-mC-mC-mU-mG-mU-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 775), and wherein the antisense strand comprises the sequence and all of the modifications of 5′-[MePhosphonate-40-mU]-S-fA-S-fC-fA-fG-mG-fA-mU-mC-fU-mG-mC-mU-fU-mC-mU-mC-mU-mU-mC-S-mG-S-mG-3′ (SEQ ID NO: 820), wherein mC, mA, mG, mU=2′-OMe ribonucleosides; fA, fC, fG, fU=2′F ribonucleosides; “-”-phosphodiester linkage, “—S-”-phosphorothioate linkage, and wherein ademA-GalNAc=

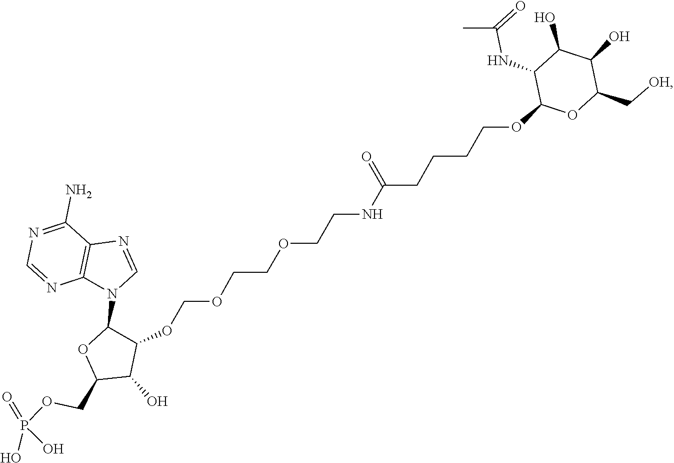 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

96. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mC-S-mA-mG-mA-mU-mG-mU-fG-fU-fC-fU-mG-mC-mU-mA-mC-mA-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 779), and wherein the antisense strand comprises the sequence and all of the modifications of 5′-[MePhosphonate-40-mU]-S-fU-S-fC-S-fU-fG-mU-fA-mG-mC-fA-mG-mA-mC-fA-mC-mA-mU-mC-mU-mG-S-mG-S-mG-3′ (SEQ ID NO: 824), wherein mC, mA, mG, mU-2′-OMe ribonucleosides; fA, fC, fG, fU-2′F ribonucleosides; “-”=phosphodiester linkage, “—S-”-phosphorothioate linkage, and wherein ademA-GalNAc=

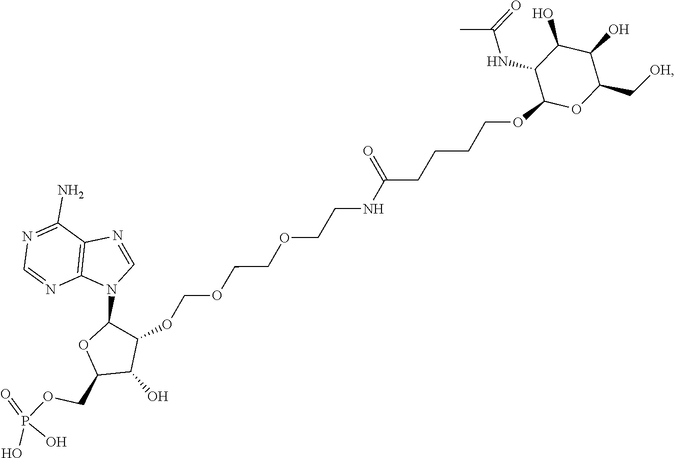 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

97. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mG-S-mA-mC-mU-mU-mU-mG-fA-fG-fA-fA-mG-mG-mU-mU-mG-mA-mU-mC-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 780), and wherein the antisense strand comprises the sequence and all of the modifications of 5′-[MePhosphonate-40-mU]-S-fG-S-fA-S-fU-fC-mA-fA-mC-mC-fU-mU-mC-mU-fC-mA-mA-mA-mG-mU-mC-S-mG-S-mG-3′ (SEQ ID NO: 825), wherein mC, mA, mG, mU=2′-OMe ribonucleosides; fA, fC, fG, fU=2′F ribonucleosides; “-”=phosphodiester linkage, “—S-”-phosphorothioate linkage, and wherein ademA-GalNAc=

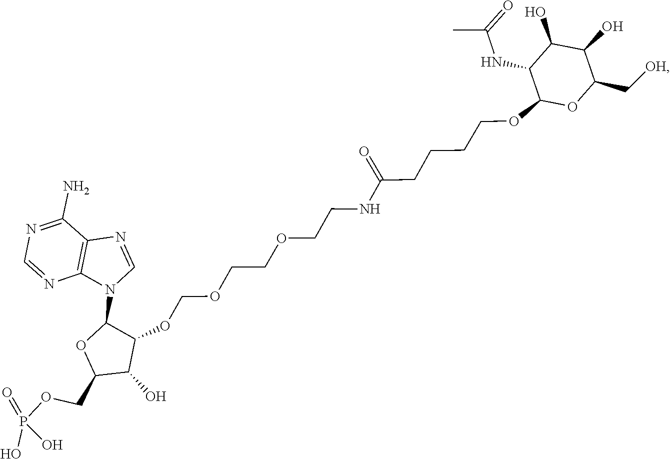 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

98. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, e.g. KHK mRNA, wherein the sense strand comprises the sequence and all of the modifications of 5′-mU-S-mG-mU-mU-mU-mG-mU-fC-fA-fG-fC-mA-mA-mA-mG-mA-mU-mG-mU-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 785), and wherein the antisense strand comprises the sequence and all of the modifications of 5′-[MePhosphonate-40-mU]-S-fA-S-fC-fA-fU-mC-fU-mU-mU-fG-mC-mU-mG-fA-mC-mA-mA-mA-mC-mA-S-mG-S-mG-3′ (SEQ ID NO: 830), wherein mC, mA, mG, mU=2′-OMe ribonucleosides; fA, fC, fG, fU=2′F ribonucleosides; “-”=phosphodiester linkage, “—S-”-phosphorothioate linkage, and wherein ademA-GalNAc=

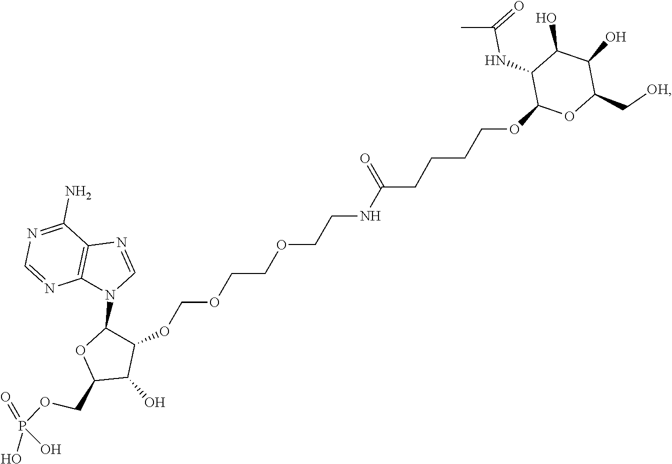 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

or a pharmaceutically acceptable salt thereof.

99. A dsRNAi oligonucleotide for inhibiting expression of KHK, wherein said dsRNAi comprises a sense strand comprising SEQ ID NO: 775 and an antisense strand comprising SEQ ID NO: 820, the antisense strand comprising a region of complementarity to a KHK RNA transcript, e.g. KHK mRNA, wherein said dsRNAi is in the form of a conjugate having the structure as depicted in FIG. 10A continuing to FIG. 10B, or a pharmaceutically acceptable salt thereof.

100. A dsRNAi oligonucleotide for inhibiting expression of KHK, wherein said dsRNAi comprises a sense strand comprising SEQ ID NO: 779 and an antisense strand comprising SEQ ID NO: 824, the antisense strand comprising a region of complementarity to a KHK RNA transcript, e.g. KHK mRNA, wherein said dsRNAi is in the form of a conjugate having the structure as depicted FIG. 11A continuing to FIG. 11B, or a pharmaceutically acceptable salt thereof.

101. A dsRNAi oligonucleotide for inhibiting expression of KHK, wherein said dsRNAi comprises a sense strand comprising SEQ ID NO: 780 and an antisense strand comprising SEQ ID NO: 825, the antisense strand comprising a region of complementarity to a KHK RNA transcript, e.g. KHK mRNA, wherein said dsRNAi is in the form of a conjugate having the structure as depicted in FIG. 12A continuing to FIG. 12B, or a pharmaceutically acceptable salt thereof.

102. A dsRNAi oligonucleotide for inhibiting expression of KHK, wherein said dsRNAi comprises a sense strand comprising SEQ ID NO: 782 and an antisense strand comprising SEQ ID NO: 827, the antisense strand comprising a region of complementarity to a KHK RNA transcript, e.g. KHK mRNA, wherein said dsRNAi is in the form of a conjugate having the structure FIG. 13A continuing to FIG. 13B, or a pharmaceutically acceptable salt thereof.

103. A dsRNAi oligonucleotide for inhibiting expression of KHK, wherein said dsRNAi comprises a sense strand comprising SEQ ID NO: 785 and an antisense strand comprising SEQ ID NO: 830, the antisense strand comprising a region of complementarity to a KHK RNA transcript, e.g. KHK mRNA, wherein said dsRNAi is in the form of a conjugate having the structure FIG. 14A continuing to FIG. 14B, or a pharmaceutically acceptable salt thereof.

104. A dsRNAi oligonucleotide for inhibiting expression of KHK, wherein said dsRNAi comprises a sense strand comprising SEQ ID NO: 804 and an antisense strand comprising SEQ ID NO: 849, the antisense strand comprising a region of complementarity to a KHK RNA transcript, e.g. KHK mRNA, wherein said dsRNAi is in the form of a conjugate having the structure of FIG. 15A continuing to FIG. 15B, or a pharmaceutically acceptable salt thereof.

105. The RNAi oligonucleotide of any one of clauses 1-104, wherein expression of KHK is reduced or inhibited in vivo.

106. The RNAi oligonucleotide of any one of clauses 1-105, wherein the oligonucleotide is a Dicer substrate.

107. The RNAi oligonucleotide of any one of clauses 1-105, wherein the oligonucleotide is a Dicer substrate that, upon endogenous Dicer processing, yields double-stranded nucleic acids of 19-23 nucleotides in length capable of reducing KHK expression in a mammalian cell.

108. A cell containing the RNAi oligonucleotide of any one of the preceding clauses.

109. A method for treating a subject having a disease, disorder or condition associated with KHK expression, the method comprising administering to the subject a therapeutically effective amount of the RNAi oligonucleotide of any one of clauses 1-107, or pharmaceutically acceptable salt thereof, or pharmaceutical composition thereof, thereby treating the subject.

110. A pharmaceutical composition comprising the RNAi oligonucleotide of any one of clauses 1-107, or a pharmaceutically acceptable salt thereof, and at least one pharmaceutically acceptable carrier, delivery agent or excipient.

111. A method of delivering an oligonucleotide to a subject, the method comprising administering the pharmaceutical composition of clause 110 to the subject.

112. An in vitro or in vivo method for modulating, e.g. inhibiting or reducing, KHK expression in a target cell expressing KHK, the method comprising administering the pharmaceutical composition of clause 110 in an effective amount to the target cell.

113. A method for reducing KHK expression in a cell, a population of cells or a subject, the method comprising the step of:
i. contacting the cell or the population of cells with the RNAi oligonucleotide, or a pharmaceutically acceptable salt thereof, of any one of clauses 1-107, or the pharmaceutical composition of clause 110; or
ii. administering to the subject the RNAi oligonucleotide, or a pharmaceutically acceptable salt thereof of any one of clauses 1-107, or the pharmaceutical composition of clause 110.

114. The method of clause 113, wherein reducing KHK expression comprises reducing an amount or level of KHK mRNA, an amount or level of KHK protein, or both.

115. The method of any one of clauses 111 and 113-114, wherein the subject has a disease, disorder or condition associated with KHK expression.

116. The method of clause 115, wherein the disease, disorder or condition associated with KHK expression is non-alcoholic fatty liver disease (NAFLD) and non-alcoholic steatohepatitis (NASH).

117. The method of any one of clauses 109 and 111-116, wherein the RNAi oligonucleotide, or pharmaceutical composition, is administered in combination with a second composition or therapeutic agent.

118. A method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strand comprise nucleotide sequences selected from the group consisting of:
(a) SEQ ID NOs: 886 and 912, respectively;
(b) SEQ ID NOs: 887 and 913, respectively;
(c) SEQ ID NOs: 910 and 937, respectively;
(d) SEQ ID NOs: 888 and 914, respectively;
(e) SEQ ID NOs: 889 and 915, respectively;
(f) SEQ ID NOs: 890 and 916, respectively;
(g) SEQ ID NOs: 891 and 917, respectively;
(h) SEQ ID NOs: 877 and 884, respectively;
(i) SEQ ID NOs: 878 and 930, respectively;
(j) SEQ ID NOs: 876 and 883, respectively;
(k) SEQ ID NOs: 875 and 882, respectively;
(l) SEQ ID NOs: 892 and 918, respectively;
(m) SEQ ID NOs: 893 and 919, respectively;
(n) SEQ ID NOs: 894 and 920, respectively;
(0) SEQ ID NOs: 904 and 931, respectively;
(p) SEQ ID NOs: 895 and 921, respectively;
(q) SEQ ID NOs: 905 and 932, respectively;
(r) SEQ ID NOs: 896 and 922, respectively;
(s) SEQ ID NOs: 911 and 938, respectively;
(t) SEQ ID NOs: 906 and 933, respectively;
(u) SEQ ID NOs: 897 and 923, respectively;
(v) SEQ ID NOs: 907 and 934, respectively;
(w) SEQ ID NOs: 908 and 935, respectively;
(x) SEQ ID NOs: 903 and 929, respectively;
(y) SEQ ID NOs: 901 and 927, respectively;
(z) SEQ ID NOs: 874 and 881, respectively;
(aa) SEQ ID NOs: 902 and 928, respectively;
(bb) SEQ ID NOs: 873 and 880, respectively;
(cc) SEQ ID NOs: 872 and 879, respectively;
(dd) SEQ ID NOs: 898 and 924, respectively;
(ee) SEQ ID NOs: 899 and 925, respectively
(ff) SEQ ID NOs: 900 and 926, respectively; and
(gg) SEQ ID NOs: 909 and 936, respectively.

119. The method of clause 118, wherein the sense and antisense strands comprise the nucleotide sequences set forth in SEQ ID NOs: 909 and 936, respectively.

120. The method of clause 118, wherein the sense and antisense strands comprise the nucleotide sequences set forth in SEQ ID NOs: 894 and 920, respectively.

121. The method of clause 118, wherein the sense and antisense strands comprise the nucleotide sequences set forth in SEQ ID NOs: 897 and 923, respectively.

122. The method of clause 118, wherein the sense and antisense strands comprise the nucleotide sequences set forth in SEQ ID NOs: 892 and 918, respectively.

123. The method of clause 118, wherein the sense and antisense strands comprise the nucleotide sequences set forth in SEQ ID NOs: 891 and 917, respectively.

124. The method of clause 118, wherein the sense and antisense strands comprise the nucleotide sequences set forth in SEQ ID NOs: 887 and 913, respectively.

125. A method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strands are selected from the group consisting of:
(a) SEQ ID NOs: 774 and 819, respectively;
(b) SEQ ID NOs: 775 and 820, respectively;
(c) SEQ ID NOs: 776 and 821, respectively;
(d) SEQ ID NOs: 777 and 822, respectively;
(e) SEQ ID NOs: 778 and 823, respectively;
(f) SEQ ID NOs: 779 and 824, respectively;
(g) SEQ ID NOs: 780 and 825, respectively;
(h) SEQ ID NOs: 781 and 826, respectively;
(i) SEQ ID NOs: 782 and 827, respectively;
(j) SEQ ID NOs: 783 and 828, respectively;
(k) SEQ ID NOs: 784 and 829, respectively;
(l) SEQ ID NOs: 785 and 830, respectively;
(m) SEQ ID NOs: 786 and 831, respectively;
(n) SEQ ID NOs: 787 and 832, respectively;
(o) SEQ ID NOs: 788 and 833, respectively;
(p) SEQ ID NOs: 789 and 834, respectively;
(q) SEQ ID NOs: 790 and 835, respectively;
(r) SEQ ID NOs: 791 and 836, respectively;
(s) SEQ ID NOs: 792 and 837, respectively;
(t) SEQ ID NOs: 793 and 838, respectively;
(u) SEQ ID NOs: 794 and 839, respectively;
(v) SEQ ID NOs: 795 and 840, respectively;
(w) SEQ ID NOs: 796 and 841, respectively;
(x) SEQ ID NOs: 797 and 842, respectively;
(y) SEQ ID NOs: 798 and 843, respectively;
(z) SEQ ID NOs: 799 and 844, respectively;
(aa) SEQ ID NOs: 800 and 845, respectively;
(bb) SEQ ID NOs: 801 and 846, respectively;
(cc) SEQ ID NOs: 802 and 847, respectively;
(dd) SEQ ID NOs: 803 and 848, respectively; and
(ee) SEQ ID NOs: 804 and 849, respectively.

126. The method of clause 125, wherein the sense and antisense strands comprise the nucleotide sequences set forth in SEQ ID NOs: 804 and 849, respectively.

127. The method of clause 125 wherein the sense and antisense strands comprise the nucleotide sequences set forth in SEQ ID NOs: 782 and 827, respectively.

128. The method of clause 125, wherein the sense and antisense strands comprise the nucleotide sequences set forth in SEQ ID NOs: 775 and 820, respectively.

129. The method of clause 125, wherein the sense and antisense strands comprise the nucleotide sequences set forth in SEQ ID NOs: 779 and 824, respectively.

130. The method of clause 125, wherein the sense and antisense strands comprise the nucleotide sequences set forth in SEQ ID NOs: 780 and 825, respectively.

131. The method of clause 125, wherein the sense and antisense strands comprise the nucleotide sequences set forth in SEQ ID NOs: 785 and 830, respectively.

132. A method for treating a subject having a disease, disorder or condition associated with KHK expression, the method comprising administering to the subject a therapeutically effective amount of an RNAi oligonucleotide comprising a sense strand and an antisense strand, or a pharmaceutically acceptable salt thereof, wherein the sense strand and antisense strands are selected from the group consisting of:
(a) SEQ ID NOs: 805 and 850, respectively;
(b) SEQ ID NOs: 806 and 851, respectively;
(c) SEQ ID NOs: 807 and 852, respectively;
(d) SEQ ID NOs: 808 and 853, respectively;
(e) SEQ ID NOs: 809 and 854, respectively;
(f) SEQ ID NOs: 810 and 855, respectively;
(g) SEQ ID NOs: 811 and 856, respectively;
(h) SEQ ID NOs: 812 and 857, respectively;
(i) SEQ ID NOs: 813 and 858, respectively;
(j) SEQ ID NOs: 814 and 859, respectively;
(k) SEQ ID NOs: 815 and 860, respectively;
(l) SEQ ID NOs: 816 and 861, respectively;
(m) SEQ ID NOs: 817 and 862, respectively and;
(n) SEQ ID NOs: 818 and 863, respectively.

133. The method of clause 132, wherein the sense and antisense strands comprise the nucleotide sequences set forth in SEQ ID NOs: 805 and 850, respectively.

134. The method of clause 132, wherein the sense and antisense strands comprise the nucleotide sequences set forth in SEQ ID NOs: 809 and 854, respectively.

135. The method of clause 132, wherein the sense and antisense strands comprise the nucleotide sequences set forth in SEQ ID NOs: 810 and 855, respectively.

136. The method of clause 132, wherein the sense and antisense strands comprise the nucleotide sequences set forth in SEQ ID NOs: 812 and 857, respectively.

137. The method of clause 132, wherein the sense and antisense strands comprise the nucleotide sequences set forth in SEQ ID NOs: 815 and 860, respectively.

138. The method of clause 132, wherein the sense and antisense strands comprise the nucleotide sequences set forth in SEQ ID NOs: 818 and 863, respectively.

139. The method of any one of clauses 118-138, wherein the disease, disorder or condition associated with KHK expression is non-alcoholic fatty liver disease (NAFLD) and non-alcoholic steatohepatitis (NASH).

140. The method of any one of clauses 109, 111 and 113-132, wherein the dsRNA is administered at a concentration of 0.01 mg/kg-5 mg/kg bodyweight of the subject.

141. Use of the RNAi oligonucleotide of any one of clauses 1-107, or the pharmaceutical composition of clause 110, in the manufacture of a medicament for the treatment of a disease, disorder or condition associated with KHK expression, optionally for the treatment of non-alcoholic fatty liver disease (NAFLD) and non-alcoholic steatohepatitis (NASH).

142. The RNAi oligonucleotide of any one of clauses 1-107, or the pharmaceutical composition of clause 110, for use, or adaptable for use, in the treatment of a disease, disorder or condition associated with KHK expression, optionally for the treatment of non-alcoholic fatty liver disease (NAFLD) and non-alcoholic steatohepatitis (NASH).

143. A kit comprising the RNAi oligonucleotide of any one of clauses 1-107, an optional pharmaceutically acceptable carrier, and a package insert comprising instructions for administration to a subject having a disease, disorder or condition associated with KHK expression.

144. The use of clause 141, the RNAi oligonucleotide or pharmaceutical composition for use, or adaptable for use, of clause 142, or the kit of clause 143, wherein the disease, disorder or condition associated with KHK expression is non-alcoholic fatty liver disease (NAFLD) and non-alcoholic steatohepatitis (NASH).

145. An oligonucleotide for reducing KHK expression, the oligonucleotide comprising a nucleotide sequence of 15-50 nucleotides in length, wherein the nucleotide sequence comprises a region of complementarity to a KHK mRNA target sequence of any one of SEQ ID NOs: 4-387, and wherein the region of complementarity is at least 15 contiguous nucleotides.

146. The oligonucleotide of clause 145, wherein the oligonucleotide is single stranded.

147. The oligonucleotide of clause 145 or 146, wherein the oligonucleotide is an antisense oligonucleotide.

148. The oligonucleotide of any one of clauses 145-147, wherein the nucleotide sequence is 15-30 nucleotides in length.

149. The oligonucleotide of any one of clauses 145-148, wherein the nucleotide sequence is 20-25 nucleotides in length.

150. The oligonucleotide of any one of clauses 145-149, wherein the nucleotide sequence is 22 nucleotides in length.

151. The oligonucleotide of any one of clauses 145-150, wherein the region of complementarity is 19 contiguous nucleotides in length.

152. The oligonucleotide of any one of clauses 145-150, wherein the region of complementarity is 20 contiguous nucleotides in length.

153. The oligonucleotide of any one of clauses 145-152, wherein the nucleotide sequence comprises at least one modification.

154. The oligonucleotide of any one of clauses 145-153, wherein the nucleotide sequence comprises a nucleotide sequence selected from the group consisting of SEQ ID NOs: 879-884 and 912-938.

155. The oligonucleotide of any one of clauses 145-153, wherein the nucleotide sequence comprises the nucleotide sequence set forth in SEQ ID NO: 909.

156. The oligonucleotide of any one of clauses 145-153, wherein the nucleotide sequence comprises the nucleotide sequence set forth in SEQ ID NO: 894.

157. The oligonucleotide of any one of clauses 145-153, wherein the nucleotide sequence comprises the nucleotide sequence set forth in SEQ ID NO: 897.

158. The oligonucleotide of any one of clauses 145-153, wherein the nucleotide sequence comprises the nucleotide sequence set forth in SEQ ID NO: 892.

159. The oligonucleotide of any one of clauses 145-153, wherein the nucleotide sequence comprises the nucleotide sequence set forth in SEQ ID NO: 891.

160. The oligonucleotide of any one of clauses 145-153, wherein the nucleotide sequence comprises the nucleotide sequence set forth in SEQ ID NO: 887.

161. A cell comprising the oligonucleotide of any one of clauses 145-160.

162. A pharmaceutical composition comprising the oligonucleotide of any one of clauses 145-160, or a pharmaceutically acceptable salt thereof, and at least one pharmaceutically acceptable carrier, delivery agent or excipient.

163. A method for treating a subject having a disease, disorder or condition associated with KHK expression, the method comprising administering to the subject a therapeutically effective amount of the oligonucleotide of any one of clauses 145-160, or pharmaceutical composition of clause 162.

164. A method of delivering an oligonucleotide to a subject, the method comprising administering the pharmaceutical composition of clause 162 to the subject.

165. A method for reducing KHK expression in a cell, a population of cells or a subject, the method comprising the step of:
i. contacting the cell or the population of cells with the oligonucleotide of any one of clauses 145-160, or the pharmaceutical composition of clause 162; or
ii. administering to the subject the oligonucleotide of any one of clauses 145-160, or the pharmaceutical composition of clause 162.

166. The method of clause 165, wherein reducing KHK expression comprises reducing an amount or level of KHK mRNA, an amount or level of KHK protein, or both.

167. The method of any one of clauses 164-166, wherein the subject has a disease, disorder or condition associated with KHK expression.

168. The method of clause 167, wherein the disease, disorder or condition associated with KHK expression is non-alcoholic fatty liver disease (NAFLD) and non-alcoholic steatohepatitis (NASH).

169. The method of any one of clauses 163-168, wherein the oligonucleotide, or pharmaceutical composition, is administered in combination with a second composition or therapeutic agent.

170. Use of the oligonucleotide of any one of clauses 145-160, or the pharmaceutical composition of clause 161, in the manufacture of a medicament for the treatment of a disease, disorder or condition associated with KHK expression, optionally for the treatment of non-alcoholic fatty liver disease (NAFLD) and non-alcoholic steatohepatitis (NASH).

171. The oligonucleotide of any one of clauses 145-160, or the pharmaceutical composition of clause 161, for use, or adaptable for use, in the treatment of a disease, disorder or condition associated with KHK expression, optionally for the treatment of non-alcoholic fatty liver disease (NAFLD) and non-alcoholic steatohepatitis (NASH).

172. A kit comprising the oligonucleotide of any one of clauses 145-160, an optional pharmaceutically acceptable carrier, and a package insert comprising instructions for administration to a subject having a disease, disorder or condition associated with KHK expression.

173. The use of clause 170, the RNAi oligonucleotide or pharmaceutical composition for use, or adaptable for use, of clause 171, or the kit of clause 172, wherein the disease, disorder or condition associated with KHK expression is non-alcoholic fatty liver disease (NAFLD) and non-alcoholic steatohepatitis (NASH).

174. A double stranded ribonucleic acid (dsRNA) agent for inhibiting expression of KHK, wherein the dsRNA agent comprises a sense strand and an antisense strand forming a duplex region, wherein the sense strand comprises at least 15 contiguous nucleotides differing by no more than 3 nucleotides from a nucleotide sequence selected from SEQ ID

NOs: 4-387, and the antisense strand comprises at least 15 contiguous nucleotides differing by no more than 3 nucleotides from a nucleotide sequence selected from SEQ ID NOs: 388-771, or a pharmaceutically acceptable salt thereof.

175. A double stranded ribonucleic acid (dsRNA) agent for inhibiting expression of KHK, wherein the dsRNA agent comprises a sense strand and an antisense strand forming a duplex region, wherein the sense strand comprises at least 15 contiguous nucleotides differing by no more than 3 nucleotides from a nucleotide sequence selected from SEQ ID NOs: 872-878 and 886-911, and the antisense strand comprises at least 15 contiguous nucleotides differing by no more than 3 nucleotides from a nucleotide sequence selected from SEQ ID NOs: 879-884 and 912-938, or a pharmaceutically acceptable salt thereof.

176. A pharmaceutical composition comprising the dsRNA agent of clause 174 or 175, and a pharmaceutically acceptable diluent, solvent, carrier, salt, and/or adjuvant.

177. An in vitro or in vivo method for reducing or inhibiting KHK expression in a target cell expressing KHK, the method comprising administering the pharmaceutical composition of clause 176 in an effective amount to the target cell.

178. A method for treating or preventing a disease associated with KHK expression, comprising administering a therapeutically or prophylactically effective amount of the pharmaceutical composition of clause 176 to a subject suffering from or susceptible to the disease.

179. The method of any one of clauses 109 and 113-140, wherein a single dose of one or more RNAi oligonucleotides of any one of clauses 1-107, or pharmaceutically acceptable salts thereof, or the pharmaceutical composition of any one of clauses 110, 162, or 176 is administered such that an amount or level of KHK mRNA and/or KHK protein is reduced in the subject when compared to KHK expression prior to administration of the one or more RNAi oligonucleotides, or pharmaceutically acceptable salts thereof, or the pharmaceutical composition and/or when compared to KHK expression in a subject not receiving the one or more RNAi oligonucleotides, or pharmaceutically acceptable salts thereof, or pharmaceutical composition or receiving one or more control oligonucleotides, pharmaceutical compositions or treatments, and wherein said reduction remains detectable at day 28, 56, and/or 84 after the single dose administration.

180. The method of clause 179, wherein the amount or level of KHK mRNA and/or KHK protein is reduced by at least about 30%, by at least about 50%, or by at least about 70%.

181. The method of any one of clauses 179-180, wherein the dose is administered subcutaneously.

1. A double stranded RNAi oligonucleotide for reducing ketohexokinase (KHK) expression, the oligonucleotide comprising:
an antisense strand and a sense strand, wherein the antisense strand and the sense strand form a duplex region, wherein the antisense strand comprises a region of complementarity to a KHK mRNA target sequence of any one of SEQ ID NOs: 4-387 and wherein the region of complementarity is at least 15 contiguous nucleotides in length, or a pharmaceutically acceptable salt thereof,
wherein the sense strand comprises a sequence set forth in any one of SEQ ID NOs: 4-387 and/or the antisense strand comprises a sequence set forth in any one of SEQ ID NOs: 388-771.

2. A double stranded RNAi oligonucleotide for inhibiting expression of KHK, wherein said double stranded RNAi oligonucleotide comprises a sense strand and an antisense strand forming a duplex region, wherein said sense strand comprises at least 15 contiguous nucleotides differing by no more than 3 nucleotides from any one of the nucleotide sequences of SEQ ID NO: 4-387 and said antisense strand comprises at least 15 contiguous nucleotides differing by no more than 3 nucleotides from any one of the nucleotide sequences of SEQ ID NO: 388-771, or a pharmaceutically acceptable salt thereof,
wherein the sense strand is 18 to 36 nucleotides in length and/or the antisense strand is 15-30 nucleotides in length.

3. A double stranded RNAi (dsRNAi) oligonucleotide for reducing or inhibiting ketohexokinase (KHK) expression, the oligonucleotide comprising:
(i) an antisense strand of 19-30 nucleotides in length, wherein the antisense strand comprises a nucleotide sequence comprising a region of complementarity to a KHK mRNA target sequence, wherein the region of complementarity is selected from SEQ ID NOs: 948-953; and
(ii) a sense strand of 19-50 nucleotides in length comprising a region of complementarity to the antisense strand,
wherein the antisense and sense strands are separate strands which form an asymmetric duplex region having an overhang of 1-4 nucleotides at the 3′ terminus of the antisense strand.

4. The RNAi oligonucleotide of claim 1, wherein the region of complementarity comprised by the antisense strand is at least 19 contiguous nucleotides in length.

5. The RNAi oligonucleotide of claim 1, wherein the duplex region is at least 20 nucleotides in length.

6. The RNAi oligonucleotide of claim 1, wherein the sense strand comprises at its 3′ end a stem-loop set forth as: S1-L-S2, wherein S1 is complementary to S2, and wherein L forms a loop between S1 and S2 of 3 to 5 nucleotides in length.

7. The RNAi oligonucleotide of claim 6, wherein at least one nucleotide of the loop is conjugated to one or more targeting ligands, and wherein each targeting ligand comprises an N-acetylgalactosamine (GalNAc) moiety.

8. The RNAi oligonucleotide of claim 3, wherein the overhang is 2 nucleotides in length and is selected from AA, GG, AG, and GA.

9. The RNAi oligonucleotide of claim 1, wherein (i) all the nucleotides of the oligonucleotide are modified, (ii) 10-15%, 10%, 11%, 12%, 13%, 14% or 15% of the nucleotides of the sense strand comprise a 2′-fluoro modification, and/or (iii) about 25-35%, 25%, 26%, 27%, 28%, 29%, 30%, 31%, 32%, 33%, 34% or 35% of the nucleotides of the antisense strand comprise a 2′-fluoro modification.

10. The RNAi oligonucleotide of claim 1, wherein the antisense strand comprises at least one phosphorothioate linkage between positions 1 and 2, between positions 2 and 3, and between positions 3 and 4, wherein positions are numbered 1-4 from 5′ to 3′.

11. The RNAi oligonucleotide of claim 1, wherein the 4′-carbon of the sugar of the 5′-terminal nucleotide of the antisense strand comprises a 4′-phosphate analog comprising 5′-methoxyphosphonate-4′-oxy.

12. The RNAi oligonucleotide of claim 1, wherein the antisense strand is 22 nucleotides in length and/or wherein the sense strand is 36 nucleotides in length.

13. The RNAi oligonucleotide of claim 1, wherein the sense and antisense strands comprise the nucleotide sequences set forth in
(a) SEQ ID NOs: 887 and 913, respectively, or
(b) SEQ ID NOs: 891 and 917, respectively, or
(c) SEQ ID NOs: 892 and 918, respectively, or
(d) SEQ ID NOs: 894 and 920, respectively, or
(e) SEQ ID NOs: 897 and 923, respectively, or
(f) SEQ ID NOs: 909 and 936, respectively.

14. A double stranded RNAi oligonucleotide (dsRNAi) for inhibiting expression of KHK, wherein said dsRNAi comprises a sense strand and an antisense strand, the antisense strand comprising a region of complementarity to a KHK RNA transcript, wherein
(a) the sense strand comprises the sequence and all of the modifications of 5-mG-S-mA-mA-mG-mA-mG-mA-fA-fG-fC-fA-mG-mA-mU-mC-mC-mU-mG-mU-mA-mG-mC-mA-mG-mC-mC-mG-[ademA-GalNAc]-[ademA-GalNAc]-[ademA-GalNAc]-mG-mG-mC-mU-mG-mC-3 (SEQ ID NO: 775), and the antisense strand comprises the sequence and all of the modifications of 5-[MePhosphonate-40-mU]-S-fA-S-fC-fA-fG-mG-fA-mU-mC-fU-mG-mC-mU-fU-mC-mU-mC-mU-mU-mC-S-mG-S-mG-3′ (SEQ ID NO: 820); or
(b) the sense strand comprises the sequence and all of the modifications of 5-mC-S-mA-mG-mA-mU-mG-mU-fG-fU-fC-fU-mG-mC-mU-mA-mC-mA-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 779), and
the antisense strand comprises the sequence and all of the modifications of 5-[MePhosphonate-40-mU]-S-fU-S-fC-S-fU-fG-mU-fA-mG-mC-fA-mG-mA-mC-fA-mC-mA-mU-mC-mU-mG-S-mG-S-mG-3 (SEQ ID NO: 824); or
(c) the sense strand comprises the sequence and all of the modifications of 5-mG-S-mA-mC-mU-mU-mU-mG-fA-fG-fA-fA-mG-mG-mU-mU-mG-mA-mU-mC-mA-mG-mC-mA-mG-mC-mC-mG-[ademA-GalNAc]-[ademA-GalNAc]-[ademA-GalNAc]-mG-mG-mC-mU-mG-mC-3 (SEQ ID NO: 780), and
the antisense strand comprises the sequence and all of the modifications of 5 -[MePhosphonate-40-mU]-S-fG-S-fA-S-fU-fC-mA-fA-mC-mC-fU-mU-mC-mU-fC-mA-mA-mA-mG-mU-mC-S-mG-S-mG-3 (SEQ ID NO: 825); or
(d) the sense strand comprises the sequence and all of the modifications of 5′-mU-S-mU-mU-mG-mA-mG-mA-fA-fG-fG-fU-mU-mG-mA-mU-mC-mU-mG-mA-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 782), and
the antisense strand comprises the sequence and all of the modifications of 5′ [MePhosphonate-40-mU]-S-fU-S-fC-S-fA-fG-mA-fU-mC-mA-fA-mC-mC-mU-fU-mC-mU-mC-mA-mA-mA-S-mG-S-mG-3′ (SEQ ID NO: 827); or
(e) the sense strand comprises the sequence and all of the modifications of 5-mU-S-mG-mU-mU-mU-mG-mU-fC-fA-fG-fC-mA-mA-mA-mG-mA-mU-mG-mU-mA-mG-mC-mA-mG-mC-mC-mG-[ademA-GalNAc]-[ademA-GalNAc]-[ademA-GalNAc]-mG-mG-mC-mU-mG-mC-3 (SEQ ID NO: 785), and
the antisense strand comprises the sequence and all of the modifications of 5-[MePhosphonate-40-mU]-S-fA-S-fC-fA-fU-mC-fU-mU-mU-fG-mC-mU-mG-fA-mC-mA-mA-mA-mC-mA-S-mG-S-mG-3′ (SEQ ID NO: 830); or
(f) the sense strand comprises the sequence and all of the modifications of 5′-mG-S-mC-mA-mG-mG-mA-mA-fG-fC-fA-fC-mU-mG-mA-mG-mA-mU-mU-mC-mA-mG-mC-mA-mG-mC-mC-mG-[ademA-GalNAc]-[ademA-GalNAc]-[ademA-GalNAc]-mG-mG-mC-mU-mG-mC-3′ (SEQ ID NO: 804), and
the antisense strand comprises the sequence and all of the modifications of 5′-[MePhosphonate-40-mU]-S-fG-S-fA-S-fA-fU-mC-fU-mC-mA-fG-mU-mG-mC-fU-mU-mC-mC-mU-mG-mC-S-mG-S-mG-3′ (SEQ ID NO: 849);
wherein mC, mA, mG, mU=2′-OMe ribonucleosides; fA, fC, fG, fU=2′F ribonucleosides; “-“=phosphodiester linkage, “—S-”=phosphorothioate linkage, and wherein ademA-GalNAc= 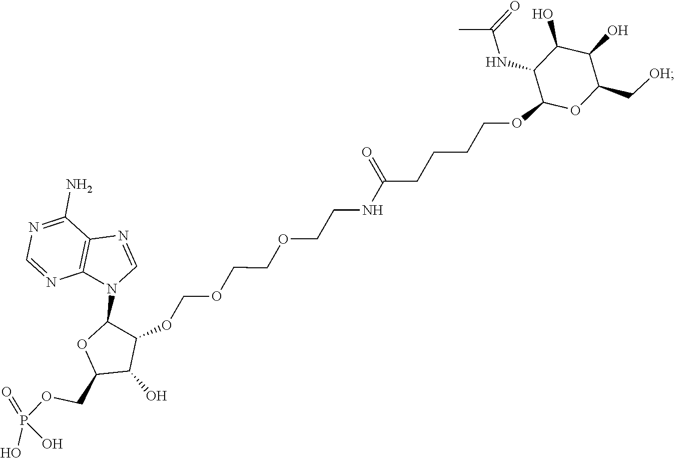 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]
or a pharmaceutically acceptable salt thereof.

15. A double stranded RNAi oligonucleotide (dsRNAi) for inhibiting expression of KHK, wherein said dsRNAi comprises
(a) a sense strand comprising SEQ ID NO: 775 and an antisense strand comprising SEQ ID NO: 820, the antisense strand comprising a region of complementarity to a KHK RNA transcript, e.g. KHK mRNA, wherein said dsRNAi is in the form of a conjugate having the structure depicted in FIG. 10A continuing to FIG. 10B; or
(b) a sense strand comprising SEQ ID NO: 779 and an antisense strand comprising SEQ ID NO: 824, the antisense strand comprising a region of complementarity to a KHK RNA transcript, e.g. KHK mRNA, wherein said dsRNAi is in the form of a conjugate having the structure depicted in FIG. 11A continuing to FIG. 11B; or
(c) a sense strand comprising SEQ ID NO: 780 and an antisense strand comprising SEQ ID NO: 825, the antisense strand comprising a region of complementarity to a KHK RNA transcript, e.g. KHK mRNA, wherein said dsRNAi is in the form of a conjugate having the structure depicted in depicted in FIG. 12A continuing to FIG. 12B; or
(d) a sense strand comprising SEQ ID NO: 782 and an antisense strand comprising SEQ ID NO: 827, the antisense strand comprising a region of complementarity to a KHK RNA transcript, e.g. KHK mRNA, wherein said dsRNAi is in the form of a conjugate having the structure depicted in FIG. 13A continuing to FIG. 13B; or
(e) a sense strand comprising SEQ ID NO: 785 and an antisense strand comprising SEQ ID NO: 830, the antisense strand comprising a region of complementarity to a KHK RNA transcript, e.g. KHK mRNA, wherein said dsRNAi is in the form of a conjugate having the structure depicted in FIG. 14A continuing to FIG. 14B; or
(f) a sense strand comprising SEQ ID NO: 804 and an antisense strand comprising SEQ ID NO: 849, the antisense strand comprising a region of complementarity to a KHK RNA transcript, e.g. KHK mRNA, wherein said dsRNAi is in the form of a conjugate having the structure depicted in FIG. 15A continuing to FIG. 15B;
or a pharmaceutically acceptable salt thereof.

16. A pharmaceutical composition comprising the dsRNAi oligonucleotide of claim 1, or a pharmaceutically acceptable salt thereof, and at least one pharmaceutically acceptable carrier, delivery agent or excipient.

17. A method for treating a disease, disorder or condition associated with KHK expression, comprising administering to a patient in need thereof a pharmaceutically effective amount of the RNAi oligonucleotide of claim 1.

18. The method of claim 17, wherein the disease, disorder or condition is selected from the group consisting of non-alcoholic fatty liver disease (NAFLD) and non-alcoholic steatohepatitis (NASH).

19. The method of claim 18, further comprising administering the RNAi in combination with a second therapeutic agent.

20. A method for reducing KHK expression in a cell, a population of cells or a subject, the method comprising the step of:
i. contacting the cell or the population of cells with the RNAi oligonucleotide, or a pharmaceutically acceptable salt thereof, of claim 1; or
ii. administering to the subject the RNAi oligonucleotide, or a pharmaceutically acceptable salt thereof of claim 1.
